(19) United States
(12) Patent Application Publication
(10) Pub. No.: US 2025/0034171 A1
(43) Pub. Date: Jan. 30, 2025

(54) IMIDAZOPYRIDINE AMIDES AND RELATED COMPOUNDS FOR USE IN THE TREATMENT OF BACTERIAL INFECTIONS

(71) Applicant: JANSSEN SCIENCES IRELAND UNLIMITED COMPANY, Co. Cork (IE)

(72) Inventors: José Manuel BARTOLOMÉ-NEBREDA, Toledo (ES); María Luz MARTÍN-MARTÍN, Toledo (ES); Dirk Antonie LAMPRECHT, Turnhout (BE); Bart Henri Theresia STOOPS, Wecheldeerzande (BE); Katie Ingrid Eduard AMSSOMS, Houve (BE); Guido Alfons F. VERNIEST, Knesselare (BE)

(21) Appl. No.: 18/705,114

(22) PCT Filed: Oct. 27, 2022
(86) PCT No.: PCT/EP2022/080058

§ 371 Date: Apr. 26, 2024

(30) Foreign Application Priority Data
Oct. 28, 2021 (EP) 21205213.8

(51) Int. Cl. C07D 519/00; A61K 31/437; A61K 31/444; A61K 31/501; A61K 31/519; A61K 45/06

(52) U.S. Cl. CPC C07D 519/00; A61K 31/437; A61K 31/444; A61K 31/501; A61K 31/519; A61K 45/06

(57) ABSTRACT

The present invention relates to the following compounds (I) wherein the integers are as defined in the description, and where the compounds may be useful as medicaments, for instance for use in the treatment of tuberculosis (e.g. in combination).

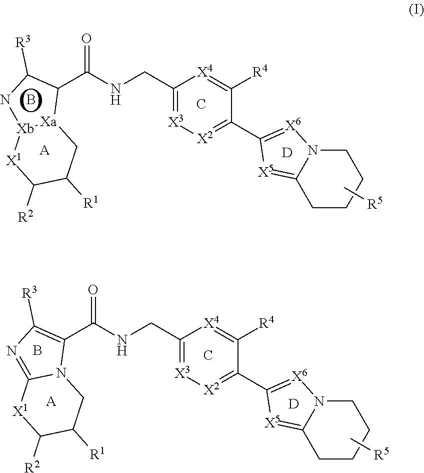

The present invention relates to novel compounds. The invention also relates to such compounds for use as a pharmaceutical and further for the use in the treatment of bacterial diseases, including diseases caused by pathogenic mycobacteria such as Mycobacterium tuberculosis. Such compounds may work by interfering with ATP synthase in M. tuberculosis, with the inhibition of cytochrome bc₁ activity as the primary mode of action.

Hence, primarily, such compounds are antitubercular agents.

### BACKGROUND OF THE INVENTION

Mycobacterium tuberculosis is the causative agent of tuberculosis (TB), a serious and potentially fatal infection with a world-wide distribution. Estimates from the World Health Organization indicate that more than 8 million people contract TB each year, and 2 million people die from tuberculosis yearly. In the last decade, TB cases have grown 20% worldwide with the highest burden in the most impoverished communities. If these trends continue, TB incidence will increase by 41% in the next twenty years. Fifty years since the introduction of an effective chemotherapy, TB remains after AIDS, the leading infectious cause of adult mortality in the world. Complicating the TB epidemic is the rising tide of multi-drug-resistant strains, and the deadly symbiosis with HIV. People who are HIV-positive and infected with TB are 30 times more likely to develop active TB than people who are HIV-negative and TB is responsible for the death of one out of every three people with HIV/AIDS worldwide.

Existing approaches to treatment of tuberculosis all involve the combination of multiple agents. For example, the regimen recommended by the U.S. Public Health Service is a combination of isoniazid, rifampicin and pyrazinamide for two months, followed by isoniazid and rifampicin alone for a further four months. These drugs are continued for a further seven months in patients infected with HIV. For patients infected with multi-drug resistant strains of M. tuberculosis, agents such as ethambutol, streptomycin, kanamycin, amikacin, capreomycin, ethionamide, cycloserine, ciprofoxacin and ofloxacin are added to the combination therapies. There exists no single agent that is effective in the clinical treatment of tuberculosis, nor any combination of agents that offers the possibility of therapy of less than six months' duration.

There is a high medical need for new drugs that improve current treatment by enabling regimens that facilitate patient and provider compliance. Shorter regimens and those that require less supervision are the best way to achieve this. Most of the benefit from treatment comes in the first 2 months, during the intensive, or bactericidal, phase when four drugs are given together; the bacterial burden is greatly reduced, and patients become noninfectious. The 4- to 6-month continuation, or sterilizing, phase is required to eliminate persisting bacilli and to minimize the risk of relapse. A potent sterilizing drug that shortens treatment to 2 months or less would be extremely beneficial. Drugs that facilitate compliance by requiring less intensive supervision also are needed. Obviously, a compound that reduces both the total length of treatment and the frequency of drug administration would provide the greatest benefit.

Complicating the TB epidemic is the increasing incidence of multi-drug-resistant strains or MDR-TB. Up to four percent of all cases worldwide are considered MDR-TB—those resistant to the most effective drugs of the four-drug standard, isoniazid and rifampin. MDR-TB is lethal when untreated and cannot be adequately treated through the standard therapy, so treatment requires up to 2 years of “second-line” drugs. These drugs are often toxic, expensive and marginally effective. In the absence of an effective therapy, infectious MDR-TB patients continue to spread the disease, producing new infections with MDR-TB strains. There is a high medical need for a new drug with a new mechanism of action, which is likely to demonstrate activity against drug resistant, in particular MDR strains.

The term “drug resistant” as used hereinbefore or hereinafter is a term well understood by the person skilled in microbiology. A drug resistant Mycobacterium is a Mycobacterium which is no longer susceptible to at least one previously effective drug; which has developed the ability to withstand antibiotic attack by at least one previously effective drug. A drug resistant strain may relay that ability to withstand to its progeny. Said resistance may be due to random genetic mutations in the bacterial cell that alters its sensitivity to a single drug or to different drugs.

MDR tuberculosis is a specific form of drug resistant tuberculosis due to a bacterium resistant to at least isoniazid and rifampicin (with or without resistance to other drugs), which are at present the two most powerful anti-TB drugs. Thus, whenever used hereinbefore or hereinafter “drug resistant” includes multi drug resistant.

Another factor in the control of the TB epidemic is the problem of latent TB. In spite of decades of tuberculosis (TB) control programs, about 2 billion people are infected by M. tuberculosis, though asymptomatically. About 10% of these individuals are at risk of developing active TB during their lifespan. The global epidemic of TB is fuelled by infection of HIV patients with TB and rise of multi-drug resistant TB strains (MDR-TB). The reactivation of latent TB is a high risk factor for disease development and accounts for 32% deaths in HIV infected individuals. To control TB epidemic, the need is to discover new drugs that can kill dormant or latent bacilli. The dormant TB can get reactivated to cause disease by several factors like suppression of host immunity by use of immunosuppressive agents like antibodies against tumor necrosis factor α or interferon-γ. In case of HIV positive patients the only prophylactic treatment available for latent TB is two-three months regimens of rifampicin, pyrazinamide. The efficacy of the treatment regime is still not clear and furthermore the length of the treatments is an important constrain in resource-limited environments. Hence there is a drastic need to identify new drugs, which can act as chemoprophylatic agents for individuals harboring latent TB bacilli.

The tubercle bacilli enter healthy individuals by inhalation; they are phagocytosed by the alveolar macrophages of the lungs. This leads to potent immune response and formation of granulomas, which consist of macrophages infected with M. tuberculosis surrounded by T cells. After a period of 6-8 weeks the host immune response cause death of infected cells by necrosis and accumulation of caseous material with certain extracellular bacilli, surrounded by macrophages, epitheloid cells and layers of lymphoid tissue at the periphery. In case of healthy individuals, most of the mycobacteria are killed in these environments but a small proportion of bacilli still survive and are thought to exist in a non-replicating, hypometabolic state and are tolerant to killing by anti-TB drugs like isoniazid. These bacilli can remain in the altered physiological environments even for individual's lifetime without showing any clinical symptoms of disease. However, in 10% of the cases these latent bacilli may reactivate to cause disease. One of the hypothesis about development of these persistent bacteria is patho-physiological environment in human lesions namely, reduced oxygen tension, nutrient limitation, and acidic pH. These factors have been postulated to render these bacteria phenotypically tolerant to major anti-mycobacterial drugs.

In addition to the management of the TB epidemic, there is the emerging problem of resistance to first-line antibiotic agents. Some important examples include penicillin-resistant Streptococcus pneumoniae, vancomycin-resistant enterococci, methicillin-resistant Staphylococcus aureus, multi-resistant salmonellae.

The consequences of resistance to antibiotic agents are severe. Infections caused by resistant microbes fail to respond to treatment, resulting in prolonged illness and greater risk of death. Treatment failures also lead to longer periods of infectivity, which increase the numbers of infected people moving in the community and thus exposing the general population to the risk of contracting a resistant strain infection.

Hospitals are a critical component of the antimicrobial resistance problem worldwide. The combination of highly susceptible patients, intensive and prolonged antimicrobial use, and cross-infection has resulted in infections with highly resistant bacterial pathogens.

Self-medication with antimicrobials is another major factor contributing to resistance. Self-medicated antimicrobials may be unnecessary, are often inadequately dosed, or may not contain adequate amounts of active drug.

Patient compliance with recommended treatment is another major problem. Patients forget to take medication, interrupt their treatment when they begin to feel better, or may be unable to afford a full course, thereby creating an ideal environment for microbes to adapt rather than be killed.

Because of the emerging resistance to multiple antibiotics, physicians are confronted with infections for which there is no effective therapy. The morbidity, mortality, and financial costs of such infections impose an increasing burden for health care systems worldwide.

Therefore, there is a high need for new compounds to treat bacterial infections, especially mycobacterial infections including drug resistant and latent mycobacterial infections, and also other bacterial infections especially those caused by resistant bacterial strains.

Anti-infective compounds for treating tuberculosis have been disclosed in e.g. international patent application WO 2011/113606. Such a document is concerned with compounds that would prevent M. tuberculosis multiplication inside the host macrophage and relates to compounds with a bicyclic core, imidazopyridines, which are linked (e.g. via an amido moiety) to e.g. an optionally substituted benzyl group.

International patent application WO 2014/015167 also discloses compounds that are disclosed as being of potential use in the treatment of tuberculosis. Such compounds disclosed herein have a bicycle (a 5,5-fused bicycle) as an essential element, which is substituted by a linker group (e.g. an amido group), which itself may be attached to another bicycle or aromatic group. Such compounds in this document do not contain a series of more than three rings.

Journal article Nature Medicine, 19, 1157-1160 (2013) by Pethe et al “Discovery of Q203, a potent clinical candidate for the treatment of tuberculosis” identifies a specific compound that was tested against M. tuberculosis. This compound Q203 is depicted below.

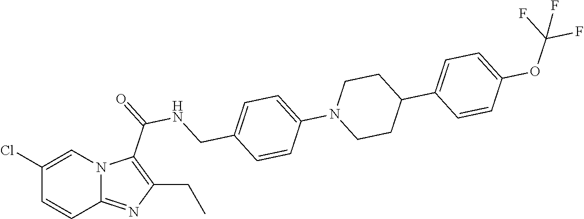 [SMILES: CCc1nc2ccc(Cl)cn2c1C(=O)NCc1ccc(N2CCC(c3ccc(OC(F)(F)F)cc3)CC2)cc1]

This clinical candidates is also discussed in journal article, J. Medicinal Chemistry, 2014, 57 (12), pp 5293-5305. It is stated to have activity against MDR tuberculosis, and have activity against the strain M. tuberculosis H37Rv at a MIC₅₀ of 0.28 nM inside macrophages. Positive control data (using known anti-TB compounds bedaquiline, isoniazid and moxifloxacin) are also reported. This document also suggests a mode of action, based on studies with mutants. It postulates that it acts by interfering with ATP synthase in M. tuberculosis, and that the inhibition of cytochrome bc₁ activity is the primary mode of action. Cytochrome bc₁ is an essential component of the electron transport chain required for ATP synthesis. It appeared that Q203 was highly active against both replicating and non-replicating bacteria.

International patent application WO 2015/014993 also discloses compounds as having activity against M. tuberculosis, as do international patent applications WO 2014/4015167, WO 2017/001660, WO 2017/001661, WO 2017/216281, WO 2017/216283 and WO 2021/048342. International patent applications WO 2013/033070 and WO 2013/033167 disclose various compounds as kinase modulators.

The purpose of the present invention is to provide compounds for use in the treatment of bacterial diseases, particularly those diseases caused by pathogenic bacteria such as Mycobacterium tuberculosis (including the latent disease and including drug resistant M. tuberculosis strains). Such compounds may also be novel and may act by interfering with ATP synthase in M. tuberculosis, with the inhibition of cytochrome bc₁ activity being considered the primary mode of action.

### SUMMARY OF THE INVENTION

There is now provided a compound of formula (I)

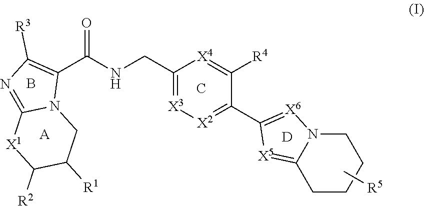

wherein
A is a 6-membered ring, which may be aromatic or non-aromatic,
X¹ represents ═N—, —CH₂— or ═C(R^{10a})—;
X², X³, X⁴, X⁵ and X⁶ each independently represent ═N— or ═C(R^{10b})—;
R¹ and R² each independently represent a substituent selected from hydrogen, halo (e.g. Cl, F), —R^{6c}, —O—R^{6d}, —C(═O)—R^{6e}, —C(═O)—N(R⁶)(R⁷), —CN and —N(R^{6a})R^{6b};
R³ represents a substituent selected from H, halo (e.g. Cl, F), and —C₁₋₃ alkyl (linear, branched or cyclic) optionally substituted by one or more substituents selected from halo (e.g. F) and —O—C₁₋₃ alkyl;
R⁴ represents a substituent selected from H, F, —C₁₋₃ alkyl and —O—C₁₋₃ alkyl;
R⁵ represents one or more substituents selected from H, —OH, —R^{8a}, —C(═O)—R^{8b}, —SO₂—R⁹, and —N(R^{11a})R^{11b}.
R⁶ and R⁷ are independently selected from H and —C₁₋₃ alkyl;
R^{6a} and R^{6b} independently represent H, C₁₋₆ alkyl or R^{6a} and R^{6b} are linked together to form a 3- to 6-membered ring;
R^{6c} and R^{6d} independently represent hydrogen or —C₁₋₄ alkyl optionally substituted by one or more substituents selected from halo (e.g. F), —O—CH₃, phenyl, —N(R^{6a})R^{6b};
R^{6e} is —C₁₋₃ alkyl;
R^{8a} represents —CN, —C₁₋₄ alkyl (linear, branched or cyclic; which alkyl group is optionally substituted by one or more substituents selected from halo, so forming e.g. —CF₃, —CHF₂, —CF₂CH₃), or, —OC₁₋₃ alkyl (optionally substituted by one or more substituents selected from halo and —O—CH₃);
R^{8b} is hydrogen or —C₁₋₃ alkyl (optionally substituted by one or more fluoro atoms);
R⁹ represents —C₁₋₄ alkyl optionally substituted by one or more substituents selected from halo (e.g. F) and —O—CH₃;
R^{10a} and R^{10b} independently represent H, halo, C₁₋₄ alkyl (itself optionally substituted by one or more, e.g. one, substituent(s) selected from fluoro, —CN, —R^{12a}, —OR^{12b}, —N(R^{12c})R^{12d} and/or —C(O)N(R^{12e})R^{12f}) or —O—C₁₋₄ alkyl (itself optionally substituted by one or more, e.g. one, substituent(s) selected from fluoro, —R^{12g}, —OR^{12h} and/or —N(R¹²ⁱ)R^{12j});
R^{11a} and R^{11b}, independently represent hydrogen, C¹⁻³ alkyl (optionally substituted by one or more fluoro atoms) or —S(O)₂R^{6c} (where R^{6c} is defined above);
R^{12a}, R^{12b}, R^{12c}, R^{12d}, R^{12e}, R^{12f}, R^{12g}, R^{12h}, R¹²ⁱ and R^{12j} independently represent hydrogen or C¹⁻³ alkyl (optionally substituted by one or more fluoro atoms);
or a pharmaceutically-acceptable salt thereof,
which compounds may be referred to herein as “compounds of the invention”.

In compounds of the invention:
R^{8a} may also represent C₁₋₄ alkyl optionally substituted by —OC₁₋₃ alkyl (and for instance, R⁵ may therefore represent —CH₂—OCH₃).

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

There is also provided a compound of formula (IA):

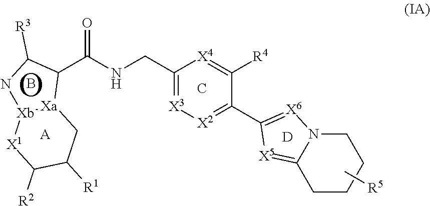

wherein
A is a 6-membered ring, which may be aromatic or non-aromatic,
B is a 5-membered aromatic ring;
one of Xₐ and X_{b} represents N and the other represents C (where each N and C is linked to the appropriate number of single/double bonds);
X¹ represents ═N—, —CH₂— or ═C(R^{10a})—.
X², X³, X⁴, X⁵ and X⁶ each independently represent ═N— or ═C(R^{10b})—;
R¹ and R² each independently represent a substituent selected from hydrogen, halo (e.g. Cl, F), —R^{6c}, —O—R^{6d}, —C(═O)—R^{6e}, —C(═O)—N(R⁶)(R⁷), —CN and —N(R^{6a})R^{6b};
R³ represents a substituent selected from H, halo (e.g. Cl, F), and —C₁₋₃ alkyl (linear, branched or cyclic) optionally substituted by one or more substituents selected from halo (e.g. F) and —O—C₁₋₃ alkyl;
R⁴ represents a substituent selected from H, F, —C₁₋₃ alkyl and —O—C₁₋₃ alkyl;
R⁵ represents one or more substituents selected from H, —OH, —R^{8a}, —C(═O)—R^{8b}, —SO₂—R⁹, and —N(R^{11a})R^{11b}.
R⁶ and R⁷ are independently selected from H and —C₁₋₃ alkyl;
R^{6a} and R^{6b} independently represent H, C₁₋₆ alkyl or R^{6a} and R^{6b} are linked together to form a 3- to 6-membered ring;
R^{6c} and R^{6d} independently represent hydrogen or —C₁₋₄ alkyl optionally substituted by one or more substituents selected from halo (e.g. F), —O—CH₃, phenyl, —N(R^{6a})R^{6b}; R^{6e} is —C₁₋₃ alkyl;
R^{8a} represents —CN, —C₁₋₄ alkyl (linear, branched or cyclic; which alkyl group is optionally substituted by one or more substituents selected from halo, so forming e.g. —CF₃, —CHF₂, —CF₂CH₃), or, —OC₁₋₃ alkyl (optionally substituted by one or more substituents selected from halo and —O—CH₃);
R^{8b} is hydrogen or —C₁₋₃ alkyl (optionally substituted by one or more fluoro atoms);
R⁹ represents —C₁₋₄ alkyl optionally substituted by one or more substituents selected from halo (e.g. F) and —O—CH₃;
R^{10a} and R^{10b} independently represent H, halo, C₁₋₄ alkyl (itself optionally substituted by one or more, e.g. one, substituent(s) selected from fluoro, —CN, —R^{12a}, —OR^{12b}, —N(R^{12c})R^{12d} and/or —C(O)N(R^{12e})R^{12f}) or —O—C₁₋₄ alkyl (itself optionally substituted by one or more, e.g. one, substituent(s) selected from fluoro, —R^{12g}, —OR^{12h} and/or —N(R¹²ⁱ)R^{12j});
R^{11a} and R^{11b}, independently represent hydrogen, C₁₋₃ alkyl (optionally substituted by one or more fluoro atoms) or —S(O)₂R^{6c} (where R^{6c} is defined above);
R^{12a}, R^{12b}, R^{12c}, R^{12d}, R^{12e}, R^{12f}, R^{12g}, R^{12h}, R¹²ⁱ and R^{12j} independently represent hydrogen or C₁₋₃ alkyl (optionally substituted by one or more fluoro atoms);
or a pharmaceutically-acceptable salt thereof,
which compounds may also be referred to herein as “compounds of the invention”.

The pharmaceutically acceptable acid addition salts as mentioned hereinabove are meant to comprise the therapeutically active non-toxic acid addition salt forms that the compounds of formula (I) are able to form. These pharmaceutically acceptable acid addition salts can conveniently be obtained by treating the base form with such appropriate acid.

For the purposes of this invention solvates, prodrugs, N-oxides and stereoisomers of compounds of the invention are also included within the scope of the invention.

The term “prodrug” of a relevant compound of the invention includes any compound that, following oral or parenteral administration, is metabolised in vivo to form that compound in an experimentally-detectable amount, and within a predetermined time (e.g. within a dosing interval of between 6 and 24 hours (i.e. once to four times daily)). For the avoidance of doubt, the term “parenteral” administration includes all forms of administration other than oral administration.

Prodrugs of compounds of the invention may be prepared by modifying functional groups present on the compound in such a way that the modifications are cleaved, in vivo when such prodrug is administered to a mammalian subject. The modifications typically are achieved by synthesising the parent compound with a prodrug substituent. Prodrugs include compounds of the invention wherein a hydroxyl, amino, sulfhydryl, carboxy or carbonyl group in a compound of the invention is bonded to any group that may be cleaved in vivo to regenerate the free hydroxyl, amino, sulfhydryl, carboxy or carbonyl group, respectively.

Examples of prodrugs include, but are not limited to, esters and carbamates of hydroxy functional groups, esters groups of carboxyl functional groups, N-acyl derivatives and N-Mannich bases. General information on prodrugs may be found e.g. in Bundegaard, H. “Design of Prodrugs” p. 1-92, Elesevier, New York-Oxford (1985).

Compounds of the invention may contain double bonds and may thus exist as E (entgegen) and Z (zusammen) geometric isomers about each individual double bond. Positional isomers may also be embraced by the compounds of the invention. All such isomers (e.g. if a compound of the invention incorporates a double bond or a fused ring, the cis- and trans-forms, are embraced) and mixtures thereof are included within the scope of the invention (e.g. single positional isomers and mixtures of positional isomers may be included within the scope of the invention).

Compounds of the invention may also exhibit tautomerism. All tautomeric forms (or tautomers) and mixtures thereof are included within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerisations. Valence tautomers include interconversions by reorganisation of some of the bonding electrons.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person.

All stereoisomers (including but not limited to diastereoisomers, enantiomers and atropisomers) and mixtures thereof (e.g. racemic mixtures) are included within the scope of the invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

The compounds of the present invention may exist in unsolvated as well as solvated forms with pharmaceutically acceptable solvents such as water, ethanol, and the like, and it is intended that the invention embrace both solvated and unsolvated forms.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵O, ¹⁷O, ¹⁸O, ³²P, ³³P, ³⁵S, ¹⁸F, ³⁶Cl, ¹²³I, and ¹²⁵I. Certain isotopically-labeled compounds of the present invention (e.g., those labeled with ³H and ¹⁴C) are useful in compound and for substrate tissue distribution assays. Tritiated (³H) and carbon-14 (¹⁴C) isotopes are useful for their ease of preparation and detectability. Further, substitution with heavier isotopes such as deuterium (i.e., ²H may afford certain therapeutic advantages resulting from greater metabolic stability (e.g., increased in vivo half-life or reduced dosage requirements) and hence may be preferred in some circumstances. ²H may also be depicted as ²D herein, and in any event both are encompassed by “hydrogen” or H according in the context of the scope of the invention. Positron emitting isotopes such as ¹⁵O, ¹³N, ¹¹C and ¹⁸F are useful for positron emission tomography (PET) studies to examine substrate receptor occupancy. Isotopically labeled compounds of the present invention can generally be prepared by following procedures analogous to those disclosed in the description/Examples hereinbelow, by substituting an isotopically labeled reagent for a non-isotopically labeled reagent.

Unless otherwise specified, C_{1-q} alkyl groups (where q is the upper limit of the range) defined herein may be straight-chain or, when there is a sufficient number (i.e. a minimum of two or three, as appropriate) of carbon atoms, be branched-chain, and/or cyclic (so forming a C_{3-q}— cycloalkyl group). Such cycloalkyl groups may be monocyclic or bicyclic and may further be bridged. Further, when there is a sufficient number (i.e. a minimum of four) of carbon atoms, such groups may also be part cyclic. Such alkyl groups may also be saturated or, when there is a sufficient number (i.e. a minimum of two) of carbon atoms, be unsaturated (forming, for example, a C_{2-q} alkenyl or a C_{2-q} alkynyl group). In a similar way, C_{1-q} alkylene groups represent C_{1-q} alkyl linker groups, i.e. —CH₂— (C₁ alkylene or methylene), —CH₂CH₂—, etc according to the number “q” of carbon atoms.

C_{3-q} cycloalkyl groups (where q is the upper limit of the range) that may be specifically mentioned may be monocyclic or bicyclic alkyl groups, which cycloalkyl groups may further be bridged (so forming, for example, fused ring systems such as three fused cycloalkyl groups). Such cycloalkyl groups may be saturated or unsaturated containing one or more double bonds (forming for example a cycloalkenyl group). Substituents may be attached at any point on the cycloalkyl group. Further, where there is a sufficient number (i.e. a minimum of four) such cycloalkyl groups may also be part cyclic.

The term “halo”, when used herein, preferably includes fluoro, chloro, bromo and iodo. Heterocyclic groups when referred to herein may include aromatic or non-aromatic heterocyclic groups, and hence encompass heterocycloalkyl and hetereoaryl. Equally, “aromatic or non-aromatic 5- or 6-membered rings” may be heterocyclic groups (as well as carbocyclic groups) that have 5- or 6-members in the ring.

Heterocycloalkyl groups that may be mentioned include non-aromatic monocyclic and bicyclic heterocycloalkyl groups in which at least one (e.g. one to four) of the atoms in the ring system is other than carbon (i.e. a heteroatom), and in which the total number of atoms in the ring system is between 3 and 20 (e.g. between three and ten, e.g between 3 and 8, such as 5- to 8-). Such heterocycloalkyl groups may also be bridged. Further, such heterocycloalkyl groups may be saturated or unsaturated containing one or more double and/or triple bonds, forming for example a C_{2-q} heterocycloalkenyl (where q is the upper limit of the range) group. C_{2-q} heterocycloalkyl groups that may be mentioned include 7-azabicyclo[2.2.1]heptanyl, 6-azabicyclo[3.1.1]heptanyl, 6-azabicyclo[3.2.1]-octanyl, 8-azabicyclo-[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo-[3.2.1]octanyl, oxetanyl, oxiranyl, piperazinyl, piperidinyl, non-aromatic pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Substituents on heterocycloalkyl groups may, where appropriate, be located on any atom in the ring system including a heteroatom. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N- or S-oxidised form. Heterocycloalkyl mentioned herein may be stated to be specifically monocyclic or bicyclic.

Aromatic groups may be aryl or heteroaryl. Aryl groups that may be mentioned include C₆₋₂₀, such as C₆₋₁₂ (e.g. C₆₋₁₀) aryl groups. Such groups may be monocyclic, bicyclic or tricyclic and have between 6 and 12 (e.g. 6 and 10) ring carbon atoms, in which at least one ring is aromatic. C₆₋₁₀ aryl groups include phenyl, naphthyl and the like, such as 1,2,3,4-tetrahydro-naphthyl. The point of attachment of aryl groups may be via any atom of the ring system. For example, when the aryl group is polycyclic the point of attachment may be via atom including an atom of a non-aromatic ring. However, when aryl groups are polycyclic (e.g. bicyclic or tricyclic), they are preferably linked to the rest of the molecule via an aromatic ring. Most preferred aryl groups that may be mentioned herein are “phenyl”.

Unless otherwise specified, the term “heteroaryl” when used herein refers to an aromatic group containing one or more heteroatom(s) (e.g. one to four heteroatoms) preferably selected from N, O and S. Heteroaryl groups include those which have between 5 and 20 members (e.g. between 5 and 10) and may be monocyclic, bicyclic or tricyclic, provided that at least one of the rings is aromatic (so forming, for example, a mono-, bi-, or tricyclic heteroaromatic group). When the heteroaryl group is polycyclic the point of attachment may be via any atom including an atom of a non-aromatic ring. However, when heteroaryl groups are polycyclic (e.g. bicyclic or tricyclic), they are preferably linked to the rest of the molecule via an aromatic ring. Heteroaryl groups that may be mentioned include 3,4-dihydro-1H-isoquinolinyl, 1,3-dihydroisoindolyl, 1,3-dihydroisoindolyl (e.g. 3,4-dihydro-1H-isoquinolin-2-yl, 1,3-dihydroisoindol-2-yl, 1,3-dihydroisoindol-2-yl; i.e. heteroaryl groups that are linked via a non-aromatic ring), or, preferably, acridinyl, benzimidazolyl, benzodioxanyl, benzodioxepinyl, benzodioxolyl (including 1,3-benzodioxolyl), benzofuranyl, benzofurazanyl, benzothiadiazolyl (including 2,1,3-benzothiadiazolyl), benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isothiochromanyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form. Heteroaryl groups mentioned herein may be stated to be specifically monocyclic or bicyclic. When heteroaryl groups are polycyclic in which there is a non-aromatic ring present, then that non-aromatic ring may be substituted by one or more ═O group. Most preferred heteroaryl groups that may be mentioned herein are 5- or 6-membered aromatic groups containing 1, 2 or 3 heteroatoms (e.g. preferably selected from nitrogen, oxygen and sulfur).

It may be specifically stated that the heteroaryl group is monocyclic or bicyclic. In the case where it is specified that the heteroaryl is bicyclic, then it may consist of a five-, six- or seven-membered monocyclic ring (e.g. a monocyclic heteroaryl ring) fused with another five-, six- or seven-membered ring (e.g. a monocyclic aryl or heteroaryl ring).

Heteroatoms that may be mentioned include phosphorus, silicon, boron and, preferably, oxygen, nitrogen and sulfur.

When “aromatic” groups are referred to herein, they may be aryl or heteroaryl. When “aromatic linker groups” are referred to herein, they may be aryl or heteroaryl, as defined herein, are preferably monocyclic (but may be polycyclic) and attached to the remainder of the molecule via any possible atoms of that linker group. However, when, specifically carbocylic aromatic linker groups are referred to, then such aromatic groups may not contain a heteroatom, i.e. they may be aryl (but not heteroaryl).

For the avoidance of doubt, where it is stated herein that a group may be substituted by one or more substituents (e.g. selected from C₁₋₆ alkyl), then those substituents (e.g. alkyl groups) are independent of one another. That is, such groups may be substituted with the same substituent (e.g. same alkyl substituent) or different (e.g. alkyl) substituents.

All individual features (e.g. preferred features) mentioned herein may be taken in isolation or in combination with any other feature (including preferred feature) mentioned herein (hence, preferred features may be taken in conjunction with other preferred features, or independently of them).

The skilled person will appreciate that compounds of the invention that are the subject of this invention include those that are stable. That is, compounds of the invention include those that are sufficiently robust to survive isolation from e.g. a reaction mixture to a useful degree of purity.

In an embodiment ring A is aromatic and:
X¹ represents ═N—, or ═CH—,
R¹ and R² each independently represent a substituent selected from hydrogen, —CH₃, —F, —Cl, —OCH₃, —NH₂, —CH₂NH₂.

Preferably, ring A is aromatic, and:
X¹ represents ═N—, and/or
R¹ and R² each independently represent a substituent selected from hydrogen, —CH₃, —F, —Cl, —OCH₃, —NH₂, —CH₂NH₂,

In another preferred configuration ring A is aromatic,
X¹ represents ═CH—, and/or
R¹ and R² each independently represent a substituent selected from hydrogen, —CH₃, —F, —Cl, —OCH₃, —NH₂, —CH₂NH₂.

In an alternative, ring A is non-aromatic,
X¹ represents —CH₂—, and/or
R¹ and R² each independently represent a substituent selected from hydrogen, —CH₃, —Cl, —OCH₃, —NH₂, —CH₂NH₂,

It is preferred that the compound of the invention comprises:
R₃ representing a substituent selected from H, —CF₃, —CHF₂, —CH₃, —CH₂CH₃, and cyclopropyl.

More precisely, compounds wherein ring A is non-aromatic:
X¹ represents —CH₂—;
R¹ and R² each independently represent a substituent selected from hydrogen, —CH₃, —Cl, —OCH₃, —NH₂, —CH₂NH₂; and/or
R₃ represents a substituent selected from H, —CF₃, —CHF₂, —CH₃, —CH₂CH₃, and cyclopropyl.

Alternatively, in another embodiment of the invention there is provided compounds wherein:
Ring A is aromatic;
X¹ represents ═N—;
R¹ and R² each independently represent a substituent selected from hydrogen, —CH₃, —Cl, —OCH₃, —NH₂, —CH₂NH₂; and/or
R₃ represents a substituent selected from H, —CF₃, —CHF₂, —CH₃, —CH₂CH₃, and cyclopropyl.

As another alternative, there is provided compounds wherein ring A is aromatic:
X¹ represents ═CH—, and/or
R¹ and R² each independently represent a substituent selected from hydrogen, —CH₃, —Cl, —OCH₃, —NH₂, —CH₂NH₂. In an embodiment —CH₃ may also specifically represent a deuterated isotope thereof, e.g. —CD₃.

In a further aspect of the invention, there is provided a compound in which ring C is selected among:

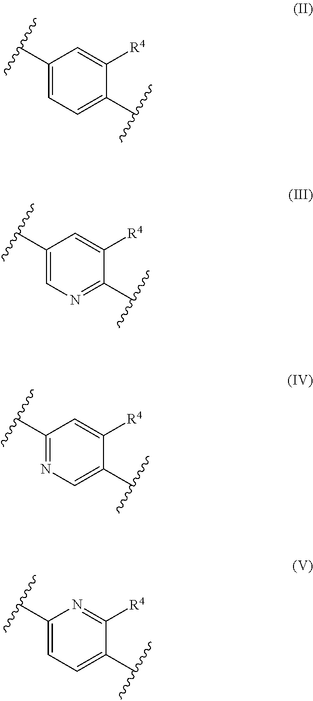

and wherein R⁴ represents, as hereinbefore disclosed, a substituent selected from H, F, —C₁₋₃ alkyl and —O—C₁₋₃ alkyl.

Within this aspect, R⁴ in an embodiment represents a substituent selected from H, F and —CH₃.

In an embodiment, Ring C may also be selected among:

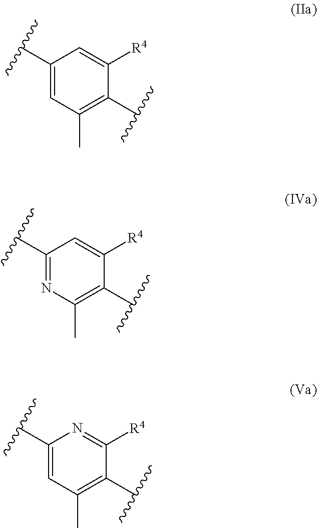

Within this aspect, R⁴ in an embodiment represents a substituent selected from F, —C₁₋₃ alkyl and —O—C₁₋₃ alkyl.

Within this aspect, R⁴ in another embodiment represents a substituent selected from F and —CH₃.

In an embodiment, the invention comprises a compound wherein ring D is selected among:

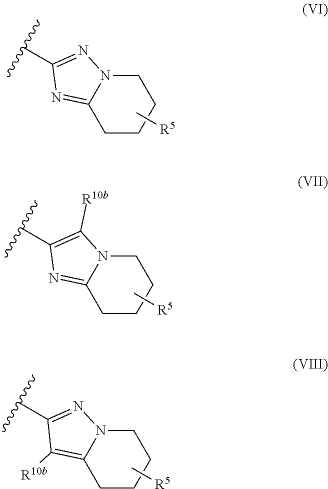

wherein, according to claim 1, R⁵ represents one or more substituents selected from H, —OH, —R^{8a}, —C(═O)—R^{8b}, —SO₂—R⁹, or —N(R^{11a})R^{11b}; and/or
R^{10b} represent H, halo, C₁₋₄ alkyl (itself optionally substituted by one or more, e.g. one, substituent(s) selected from fluoro, —CN, —R^{12a}, —OR^{12b}, —N(R^{12c})R^{12a} and/or —C(O)N(R^{12e})R^{12f}) or —O—C₁₋₄ alkyl (itself optionally substituted by one or more, e.g. one, substituent(s) selected from fluoro, —R^{12g}, —OR^{12h} and/or —N(R¹²ⁱ)R^{12j}).

In an embodiment, there is provided compounds wherein R^{10b} represents a substituent selected from H and —CH₃.

In yet another embodiment, ring D is selected among:

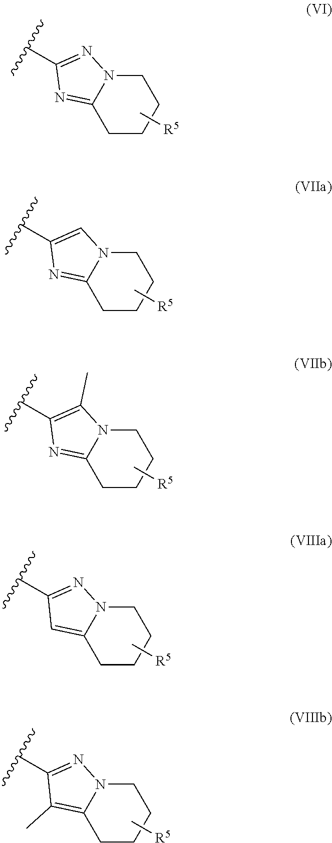

Preferably ring D is selected among:

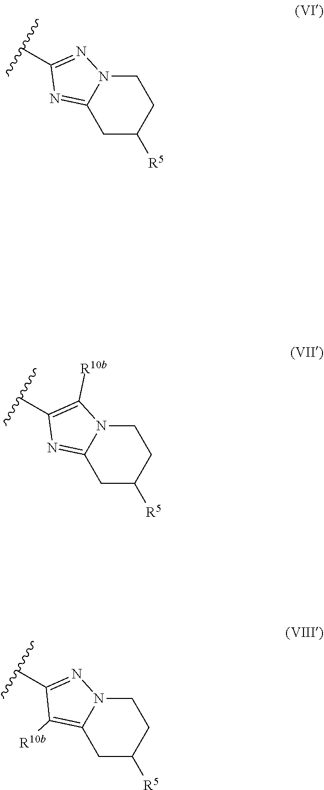

In an alternative embodiment, ring D is selected among:

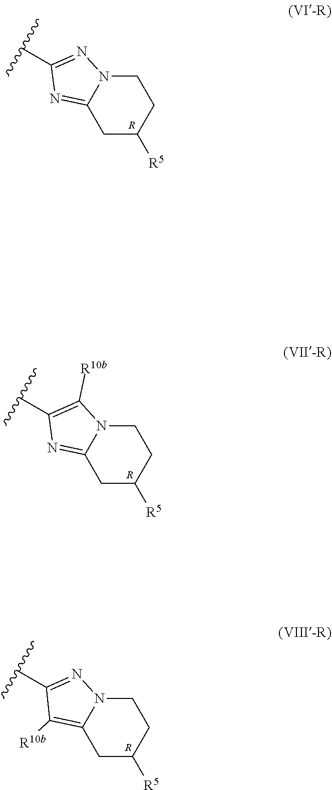

and the carbon atom to which R⁵ is attached has a R configuration.

In a second alternative embodiment, ring D is selected among:

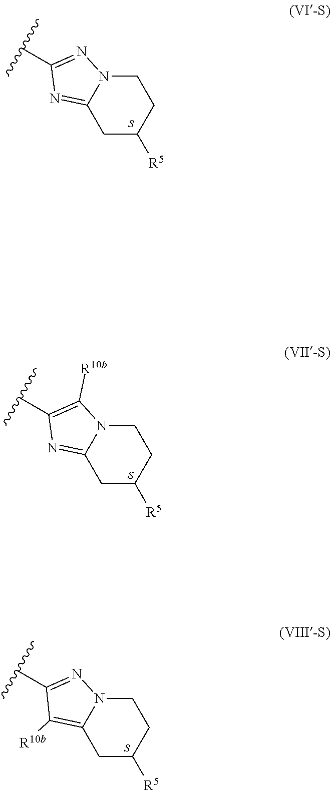

and the carbon atom to which R⁵ is attached has a S configuration.

In an embodiment, R⁵ represents H, —CH₃, —CH₂CH₃, —CH₂CH₂CH₃, cyclopropyl, —OH, —OCH₃, —OCF₃, —OCH₂CH₂OCH₃, —CF₃, —CHF₂, —CF₂CH₃, —NH₂, —NH(SO₂)CF₃, —N(CH₃)(SO₂)CF₃, and —SO₂CF₃.

In another embodiment, the invention comprises a compound wherein ring D is selected among:

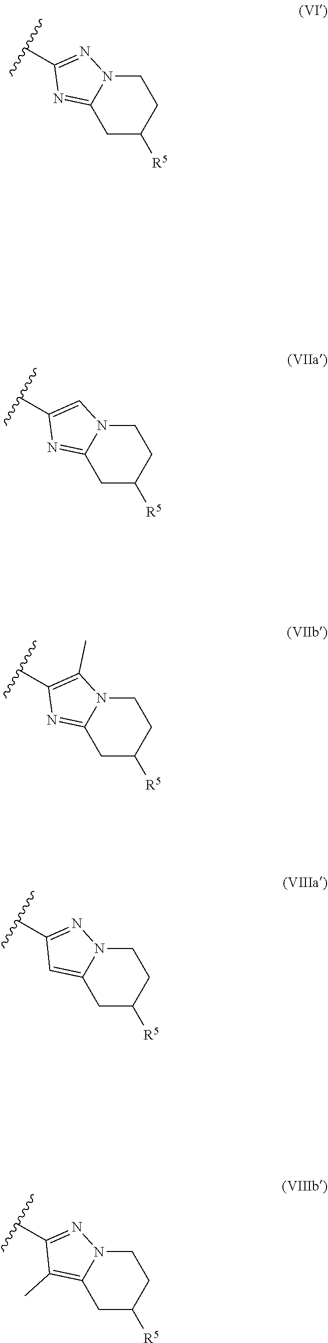

and wherein R⁵ represents H, —CH₃, —CH₂CH₃, —CH₂CH₂CH₃, cyclopropyl, —OH, —OCH₃, —OCF₃, —OCH₂CH₂OCH₃, —CF₃, —CHF₂, —CF₂CH₃, —NH₂, —NH(SO₂)CF₃, —N(CH₃)(SO₂)CF₃, and —SO₂CF₃.

Preferably the invention refers to a compound of formula (IX)

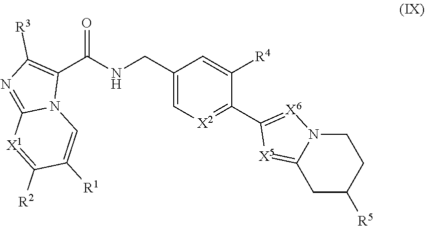

wherein
X¹ represents ═N— or ═C(R^{10a})—;
X², X⁵ and X⁶ each independently represent ═N— or ═C(R^{10b})—;
R¹ and R² each independently represent a substituent selected from hydrogen, halo (e.g. Cl, F), —R^{6c}, —O—R^{6d}, —C(═O)—R^{6e}, —C(═O)—N(R⁶)(R⁷), —CN and —N(R^{6a})R^{6b};
R³ represents a substituent selected from H, halo (e.g. Cl, F), —CF₃, —CHF₂, and —C₁₋₃ alkyl (linear, branched or cyclic) optionally substituted by one or more substituents selected from halo (e.g. F) and —O—C¹⁻³ alkyl;
R⁴ represents a substituent selected from H, F, —C₁₋₃ alkyl and —O—C₁₋₃ alkyl;
R⁵ represents H, —OH, —R^{8a}, —C(═O)—R^{8b}, —SO₂—R⁹, or —N(R^{11a})R^{11b};
R⁶ and R⁷ are independently selected from H and —C₁₋₃ alkyl;
R^{6a} and R^{6b} independently represent H, C₁₋₆ alkyl or R^{6a} and R^{6b} are linked together to form a 3- to 6-membered ring;
R^{6c} and R^{6d} independently represent hydrogen or —C₁₋₄ alkyl optionally substituted by one or more substituents selected from halo (e.g. F), —O—CH₃, phenyl, —N(R^{6a})R^{6b}; R^{6e} is —C₁₋₃ alkyl;
R^{8a} represents —CN, —C₁₋₄ alkyl (linear, branched or cyclic) optionally substituted by one or more substituents selected from halo (e.g. —CF₃, —CHF₂), —OC₁₋₃ alkyl optionally substituted by one or more substituents selected from halo and —O—CH₃;
R^{8b} is hydrogen or —C₁₋₃ alkyl (optionally substituted by one or more fluoro atoms);
R⁹ represents —C₁₋₄ alkyl optionally substituted by one or more substituents selected from halo (e.g. F) and —O—CH₃;
R^{10a} and R^{10b} independently represent H, halo, C₁₋₄ alkyl (itself optionally substituted by one or more, e.g. one, substituent(s) selected from fluoro, —CN, —R^{12a}, —OR^{12b}, —N(R^{12c})R^{12d} and/or —C(O)N(R^{12e})R^{12f}) or —O—C₁₋₄ alkyl (itself optionally substituted by one or more, e.g. one, substituent(s) selected from fluoro, —R^{12g}, —OR^{12h} and/or —N(R¹²ⁱ)R^{12j});
R^{11a} and R^{11b}, independently represent hydrogen, C¹⁻³ alkyl (optionally substituted by one or more fluoro atoms); —SO₂R^{6c};
R^{12a}, R^{12b}, R^{12c}, R^{12d}, R^{12e}, R^{12f}, R^{12g}, R^{12h}, R¹²ⁱ and R^{12j} independently represent hydrogen or C¹⁻³ alkyl (optionally substituted by one or more fluoro atoms);
or a pharmaceutically-acceptable salt thereof.

In yet a further preferred embodiment, the invention refers to a compound of formula (IX) or formula (IXA)

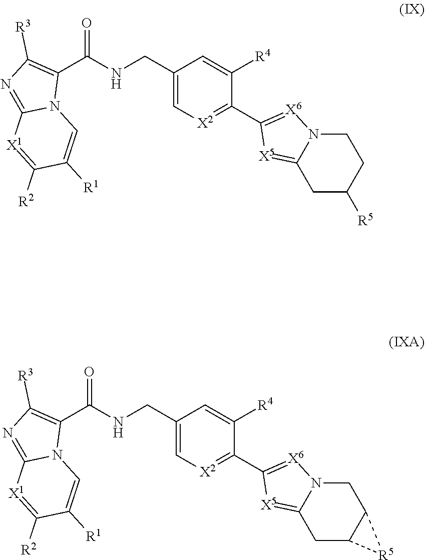

wherein
X¹ represents ═N— or ═CH—;
X², X⁵ and X⁶ each independently represent ═N—, ═CH— or ═C(CH₃)—;
R¹ and R² each independently represent a substituent selected from hydrogen, —CH₃, —F, —Cl, —OCH₃, —NH₂, and —CH₂NH₂;
R³ represents a substituent selected from H, —CF₃, —CHF₂, —CH₃, —CH₂CH₃, and cyclopropyl;
R⁴ represents a substituent selected from H, F and —CH₃;
R⁵ is linked to either the position meta or para-relative to the N atom in the ring system (in the compound of formula (IXA);
R⁵ represents H, —CH₃, —CH₂CH₃, —CH₂CH₂CH₃, cyclopropyl, —OH, —OCH₃, —OCF₃, —OCH₂CH₂OCH₃, —CF₃, —CHF₂, —CF₂CH₃, —NH₂, —NH(SO₂)CF₃, —N(CH₃)(SO₂)CF₃, or —SO₂CF₃,
or a pharmaceutically-acceptable salt thereof.

In an embodiment of the invention, R⁵ represents or (in a further embodiment) a substituent selected from C₁₋₄ alkyl (optionally substituted by one or more substituents selected from fluoro), C₃₋₄ cycloalkyl (e.g. cyclopropyl), —OH and —OC₁₋₄alkyl (where the alkyl moiety is itself optionally substituted by one or more substituents selected from fluoro and —O—C₁₋₂alkyl).

In an embodiment of the invention (including in the context of compounds of formulae (I), (IX) and (IXA):
R¹ and R² independently represent H, CH₃, F, Cl or —OCH₃;
X¹ represents ═N— or ═CH—;
R³ represents C¹⁻³ alkyl (optionally substituted by one or more fluoro atoms) or C₃₋₄ cycloalkyl (e.g. cyclopropyl);
X³ and X⁴ independently represent ═CH—;
X⁵ represents ═CH— or ═N— (and in an embodiment represents ═CH—);
R⁴ represents hydrogen, F or CH₃;
X⁵ and X⁶ independently represent ═N—, ═CH═ or ═C(CH₃)— (and in a further embodiment, one of X⁵ and X⁶ represents ═N— and the other represents ═N—, ═CH— or ═C(CH₃)—);
when R⁵ represents a substituent, it may be at the meta or para-position relative to the requisite N atom in that bicycle; and/or
R⁵ represents H (although in an embodiment represent a substituent), —CF₃, —CH₃, —CHF₂, —OCH₃, cyclopropyl, propyl, —OH, —O—CH₂CH₂OCH₃ or OCF₃,
i.e. any of the above embodiments may be taken in isolation or in combination with other embodiments disclosed herein.

In separate embodiments, compounds of formula (IA) may be depicted as compounds of formula (I) or as compounds of formula (IB),

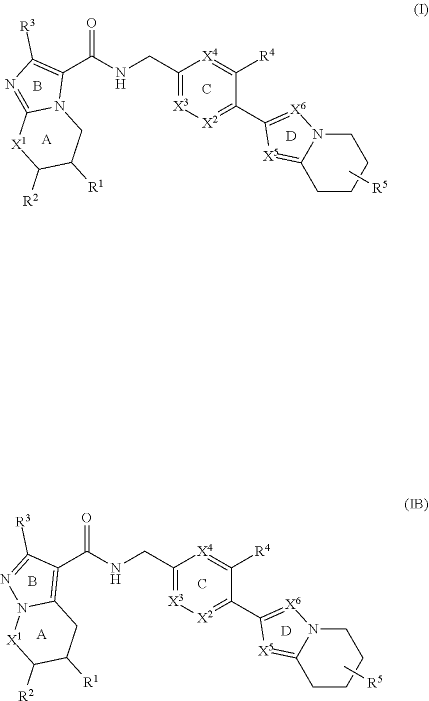

wherein all the integers are as defined herein.

In particular embodiments, the bicycle containing rings A and B may be represented by any one of the following formulae:

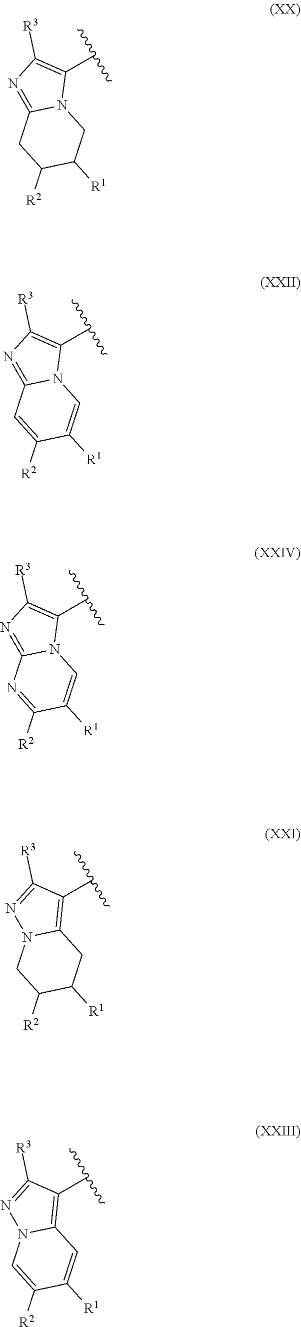

In an embodiment, R¹ and R² each represent hydrogen (and hence the 6-membered ring of rings depicted by (XX), (XXI), (XXII), (XXIII) and (XXIV) are unsubstituted). In another embodiment, R³ represents C¹⁻³ alkyl, such as ethyl.

In an embodiment, the C ring may represent unsubstituted phenyl, i.e. of the formula (XXX):

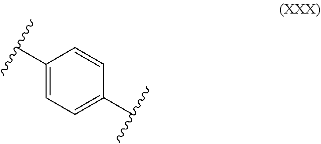 [SMILES: *c1ccc(*)cc1]

In an embodiment the D ring (or the bicycle containing the D ring) represents in separate embodiments, the formula (XXXI) or the formula (XXXII):

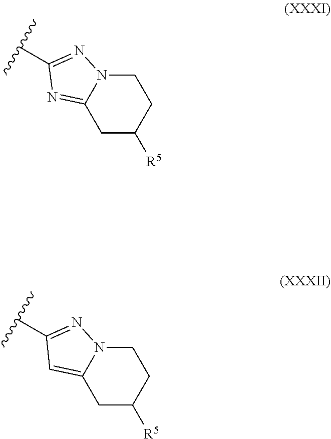

In another embodiment, R⁵ represents C¹⁻³ alkyl optionally substituted by one or more fluoro atoms; in a further embodiment, R⁵ represents —CF₃.

As mentioned herein, and for the avoidance of doubt, any of the foregoing embodiments may be used in combination with the others, for instance any of the embodiments depicting rings A and B, with any of those depicting ring C, with any of those depicting ring D as well as any of the other embodiments indicated herein (such as R⁵ substituents, etc).

The compounds according to the invention have surprisingly been shown to be suitable for the treatment of a bacterial infection including a mycobacterial infection, particularly those diseases caused by pathogenic mycobacteria such as Mycobacterium tuberculosis (including the latent and drug resistant form thereof). The present invention thus also relates to compounds of the invention as defined hereinabove, for use as a medicine, in particular for use as a medicine for the treatment of a bacterial infection including a mycobacterial infection.

Such compounds of the invention may act by interfering with ATP synthase in M. tuberculosis, with the inhibition of cytochrome bc₁ activity being the primary mode of action. Cytochrome bc₁ is an essential component of the electron transport chain required for ATP synthesis.

Further, the present invention also relates to the use of a compound of the invention, as well as any of the pharmaceutical compositions thereof as described hereinafter for the manufacture of a medicament for the treatment of a bacterial infection including a mycobacterial infection.

Accordingly, in another aspect, the invention provides a method of treating a patient suffering from, or at risk of, a bacterial infection, including a mycobacterial infection, which comprises administering to the patient a therapeutically effective amount of a compound or pharmaceutical composition according to the invention.

The compounds of the present invention also show activity against resistant bacterial strains.

Whenever used hereinbefore or hereinafter, that the compounds can treat a bacterial infection it is meant that the compounds can treat an infection with one or more bacterial strains.

The invention also relates to a composition comprising a pharmaceutically acceptable carrier and, as active ingredient, a therapeutically effective amount of a compound according to the invention. The compounds according to the invention may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, in particular, for administration orally or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules and tablets. Because of their ease in administration, tablets and capsules represent the most advantageous oral dosage unit forms in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations which are intended to be converted, shortly before use, to liquid form preparations.

Depending on the mode of administration, the pharmaceutical composition will preferably comprise from 0.05 to 99% by weight, more preferably from 0.1 to 70% by weight, even more preferably from 0.1 to 50% by weight of the active ingredient(s), and, from 1 to 99.95% by weight, more preferably from 30 to 99.9% by weight, even more preferably from 50 to 99.9% by weight of a pharmaceutically acceptable carrier, all percentages being based on the total weight of the composition.

The pharmaceutical composition may additionally contain various other ingredients known in the art, for example, a lubricant, stabilising agent, buffering agent, emulsifying agent, viscosity-regulating agent, surfactant, preservative, flavouring or colorant.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof.

The daily dosage of the compound according to the invention will, of course, vary with the compound employed, the mode of administration, the treatment desired and the mycobacterial disease indicated. However, in general, satisfactory results will be obtained when the compound according to the invention is administered at a daily dosage not exceeding 1 gram, e.g. in the range from 10 to 50 mg/kg body weight.

Given the fact that the compound of formula (I) is active against bacterial infections, the present compounds may be combined with other antibacterial agents in order to effectively combat bacterial infections.

Therefore, the present invention also relates to a combination of (a) a compound according to the invention, and (b) one or more other antibacterial agents.

The present invention also relates to a combination of (a) a compound according to the invention, and (b) one or more other antibacterial agents, for use as a medicine.

The present invention also relates to the use of a combination or pharmaceutical composition as defined directly above for the treatment of a bacterial infection.

A pharmaceutical composition comprising a pharmaceutically acceptable carrier and, as active ingredient, a therapeutically effective amount of (a) a compound according to the invention, and (b) one or more other antibacterial agents, is also comprised by the present invention.

The weight ratio of (a) the compound according to the invention and (b) the other antibacterial agent(s) when given as a combination may be determined by the person skilled in the art. Said ratio and the exact dosage and frequency of administration depends on the particular compound according to the invention and the other antibacterial agent(s) used, the particular condition being treated, the severity of the condition being treated, the age, weight, gender, diet, time of administration and general physical condition of the particular patient, the mode of administration as well as other medication the individual may be taking, as is well known to those skilled in the art. Furthermore, it is evident that the effective daily amount may be lowered or increased depending on the response of the treated subject and/or depending on the evaluation of the physician prescribing the compounds of the instant invention. A particular weight ratio for the present compound of the invention and another antibacterial agent may range from 1/10 to 10/1, more in particular from 1/5 to 5/1, even more in particular from 1/3 to 3/1.

The compounds according to the invention and the one or more other antibacterial agents may be combined in a single preparation or they may be formulated in separate preparations so that they can be administered simultaneously, separately or sequentially. Thus, the present invention also relates to a product containing (a) a compound according to the invention, and (b) one or more other antibacterial agents, as a combined preparation for simultaneous, separate or sequential use in the treatment of a bacterial infection.

The other antibacterial agents which may be combined with the compounds of the invention are for example antibacterial agents known in the art. For example, the compounds of the invention may be combined with antibacterial agents known to interfere with the respiratory chain of Mycobacterium tuberculosis, including for example direct inhibitors of the ATP synthase (e.g. bedaquiline, bedaquiline fumarate or any other compounds that may have be disclosed in the prior art, e.g. compounds disclosed in WO2004/011436), inhibitors of ndh2 (e.g. clofazimine) and inhibitors of cytochrome bd. Additional mycobacterial agents which may be combined with the compounds of the invention are for example rifampicin (=rifampin); isoniazid; pyrazinamide; amikacin; ethionamide; ethambutol; streptomycin; para-aminosalicylic acid; cycloserine; capreomycin; kanamycin; thioacetazone; PA-824; delamanid; quinolones/fluoroquinolones such as for example moxifloxacin, gatifloxacin, ofloxacin, ciprofloxacin, sparfloxacin; macrolides such as for example clarithromycin, amoxycillin with clavulanic acid; rifamycins; rifabutin; rifapentin; as well as others, which are currently being developed (but may not yet be on the market; see e.g. http://www.newtbdrugs.org/pipeline.php).

Compounds of the invention (including forms and compositions/combinations comprising compounds of the invention) may have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise. For instance, compounds of the invention may advantages associated with: lower cardiotoxicity; no reactive metabolite formation (e.g. that may cause toxicity issues, e.g. genotoxicity); no formation of degradants (e.g. that are undesired or may elicit unwanted side-effects); and/or faster oral absorption and improved bioavailability. Certain compounds of the invention may also have advantages over certain other compounds of the invention, for instance certain compounds (e.g. those in which R¹ and R² each represent hydrogen (and hence the 6-membered ring of rings depicted by (XX), (XXI), (XXII), (XXIII) and (XXIV) are unsubstituted) may have the advantage that no or fewer undesired metabolites (e.g. oxidative metabolites) are produced (which may be observed in the case where either R¹ and/or R² represents a substituent, such as alkyl, e.g. methyl).

### General Preparation

The compounds according to the invention can generally be prepared by a succession of steps, each of which may be known to the skilled person or described herein.

### EXPERIMENTAL PART

Compounds of formula I may be prepared in accordance with the techniques employed in the examples hereinafter (and those methods know by those skilled in the art), for example by using the following techniques. Analogous reactions may be performed to prepare compounds of formula (IA), for instance reaction of compound (XL) as defined herein with a compound of formula (XI) and/or reaction of a compound of formula (XLI) as defined herein with a compound of formula (XIII).

Compounds of formula (I) may be prepared by:
(i) reaction of a compound of formula (X),

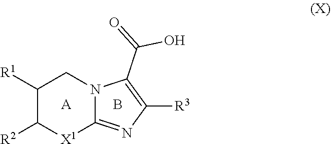

in which the integers are hereinbefore defined, with a compound of formula (XI),

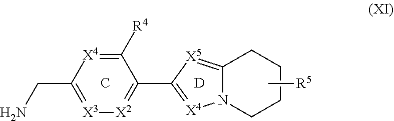

wherein the integers are as hereinbefore defined;
which reaction may be performed in the presence of a suitable coupling reagent, for instance selected from diisopropylethylamine (DIPEA), 1-[bis(dimethylamino)methylene]-1H-1,2,3-triazolo[4,5-b]pyridinium-3-oxid hexafluorophosphate (HATU), 1-(3-dimethylaminopropyl)-3-ethylcarbodiimide (EDCI), 1-hydroxybenzotriazole (HOBt), 0-(benzotriazole-1-yl)-N,N,N′,N′-tetramethyluronium tetrafluoroborate (TBTU), or a combination thereof, under suitable conditions such as those described in the examples hereinafter; for example, in the presence of a suitable coupling reagent (e.g. 1,1′-carbonyldiimidazole, N,N′-dicyclohexylcarbodiimide, 1-(3-dimethylaminopropyl)-3-ethylcarbodiimide (or hydrochloride thereof) or N,N′-disuccinimidyl carbonate), optionally in the presence of a suitable base (e.g. sodium hydride, sodium bicarbonate, potassium carbonate, pyridine, triethylamine, dimethylaminopyridine, diisopropylamine, sodium hydroxide, potassium tert-butoxide and/or lithium diisopropylamide (or variants thereof) and an appropriate solvent (e.g. tetrahydrofuran, pyridine, toluene, dichloromethane, chloroform, acetonitrile, dimethylformamide, trifluoromethylbenzene, dioxane or triethylamine). Alternatively, the carboxylic acid group of the compound of formula (X) may first be converted under standard conditions to the corresponding acyl chloride (e.g. in the presence of POCl₃, PCl₅, SOCl₂ or oxalyl chloride), which acyl chloride is then reacted with a compound of formula (XI), for example under similar conditions to those mentioned above;
or
(ii) coupling of a compound of formula (XII),

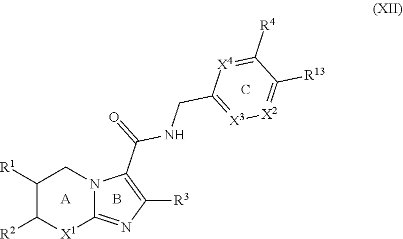

wherein the integers are as hereinbefore defined, and R¹³ represents a suitable group, e.g. a suitable leaving group such as chloro, bromo, iodo or a sulfonate group (for example a type of group that may be deployed for a coupling), with a compound of formula (XIII),

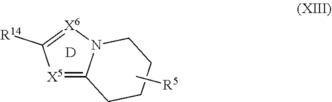

wherein R⁴ is as hereinbefore defined, and R¹⁴ represents a suitable group, e.g. a suitable leaving group under standard conditions, for example optionally in the presence of an appropriate metal catalyst (or a salt or complex thereof) such as Pd(dba)₂, Pd(OAc)₂, Cu, Cu(OAc)₂, CuI, NiCl₂ or the like, with an optional additive such as Ph₃P, X-phos or the like, in the presence of an appropriate base (e.g. t-BuONa, or the like) in a suitable solvent (e.g. dioxane or the like) under reaction conditions known to those skilled in the art.

It will be appreciated by those skilled in the art that some compounds of formula (I) may be converted to other compounds of formula (I).

Other compounds that may be used for preparation of compounds of formula (IA):

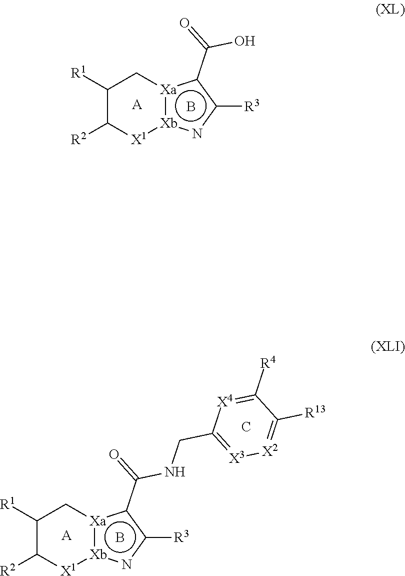

It is evident that in the foregoing and in the following reactions, the reaction products may be isolated from the reaction medium and, if necessary, further purified according to methodologies generally known in the art, such as extraction, crystallization and chromatography. It is further evident that reaction products that exist in more than one enantiomeric form, may be isolated from their mixture by known techniques, in particular preparative chromatography, such as preparative HPLC, chiral chromatography. Individual diastereoisomers or individual enantiomers can also be obtained by Supercritical Fluid Chromatography (SCF).

The starting materials and the intermediates are compounds that are either commercially available or may be prepared according to conventional reaction procedures generally known in the art.

### EXAMPLES

### 1—General Information

### Abbreviations

ACN: Acetonitrile
Cs₂CO₃: Cesium carbonate
DCM: Dichloromethane
DIPE: Diisopropyl ether
DIPEA: N,N-diisopropylethylamine
DMF: Dimethylformamide
DMSO: Dimethylsulfoxide
Et₂O: Diethyl ether
EtOAc: Ethyl acetate
Et₃N or TEA: Triethyl amine
h: hour
H₂: Dihydrogen gas
HATU: 1-[Bis(dimethylamino)methylene]-1H-1,2,3-triazolo[4,5-b]pyridinium 3-oxid hexafluorophosphate
HCl: Hydrochloric acid
i-PrOH: Isopropyl alcohol
K₂CO₃: Potassium carbonate
LiOH: Lithium hydroxide
MeOH: methanol
MgSO₄: Magnesium sulphate
min: minute
N₂: Nitrogen
NaHCO₃: Sodium bicarbonate
NaOH: Sodium hydroxide
NH₄C: Ammonium chloride
NH₄HCO₃: Ammonium bicarbonate
NMR: Nuclear magnetic resonance
PdCl₂(PPh₃)₂: Dichlorobis(triphenylphosphine)palladium(II)
Pd(OAc)₂: Palladium(II) acetate
rt: Room temperature
THF: Tetrahydrofuran
TFA: Trifluoroacetic acid
THF: Tetrahydrofuran
PdCl₂(dppf)₂: [1,1′-Bis(diphenylphosphino)ferrocene]dichloropalladium(II)
KOtBu: Potassium tert-butoxide
   Melting Points Melting points were recorded using a differential scanning calorimeter DSC 1 Mettler Toledo.

Melting points were measured with a temperature gradient of 10° C. per min from 25 to 350° C. Values are peak values. Unless indicated, this method is used.

An alternative method is with open capilliary tubes on a Mettler Toledo MP50, which may be indicated at “MT”. With this method, melting points are measured with a temperature gradient of 10° C./minute. Maximum temperature is 300° C. The melting point data is read from a digital display and checked from a video recording system.

### ¹H NMR

¹H NMR spectra were recorded on a Bruker Avance DRX 400 spectrometer or Bruker Advance III 400 spectrometer using internal deuterium lock and equipped with reverse double-resonance (¹H, 13C, SEI) probe head with z gradients and operating at 400 MHz for proton and 100 MHz for carbon and a Bruker Avance 500 MHz spectrometer equipped with a Bruker 5 mm BBFO probe head with z gradients and operating at 500 MHz for proton and 125 MHz for carbon.

NMR spectra were recorded at ambient temperature unless otherwise stated.

Data are reported as follow: chemical shift in parts per million (ppm) relative to TMS (δ=0 ppm) on the scale, integration, multiplicity (s=singulet, d=doublet, t=triplet, q=quartet, quin=quintuplet, sex=sextuplet, m=multiplet, b=broad, or a combination of these), coupling constant(s) J in Hertz (Hz).

### Absolute Configuration

Virtual Circula Dicroism (VCD) and Infrared Spectra were used to determine absolute configuration.

### Experimental

IR and VCD spectra were recorded on a dual PEM ChiralIR-2X spectrometer (Biotools Inc., Jupiter, FL). Measurements were done in DMSO-d6 with a concentration of 3.8 mg/125 μL for A/B and E/F, 1.1 mg/175 μL for C/D. A cell with 100 μm path length and BaF₂ windows was used. Both the sample and the virtual racemate spectrum were recorded with a resolution of 4 cm⁻¹, totalling 60000 scans or 20 hours of measurement time each with both PEMs optimized at 1400 cm⁻¹. The baseline corrected VCD spectrum was obtained by combining the raw data for the enantiomer with the spectrum of the corresponding virtual racemate.

Computational A thorough conformational search is performed at molecular mechanics level using Macromodel (version 13.3) with a mixed torsional/low-mode sampling and the OPLS4 force field. The located minima were optimized using Jaguar (version 11.2) on the B3LYP-D3/6-31G** level with a Poisson-Boltzmann continuum solvation model to mimic a DMSO solvent. All conformations within 10 kJ/mol interval were used to simulate VCD and IR spectra from calculated rotatory strength and wavenumbers (scaled by a factor 0.975) with Lorentzian curves using half width at half-height values of 4 cm⁻¹.

Assignment was done after visual comparison between experimental and measured IR and VCD spectra.

### 2—Synthesis

### Synthesis of Intermediates I

### Synthesis of Intermediate I-1

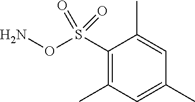

Perchloric acid [7601-90-3] was added dropwise over 15 min to a suspension of ethyl 0-(2-mesitylenesulfonyl)acethydroxamate [38202-27-6] (13 g, 45.56 mmol) in 1,4-dioxane (45 mL) at 0° C. (internal temperature maintained below 15° C.). The mixture was stirred at 0° C. for 1 h. Then ice-water (45 mL) and DCM (45 mL) were added, and the organic layer was separated to yield intermediate I-1 as a 1M DCM solution that was used in the next step without any further treatment (CAUTION, do not remove the solvent, explosive when dry).

### Synthesis of Intermediate I-2

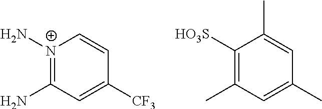

2-Amino-4-(trifluoromethyl)pyridine [106447-97-6] (2.28 g, 32.54 mmol) was added portionwise to a 1M solution of intermediate I-1 in DCM (45.56 mL, 45.56 mmol) in a round bottom flask under nitrogen at 0° C. The mixture was stirred at rt for 16 h. The suspension was diluted with diethyl ether (10 ml) and the solid formed was filtered off and washed with additional diethyl ether to yield intermediate I-2 as a white solid (11.51 g, 86%).

### Synthesis of Intermediate I-3

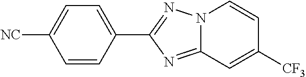 [SMILES: N#Cc1ccc(-c2nc3cc(C(F)(F)F)ccn3n2)cc1]

4-Cyanobenzoyl chloride [6068-72-0] (8.30 g, 21.79 mmol) was added to a solution of intermediate I-2 (8.25 g, 21.79 mmol) in pyridine (88 mL) at 0° C. The mixture was stirred at 90° C. for 16 h and then water was added. The solid formed was filtered off and washed with water (×3) and diethyl ether to yield intermediate I-3 as a beige solid (5.03 g, 79%).

### Synthesis of Intermediate I-4

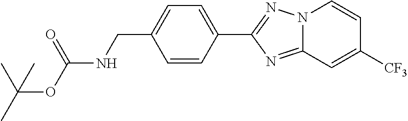 [SMILES: CC(C)(C)OC(=O)NCc1ccc(-c2nc3cc(C(F)(F)F)ccn3n2)cc1]

Sodium borohydride [16940-66-2] (1.99 g, 52.59 mmol) was added portionwise to a suspension of intermediate I-3 (5.05 g, 17.53 mmol), nickel (II) chloride hexahydrate [7791-20-0] (4.17 g, 17.53 mmol) and di-tertbutyl dicarbonate [24424-99-5] (12.08 mL, 52.59 mmol) in dry methanol (60 mL) at −5° C. under N₂. The reaction mixture was stirred at rt for 16 h. Water and 1 mL of aqueous NH₃ were added and the mixture was extracted with DCM. The combined organic layers were washed with water, separated, dried (MgSO₄), filtered and concentrated in vacuo. The crude product was purified by flash column chromatography (silica; EtOAc in DCM 0/100 to 15/85). The desired fractions were collected and concentrated in vacuo to yield intermediate I-4 as a yellowish solid (7.50 g, 97%).

### Synthesis of Intermediate I-5

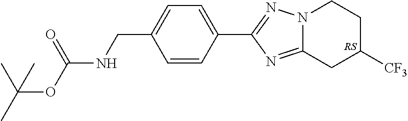 [SMILES: CC(C)(C)OC(=O)NCc1ccc(-c2nc3n(n2)CCC(C(F)(F)F)C3)cc1]

Palladium(II) hydroxide on carbon [12135-22-7] (2.17 g, 18.71 mmol) was added portionwise to a solution of intermediate I-4 (7.50 g, 17.01 mmol) in a mixture of methanol (120 mL) and EtOAc (20 mL) at 0° C. under N2. Then H2 was added, and the mixture was stirred at rt for 18 h. Then further palladium(II)hydroxide [12135-22-7] (0.95 g, 6.81 mmol) was added at 0° C. under N2. H2 was added, and the mixture was stirred under at rt for a further 4 h. The mixture was filtered through a pad of Celite® and the solvents were concentrated in vacuo. The crude product was purified by flash column chromatography (silica; EtOAc in heptane 0/100 to 40/60). The desired fractions were collected and concentrated in vacuo to yield intermediate I-5 as a white solid (1.78 g, 40%).

### Synthesis of Intermediate I-6

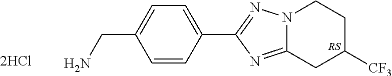 [SMILES: NCc1ccc(-c2nc3n(n2)CCC(C(F)(F)F)C3)cc1]

A 4 M solution of HCl in dioxane [7647-01-0] (10.52 mL, 42.08 mmol) was added to a stirred solution of intermediate I-5 (2.78 g, 7.01 mmol) in DCM (35 mL) at rt. The reaction mixture was stirred at rt for 16 h. The solvents were evaporated in vacuo to yield intermediate I-6 as a white solid used in the next step without any further purification (2.73 g, quantitative).

### Synthesis of Intermediate I-7

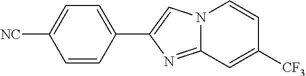 [SMILES: N#Cc1ccc(-c2cn3ccc(C(F)(F)F)cc3n2)cc1]

NaHCO3 (0.57 g, 6.79 mmol) was added to a stirred solution of 2-amino-4-(trifluoromethyl)pyridine [106447-97-6] (1.0 g, 6.17 mmol) and 4-(bromoacetyl)benzonitrile [20099-89-2] (1.80 g, 8.02 mmol) in ethanol (12 mL) at rt. The mixture was stirred at reflux for 18 h and then water was added. The solid formed was filtered off and washed with water and diethyl ether to yield intermediate I-7 as a beige solid (1.48 g, 82%).

### Synthesis of Intermediate I-8

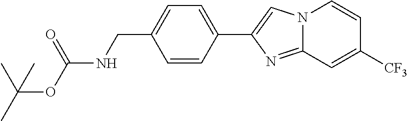 [SMILES: CC(C)(C)OC(=O)NCc1ccc(-c2cn3ccc(C(F)(F)F)cc3n2)cc1]

Sodium borohydride [16940-66-2] (0.58 g, 15.41 mmol) was added portionwise to a suspension of intermediate I-7 (1.48 g, 5.14 mmol), nickel (II) chloride hexahydrate [7791-20-0] (1.22 g, 15.41 mmol) and di-tertbutyl dicarbonate [24424-99-5] (3.54 mL, 15.41 mmol) in dry methanol (15.4 mL) at −5° C. under N2. The reaction mixture was stirred at rt for 16 h. Water and 1 mL of aqueous NH3 were added and the mixture was extracted with DCM. The combined organic layers were washed with water, separated, dried (MgSO4), filtered and concentrated in vacuo to yield intermediate I-8 as a brown sticky oil (2.05 g, quantitative).

### Synthesis of Intermediate I-9

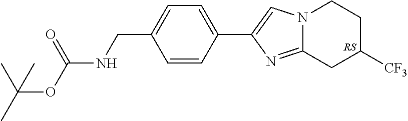 [SMILES: CC(C)(C)OC(=O)NCc1ccc(-c2cn3c(n2)CC(C(F)(F)F)CC3)cc1]

Platinum(IV) oxide [1314-15-4] (1.22 g, 5.37 mmol) was added to a solution of intermediate I-8 (2.1 g, 5.37 mmol), in a mixture of ethanol (70 mL) and dry THE (70 mL) at 0° C. under N2. Then H2 was added, and the mixture was stirred at rt for 16 h. The mixture was filtered through a pad of Celite® and the solvents were concentrated in vacuo. The crude product was purified by flash column chromatography (silica; EtOAc in DCM 0/100 to 20/80). The desired fractions were collected and concentrated in vacuo to yield intermediate I-9 as a white solid (0.45 g, 19%).

### Synthesis of Intermediate I-10

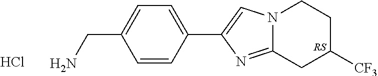 [SMILES: Cl ; NCc1ccc(-c2cn3c(n2)CC(C(F)(F)F)CC3)cc1]

A 4 M solution of HCl in dioxane [7647-01-0] (1.33 mL, 5.31 mmol) was added to a stirred solution of intermediate I-9 (175 mg, 0.44 mmol) in DCM (2 mL) at rt. The reaction mixture was stirred at rt for 18 h. The solvents were evaporated in vacuo to yield intermediate I-10 as a yellowish solid used in the next step without any further purification (146 mg, quantitative).

### Synthesis of Intermediate I-11

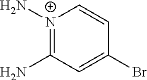

2-Amino-4-bromopyridine [84249-14-9] (5 g, 28.90 mmol) was added portionwise to a 1M solution of intermediate I-1 in DCM (57.80 mL, 57.80 mmol) in a round bottom flask under nitrogen at 0° C. The mixture was stirred at rt for 16 h. The suspension was diluted with diethyl ether (30 ml) and the solid formed was filtered off and washed with additional diethyl ether to yield intermediate I-11 as a white solid (6.28 g, 87%).

### Synthesis of Intermediate I-12

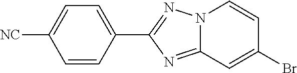 [SMILES: N#Cc1ccc(-c2nc3cc(Br)ccn3n2)cc1]

4-Cyanobenzoyl chloride [6068-72-0] (6.62 g, 40 mmol) was added to a solution of intermediate I-11 (7.77 g, 20 mmol) in pyridine (24 mL) at 0° C. The mixture was stirred at 90° C. for 8 h and then water was added. The solid formed was filtered off and washed with water (×3) and diethyl ether to yield intermediate I-12 as a white solid (3.02 g, 46%).

### Synthesis of Intermediate I-13

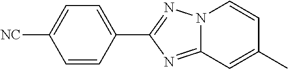 [SMILES: Cc1ccn2nc(-c3ccc(C#N)cc3)nc2c1]

Pd(dppf)₂Cl₂ [65464-05-4] (136 mg, 0.17 mmol) was added to a solution of intermediate I-12 (0.5 g, 1.67 mmol) in a mixture of dry 1,4-dioxane (4 mL) and heptane (4 mL) in a sealed tube under N₂. Then, a 2 M solution of dimethylzinc solution in toluene [544-97-8] (2.51 mL, 5.01 mmol) was added at rt under N₂ and the mixture was stirred at 55° C. for 16 h. The solvents were evaporated in vacuo and the crude product was purified by flash column chromatography (silica; EtOAc in heptane 0/100 to 100/0). The desired fractions were collected and concentrated in vacuo to yield intermediate I-13 as an orange solid (391 mg, 99%).

### Synthesis of Intermediate I-14

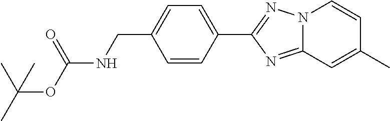 [SMILES: Cc1ccn2nc(-c3ccc(CNC(=O)OC(C)(C)C)cc3)nc2c1]

Sodium borohydride [16940-66-2] (189 mg, 5 mmol) was added portionwise to a suspension of intermediate I-13 (391 mg, 1.67 mmol), nickel (II) chloride hexahydrate [7791-20-0] (198 mg, 0.83 mmol) and di-tertbutyl dicarbonate [24424-99-5] (0.77 mL, 3.34 mmol) in a mixture of dry methanol (22 mL) and 1,4-dioxane (10 mL) at 0° C. under N2. The reaction mixture was stirred at rt for 16 h. A saturated NH4Cl aqueous solution and 1 mL of aqueous NH3 were added and the mixture was extracted with DCM. The combined organic layers were washed with water, separated, dried (MgSO4), filtered and concentrated in vacuo. The crude product was purified by flash column chromatography (silica; EtOAc in heptane 0/100 to 30/70). The desired fractions were collected and concentrated in vacuo to yield intermediate I-14 as a white solid (380 mg, 67%).

### Synthesis of Intermediate I-15

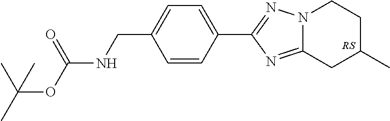 [SMILES: CC1CCn2nc(-c3ccc(CNC(=O)OC(C)(C)C)cc3)nc2C1]

Palladium(II) hydroxide [12135-22-7] (77 mg, 0.55 mmol) was added to a solution of intermediate I-14 (370 mg, 1.1 mmol), in a mixture of methanol (5 mL) and EtOAc (1 mL) at 0° C. under N2. Then H2 was added, and the mixture was stirred at rt for 16 h. The mixture was filtered through a pad of Celite® and the solvents were concentrated in vacuo to yield intermediate I-15 as a yellow solid used in the next step without any further purification (350 mg, 84%).

### Synthesis of Intermediate I-16

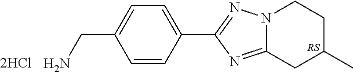 [SMILES: CC1CCn2nc(-c3ccc(CN)cc3)nc2C1]

A 4 M solution of HCl in dioxane [7647-01-0] (1.55 mL, 6.2 mmol) was added to a stirred solution of intermediate I-15 (350 mg, 1.02 mmol) in DCM (15 mL) at rt. The reaction mixture was stirred at rt for 16 h. The solvents were evaporated in vacuo to yield intermediate I-16 as a white solid used in the next step without any further purification (340 mg, 99%).

### Synthesis of Intermediate I-17a

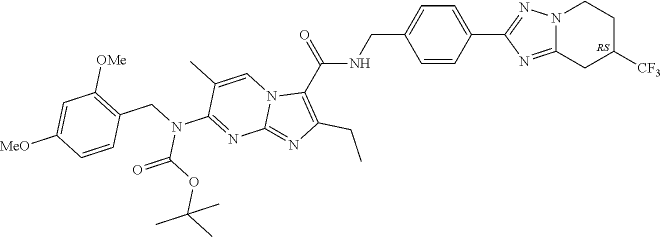 [SMILES: CCc1nc2nc(N(Cc3ccc(OC)cc3OC)C(=O)OC(C)(C)C)c(C)cn2c1C(=O)NCc1ccc(-c2nc3n(n2)CCC(C(F)(F)F)C3)cc1]

Intermediate I-6 (113 mg, 0.34 mmol) was added to a stirred mixture of intermediate II-15 (160 mg, 0.34 mmol), HATU [148893-10-1] (130 mg, 0.34 mmol), and DIPEA [7087-68-5](0.24 mL, 1.36 mmol) in DMF (1.7 mL) at rt. The mixture was stirred at rt for 19 h. Then, a saturated NaHCO3 aqueous solution was added, and the mixture was extracted with DCM. The combined organic layers were dried (MgSO4), filtered and the solvents evaporated in vacuo. The crude product was purified by flash column chromatography (silica; EtOAc in heptane to 80/20). The desired fractions were collected, concentrated in vacuo to yield intermediate I-17a as a white solid (179 mg, 67%).

### Synthesis of Intermediate I-17b

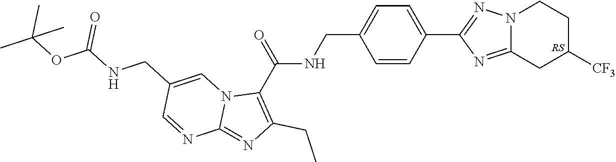 [SMILES: CCc1nc2ncc(CNC(=O)OC(C)(C)C)cn2c1C(=O)NCc1ccc(-c2nc3n(n2)CCC(C(F)(F)F)C3)cc1]

HATU [148893-10-1] (222 mg, 0.58 mmol) and DIPEA [7087-68-5] (0.54 mL, 3.08 mmol) was added to a solution of intermediate II-17d (187 mg, 0.494 mmol) in DMF (5 mL) at rt. The mixture was stirred at rt for 10 min and then I-6 (187 mg, 0.49 mmol) was added, and the mixture reaction was stirred at rt for 16 h. A saturated NaHCO3 aqueous solution was added, and the mixture was extracted with EtOAc (×3). The combined organic layers were dried (MgSO4), filtered and concentrated in vacuo and the excess of DMF was co-distilled with toluene (10 mL×3). The crude product was purified by flash column chromatography (silica; DCM/MeOH 9:1 in DCM from 0/100 to 30/70). The desired fractions were collected and evaporated in vacuo to yield intermediate I-17b as a beige solid (165 mg, 61%, 45% purity).

### Synthesis of Intermediate I-18

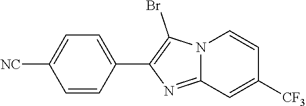 [SMILES: N#Cc1ccc(-c2nc3cc(C(F)(F)F)ccn3c2Br)cc1]

N-Bromosuccinimide [128-08-5] (205 mg, 1.15 mmol) was added portionwise to a solution of intermediate I-7a (300 mg, 1.04 mmol) and DCM (5.3 mL) at 0° C. under N2. The mixture was stirred at room temperature for 1 h. Then water was added, and the mixture was extracted with EtOAc. The organic layer was separated, dried (MgSO4), filtered and the solvents evaporated in vacuo to yield intermediate I-18 as a beige solid (386 mg, quantitative).

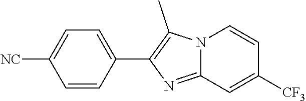 [SMILES: Cc1c(-c2ccc(C#N)cc2)nc2cc(C(F)(F)F)ccn12]

Methylboronic acid [13061-96-6] (192 mg, 2.36 mmol) and E were added to a solution of intermediate I-18 (288 mg, 0.79 mmol) in a mixture of water (0.7 mL) and 1,4-dioxane (2.6 mL) at rt. N2 was bubbled for 10 min and then tetrakis(triphenylphosphine)palladium(0) [14221-01-3] (228 mg, 0.20 mmol) and Na2CO3 (167 mg, 1.58 mmol) were added and the mixture was stirred at 120° C. for 17 h. Then additional methylboronic acid [13061-96-6] (47 mg, 0.39 mmol) and tetrakis(triphenylphosphine)palladium(0) [14221-01-3] (46 mg, 0.04 mmol) were added and the mixture was stirred at 120° C. for 16 h. Then methylboronic acid [13061-96-6] (47 mg, 0.39 mmol) and tetrakis(triphenylphosphine)palladium(0) [14221-01-3](46 mg, 0.04 mmol) were added and the mixture was stirred at 120° C. for 16 h. Then methylboronic acid [13061-96-6] (47 mg, 0.39 mmol) and tetrakis(triphenylphosphine)palladium(0) [14221-01-3] (46 mg, 0.04 mmol) added and the mixture was stirred at 120° C. for a further 16 h. Water was added and the mixture was extracted with EtOAc. The combined organic layers were dried (MgSO4), filtered and concentrated in vacuo. The crude product was purified by flash column chromatography (silica; EtOAc in heptane from 0/100 to 10/90). The desired fractions were collected, and the solvent was evaporated in vacuo to yield intermediate 119 as a yellow solid (340 mg, 82%).

### Synthesis of Intermediate I-20

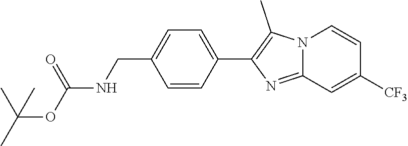 [SMILES: Cc1c(-c2ccc(CNC(=O)OC(C)(C)C)cc2)nc2cc(C(F)(F)F)ccn12]

Sodium borohydride [16940-66-2] (129 mg, 3.41 mmol) was added portionwise to a suspension of intermediate I-19 (340 mg, 1.13 mmol), nickel (II) chloride hexahydrate [7791-20-0] (147 g, 1.13 mmol) and di-tertbutyl dicarbonate [24424-99-5] (0.78 mL, 3.87 mmol) in dry methanol (11 mL) at 0° C. under N2. The reaction mixture was stirred at rt for 16 h. Water was added, and the mixture was extracted with EtOAc (3×). The combined organic layers were dried (MgSO4), filtered and concentrated in vacuo. The crude product was purified by flash column chromatography (silica; EtOAc in heptane to 60/40). The desired fractions were collected and concentrated in vacuo to yield intermediate I-20 as a yellow solid (207 mg, 24%).

### Synthesis of Intermediate I-21

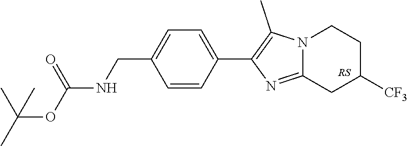 [SMILES: Cc1c(-c2ccc(CNC(=O)OC(C)(C)C)cc2)nc2n1CCC(C(F)(F)F)C2]

Palladium(II) hydroxide on carbon [12135-22-7] (262 mg, 0.37 mmol) was added to a solution of intermediate I-20 (505 mg g, 1.25 mmol) in methanol (8.6 mL) at 0° C. under N2.

Then H2 was added, and the mixture was stirred at rt for 5 h. Then more Palladium(II) hydroxide on carbon [12135-22-7] (262 mg, 0.37 mmol) was added at 0° C. under N2. Then H2 was added, and the mixture was stirred at rt for 16 h. Then more Palladium(II) hydroxide on carbon [12135-22-7] (262 mg, 0.37 mmol) was added at 0° C. under N2. Then H2 was added, and the mixture was stirred at rt for a further 6 h. The mixture was filtered through a pad of Celite® and the solvent was concentrated in vacuo. The crude product was purified by flash column chromatography (silica; EtOAc in heptane 0/100 to 55/45). The desired fractions were collected and concentrated in vacuo to yield intermediate I-21 as a white solid (428 mg, 83%).

### Synthesis of Intermediate I-22

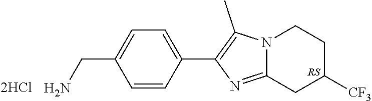 [SMILES: Cc1c(-c2ccc(CN)cc2)nc2n1CCC(C(F)(F)F)C2]

A 4 M solution of HCl in dioxane [7647-01-0] (2.7 mL, 10.8 mmol) was added to a stirred solution of intermediate I-21 (428 mg, 1.04 mmol) in DCM (3 mL) at rt. The reaction mixture was stirred at rt for 16 h. The solvents were evaporated in vacuo to yield intermediate I-22 as a white solid used in the next step without any further purification (412 mg, 97%).

### Synthesis of Intermediate I-23

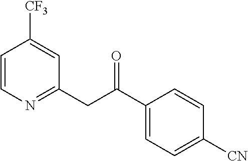 [SMILES: N#Cc1ccc(C(=O)Cc2cc(C(F)(F)F)ccn2)cc1]

A 2.5 M solution of n-butyllithium in hexanes [109-72-8] (3 mL, 7.5 mmol) was added dropwise to a solution of 2-methyl-4-(trifluoromethyl)pyridine [106877-17-2] (800 mg, 4.9 mmol) in THE (25 mL at −78° C. under N2. The mixture was stirred at −78° C. for 30 min. Then ethyl 4-cyanobenzoate [7153-22-2] (0.96 g, 1.37 mmol) in THE (2 mL) was added dropwise. The reaction mixture was stirred at −78° C. for 2 h. Water was added and the mixture was extracted with EtOAc. The crude was purified by flash column chromatography (silica; EtOAc in DCM from 0/100 to 10/90). The desired fractions were collected and the solvents evaporated in vacuo to yield intermediate I-23 as a yellow solid.

### Synthesis of Intermediate I-24

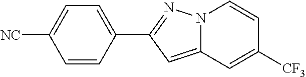 [SMILES: N#Cc1ccc(-c2cc3cc(C(F)(F)F)ccn3n2)cc1]

A 1M solution of intermediate I-1 in DCM (5.6 mL, 5.6 mmol) was added to a solution of intermediate I-23 (810 mg, 2.8 mmol) in DCM (30 mL) at rt under N2and the mixture was stirred at rt for 48 h. The reaction mixture was washed with a saturated NaHCO3 aqueous solution. The organic layer was dried (MgSO4), filtered and concentrated in vacuo. The crude material was purified by flash column chromatography (silica, EtOAc in DCM from 100/0 to 0/100). The desired fractions were combined and the solvents were removed in vacuo to yield intermediate I-24 as a yellow solid.

### Synthesis of Intermediate I-25

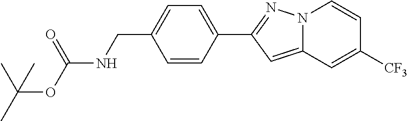 [SMILES: CC(C)(C)OC(=O)NCc1ccc(-c2cc3cc(C(F)(F)F)ccn3n2)cc1]

Sodium borohydride [16940-66-2] (137 mg, 0.66 mmol) was added portionwise to a suspension of intermediate I-24 (349 mg, 1.22 mmol), nickel (II) chloride hexahydrate [7791-20-0] (145 mg, 0.61 mmol) and di-tertbutyl dicarbonate [24424-99-5] (0.56 mL, 2.43 mmol) in a mixture of dry methanol (30 mL) and 1,4-dioxane (15 mL) at 0° C. under N2. The reaction mixture was stirred at rt for 10 min and then more nickel (II) chloride hexahydrate [7791-20-0] (145 g, 0.61 mmol) and di-tertbutyl dicarbonate [24424-99-5] (0.56 mL, 2.43 mmol) and sodium borohydride [16940-66-2] (137 mg, 0.66 mmol) were added. The mixture was stirred at rt for 16 h and then a saturated NH4Cl aqueous solution and a NH3 aqueous solution (1 mL) were added, and the mixture was extracted with DCM. The combined organic layers were dried (MgSO4), filtered and concentrated in vacuo. The crude product was purified by flash column chromatography (silica; EtOAc in heptane to 30/70). The desired fractions were collected and concentrated in vacuo to yield intermediate I-25 as a white solid (492 mg, 98%).

### Synthesis of Intermediate I-26

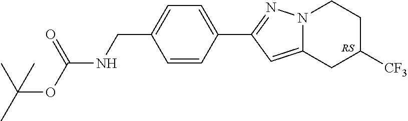 [SMILES: CC(C)(C)OC(=O)NCc1ccc(-c2cc3n(n2)CCC(C(F)(F)F)C3)cc1]

Palladium on carbon (10% w/w) 50% H2O [7440-05-3] (1 g, 0.94 mmol) was added to a solution of intermediate I-25 (492 mg g, 1.26 mmol) in a mixture of ethanol (23 mL) and THE (23 mL) at 0° C. under N2. H2 was added, and the mixture was stirred at rt for 16 h. Then more Palladium on carbon (10% w/w) 50% H2O [7440-05-3] (1 g, 0.94 mmol) was added at 0° C. under N2. H2 was added, and the mixture was stirred at rt for a further 16 h. The mixture was filtered through a pad of Celite® and the solvents were concentrated in vacuo. The crude product was purified by flash column chromatography (silica; EtOAc in heptane 0/100 to 40/60). The desired fractions were collected and concentrated in vacuo to yield intermediate I-26 as a white solid (277 mg, 55%).

### Synthesis of Intermediate I-27

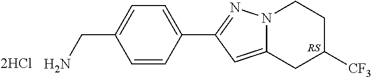 [SMILES: NCc1ccc(-c2cc3n(n2)CCC(C(F)(F)F)C3)cc1]

A 4 M solution of HCl in dioxane [7647-01-0] (1 mL, 4 mmol) was added to a stirred solution of intermediate I-26 (135 mg, 1.04 mmol) in DCM (5 mL) at rt. The reaction mixture was stirred at rt for 16 h. The solvents were evaporated in vacuo to yield intermediate I-27 as a white solid used in the next step without any further purification (126 mg, quantitative).

### Synthesis of Intermediate I-28

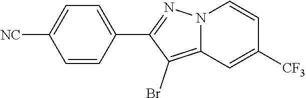 [SMILES: N#Cc1ccc(-c2nn3ccc(C(F)(F)F)cc3c2Br)cc1]

N-Bromosuccinimide [128-08-5] (136 mg, 0.77 mmol) was added portionwise to a solution of intermediate I-24 (200 mg, 0.7 mmol) and DCM (7 mL) at rt. The mixture was stirred at room temperature for 2 h. Then water was added, and the mixture was extracted with DCM. The organic layer was separated, dried (MgSO4), filtered and the solvents evaporated in vacuo and the crude product purified by flash column chromatography (silica; EtOAc in heptane 0/100 to 15/85). The desired fractions were collected and the solvents were evaporated in vacuo to yield intermediate I-28 as a yellow solid (191 mg, 71%).

### Synthesis of Intermediate I-29

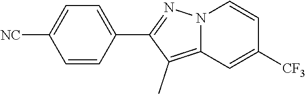 [SMILES: Cc1c(-c2ccc(C#N)cc2)nn2ccc(C(F)(F)F)cc12]

A 2M Dimethylzinc solution in toluene [544-97-8] (0.35 mL, 0.7 mmol) was added to a stirred solution of intermediate I-28 (170 mg, 0.46 mmol) in 1,4-dioxane (5 mL) at rt under N2. Then tetrakis(triphenylphosphine)palladium(0) [14221-01-3] (53 mg, 0.09 mmol) and the mixture was stirred at 55° C. for 16 h. Then additional A 2M Dimethylzinc solution in toluene [544-97-8] (0.35 mL, 0.7 mmol) and tetrakis(triphenylphosphine)palladium(0) [14221-01-3](53 mg, 0.09 mmol) were added and the mixture was stirred at 55° C. for a further 16 h. Water was added and the mixture was extracted with EtOAc (3×). The combined organic layers were dried (MgSO4), filtered and concentrated in vacuo. The crude product was purified by flash column chromatography (silica; EtOAc in heptane from 0/100 to 20/80). The desired fractions were collected, and the solvent was evaporated in vacuo to yield intermediate I-29 as a yellow solid (113 mg, 80%).

### Synthesis of Intermediate I-30

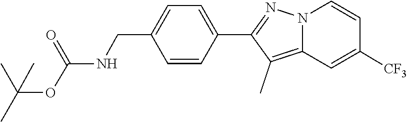 [SMILES: Cc1c(-c2ccc(CNC(=O)OC(C)(C)C)cc2)nn2ccc(C(F)(F)F)cc12]

Sodium borohydride [16940-66-2] (43 mg, 1.12 mmol) was added portionwise to a suspension of intermediate I-29 (113 mg, 0.38 mmol), nickel (II) chloride hexahydrate [7791-20-0] (89 mg, 0.38 mmol) and di-tertbutyl dicarbonate [24424-99-5] (0.26 mL, 1.13 mmol) in a mixture of dry methanol (6 mL) and dry 1,4-dioxane (3 mL) at 0° C. under N2. The reaction mixture was stirred at rt for 16 h and then a saturated NH4Cl aqueous solution and a NH3 aqueous solution (1 mL) were added, and the mixture was extracted with DCM. The combined organic layers were dried (MgSO4), filtered and concentrated in vacuo. The crude product was purified by flash column chromatography (silica; EtOAc in heptane to 30/70) The desired fractions were collected and concentrated in vacuo to yield intermediate I-30 as a white solid (106 mg, 66%).

### Synthesis of Intermediate I-31

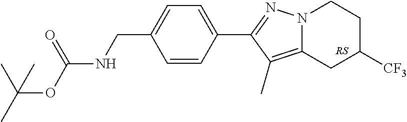 [SMILES: Cc1c(-c2ccc(CNC(=O)OC(C)(C)C)cc2)nn2c1CC(C(F)(F)F)CC2]

Intermediate I-31 was prepared according to an analogous procedure to the one used for the synthesis of intermediate I-26 using intermediate I-30 (90 mg, 0.22 mmol) as starting material (63 mg, 66%).

### Synthesis of Intermediate I-32

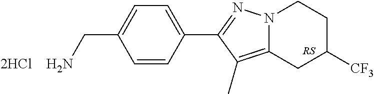 [SMILES: Cc1c(-c2ccc(CN)cc2)nn2c1CC(C(F)(F)F)CC2]

Intermediate I-32 was prepared according to an analogous procedure to the one used for the synthesis of intermediate I-27 using intermediate I-31 (63 mg, 0.15 mmol) as starting material (59 mg, 99%).

### Synthesis of Intermediate I-33

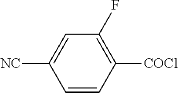 [SMILES: N#Cc1ccc(C(=O)Cl)c(F)c1]

4-Cyano-2-fluorobenzoic acid [164149-28-4] (1 g, 6.06 mmol) was dissolved in dry DCM (12 mL) and cooled to 0° C. under N2. Then oxalyl chloride [79-37-8] (0.77 mL, 9.08 mmol) followed by dry DMF (0.1 mL) were added, and the mixture was stirred at rt for 1 h. The solvents were removed in vacuo to intermediate I-33 as a yellowish solid that was used in the next reaction step without any further purification (1.11 g, quantitative).

### Synthesis of Intermediate I-34

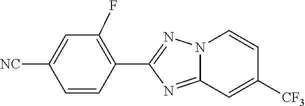 [SMILES: N#Cc1ccc(-c2nc3cc(C(F)(F)F)ccn3n2)c(F)c1]

Intermediate I-34 was prepared according to an analogous procedure to the one used for the synthesis of intermediate I-3 using intermediate I-33 (1.11 g, 6.06 mmol) as starting material (0.66 g, 67%).

### Synthesis of Intermediate I-35

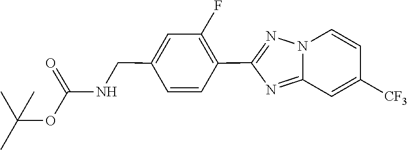 [SMILES: CC(C)(C)OC(=O)NCc1ccc(-c2nc3cc(C(F)(F)F)ccn3n2)c(F)c1]

Intermediate I-35 was prepared according to an analogous procedure to the one used for the synthesis of intermediate I-4 using intermediate I-34 (0.66 g, 2.16 mmol) as starting material (0.97 g, 99%).

### Synthesis of Intermediate I-36

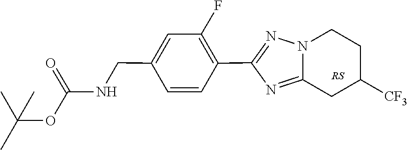 [SMILES: CC(C)(C)OC(=O)NCc1ccc(-c2nc3n(n2)CCC(C(F)(F)F)C3)c(F)c1]

Palladium(II) hydroxide on carbon [12135-22-7] (0.33 g, 3.36 mmol) was added portionwise to a solution of intermediate I-35 (0.97 g, 3.36 mmol) in methanol (20 mL) at 0° C. under N2. Then H2 was added, and the mixture was stirred at rt for 18 h. The mixture was filtered through a pad of Celite® and the solvents were concentrated in vacuo. The crude product was purified by flash column chromatography (silica; EtOAc in heptane 0/100 to 40/60). The desired fractions were collected and concentrated in vacuo to yield intermediate I-36 as a white solid (0.57 g, 79%).

### Synthesis of Intermediate I-37

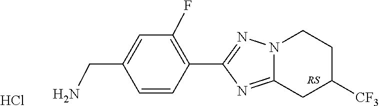 [SMILES: NCc1ccc(-c2nc3n(n2)CCC(C(F)(F)F)C3)c(F)c1 ; Cl]

Intermediate I-37 was prepared according to an analogous procedure to the one used for the synthesis of intermediate I-6 using intermediate I-36 (0.56 g, 1.35 mmol) as starting material (0.47 g, quantitative).

### Synthesis of Intermediate I-38

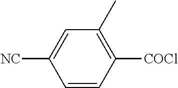

Intermediate I-38 was prepared according to an analogous procedure to the one used for the synthesis of intermediate I-33 using intermediate 4-cyano-2-methylbenzoic acid (2.5 g, 15.51 mmol) as starting material (2.79 g, quantitative).

### Synthesis of Intermediate I-39

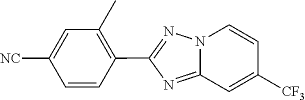 [SMILES: Cc1cc(C#N)ccc1-c1nc2cc(C(F)(F)F)ccn2n1]

Intermediate I-39 was prepared according to an analogous procedure to the one used for the synthesis of intermediate I-3 using intermediate I-38 (2.93 g, 7.76 mmol) as starting material (1.35 g, 55%).

### Synthesis of Intermediate I-40

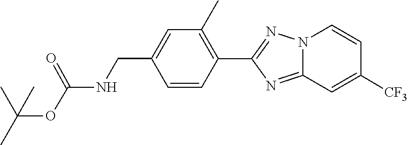 [SMILES: Cc1cc(CNC(=O)OC(C)(C)C)ccc1-c1nc2cc(C(F)(F)F)ccn2n1]

Intermediate I-40 was prepared according to an analogous procedure to the one used for the synthesis of intermediate I-14 using intermediate I-39 (1.35 g, 4.46 mmol) as starting material (0.82 g, 44%).

### Synthesis of Intermediate I-41

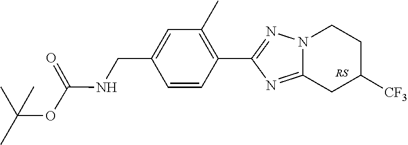 [SMILES: Cc1cc(CNC(=O)OC(C)(C)C)ccc1-c1nc2n(n1)CCC(C(F)(F)F)C2]

Palladium(II) hydroxide on carbon [12135-22-7] (70 mg, 0.5 mmol) was added portionwise to a solution of intermediate I-40 (0.40 g, 0.98 mmol) in a mixture of methanol (5 mL) and EtOAc (1 mL) at 0° C. under N2. Then H2 was added, and the mixture was stirred at rt for 18 h. The mixture was filtered through a pad of Celite® and the solvents were concentrated in vacuo to yield intermediate I-41 as a yellow solid (0.40 g, 89%).

### Synthesis of Intermediate I-42

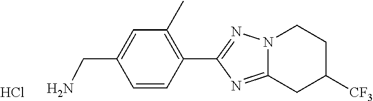 [SMILES: Cl ; Cc1cc(CN)ccc1-c1nc2n(n1)CCC(C(F)(F)F)C2]

Intermediate I-42 was prepared according to an analogous procedure to the one used for the synthesis of intermediate I-6 using intermediate I-41 (0.4 g, 0.97 mmol) as starting material (0.34 g, quantitative).

### Synthesis of Intermediate I-43

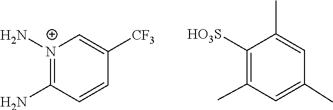

Intermediate I-42 was prepared according to an analogous procedure to the one used for the synthesis of intermediate I-2 using 2-amino-4-(trifluoromethyl)pyridine [74784-70-6] (2 g, 13.34 mmol) as starting material (3.95 g, 79%).

### Synthesis of Intermediate I-44

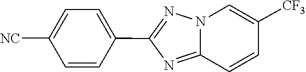 [SMILES: N#Cc1ccc(-c2nc3ccc(C(F)(F)F)cn3n2)cc1]

Intermediate I-44 was prepared according to an analogous procedure to the one used for the synthesis of intermediate I-3 using intermediate I-43 (3.95 g, 10.23 mmol) as starting material (2.2 g, 73%).

### Synthesis of Intermediate I-45

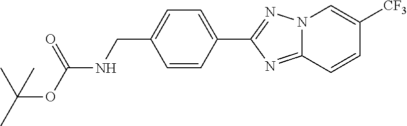 [SMILES: CC(C)(C)OC(=O)NCc1ccc(-c2nc3ccc(C(F)(F)F)cn3n2)cc1]

Intermediate I-45 was prepared according to an analogous procedure to the one used for the synthesis of intermediate I-4 using intermediate I-44 (2.2 g, 7.63 mmol) as starting material (1.1 g, 35%).

### Synthesis of Intermediate I-46

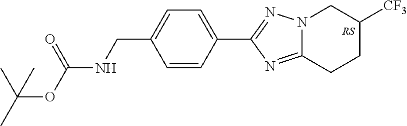 [SMILES: CC(C)(C)OC(=O)NCc1ccc(-c2nc3n(n2)CC(C(F)(F)F)CC3)cc1]

Intermediate I-46 was prepared according to an analogous procedure to the one used for the synthesis of intermediate I-26 using intermediate I-45 (0.3 g, 0.76 mmol) as starting material (196 mg, 64%).

### Synthesis of Intermediate I-47

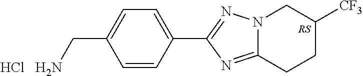 [SMILES: Cl ; NCc1ccc(-c2nc3n(n2)CC(C(F)(F)F)CC3)cc1]

Intermediate I-47 was prepared according to an analogous procedure to the one used for the synthesis of intermediate I-6 using intermediate I-46 (196 mg, 0.49 mmol) as starting material (173 mg, quantitative).

### Synthesis of Intermediate I-47

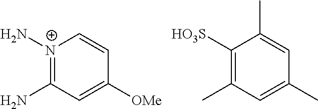

Intermediate I-47 was prepared according to an analogous procedure to the one used for the synthesis of intermediate I-2 using 4-(difluoromethyl)pyridine-2-amine [1346536-47-7] (0.5 g, 3.47 mmol) as starting material (1.27 g, 91%).

### Synthesis of Intermediate I-48

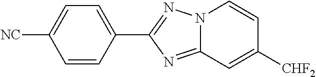 [SMILES: N#Cc1ccc(-c2nc3cc(C(F)F)ccn3n2)cc1]

Intermediate I-48 was prepared according to an analogous procedure to the one used for the synthesis of intermediate I-3 using intermediate I-47 (1.27 g, 3.16 mmol) as starting material (0.61 g, 67%).

### Synthesis of Intermediate I-49

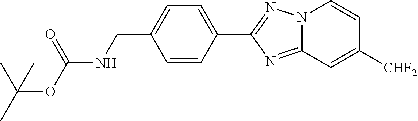 [SMILES: CC(C)(C)OC(=O)NCc1ccc(-c2nc3cc(C(F)F)ccn3n2)cc1]

Sodium borohydride [16940-66-2] (255 mg, 6.74 mmol) was added portionwise to a suspension of intermediate I-48 (0.61 g, 2.25 mmol), nickel (II) chloride hexahydrate [7791-20-0] (0.53 mg, 2.25 mmol) and di-tertbutyl dicarbonate [24424-99-5] (1.55 mL, 6.75 mmol) in a mixture of dry methanol (12 mL) and dry 1,4-dioxane (6 mL) at 0° C. under N2. The reaction mixture was stirred at rt for 16 h and then water and a NH3 aqueous solution (3 mL) were added, and the mixture was extracted with DCM. The combined organic layers were dried (MgSO4), filtered and concentrated in vacuo. The crude product was purified by flash column chromatography (silica; EtOAc in heptane to 20/80). The desired fractions were collected and concentrated in vacuo to yield intermediate I-49 as a white solid (849 mg, 45%).

### Synthesis of Intermediate I-50

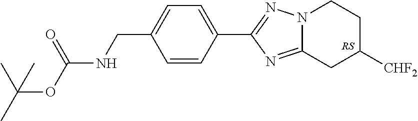 [SMILES: CC(C)(C)OC(=O)NCc1ccc(-c2nc3n(n2)CCC(C(F)F)C3)cc1]

Palladium(II) hydroxide on carbon (20% w/w) 50% H2O [7440-05-3] (0.42 g, 0.60 mmol) was added portionwise to a solution of intermediate I-49 (0.97 g, 3.36 mmol) in a mixture of ethanol (13.5 mL) and dry THF (13.5 mL) at 0° C. under N2. Then H2 was added, and the mixture was stirred at rt for 2 h. The mixture was filtered through a pad of Celite® and the solvents were concentrated in vacuo. The crude product was purified by flash column chromatography (silica; EtOAc in heptane 0/100 to 75/25). The desired fractions were collected and concentrated in vacuo to yield intermediate I-50 as a white solid (223 mg, 73%).

### Synthesis of Intermediate I-51

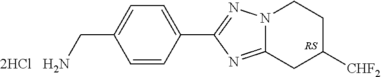 [SMILES: "2HCl" ; NCc1ccc(-c2nc3n(n2)CCC(C(F)F)C3)cc1]

Intermediate I-51 was prepared according to an analogous procedure to the one used for the synthesis of intermediate I-6 using intermediate I-50 (223 mg, 0.59 mmol) as starting material (209 mg, quantitative).

### Synthesis of Intermediate I-52

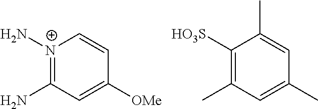

Intermediate I-52 was prepared according to an analogous procedure to the one used for the synthesis of intermediate I-2 using 2-amino-4-methoxypyridine [10201-73-7] (0.5 g, 4.03 mmol) as starting material (0.81 g, 51%, 87% purity).

### Synthesis of Intermediate I-53

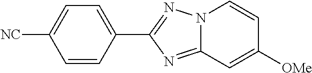 [SMILES: COc1ccn2nc(-c3ccc(C#N)cc3)nc2c1]

Intermediate I-53 was prepared according to an analogous procedure to the one used for the synthesis of intermediate I-3 using intermediate I-52 (0.8 g, 2.35 mmol) as starting material (0.33 g, 56%).

### Synthesis of Intermediate I-54

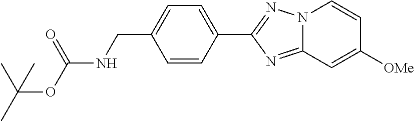 [SMILES: COc1ccn2nc(-c3ccc(CNC(=O)OC(C)(C)C)cc3)nc2c1]

Intermediate I-54 was prepared according to an analogous procedure to the one used for the synthesis of intermediate I-4 using intermediate I-53 (0.33 g, 1.32 mmol) as starting material (0.33 g, 67%).

### Synthesis of Intermediate I-55

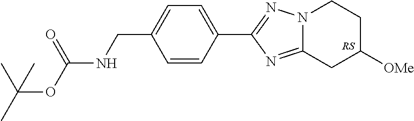 [SMILES: COC1CCn2nc(-c3ccc(CNC(=O)OC(C)(C)C)cc3)nc2C1]

Intermediate I-55 was prepared according to an analogous procedure to the one used for the synthesis of intermediate I-36 using intermediate I-54 (0.40 g, 1.09 mmol) as starting material (0.25 g, 63%).

### Synthesis of Intermediate I-56

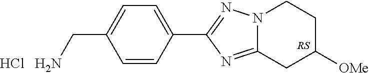 [SMILES: Cl ; COC1CCn2nc(-c3ccc(CN)cc3)nc2C1]

Intermediate I-56 was prepared according to an analogous procedure to the one used for the synthesis of intermediate I-6 using intermediate I-55 (0.25 g, 0.68 mmol) as starting material (201 mg, 99%).

### Synthesis of Intermediate I-57

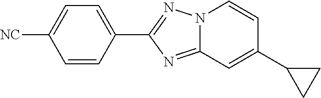 [SMILES: N#Cc1ccc(-c2nc3cc(C4CC4)ccn3n2)cc1]

Pd(OAc)2 [3375-31-3] (19 mg, 0.08 mmol) and SPhos [657408-07-6] (34 mg, 0.08 mmol) were added to a stirred solution of intermediate I-12 (0.5 g, 1.67 mmol), cyclopropyl boronic acid [411235-57-9] (144 mg, 1.77 mmol) and K3PO4 (1.77 g, 8.36 mmol) in dry 1,4-dioxane (4 mL) in a sealed tube under N2. The mixture was stirred at 95° C. for 16 h. Then water was added, and the mixture was extracted with EtOAc (×3). The combined organic layer was separated, dried (MgSO4), filtered and concentrated in vacuo to yield intermediate I-13 as a white solid (317 mg, 70%).

### Synthesis of Intermediate I-58

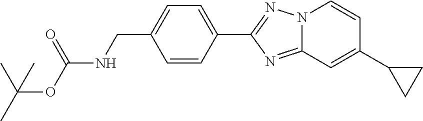 [SMILES: CC(C)(C)OC(=O)NCc1ccc(-c2nc3cc(C4CC4)ccn3n2)cc1]

Intermediate I-58 was prepared according to an analogous procedure to the one used for the synthesis of intermediate I-14 using intermediate I-57 (287 mg, 1.1 mmol) as starting material (318 mg, 78%).

### Synthesis of Intermediate I-59

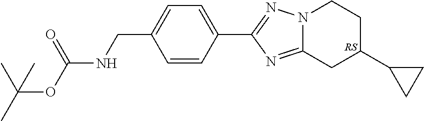 [SMILES: CC(C)(C)OC(=O)NCc1ccc(-c2nc3n(n2)CCC(C2CC2)C3)cc1]

Palladium(II) hydroxide on carbon [12135-22-7] (31 mg, 0.11 mmol) was added to a solution of intermediate I-58 (167 mg, 0.45 mmol), in a mixture of ethanol (2 mL), THE (2 mL) and acetic acid (0.2 mL) at 0° C. under N2. Then H2 was added, and the mixture was stirred at rt for 16 h. The mixture was filtered through a pad of Celite® and the solvents were concentrated in vacuo to yield intermediate I-15 as a yellow solid used in the next step without any further purification (166 mg, 51%, 50% purity).

### Synthesis of Intermediate I-60

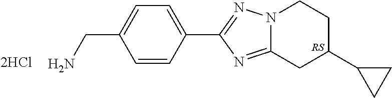 [SMILES: "2HCl" ; NCc1ccc(-c2nc3n(n2)CCC(C2CC2)C3)cc1]

Intermediate I-60 was prepared according to an analogous procedure to the one used for the synthesis of intermediate I-16 using intermediate I-59 (166 mg, 0.45 mmol) as starting material (154 mg, 32%, 32% purity).

### Synthesis of Intermediate I-61

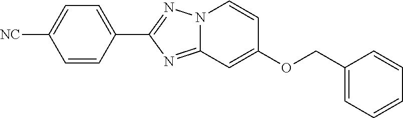 [SMILES: N#Cc1ccc(-c2nc3cc(OCc4ccccc4)ccn3n2)cc1]

Pd(OAc)2 [3375-31-3] (170 mg, 0.76 mmol) and BredttPhos [1070663-78-3] (410 mg, 0.76 mmol) were added to a stirred solution of intermediate I-12 (3.8 g, 19.03 mmol), benzyl alcohol [100-51-6] (2 mL, 1.77 mmol) and Cs2CO3 (6.2 g, 8.36 mmol) in toluene (22 mL) in a sealed tube under N2. The mixture was stirred at 75° C. for 16 h. Then water was added, and the mixture was extracted with DCM (×3). The combined organic layer was separated, dried (MgSO4), filtered and concentrated in vacuo to yield intermediate I-61 as a yellow solid (8 g, 47%, 55% purity).

### Synthesis of Intermediate I-62

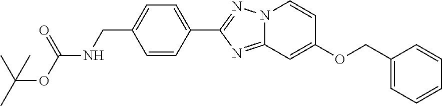 [SMILES: CC(C)(C)OC(=O)NCc1ccc(-c2nc3cc(OCc4ccccc4)ccn3n2)cc1]

Intermediate I-62 was prepared according to an analogous procedure to the one used for the synthesis of intermediate I-4 using intermediate I-61 (3.5 g, 7.29 mmol) as starting material (0.87 g, 27%).

### Synthesis of Intermediate I-63

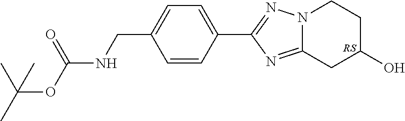 [SMILES: CC(C)(C)OC(=O)NCc1ccc(-c2nc3n(n2)CCC(O)C3)cc1]

Intermediate I-63 was prepared according to an analogous procedure to the one used for the synthesis of intermediate I-9 using intermediate I-62 (0.87 g, 2.01 mmol) as starting material (488 mg, 70%).

### Synthesis of Intermediate I-64

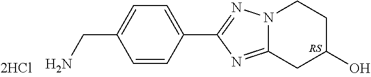 [SMILES: NCc1ccc(-c2nc3n(n2)CCC(O)C3)cc1]

Intermediate I-64 was prepared according to an analogous procedure to the one used for the synthesis of intermediate I-5 using intermediate I-63 (100 mg, 0.29 mmol) as starting material (93 mg, quantitative).

### Synthesis of Intermediate I-65

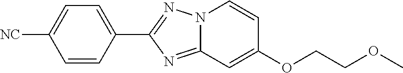 [SMILES: COCCOc1ccn2nc(-c3ccc(C#N)cc3)nc2c1]

Intermediate I-65 was prepared according to an analogous procedure to the one used for the synthesis of intermediate I-61 using 2-methoxyethanol [109-86-4] (0.40 mL, 5.01 mmol) as starting material (0.97 g, 85%, 86% purity).

### Synthesis of Intermediate I-66

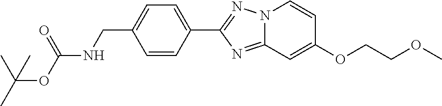 [SMILES: COCCOc1ccn2nc(-c3ccc(CNC(=O)OC(C)(C)C)cc3)nc2c1]

Intermediate I-66 was prepared according to an analogous procedure to the one used for the synthesis of intermediate I-4 using intermediate I-65 (0.97 g, 2.83 mmol) as starting material (0.6 g, 52%).

### Synthesis of Intermediate I-67

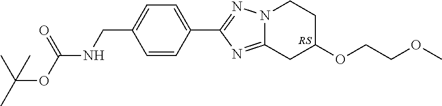 [SMILES: COCCOC1CCn2nc(-c3ccc(CNC(=O)OC(C)(C)C)cc3)nc2C1]

Palladium on carbon (10% w/w) 50% H₂O [7440-05-3] (0.4 g, 0.38 mmol) was added to a solution of intermediate I-66 (200 mg g, 0.50 mmol) in a mixture of methanol (15 mL) and EtOAc (2 mL) at 0° C. under N₂. H₂ was added, and the mixture was stirred at 50° C. for 16 h. Then more Palladium on carbon (10% w/w) 50% H₂O [7440-05-3] (0.4 g, 0.38 mmol) was added at 0° C. under N₂. H₂ was added, and the mixture was stirred at rt for a further 16 h. The mixture was filtered through a pad of Celite® and the solvents were concentrated in vacuo. The crude product was purified by flash column chromatography (silica; EtOAc in DCM 0/100 to 100/0). The desired fractions were collected and concentrated in vacuo to yield intermediate I-67 as a white solid (77 mg, 36%).

### Synthesis of Intermediate I-68

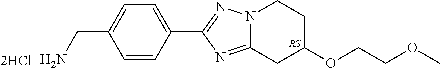 [SMILES: COCCOC1CCn2nc(-c3ccc(CN)cc3)nc2C1]

Intermediate I-68 was prepared according to an analogous procedure to the one used for the synthesis of intermediate I-6 using intermediate I-67 (213 mg, 0.53 mmol) as starting material (199 mg, quantitative).

### Synthesis of Intermediate I-69

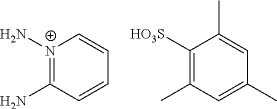

Intermediate I-69 was prepared according to an analogous procedure to the one used for the synthesis of intermediate I-2 using 2-aminopyridine [504-29-0] (1.75 g, 18.57 mmol) as starting material (5.17 g, 89%).

### Synthesis of Intermediate I-70a

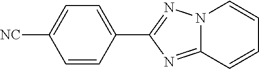 [SMILES: N#Cc1ccc(-c2nc3ccccn3n2)cc1]

Intermediate I-70a was prepared according to an analogous procedure to the one used for the synthesis of intermediate I-3 using intermediate I-69 (2 g, 6.44 mmol) as starting material (1.09 g, 76%).

### Synthesis of Intermediate I-70b

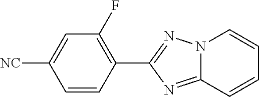 [SMILES: N#Cc1ccc(-c2nc3ccccn3n2)c(F)c1]

Intermediate I-70b was prepared according to an analogous procedure to the one used for the synthesis of intermediate I-3 using intermediate I-33 (3.75 g, 20.43 mmol) as starting material (1.6 g, 65%).

### Synthesis of Intermediate I-71a

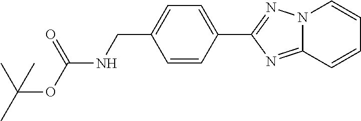 [SMILES: CC(C)(C)OC(=O)NCc1ccc(-c2nc3ccccn3n2)cc1]

Intermediate I-71a was prepared according to an analogous procedure to the one used for the synthesis of intermediate I-4 using intermediate I-70a (1.08 g, 4.90 mmol) as starting material (1.37 g, 73%, 85% purity).

### Synthesis of Intermediate I-71b

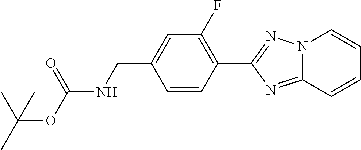 [SMILES: CC(C)(C)OC(=O)NCc1ccc(-c2nc3ccccn3n2)c(F)c1]

Intermediate I-71b was prepared according to an analogous procedure to the one used for the synthesis of intermediate I-4 using intermediate I-70b (1.59 g, 6.67 mmol) as starting material (1.95 g, 79%).

### Synthesis of Intermediate I-72a

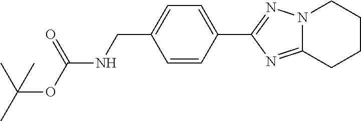 [SMILES: CC(C)(C)OC(=O)NCc1ccc(-c2nc3n(n2)CCCC3)cc1]

Intermediate I-72a was prepared according to an analogous procedure to the one used for the synthesis of intermediate I-5 using intermediate I-71a (1.34 g, 3.51 mmol) as starting material (1 g, 84%).

### Synthesis of Intermediate I-72b

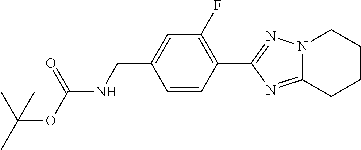 [SMILES: CC(C)(C)OC(=O)NCc1ccc(-c2nc3n(n2)CCCC3)c(F)c1]

Intermediate I-72b was prepared according to an analogous procedure to the one used for the synthesis of intermediate I-5 using intermediate I-71b (1.93 g, 5.19 mmol) as starting material (1.25 g, 68%).

### Synthesis of Intermediate I-73a

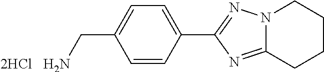 [SMILES: "2HCl" ; NCc1ccc(-c2nc3n(n2)CCCC3)cc1]

Intermediate I-73a was prepared according to an analogous procedure to the one used for the synthesis of intermediate I-6 using intermediate I-72a (1 g, 2.95 mmol) as starting material (0.93 g, quantitative).

### Synthesis of Intermediate I-73b

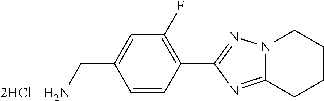 [SMILES: "2HCl" ; NCc1ccc(-c2nc3n(n2)CCCC3)c(F)c1]

Intermediate I-73b was prepared according to an analogous procedure to the one used for the synthesis of intermediate I-6 using intermediate I-72b (1.25 g, 3.5 mmol) as starting material (1.13 g, quantitative).

### Synthesis of Intermediate I-74

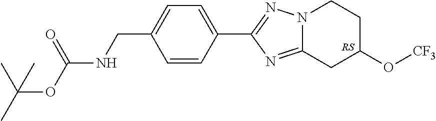 [SMILES: CC(C)(C)OC(=O)NCc1ccc(-c2nc3n(n2)CCC(OC(F)(F)F)C3)cc1]

Trimethyl(trifluoromethyl)silane [81290-20-2] (0.42 uL, 2.6 mmol) was added to a mixture of intermediate I-63 (150 mg, 0.4 mmol), N-fluoro-N′-(chloromethyl)triethylenediamine bis(tetrafluoroborate) [140681-55-6] (500 mg, 1.4 mmol), silver trifluoromethanesulfonate [2923-28-6] (700 mg, 2.7 mmol), 2-fluoropyridine (250 uL, 2.9 mmol) and potassium fluoride (230 mg, 4 mmol) in EOAc (15 mL). The reaction mixture was stirred at rt for 4 d in the dark.

The reaction mixture was filtered through a Celite® pad and the pad washed with EtOAc. The solvent was evaporated in vacuo, and the crude product was purified by flash column chromatography (silica, DCM/MeOH (9:1) in DCM from 100/0 to 0/100). The desired fractions were collected and concentrated in vacuo to yield intermediate I-74 as a yellow solid (105 mg, 47%, 80% purity).

### Synthesis of Intermediate I-75

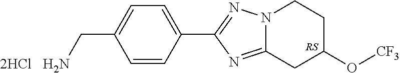 [SMILES: "2HCl" ; NCc1ccc(-c2nc3n(n2)CCC(OC(F)(F)F)C3)cc1]

Intermediate I-75 was prepared according to an analogous procedure to the one used for the synthesis of intermediate I-6 using intermediate I-74 (100 mg, 0.24 mmol) as starting material (136 mg, quantitative).

### Synthesis of Intermediate I-76

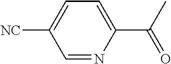

Tributyl(1-ethoxyvinyl)tin [97674-02-7] (5.9 mL, 14.46 mmol) was added to a stirred solution of 6-chloro-3-pyridinecarbonitrile [623-00-7] (2 g, 14.43 mmol) and bis(triphenylphosphine)palladium(II) chloride [13965-03-2] in dry toluene (20 mL) at rt under N₂. The mixture was stirred at 130° C. for 2 h. Then the mixture was cooled down to 0° C. with and ice bath and a 6M HCl aqueous solution (5.3 mL) was added. The mixture was stirred at rt for 1 h. The reaction was cooled down to 0° C. with ice bath and the reaction was brought to pH 8 by a 4M NaOH aqueous solution and a NaHCO₃ saturated aqueous solution addition. The resulting suspension was filtered through a Celite® pad. The filtrate was extracted with EtOAc, dried (MgSO₄), filtered and concentrated in vacuo. The crude product was purified by flash column chromatography (silica; EtOAc in heptane 0/100 to 40/60). The desired fractions were collected and concentrated in vacuo to yield intermediate I-76 as a yellow solid (1.78 g, 83%).

### Synthesis of Intermediate I-77

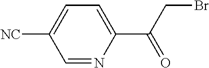 [SMILES: N#Cc1ccc(C(=O)CBr)nc1]

Pyridinium tribromide [39416-48-3] (3.54 g, 11.06 mmol) was added to a solution of intermediate I-76 (1.62 g, 11.05 mmol) in THE (50 mL) at rt. The reaction mixture was stirred at rt for 16 h and then diluted with EtOAc and a saturated Na₂S₂O₃ aqueous solution. The aqueous layer was extracted with EtOAc (×3) and the combined organic extracts were dried (MgSO₄), filtered and the solvent evaporated in vacuo. The crude product was purified by flash column chromatography (silica, EtOAc in heptane 0/100 to 30/70). The desired fractions were collected and concentrated in vacuo to yield intermediate I-77 as a yellow solid (0.68 g, 25%).

### Synthesis of Intermediate I-78

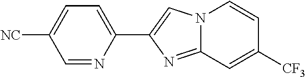 [SMILES: N#Cc1ccc(-c2cn3ccc(C(F)(F)F)cc3n2)nc1]

NaHCO₃ (218 mg, 2.60 mmol) was added to a solution of 2-amino-4-(trifluoromethyl)pyridine [106447-97-6] (375 mg, 2.31 mmol) and intermediate I-77 (680 mg, 3.02 mmol) in ethanol (16 mL) at rt. The mixture was stirred at reflux for 16 hours. H₂O was added and the precipitate formed was filtered off and washed with water and diethyl ether. The solid was dried in vacuo to afford Intermediate I-78 (380 mg, 57%) as a brown solid. The filtrate was extracted with DCM and the organic layer was dried (MgSO₄), filtered and the solvent concentrated in vacuo. The residue was purified by flash column chromatography (silica, EtOAc in heptane 0/100 to 30/70). The desired fractions were collected and concentrated in vacuo to yield further intermediate I-78 as a yellow solid (96 mg, 14%).

### Synthesis of Intermediate I-79

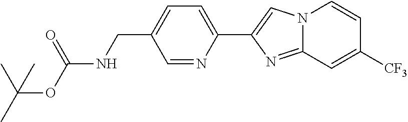 [SMILES: CC(C)(C)OC(=O)NCc1ccc(-c2cn3ccc(C(F)(F)F)cc3n2)nc1]

Sodium borohydride [16940-66-2] (187 mg, 4.94 mmol) was added portionwise to a suspension of intermediate I-78 (476 mg, 1.65 mmol), nickel (II) chloride hexahydrate [7791-20-0] (393 g, 1.65 mmol) and di-tertbutyl dicarbonate [24424-99-5] (1.14 mL, 4.96 mmol) in a mixture of dry methanol (8 mL) and 1,4-dioxane (4 mL) at 0° C. under N₂. The reaction mixture was stirred at rt for 16 h. Water and 3 mL of aqueous NH₃ were added and the mixture was extracted with DCM (×3). The combined organic layers were washed with water, separated, dried (MgSO₄), filtered and concentrated in vacuo. The crude product was purified by flash column chromatography (silica, EtOAc in heptane 0/100 to 40/60). The desired fractions were collected and concentrated in vacuo to yield intermediate I-79 as a brown solid (349 mg, 53%).

### Synthesis of Intermediate I-80

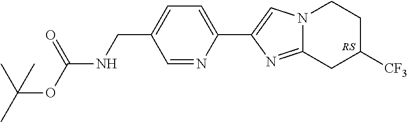 [SMILES: CC(C)(C)OC(=O)NCc1ccc(-c2cn3c(n2)CC(C(F)(F)F)CC3)nc1]

Intermediate I-80 was prepared according to an analogous procedure to the one used for the synthesis of intermediate I-9 using intermediate I-79 (306 mg, 0.78 mmol) as starting material (175 mg, 56%).

### Synthesis of Intermediate I-81

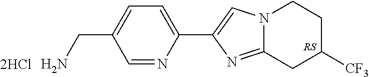 [SMILES: "2HCl" ; NCc1ccc(-c2cn3c(n2)CC(C(F)(F)F)CC3)nc1]

Intermediate I-81 was prepared according to an analogous procedure to the one used for the synthesis of intermediate I-10 using intermediate I-80 (175 mg, 0.44 mmol) as starting material (171 mg, quantitative).

### Synthesis of Intermediates II

Synthesis of intermediate II-1a

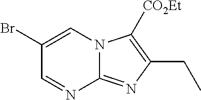 [SMILES: CCOC(=O)c1c(CC)nc2ncc(Br)cn12]

Boron trifluoride diethyl etherate [109-63-7] (0.35 mL, 2.87 mmol) was added dropwise to a solution of 2-amino-5-bromopyrimidine [7752-82-1] (5 g, 28.74 mmol), ethyl propionylacetate [4949-44-4] (6.33 mL, 43.10 mmol) and (diacetoxyiodo)benzene [3240-34-4](13.88 g, 43.09 mmol) in dry 2-methyltetrahydrofuran (125 mL), in a 2-neck round bottom flask equipped with a condenser, at rt under N₂. The mixture was stirred at 60° C. for 16 h. A saturated NaHCO₃ aqueous solution was added, and the mixture was extracted with EtOAc. The organic layer was separated, dried (MgSO₄), filtered and the solvents evaporated in vacuo. The crude product was purified by flash column chromatography (silica; EtOAc in heptane 0/100 to 40/60). The desired fractions were collected and concentrated in vacuo to yield intermediate II-1a as a yellow solid (3.75 g, 43%).

### Synthesis of Intermediate II-1b

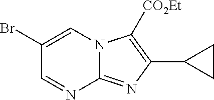 [SMILES: CCOC(=O)c1c(C2CC2)nc2ncc(Br)cn12]

Intermediate II-1b was prepared according to an analogous procedure to the one used for the synthesis of intermediate II-1a using Ethyl-3-cyclopropyl-3-oxopropionate [24922-02-9](3.82 mL, 25.86 mmol) as starting material (2.68 mg, 47%).

### Synthesis of Intermediate II-1c

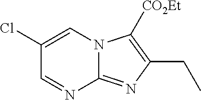 [SMILES: CCOC(=O)c1c(CC)nc2ncc(Cl)cn12]

Boron trifluoride diethyl etherate [109-63-7] (0.2 mL, 1.62 mmol) was added dropwise to a solution of 2-amino-5-chloropyrimidine [5428-89-7] (2.0 g, 15.438 mmol), ethyl propionylacetate [4949-44-4] (3.14 mL, 21.38 mmol) and (diacetoxyiodo)benzene [3240-34-4] (7.5 g, 23.29 mmol) in dry 2-methyltetrahydrofuran (75 mL) at 0° C. under N₂. The mixture was stirred at rt for 16 h and then was poured into a 10% NaHCO₃ aqueous solution and extracted with EtOAc. The organic layer was separated, dried (MgSO₄), filtered, and the solvents were evaporated in vacuo. The crude was purified by flash column chromatography (silica; EtOAc in heptane 0/100 to 20/80). The desired fractions were collected and concentrated in vacuo to yield intermediate II-1c as a yellow solid.

### Synthesis of Intermediate II-1d

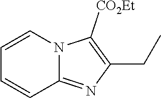 [SMILES: CCOC(=O)c1c(CC)nc2ccccn12]

Boron trifluoride diethyl etherate [109-63-7] (66 μL, 0.53 mmol) was added dropwise to a solution of 2-aminopyridine [4949-44-4] (1 g, 10.62 mmol), ethyl propionylacetate [4949-44-4] (2.34 mL, 15.94 mmol) and (diacetoxyiodo)benzene [3240-34-4] 1.71 g, 5.31 mmol) in dry 2-methyltetrahydrofuran (25 mL), at 5° C. under N₂. The mixture was stirred at 5° C. for 15 min and then allowed to slowly warm to rt and stirred for a further 5 h. A saturated NaHCO₃ aqueous solution was added. The organic layer was separated, dried (MgSO₄), filtered and the solvents evaporated in vacuo. The crude product was purified by flash column chromatography (silica; EtOAc in heptane 0/100 to 50/50). The desired fractions were collected and concentrated in vacuo to yield intermediate II-1d as a white solid (0.78 g, 32%).

### Synthesis of Intermediate II-2a

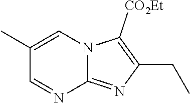 [SMILES: CCOC(=O)c1c(CC)nc2ncc(C)cn12]

A 2M trimethylaluminum solution in hexane [75-24-1] (22.05 mL, 44.11 mmol) was added dropwise to a solution of intermediate II-1a (3.78 g, 12.60 mmol) and tetrakis(triphenylphosphine)-palladium(0) [14221-01-3] in dry THF (90 mL) at rt under N₂ and the mixture was stirred at 65° C. for 2 h. The mixture was cooled to 0° C. and diluted with DCM. Then 10 mL of water was added dropwise. The resulting mixture was filtered through of pad of Celite® and the pad was washed with EtOAc. Then anhydrous MgSO₄ was added to the filtrate. The filtrate was filtered, and the solvents evaporated in vacuo. The crude product was purified by flash column chromatography (silica; EtOAc in heptane 0/100 to 40/60). The desired fractions were collected and concentrated in vacuo to intermediate II-2a as a yellow solid (2.45 g, 75%).

### Synthesis of Intermediate II-2b

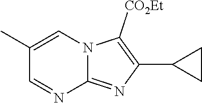 [SMILES: CCOC(=O)c1c(C2CC2)nc2ncc(C)cn12]

Intermediate II-2b was prepared according to an analogous procedure to the one used for the synthesis of intermediate II-1a using intermediate II-1b (2.57 mL, 8.10 mmol) as starting material (1.46 mg, 72%).

### Synthesis of Intermediate II-3a

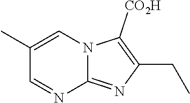

Sodium hydroxide [1310-73-2] (1.13 g, 28.29 mmol) was added to a solution of intermediate II-2a in a mixture of ethanol (74 mL) and water (19 mL) at rt. The mixture was stirred at 50° C. for 2 h. The reaction mixture was brought to pH 7 by a 1M HCl aqueous solution addition and concentrated in vacuo to yield intermediate II-3a as a pale orange solid used in next step without any further purification (2.76 g, quantitative, 90% purity).

### Synthesis of Intermediate II-4a

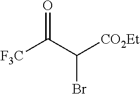 [SMILES: CCOC(=O)C(Br)C(=O)C(F)(F)F]

N-Bromosuccinimide [128-08-5] (14.69 g, 82.56 mmol) was added to a stirred solution of ethyl 4,4,4-trifluoroacetoacetate [372-31-6] (14.48 g, 78.63 mmol) in DMSO (72 mL). The reaction mixture was stirred at rt for 1 hour. A saturated NH₄Cl aqueous solution was added, and the mixture was extracted with EtOAc. The organic layer was washed with brine, dried (MgSO₄), filtered and concentrated in vacuo to yield intermediate II-4a as a colourless oil used in the next step without any further purification (14.1 g, 55%, 80% purity).

### Synthesis of Intermediate II-4b

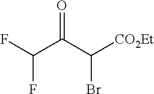 [SMILES: CCOC(=O)C(Br)C(=O)C(F)F]

Bromine [7726-95-6] (0.70 mL, 13.75 mmol) was added dropwise to a mixture of ethyl 4,4-difluoroacetoacetate [7726-95-6] (1.8 mL, 13.75 mmol), CaCO₃ (1.6 g, 15.95 mmol) in dry methanol (20 mL) at 0° C. The mixture was stirred at rt for 1 h and then concentrated in vacuo. The crude product was triturated with diethyl ether, the solid formed was filtered off and the filtrate was concentrated in vacuo to yield II-4b as a yellow oil (3.37, quantitative).

### Synthesis of Intermediate II-5a

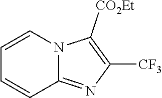 [SMILES: CCOC(=O)c1c(C(F)(F)F)nc2ccccn12]

2-Aminopyridine [504-29-0] (3.2 g, 34 mmol) was added to a stirred solution of intermediate II-4a (18 g, 68.43 mmol) in ethanol (100 mL) at rt in a sealed tube. The reaction mixture was stirred at 80° C. for 48 hours and then the solvent was evaporated in vacuo. A saturated NaHCO₃ aqueous solution was added, and the mixture was extracted with EtOAc. The organic layer was washed with brine, dried (MgSO₄), filtered and concentrated in vacuo. The crude product was purified by flash column chromatography (silica; EtOAc in heptane 100/0 to 80/20). The desired fractions were collected and concentrated in vacuo to intermediate II-5a as a pale yellow solid (2.0 g, 22%).

### Synthesis of Intermediate II-5b

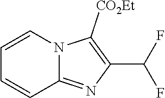 [SMILES: CCOC(=O)c1c(C(F)F)nc2ccccn12]

2-Aminopyridine [504-29-0] (0.51 g, 5.44 mmol) was added to a stirred solution of intermediate II-4b (2 g, 8.16 mmol) in ethanol (30 mL) at rt. The reaction mixture was stirred at 65° C. for 16 hours. A saturated NaHCO₃ aqueous solution was added, and the mixture was extracted with EtOAc. The organic layer was washed with brine, dried (MgSO₄), filtered and concentrated in vacuo. The crude product was purified by flash column chromatography (silica; EtOAc in heptane 100/0 to 40/26). The desired fractions were collected and concentrated in vacuo to intermediate II-5a as a colourless solid (0.81 g, 59%).

### Synthesis of Intermediate II-6a

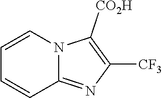 [SMILES: O=C(O)c1c(C(F)(F)F)nc2ccccn12]

A 1M solution of aqueous sodium hydroxide [1310-73-2] (1.32 mL, 1.32 mmol) was added to a solution of intermediate II-5a (113 mg, 0.44 mmol) in a mixture of ethanol (3 mL) and water (1 mL) at rt. The mixture was stirred at rt for 16 h. The reaction mixture was brought to pH 5 by a 1M HCl aqueous solution addition and concentrated in vacuo to yield intermediate II-6a as a white solid used in next step without any further purification (101 mg, quantitative).

### Synthesis of Intermediate II-6b

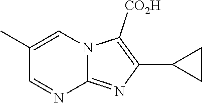 [SMILES: Cc1cnc2nc(C3CC3)c(C(=O)O)n2c1]

Intermediate II-6b was prepared according to an analogous procedure to the one used for the synthesis of intermediate II-6a using intermediate II-2b (120 mg, 0.49 mmol) as starting material (106 mg, quantitative).

### Synthesis of Intermediate II-7a

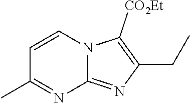 [SMILES: CCOC(=O)c1c(CC)nc2nc(C)ccn12]

KHCO₃ (688 mg, 6.87 mmol) and ethyl propionylacetate [4949-44-4] (0.98 mL, 6.87 mmol) were added to a stirred solution of 2-amino-4-methylpyrimidine [108-52-1] (500 mg, 4.58 mmol) in dry ACN (9.18 mL) at rt in a sealed tube. The reaction mixture was stirred at 80° C. for 16 hours and then a saturated NaHCO₃ aqueous solution was added, and the mixture was extracted with EtOAc (3×). The combined organic layers were dried (MgSO₄), filtered and concentrated in vacuo. The crude product was purified by flash column chromatography (silica; DCM/MeOH 9:1 in DCM 0/100 to 20/80). The desired fractions were collected and concentrated in vacuo to intermediate II-7a as a brown solid (206 mg, 19%).

### Synthesis of Intermediate II-7b

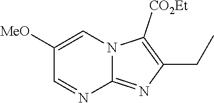 [SMILES: CCOC(=O)c1c(CC)nc2ncc(OC)cn12]

Intermediate II-7b was prepared according to an analogous procedure to the one used for the synthesis of intermediate II-7a using 2-amino-4-methylpyrimidine [13418-77-4] (500 mg, 4 mmol) as starting material (407 mg, 40%).

### Synthesis of Intermediate II-7c

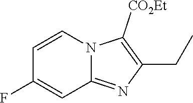 [SMILES: CCOC(=O)c1c(CC)nc2cc(F)ccn12]

Ethyl propionylacetate [4949-44-4] (1.87 mL, 12.71 mmol) and tetrabromoethane [558-13-4](11.24 g, 33.90 mmol) were added to a stirred solution of 4-fluoro-2-aminopiridine [944401-77-8] (1 g, 8.47 mmol) in dry ACN (15.25 mL) at rt in a sealed tube. The reaction mixture was stirred at 80° C. for 20 hours and then additional Ethyl propionylacetate [4949-44-4] (1.24 mL, 8.47 mmol) and tetrabromoethane [558-13-4] (2.81 g, 8.48 mmol). The reaction mixture was stirred at 80° C. for 5 hours and then additional Ethyl propionylacetate [4949-44-4] (0.62 mL, 4.24 mmol) and tetrabromoethane [558-13-4] (1.41 g, 4.24 mmol). The reaction mixture was stirred at 80° C. for a further 16 hours and then was poured onto a 10% NaHCO₃ aqueous solution and extracted with EtOAc (3×). The combined organic layers were dried (MgSO₄), filtered and concentrated in vacuo. The crude product was purified by flash column chromatography (silica; DCM/MeOH 9:1 in DCM 0/100 to 20/80). The desired fractions were collected and concentrated in vacuo to intermediate II-7c as a pale yellow solid (529 mg, 25%).

### Synthesis of Intermediate II-7d

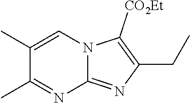 [SMILES: CCOC(=O)c1c(CC)nc2nc(C)c(C)cn12]

KHCO₃ (610 mg, 69 mmol) and ethyl propionylacetate [4949-44-4] (0.87 mL, 6.09 mmol) were added to a stirred solution of 4,5-dimethyl-2-pyrimidinamine [1193-74-4] (500 mg, 4.06 mmol) in dry ACN (8.14 mL). Then bromotrichloromethane [75-62-7] (1.2 mL, 12.18 mmol) was and the reaction mixture was stirred at 80° C. for 16 hours and then additional Ethyl propionylacetate [4949-44-4] (0.43 mL, 3.04 mmol) and bromotrichloromethane [75-62-7](0.6 mL, 6.09 mmol) were added. The reaction mixture was stirred at 80° C. for a further 16 hours and then a saturated NaHCO₃ aqueous solution was added, and the mixture was extracted with EtOAc (3×). The combined organic layers were dried (MgSO₄), filtered and concentrated in vacuo. The crude product was purified by flash column chromatography (silica; MeOH in DCM 0/100 to 2/98). The desired fractions were collected and concentrated in vacuo to intermediate II-7d as a brown solid (219 mg, 20%).

### Synthesis of Intermediate II-7f

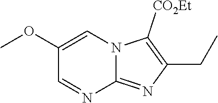 [SMILES: CCOC(=O)c1c(CC)nc2ncc(OC)cn12]

Intermediate II-7f was prepared according to an analogous procedure to the one used for the synthesis of intermediate II-7d using 5-methoxy-4-methylpyrimidin-2amine [1749-71-9] (0.5 g, 3.59 mmol) as starting material (0.23 g, 24%).

### Synthesis of Intermediate II-7g

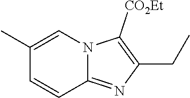 [SMILES: CCOC(=O)c1c(CC)nc2ccc(C)cn12]

Intermediate II-7g was prepared according to an analogous procedure to the one used for the synthesis of intermediate II-7d using 5-methyl-pyridin-2-ylamine [1603-41-4] (5 g, 46.24 mmol) as starting material (8.85 g, 82%).

### Synthesis of Intermediate II-8a

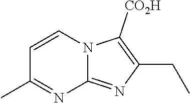

Sodium hydroxide [1310-73-2] (72 mg, 1.8 mmol) was added to a solution of intermediate II-7a (140 mg, 0.6 mmol) in a mixture of ethanol (4.7 mL) and water (1.2 mL) at rt. The mixture was stirred at 50° C. for 2 h. The reaction mixture was brought to pH 7 by a 1M HCl aqueous solution addition and concentrated in vacuo to yield intermediate II-8a as a pale orange solid used in next step without any further purification (2.76 g, quantitative, 90% purity).

### Synthesis of Intermediate II-8b

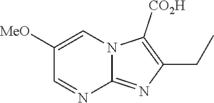 [SMILES: CCc1nc2ncc(OC)cn2c1C(=O)O]

Intermediate II-8b was prepared according to an analogous procedure to the one used for the synthesis of intermediate II-8a using intermediate II-7b (150 mg, 0.6 mmol) as starting material (135 mg, quantitative).

### Synthesis of Intermediate II-8c

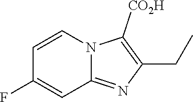

Intermediate II-8c was prepared according to an analogous procedure to the one used for the synthesis of intermediate II-8a using intermediate II-7c (248 mg, 1.04 mmol) as starting material (219 mg, quantitative).

### Synthesis of Intermediate II-8d

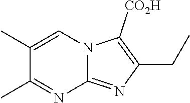 [SMILES: CCc1nc2nc(C)c(C)cn2c1C(=O)O]

Intermediate II-8d was prepared according to an analogous procedure to the one used for the synthesis of intermediate II-8a using intermediate II-7d (110 mg, 0.44 mmol) as starting material (115 mg, 99%).

### Synthesis of Intermediate II-8e

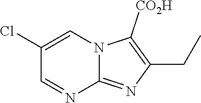

A 15% K₂CO₃ aqueous solution (1.2 mL, 1.30 mmol) was added to a solution of intermediate II-1d (143 mg, 0.56 mmol) in EtOH (1.5 mL) at rt in a screw cap vial. The mixture was stirred at 90° C. for 16 h and then was brought to pH 3-4 by a 2M HCl aqueous solution addition. The solvents were evaporated in vacuo to yield intermediate II-8e as a brownish solid (108 mg, 85%).

### Synthesis of Intermediate II-8f

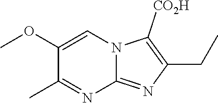 [SMILES: CCc1nc2nc(C)c(OC)cn2c1C(=O)O]

Intermediate II-8f was prepared according to an analogous procedure to the one used for the synthesis of intermediate II-8a using intermediate II-7f (95 mg, 0.36 mmol) as starting material (112 mg, quantitative).

### Synthesis of Intermediate II-9

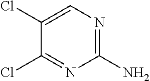

Trichloroisocyanuric acid [87-90-1] (1.54 g, 7.72 mmol) was added to a solution of 2-amino-4-chloropyrimidine [3993-78-0] (2 g, 15.44 mmol) and acid acetic (2 mL, 34.94 mmol) in water (18 mL) at rt. Then the mixture was stirred at 50° C. for 15 h and then cooled to rt and poured into a flask containing ice. The mixture was basified by addition of a 10M NaOH aqueous solution and stirred for 4 h. The solid formed was collected by filtration and washed with water (14 mL). The solid was suspended in 24 ml of a 0.5M NaOH aqueous solution and stirred for 1 h. The solid was collected by filtration and washed with 24 ml of water. The solid was dissolved with EtOAc and concentrated in vacuo. Then diethyl ether was added, and the mixture concentrated again in vacuo again to yield intermediate II-9 as a beige solid (1.7 g, 60%).

### Synthesis of Intermediate II-10

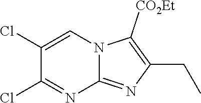 [SMILES: CCOC(=O)c1c(CC)nc2nc(Cl)c(Cl)cn12]

Ethyl propionylacetate [4949-44-4] (2.43 mL, 16.59 mmol) and (diacetoxyiodo)benzene [3240-34-4] (5.01 g, 15.54 mmol) were added to a solution of intermediate II-9 (1.7 g, 10.37 mmol) in dry 2-methyltetrahydrofuran (49.4 mL) at 0° C. under N₂. Then boron trifluoride diethyl etherate [109-63-7] (0.13 mL, 1.03 mmol) was added dropwise. The mixture was stirred at 5° C. for 5 min and then at rt for 2 h. Further ethyl propionylacetate [4949-44-4](1.21 mL, 8.29 mmol), (diacetoxyiodo)benzene [3240-34-4] (2.50 g, 7.78 mmol) and boron trifluoride diethyl etherate [109-63-7] (0.06 mL, 0.48 mmol) were added at 0° C. and the mixture was stirred at 5° C. for 5 min and then at rt for 2 h. The mixture was poured into a 10% NaHCO₃ aqueous solution and extracted with EtOAc. The organic layer was dried (MgSO₄), filtered and the solvents evaporated in vacuo. The crude was purified by flash column chromatography (silica; EtOAc in heptane 0/100 to 15/85). The desired fractions were collected and concentrated in vacuo. The residue was repurified by reverse phase HPLC (Phenomenex Gemini; C18 100×30 mm 5 μm Column; from 59% (25 mM NH₄HCO₃)/41% (ACN:MeOH 1:1) to 17% (25 mM NH₄HCO₃)/83% (ACN:MeOH 1:1). The desired fractions were collected and concentrated in vacuo to remove the organic solvents and the resulting aqueous layer was extracted with DCM. The organic layer was separated, dried (MgSO₄), filtered and the solvents evaporated in vacuo to intermediate II-10 as a white solid (510 mg, 16%).

### Synthesis of Intermediate II-11

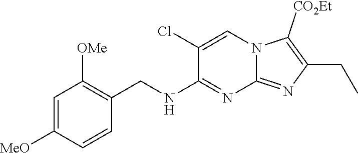 [SMILES: CCOC(=O)c1c(CC)nc2nc(NCc3ccc(OC)cc3OC)c(Cl)cn12]

2,4-Dimethoxybenzylamine [20781-20-8] (0.36 mL, 2.4 mmol) was added to a solution of intermediate II-10 (485 mg, 1.6 mmol) in dry 1,4-dioxane at rt. The suspension was stirred at rt for 2 h. and then more 2,4-dimethoxybenzylamine [20781-20-8] (0.18 mL, 1.2 mmol) was added and the mixture was stirred at 50° C. for 16 h. The solvent was removed in vacuo and then water was added, and the mixture was extracted with DCM. The organic layer was separated, dried (MgSO₄), filtered and the solvents evaporated in vacuo. The crude product was purified by flash column chromatography (silica; EtOAc in heptane 0/100 to 25/75). The desired fractions were collected and concentrated in vacuo to yield intermediate II-11 as a white solid (544 mg, 80%).

### Synthesis of Intermediate II-12a

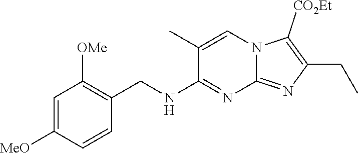 [SMILES: CCOC(=O)c1c(CC)nc2nc(NCc3ccc(OC)cc3OC)c(C)cn12]

Methylboronic acid [13061-96-6] (37 mg, 0.62 mmol) and K₃PO₄ (176 mg, 0.83 mmol) were added to a solution of intermediate II-11 in a mixture of water (0.33 mL and toluene (1.66 mL). The mixture was purged with N₂ for 10 min and then Palladium(II) acetate [3375-31-3](9 mg, 0.042 mmol) and SPhos [657408-07-6] (26 mg, 0.062 mmol) were added at rt and the reaction mixture was stirred at 110° C. for 2 hours. The solvents were removed under vacuum and the crude product was purified by flash column chromatography (silica; EtOAc in heptane 0/100 to 50/50). The desired fractions were collected and concentrated in vacuo to yield intermediate II-12a as a colourless oil.

### Synthesis of Intermediate II-12b

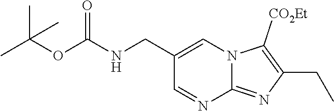 [SMILES: CCOC(=O)c1c(CC)nc2ncc(CNC(=O)OC(C)(C)C)cn12]

Experiment set-up in 6 batches of 50 mg of intermediate II-1a each.

A mixture of intermediate II-1a (250 mg, 0.85 mmol), potassium (((tert-butoxycarbonyl)amino)methyl)trifluoroborate [1314538-55-0] (240 mg, 1 mmol), [4,4′-bis (1,1-dimethylethyl)-2,2′-bipyridine] nickel (II) dichloride [1034901-50-2] (35 mg, 0.085 mmol), Ir[dF(CF3)ppy]2(dtbbpy)PF6 [870987-63-6] (25 mg, 0.02 mmol), Cs2CO3 (430 mg, 1.3 mmol) in dioxane (4 mL) in a screw top vial was degassed by bubbling N2 for 5 min. Then the vial was sealed, and the reaction mixture was irradiated with blue LED light at rt for 48 h. The reaction mixture was diluted with EtOAc and washed with water. The organic layer was separated, dried (MgSO4), filtered, and the solvent evaporated in vacuo. The crude was purified by flash column chromatography (silica, EtOAc in heptane from 00/100 to 100/0). The desired fractions were collected and the solvents removed in vacuo to yield intermediate II-12b as a yellow solid (160 mg, 23%, 50% purity).

### Synthesis of Intermediate II-13

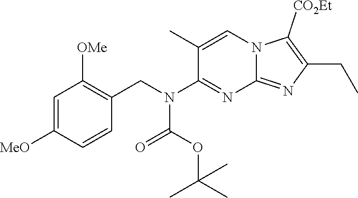 [SMILES: CCOC(=O)c1c(CC)nc2nc(N(Cc3ccc(OC)cc3OC)C(=O)OC(C)(C)C)c(C)cn12]

Di-tertbutyl dicarbonate [24424-99-5] (0.12 mL, 0.51 mmol) was added to a stirred solution of intermediate II-12a (136 mg, 0.34 mmol), triethylamine [121-44-8] (0.14 mL, 1.02 mmol) and 4-(dimethylamino)pyridine [1122-58-3] (2 mg, 0.017 mmol), in 1,4-dioxan (1.16 mL) at rt. The mixture was stirred at rt for 16 h and then more triethylamine [121-44-8] (47 μL, 0.34 mmol) and di-tertbutyl dicarbonate [24424-99-5] (78 μL, 0.34 mmol) were added and the mixture was stirred at 50° C. for 20 h. Additional triethylamine [121-44-8] (71 μL, 0.51 mmol) and di-tertbutyl dicarbonate [24424-99-5] (117 μL, 0.51 mmol) were added and the mixture was stirred at 80° C. for 2 h. Then more triethylamine [121-44-8] (94 μL, 0.68 mmol), 4-(dimethylamino)pyridine [1122-58-3] (2 mg, 0.017 mmol) and di-tertbutyl dicarbonate [24424-99-5] (117 μL, 0.51 mmol) were added and the reaction mixture was stirred at 100° C. for a further 16 h. The reaction mixture was diluted with water and brine and extracted with DCM (×3). The combined organic layers were dried (MgSO₄), filtered and concentrated in vacuo. The crude product was purified by flash column chromatography (silica; EtOAc in heptane from 0/100 to 30/70). The desired fractions were collected and concentrated in vacuo to yield intermediate II-14 as a beige solid (170 mg, 86%, 86% purity).

### Synthesis of Intermediate II-15

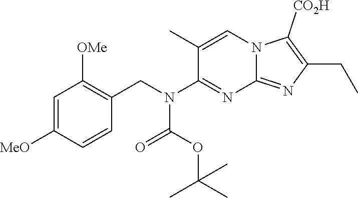 [SMILES: CCc1nc2nc(N(Cc3ccc(OC)cc3OC)C(=O)OC(C)(C)C)c(C)cn2c1C(=O)O]

Lithium hydroxide monohydrate [1310-66-3] (43 mg, 1.02 mmol) was added to a solution of intermediate II-14 (170 mg, 0.34 mmol) in a mixture of ethanol (5.12 mL) and water (1.71 mL) at rt. The reaction mixture was stirred at rt for 16 h. Then the mixture was brought to pH 7 by a 1M HCl aqueous solution addition. The solvent was evaporated in vacuo to yield intermediate II-15 as an orange solid that was used in the next step without any further purification (198 mg, quantitative).

### Synthesis of Intermediate II-16

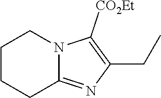 [SMILES: CCOC(=O)c1c(CC)nc2n1CCCC2]

Platinum(IV) oxide [1314-15-4] (73 mg, 0.32 mmol) was added to a stirred solution of intermediate II-1d (0.78 g, 3.56 mmol) in methanol (8 mL) under N₂. Then a concentrated HCl aqueous solution (2μ was added, and the resulting suspension was stirred at room temperature under H₂ atmosphere for 16 h. The reaction was filtered through a pad of Celite® and the filtrate was evaporated in vacuo to yield intermediate II-16 as a colourless oil (785 mg, 94%).

### Synthesis of Intermediate II-17a

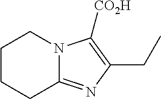

Lithium hydroxide monohydrate [1310-66-3] (85 mg, 2.02 mmol) was added to a stirred solution of intermediate II-16 (300 mg, 1.35 mmol), in a mixture of ethanol (5 mL) and water (2 mL) at rt. The mixture was stirred at 50° C. for 16 h and then the solvents were evaporated in vacuo to yield intermediate II-17a as a brown solid (262 mg, quantitative).

### Synthesis of Intermediate II-17b

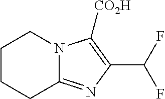 [SMILES: O=C(O)c1c(C(F)F)nc2n1CCCC2]

Lithium hydroxide monohydrate [1310-66-3] (29 mg, 0.69 mmol) was added to a stirred solution of intermediate II-5b (111 mg, 0.46 mmol), in a mixture of THE (4 mL) and water (1.5 mL) at rt. The mixture was stirred at rt for 16 h and then neutralized by a 1M HCl aqueous solution addition. The solvents were evaporated in vacuo to yield intermediate II-17b as a white solid (120 mg, quantitative).

### Synthesis of Intermediate II-17c

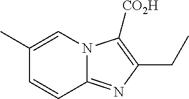

Lithium hydroxide monohydrate [1310-66-3] (135 mg, 3.23 mmol) was added to a stirred solution of intermediate II-Id (250 mg, 1.08 mmol), in a mixture of ethanol (4.4 mL) and water (2.2 mL) at rt. The mixture was stirred at 50° C. for 16 h and then neutralized by a 1M HCl aqueous solution addition. The solvents were evaporated in vacuo to yield intermediate II-17c as an orange solid (448 mg, quantitative).

### Synthesis of Intermediate II-17d

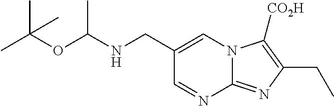 [SMILES: CCc1nc2ncc(CNC(C)OC(C)(C)C)cn2c1C(=O)O]

Sodium hydroxide (61 mg, 0.3 mmol), was added to a solution of intermediate II-12b (172 mg, 0.49 mmol) at rt. The mixture was stirred at rt for 16 h. The reaction mixture was brought to pH 7 by addition of a 1M HCl aqueous solution and the solvent was evaporated in vacuo to yield intermediate II-17d as a yellow solid that was used in the next step without any further purification (187 mg, 80%, 80% purity)

### Synthesis of Intermediates I

Synthesis of intermediate I-82

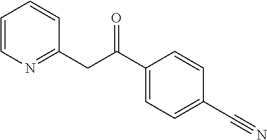 [SMILES: N#Cc1ccc(C(=O)Cc2ccccn2)cc1]

Intermediate I-82 was prepared according to an analogous procedure to the one used for the synthesis of intermediate I-23 using 2-Methylpyridine [109-06-8] (2.1 mL, 2.13 mmol) and Ethyl 4-cyanobenzoate [7153-22-2] (4.1 g, 23.4 mmol) as starting material. (1.8 g, 36%) as a bright yellow solid.

### Synthesis of Intermediate I-83

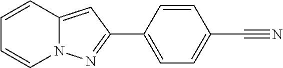 [SMILES: N#Cc1ccc(-c2cc3ccccn3n2)cc1]

Intermediate I-83 was prepared according to an analogous procedure to the one used for the synthesis of intermediate I-24 using 2 intermediate I-23 (1.8 g, 8.1 mmol) and intermediate I-1 (5.8 g, 20.2 mmol) as starting material. (179 mg, 9.6%) as a yellow solid.

### Synthesis of Intermediate I-84

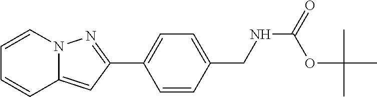 [SMILES: CC(C)(C)OC(=O)NCc1ccc(-c2cc3ccccn3n2)cc1]

Intermediate I-84 was prepared according to an analogous procedure to the one used for the synthesis of intermediate I-25 using intermediate I-83 (176 mg, 0.8 mmol) as starting material. (219 mg, 83.5%) as a white solid.

### Synthesis of Intermediate I-85

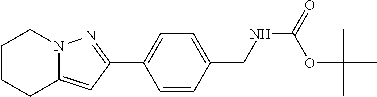 [SMILES: CC(C)(C)OC(=O)NCc1ccc(-c2cc3n(n2)CCCC3)cc1]

Intermediate I-85 was prepared according to an analogous procedure to the one used for the synthesis of intermediate I-26 using intermediate I-84 (206 mg, 0.6 mmol) as starting material. (100 mg, 47.5%) as a pale yellow solid.

### Synthesis of Intermediate I-86

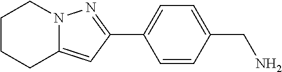 [SMILES: NCc1ccc(-c2cc3n(n2)CCCC3)cc1]

Intermediate I-86 was prepared according to an analogous procedure to the one used for the synthesis of intermediate I-27 using intermediate I-85 (97 mg, 0.3 mmol) as starting material. (89 mg, 99.1%) as a white solid.

Synthesis of intermediates I-87 (S) and I-88 (R)

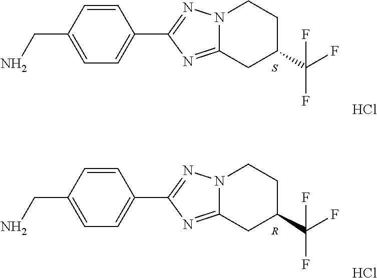

Intermediate I-6 (3 g, 8.125 mmol) was purified by chiral SFC on a Jasco SFC prep system using an Phenomenex Lux Cellulose-1 250 mm long×30 mm I.D. 5 m particle size, on isocratic mode at 100 ml/min of CO₂ (65%)−Methanol (40%)+0.1% DEA, at 30° C., BPR 150 Bar. Acquisition frequency was set to 220 nm for the DAD detector. The desired fractions were collected, evaporated, and dried under vacuo to yield intermediate I-87 (S) (1.3 g, 42.7%) and intermediate I-88 (R) (1.1 g, 34.7%) as white solids.

### Synthesis of Intermediate I-89

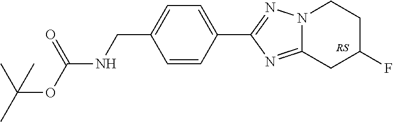

(Diethylamino)sulfur trifluoride [38078-09-0] (58 uL, 0.439 mmol) as added dropwise at −78° C. to a solution of I-63 (72 mg, 0.210 mmol) in anhydrous DCM (3 mL). The reaction mixture was slowly warmed up to rt for 2h. Saturated aqueous NaHCO₃ solution was added, and the mixture was extracted with DCM (×3). The combined organic layers were dried over MgSO4, filtered and concentrated in vacuo to yield intermediate I-89 (72 mg, 95%) as a yellow solid

### Synthesis of Intermediate I-90

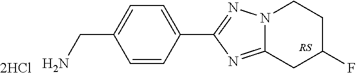

HCl 4M in dioxane (369 uL, 1.476 mmol) was added to a stirred solution of I-89 (85 mg, 0.245 mmol) in DCM (3 mL) in a round bottom flask at rt. The reaction mixture was stirred at rt for 16 h. The reaction was concentrated in vacuo to yield intermediate I-90 (78 mg, 99%) as a white solid.

### Synthesis of Intermediate I-91

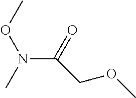

To a solution of 2-methoxyacetyl chloride [38870-89-2] (2.1 mL, 22.969 mmol) in DCM (25 mL) cooled to 0° C., was added N,O-Dimethylhydroxylamine hydrochloride [6638-79-5] (1.5 g, 24.557 mmol) and triethylamine [121-44-8] (9.6 mL, 68.876 mmol) and the reaction mixture was warmed to room temperature and stirred for 16 h. The reaction mixture was poured into aqueous saturated NaHCO₃ solution and extracted with DCM. The combined organic layers were dried over anhydrous MgSO₄, filtered, and concentrated in vacuo to yield intermediate I-91 (1.6 g, 52%) as a yellow oil.

### Synthesis of Intermediate I-92

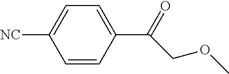

Iso-propyl magnesium chloride 2M solution in THF [1068-55-9] (4.9 mL, 9.8 mmol) was added to an ice-cooled solution on 4-iodobenzonitrile [3058-39-7] (1.9 g, 8.296 mmol) in anhydrous THE (17 mL). The solution was stirred at this temperature for 10 minutes and then cooled to −78° C. An ice-cooled solution of intermediate I-91 (1.6 g, 12.017 mmol) in anhydrous THE (8 mL) was added dropwise and the reaction was stirred at −78° C. for 1 h. Then it was stirred at rt for 1 h. The reaction mixture was treated with ammonium chloride solution and extracted with DCM. The organic phase was dried over anhydrous MgSO4, filtered, and concentrated in vacuo. The crude product was purified by flash column chromatography (silica, 25 g, Heptane/EtOAc 100:0 to 60:40). The desired fractions were collected to yield intermediate I-92 (920 mg, 63%) as a white solid.

### Synthesis of Intermediate I-93

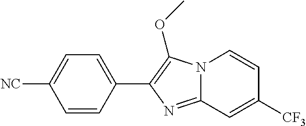 [SMILES: COc1c(-c2ccc(C#N)cc2)nc2cc(C(F)(F)F)ccn12]

A sealed tube was charged with intermediate I-92 (965 mg, 5.509 mmol), 2-amino-4-(trifluoromethyl)pyridine [106447-97-6] (2.7 g, 16.655 mmol), iodine [7553-56-2] (2.8 g, 11.032 mmol) and 1,2-dichloroethane (110 mL). The reaction mixture was stirred at 100° C. for 30 min. The mixture was diluted with an aqueous saturated sodium bisulfite solution and then extracted with DCM. The combined organic layers were dried over anhydrous MgSO₄, filtered, and concentrated in vacuo to give a red solid, that was purified by flash chromatography (silica, 80 g, ethyl acetate in heptane from 0/100 to 50/50). The desired fractions were collected and the solvents evaporated in vacuo to yield intermediate I-93 (1.4 g, 61%) as an orange solid.

### Synthesis of Intermediate I-94

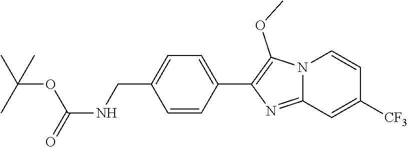 [SMILES: COc1c(-c2ccc(CNC(=O)OC(C)(C)C)cc2)nc2cc(C(F)(F)F)ccn12]

NaBH₄ [16940-66-2] (358 mg; 9.463 mmol) was added portionwise to a suspension of intermediate I-93 (1.3 g; 3.155 mmol), Nickel (II) chloride hexahydrate [7791-20-0] (750 mg; 3.155 mmol) and di-tertbutyl dicarbonate [24424-99-5] (2.2 mL; 9.463 mmol) in MeOH (13 mL) and 1,4-dioxane (6 mL) in a round bottom flask under nitrogen at 0° C. The reaction mixture was stirred at room temperature for 1 hour. H₂O and 25% aqueous ammonia were added, and the mixture was extracted with DCM. The combined organic layers were separated and dried over MgSO₄, filtered and concentrated in vacuo to give a dark orange oil, that was purified by flash chromatography (silica 80 g, ethyl acetate in DCM from 0/100 to 10/90). The desired fractions were collected to yield intermediate I-94 (834 mg, 61%) as light-yellow solid.

### Synthesis of Intermediate I-95

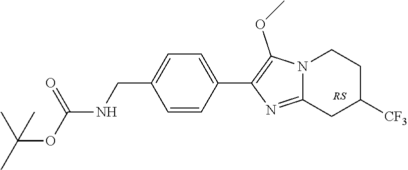 [SMILES: COc1c(-c2ccc(CNC(=O)OC(C)(C)C)cc2)nc2n1CCC(C(F)(F)F)C2]

In a round bottom flask under nitrogen atmosphere Pd/C [7440-05-3] (300 mg, 0.282 mmol) was added to a solution of intermediate I-94 (340 mg, 0.807 mmol) in methanol (12 mL) and ethyl acetate (3.2 mL) at 0° C. The mixture was stirred under H₂ atmosphere [1333-74-0] at 50° C. for 16 hours. The mixture was filtered through of pad of Celite® and the solvents were concentrated in vacuo. The crude product was purified by flash column chromatography (silica, 25 g, EtOAc in Heptane 0/100 to 50/50) and the desired fractions were collected to yield intermediate I-95 (53 mg, 15%) as a white solid.

### Synthesis of Intermediate I-96

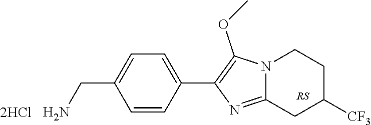 [SMILES: COc1c(-c2ccc(CN)cc2)nc2n1CCC(C(F)(F)F)C2]

HCl 4M in 1,4-dioxane [7647-01-0] (330 μL, 1.312 mmol) was added to a solution of intermediate I-95 (53 mg, 0.125 mmol) and VILL_scobos_124_1 (40 mg, 0.094) in dichloromethane (2 mL) in a round bottom flask at rt. The mixture was stirred at rt for 18 h.

The mixture was concentrated in vacuo to yield intermediate I-96 (88 mg, 99%) as yellow solid.

### Synthesis of Intermediate I-97

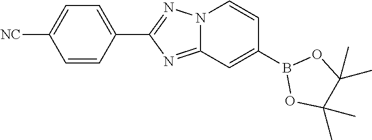 [SMILES: CC1(C)OB(c2ccn3nc(-c4ccc(C#N)cc4)nc3c2)OC1(C)C]

In a screw cap vial, Pd(dppf)Cl₂·CH₂Cl₂ [95464-05-4] (600 mg, 0.7355 mmol) was added to a solution of intermediate I-12 (2 g, 6.68 mmol), bis(pinacolato)diboron [73183-34-3] (3.3 g, 13.13 mmol) and potassium acetate [127-08-2] (2.67 g, 27.2 mmol) in dry 1,4-dioxane (20 mL) while nitrogen was bubbling for 10 min. Then, the mixture was stirred at 80° C. for 16 h. The mixture was filtered through of a pad of Celite®, rinsed with EtOAc. The solvent was concentrated in vacuo to yield intermediate I-97 (2.43 g, 79%) as a brown solid.

### Synthesis of Intermediate I-98

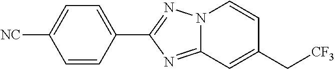 [SMILES: N#Cc1ccc(-c2nc3cc(CC(F)(F)F)ccn3n2)cc1]

A solution of intermediate I-97 (2.43 g, 1.44 mmol) and 1-Iodo-2,2,2-trifluoroethane [353-83-3] (1.38 mL, 14 mmol) in 1,4-dioxane (52 ml) was added to a suspension of Pd₂(dba)₃ [51364-51-3] (64 mg, 0.07 mmol), XantPhos [161265-03-8] (162 mg, 0.28 mmol) and Cesium carbonate [534-17-8] (4.5 g, 14.0 mmol) under nitrogen at rt. The mixture was stirred at rt for 1 min, then water (2.5 mL) was added. The mixture was stirred at 80° C. for 12 h. The mixture was cooled to rt. The mixture was diluted with water and extracted with AcOEt. The organic layer was separated, washed with brine, dried (MgSO₄), filtered and the solvents evaporated in vacuo. The crude product was purified by flash column chromatography (silica; AcOEt in heptane 0/100 to 30/70). The desired fractions were collected and concentrated in vacuo to yield intermediate I-98 (700 mg, 23%) as brown solid.

### Synthesis of Intermediate I-99

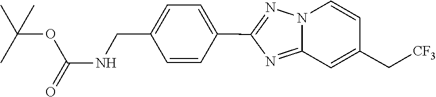 [SMILES: CC(C)(C)OC(=O)NCc1ccc(-c2nc3cc(CC(F)(F)F)ccn3n2)cc1]

Sodium borohydride [16940-66-2] (264 mg, 6.9 mmol) was added portionwise to a suspension of intermediate I-98 (700 mg, 2.3 mmol), Nickel(II) chloride hexahydrate [7791-20-0] (552 mg, 2.3 mmol) and Di-tertbutyl dicarbonate [24424-99-5] (1.6 mL, 6.9 mmol) in methanol (33 mL) and 1,4-dioxane (15 mL) in a round bottom flask at 0° C. The reaction mixture was stirred at rt for 16 h. Water and aqueous NH₃ were added, and the mixture was extracted with DCM (×3). The crude product was purified by flash column chromatography (dry load on silica, 80 g; EtOAc in heptane (from 0/100 to 30/60)). The desired fractions were collected and concentrated in vacuo to yield intermediate I-99 (404 mg, 37%) as a brown solid.

### Synthesis of Intermediate I-100a

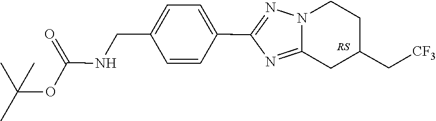 [SMILES: CC(C)(C)OC(=O)NCc1ccc(-c2nc3n(n2)CCC(CC(F)(F)F)C3)cc1]

Pd(OH)₂ [12135-22-7] (41 mg, 0.058 mmol) was added to a solution of yield intermediate I-99 (25 mg, 0.058 mmol) in MeOH (3 mL) and EtOAc (1 mL) in a round bottom flask under nitrogen atmosphere at 0° C. The mixture was stirred under H₂ atmosphere [1333-74-0] at rt for 16 hours. The mixture was filtered through of pad of celite© and solvents were concentrated in vacuo. The crude product was purified by flash column chromatography (silica 25 g; EtOAc in Heptane from 0/100 to 80/20). The desired fractions were collected and concentrated in vacuo to yield intermediate I-100a (155 mg, 43%) as a white solid.

### Synthesis of Intermediate I-100b

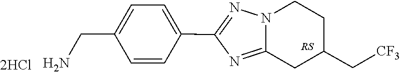 [SMILES: NCc1ccc(-c2nc3n(n2)CCC(CC(F)(F)F)C3)cc1]

HCl 4M in 1,4-dioxane [7647-01-0] (0.56 mL, 2.26 mmol, 4M) was added to a solution of intermediate I-100a (155 mg, 0.38 mmol) in DCM (3 mL) in a round bottom flask at rt. The mixture was stirred at rt for 16 h. The mixture was concentrated in vacuo to yield intermediate I-100b (152 mg, 95%) as a white solid.

### Synthesis of Intermediate I-101

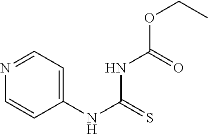

In a round bottom flask, ethoxycarbonyl isothiocyanate (9.300 mL, 78.851 mmol) was added to a solution of methyl 2-aminopyridine-4-carboxylate (10 g, 65.724 mmol) in 1,4-dioxane (70 mL) at rt. The mixture was stirred at rt for 16 hours. The solvent was removed in vacuo and the crude was triturated with diethyl ether, the solid was filtered and dried in vacuo to yield intermediate I-101 (19.356 g, 99%) as a yellow solid.

### Synthesis of Intermediate I-102

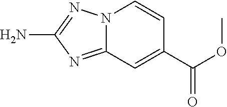

In a round bottom flask, a mixture of intermediate I-101 (19.356 g, 68.322 mmol), hydroxylamine hydrochloride (27.461 g, 395.176 mmol), DIPEA (42.1 mL, 241.707 mmol) and methanol (180 mL) was stirred at 66° C. for 16 hours. The solvent was removed in vacuo and the residue was washed with water. The solid was collected by filtration and washed with diethyl ether. Then the product was dried under vacuo to yield intermediate I-102 (7.857 g, 57%) as a grey solid.

### Synthesis of Intermediate I-103

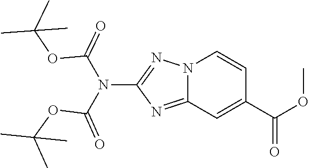 [SMILES: COC(=O)c1ccn2nc(N(C(=O)OC(C)(C)C)C(=O)OC(C)(C)C)nc2c1]

In a round bottom flask, di-tertbutyl dicarbonate [24424-99-5] (23.5 mL, 102.292 mmol) was added to a solution of intermediate I-102 (7.857 g, 40.885 mmol), 4-(dimethylamino)pyridine (504 mg, 4.125 mmol) and triethylamine (11.4 mL, 81.791 mmol) in acetonitrile (100 mL) at rt. The mixture was stirred at 70° C. for 8 hours. The reaction mixture was diluted with H₂O and brine and extracted with DCM (×3). The combined organic layers were dried over anhydrous MgSO₄, filtered, and concentrated in vacuo to give a dark oil. The crude product was purified by flash column chromatography (silica, 80 g; (AcOEt in heptane from 0/100 to 35/65)). The desired fractions were collected and concentrated in vacuo to yield intermediate I-103 (11.520 g, 71%) as a brown solid.

### Synthesis of Intermediate I-104

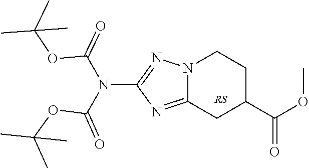 [SMILES: COC(=O)C1CCn2nc(N(C(=O)OC(C)(C)C)C(=O)OC(C)(C)C)nc2C1]

Pd/C [7440-05-3] (6.5 g, 6.1 mmol) was added to a solution of intermediate I-103 (4 g, 10.1 mmol) in methanol (60 mL) and ethyl acetate (20 mL) in a round bottom flask under nitrogen atmosphere at 0° C. The mixture was stirred under H₂ atmosphere [1333-74-0] at 50° C. for 16h. Pd/C [7440-05-3] (3.2 g, 3.0 mmol) was added to the reaction under nitrogen atmosphere at 0° C. The mixture was stirred under H₂ atmosphere [1333-74-0] at 50° C. for 16 h. The reaction was filtered through pad of Celite®. Solvent was concentrated in vacuo to give a colorless oil. The crude product was purified by flash column chromatography (silica 120 g; EtOAc in heptane 0/100 to 60/40). The desired fractions were collected and concentrated in vacuo to yield intermediate I-104 (2.6 g, 61%) as a white solid.

### Synthesis of Intermediate I-105

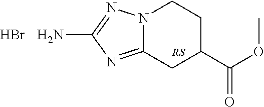

HBr [10035-10-6] (4.3 mL, 38 mmol) was added to a solution of yield intermediate I-104 (5.0 g, 12.6 mmol) in dichloromethane (38 mL) in a round bottom flask at rt. The mixture was stirred at rt for 16 h. HBr [10035-10-6] (4.3 mL, 38 mmol) was added to the reaction and the mixture was stirred at rt for 2 h. The mixture was concentrated in vacuo to yield intermediate I-105 (3.642 g, 99%) as a white solid.

### Synthesis of Intermediate I-106

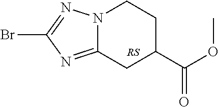

In a round bottom flask, hydrobromic acid [10035-10-6] (9.100 mL, 50.1 mmol) was added to a stirred suspension of intermediate I-105 (3.6 g, 12.3 mmol) and acetic acid [64-19-7] (11 mL, 192.1 mmol) at 0° C. Then sodium nitrite [7632-00-0] (1.0 g, 14.5 mmol) was dissolved in water (19 mL) and added dropwise at 0° C. The reaction mixture was stirred at 0° C. for 2h. Then Copper (I) Bromide [7787-70-4] (448 mg, 3.1 mmol) was added at 0° C. and the mixture was heated at 90° C. for 16 h. The reaction was cooled, and methanol (50 mL) was added at rt. The mixture was stirred at 65° C. for 16 h. The reaction mixture was cooled, quenched with saturated aqueous NaHCO₃ solution, and extracted with DCM (×3). The combined organic layers were dried over MgSO₄, filtered and concentrated in vacuo to yield intermediate I-106 as a yellowish oil (2.33 g, 69%).

### Synthesis of Intermediate I-107

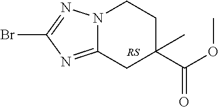

In a round bottom flask, LiHDMS [4039-32-1] (7.7 mL, 7.7 mmol) was added at −78° C. under N₂ atmosphere to a solution of intermediate I-106 (1 g, 3.845 mmol) in anhydrous THE (50 mL). The mixture was stirred at −78° C. for 15 min and then was stirred at 0° C. for 15 min. After, Iodomethane (0.6 mL, 9.638 mmol) was added at −78° C. and the mixture was stirred at rt. The mixture was diluted with DCM and excess reactants are consumed by the addition of a saturated aqueous solution of NH₄Cl. The organic layer was washed with brine and concentrated in vacuo to yield intermediate I-107 as a pale-yellow solid (1.16 mg, 99.1%).

### Synthesis of Intermediate I-108

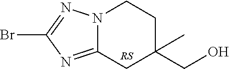

LiBH₄ [16949-15-8] (131 mg, 5.713 mmol) was added to a solution of intermediate I-107 (1.16 g, 3.809 mmol) in dry THE (60 mL) under nitrogen atmosphere and the reaction mixture was stirred at room temperature for 16 h. The mixture was diluted with DCM and excess reactants are consumed by the addition of a saturated aqueous solution of NH₄Cl. The crude product was purified by flash column chromatography (silica 20 g; EtOAc in DCM 0/100 to 100/0). The desired fractions were collected and concentrated in vacuo to yield intermediate I-108 as a white solid (345 mg, yield: 36.4%).

### Synthesis of Intermediate I-109

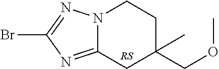

Sodium hydride [7646-69-7] (59 mg, 1.475 mmol) was added to a solution of intermediate I-108 (200 mg, 0.813 mmol) in DMF (10 mL) at 0° C. under nitrogen atmosphere. The mixture was stirred at the same temperature for 30 min followed by the addition of iodomethane [74-88-4] (76 uL, 1.221 mmol). The reaction mixture was stirred at room temperature for 16 hours. Sodium hydride [7646-69-7] (33 mg, 0.825 mmol) and iodomethane [74-88-4] (25 uL, 0.402 mmol) were added to the mixture at 0° C. The mixture was stirred at room temperature for 16 hours. The mixture was diluted with DCM and excess reactants are consumed by the addition of a saturated aqueous solution of NH₄Cl. The organic layer was washed with brine and concentrated in vacuo to yield intermediate I-109 as a yellow solid (212 mg, yield: 99.3 0%).

### Synthesis of Intermediate I-110

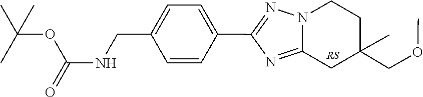 [SMILES: COCC1(C)CCn2nc(-c3ccc(CNC(=O)OC(C)(C)C)cc3)nc2C1]

In a glass pressure tube, tert-butyl N-{[4-(tetramethyl-1,3,2-dioxaborolan-2-yl)phenyl]methyl}carbamate [330794-35-9] (301 mg, 0.903 mmol), Cs₂CO₃ [534-17-8] (584 mg, 1.792 mmol) and Pd(dppf)Cl2 [95464-05-4] (100 mg, 0.122 mmol) were added to a solution of intermediate I-109 (212 mg, 0.815 mmol) in dioxane (6.4 mL) and water (2.6 mL) while bubbling with N₂. The reaction mixture was stirred at 90° C. for 16 hours. Water was added and then the mixture was extracted with EtOAc (×3). The combined organic layers were washed with brine, dried over anhydrous MgSO₄, filtered, and concentrated in vacuo. The crude product was purified by flash column chromatography (silica 25 g; DCM:MeOH 9:1 in DCM 0/100 to 50/50). The desired fractions were collected and concentrated in vacuo to yield intermediate I-110 as a brown wax (277 mg, yield: 87.1%).

### Synthesis of Intermediate I-111

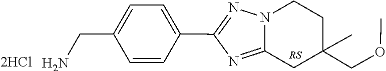 [SMILES: COCC1(C)CCn2nc(-c3ccc(CN)cc3)nc2C1]

A 4M solution of HCl in 1,4-dioxane [7647-01-0] (1.1 mL, 4.4 mmol) was added to a stirred solution of intermediate I-110 (277 mg, 0.710 mmol) in DCM (5 mL) in a round bottom flask at room temperature. The reaction mixture was stirred at room temperature for 5 min. A 4M solution of HCl in 1,4-dioxane [7647-01-0] (1.1 mL, 4.4 mmol) was added to the reaction. The mixture was stirred at room temperature for 6 hours. The crude product was concentrated in vacuo to yield intermediate I-111 as a brown solid (255 mg, yield: 99.0%).

### Synthesis of Intermediate I-112

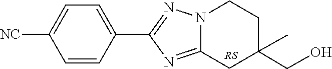 [SMILES: CC1(CO)CCn2nc(-c3ccc(C#N)cc3)nc2C1]

In a glass pressure tube, 4-cyanophenylboronic acid [126747-14-6] (525 mg, 3.57 mmol), Cs₂CO₃ [534-17-8] (2.30 g, 7.06 mmol) and Pd(dppf)Cl2 [95464-05-4] (395 mg, 0.48 mmol) were added to a solution of intermediate I-108 (790 mg, 3.42 mmol) in 1,4-dioxane (20 mL) and water (8 mL) while bubbling with N₂. The reaction mixture was stirred at 90° C. for 16 hours. Water was added and then the mixture was extracted with EtOAc (×3). The combined organic layers were washed with brine, dried over anhydrous MgSO₄, filtered and concentrated in vacuo. The crude product was purified by flash column chromatography (silica 25 g; EtOAc in DCM 0/100 to 80/20). The desired fractions were collected and concentrated in vacuo to yield intermediate I-112 as a pale brown solid (490 mg, 55%).

### Synthesis of Intermediate I-113

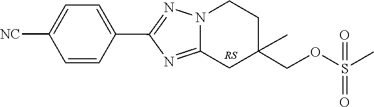 [SMILES: CC1(COS(C)(=O)=O)CCn2nc(-c3ccc(C#N)cc3)nc2C1]

Methanesulfonyl chloride [124-63-0] (0.18 mL, 2.33 mmol) was added dropwise to a stirred solution of intermediate I-112 (485 mg, 1.81 mmol), triethylamine [121-44-8] (0.37 mL, 0.280 mmol) and DMAP [1122-58-3] (12 mg, 0.10 mmol) in DCM (25 mL). The mixture was stirred at room temperature for 16 hours. The mixture was diluted with water and extracted with DCM (×3). The organic layer was dried over MgSO₄, filtered, and concentrated in vacuo to yield intermediate I-113 as a pale brown solid (562 mg, 89%).

### Synthesis of Intermediate I-114

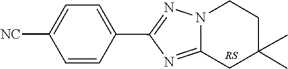 [SMILES: CC1(C)CCn2nc(-c3ccc(C#N)cc3)nc2C1]

Zinc powder [7440-66-6] (526 mg, 8.04 mmol) and sodium iodide [7681-82-5] (483 mg, 3.22 mmol) were added to a solution of intermediate I-113 (560 mg, 1.62 mmol) in DMF (15 mL). The reaction mixture was stirred at 110° C. for 18 hours. Zinc powder [7440-66-6] (5 eq., 526 mg, 8.04 mmol) and sodium iodide [7681-82-5] (2 eq., 483 mg, 3.22 mmol) were added to the reaction mixture and was stirred at 110° C. for 6 hours. Zinc powder [7440-66-6] (5 eq., 526 mg, 8.04 mmol) and sodium iodide [7681-82-5] (2 eq., 483 mg, 3.22 mmol) were added to the reaction mixture and was stirred at 110° C. for 21 hours. The reaction mixture was filtered, and the filtrate was diluted with water and extracted with EtOAc (×3). The combined organic layers were concentrated in vacuo to yield intermediate I-114 (375 mg, 83%) as a yellow solid.

### Synthesis of Intermediate I-115

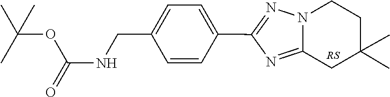 [SMILES: CC1(C)CCn2nc(-c3ccc(CNC(=O)OC(C)(C)C)cc3)nc2C1]

In a round bottom flask, sodium borohydride [16940-66-2] (167 mg, 4.41 mmol) was added portionwise to a suspension of intermediate I-114 (375 mg, 1.49 mmol), Nickel (II) chloride hexahydrate [7791-20-0] (267 mg, 1.12 mmol) and di-tertbutyl dicarbonate [24424-99-5](0.51 mL, 2.22 mmol) in dry methanol (20 mL) and dioxane (10 mL) at 0° C. The reaction mixture was stirred at room temperature for 18 hours. Water and aqueous NH₃ were added and the mixture was stirred for 10 min. The mixture was filtered through of pad of Celite® and washed with MeOH and was concentrated in vacuo the MeOH. Then, the mixture was extracted with DCM (×3). The combined organic layers were dried over anhydrous MgSO₄ and the solvents were concentrated in vacuo. The crude product was purified by flash column chromatography (silica, 25 g; EtOAc in DCM 0/100 to 50/50). The desired fractions were collected and the solvents evaporated in vacuo to yield intermediate I-115 as a white solid (291 mg, 53%).

### Synthesis of Intermediate I-116

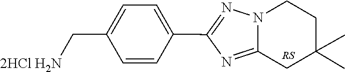

To a stirred solution of intermediate I-115 (194 mg, 0.54 mmol) in DCM (2 ml), a 4 M solution of HCl in 1,4-dioxane [7647-01-0] (1.1 ml, 4.4 mmol), was added at room temperature and the resulting mixture was stirred for 16 hours at room temperature. The solvents were removed under high vacuo to yield intermediate I-116 as a white solid (179 mg, 99%).

### Synthesis of Intermediate I-117

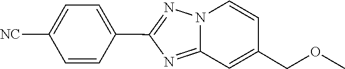 [SMILES: COCc1ccn2nc(-c3ccc(C#N)cc3)nc2c1]

In a screw cap vial, Cs₂CO₃ [534-17-8] (3.8 mg, 11.66 mmol) was added to a solution of intermediate I-12 (876 mg, 2.92 mmol) and potassium methoxymethyltrifluoroborate [910251-11-5] (946 mg, 5.9 mmol) in 1,4-dioxane (7.5 mL) and water (1 mL) at rt while bubbling N₂. Then, the mixture was bubbled with N₂ for 10 min. RuPhos Pd G3 [1445085-77-7] (245 mg, 0.29 mmol) and RuPhos [787618-22-8] (140 mg, 0.3 mmol) were added at rt and the mixture was bubbled with N₂ for 10 min. Then, the mixture was stirred at 100° C. for 16 h. The reaction was filtered through a pad of Celite® and the solvent was evaporated in vacuo. The crude was purified by flash column chromatography [silica 80 g; AcOEt in heptane from 0/100 to 50/50). The desired fractions were collected and concentrated in vacuo. The product was repurified by flash column chromatography [silica 2×25 g; AcOEt in DCM from 0/100 to 10/90). The desired fractions were collected and concentrated in vacuo to yield intermediate I-117 as a white solid (324 mg, 41%).

### Synthesis of Intermediate I-118

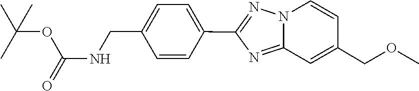 [SMILES: COCc1ccn2nc(-c3ccc(CNC(=O)OC(C)(C)C)cc3)nc2c1]

Sodium borohydride [16940-66-2] (140 mg, 3.7 mmol) was added portionwise to a suspension of intermediate I-117 (324 g, 1.22 mmol), nickel(II) chloride hexahydrate [7791-20-0] (350 mg, 1.47 mmol) and di-tertbutyl dicarbonate [24424-99-5] (0.85 mL, 2.87 mmol) in methanol (20 mL) and dioxane (10 mL) in a round bottom flask at 0° C. The reaction mixture was stirred at rt for 16 h. Water and 3 ml of aqueous NH₃ were added and the mixture was extracted with DCM (×3). The organic layer was dried over MgSO₄, filtered, and concentrated in vacuo. The crude product was purified by flash column chromatography (silica 25 g; AcOEt in heptane from 0/100 to 70/30). The desired fractions were collected and concentrated in vacuo to afford intermediate I-118 as a brown solid (336 mg, yield: 73.6%).

### Synthesis of Intermediate I-119

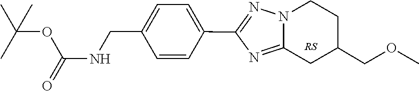 [SMILES: COCC1CCn2nc(-c3ccc(CNC(=O)OC(C)(C)C)cc3)nc2C1]

Pd(OH)₂ [12135-22-7] (1.0 eq., 119 mg, 0.847 mmol) was added to a solution of intermediate I-118 (311 mg, 0.844 mmol) in methanol (11.8 ml) and ethyl acetate (6.2 ml) in a round bottom flask under nitrogen atmosphere at 0° C. The mixture was stirred under H₂ atmosphere at room temperature for 18 hours. The mixture was filtered through of pad of celite and solvents were concentrated in vacuo. The crude product was purified by flash column chromatography (silica 25 g; EtOAc in DCM from 0/100 to 50/50). The desired fractions were collected and concentrated in vacuo to yield intermediate I-119 as a white solid (289 mg, 91%).

### Synthesis of Intermediate I-120

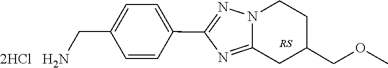 [SMILES: COCC1CCn2nc(-c3ccc(CN)cc3)nc2C1]

HCl in Dioxane [7647-01-0] (1.16 mL, 4.639 mmol) was added to a solution of intermediate I-119 (288 mg, 0.773 mmol) in dichloromethane (6 mL) in a round bottom flask at rt. The mixture was stirred at rt for 4 h. The mixture was concentrated in vacuo to yield intermediate I-120 as a white solid (251 mg, yield: 93%).

Intermediates included in next table were synthesized following similar methods as described previously.

| | Inter- |
|---|---|
| | mediate |
| Structure | Number |
| 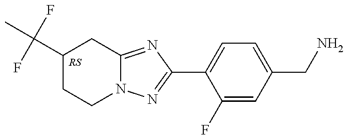 [SMILES: CC(F)(F)C1CCn2nc(-c3ccc(CN)cc3F)nc2C1] | I-121 |
| 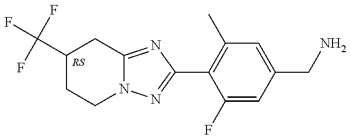 [SMILES: Cc1cc(CN)cc(F)c1-c1nc2n(n1)CCC(C(F)(F)F)C2] | I-122 |
| 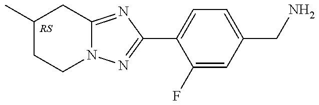 [SMILES: CC1CCn2nc(-c3ccc(CN)cc3F)nc2C1] | I-123 |
| 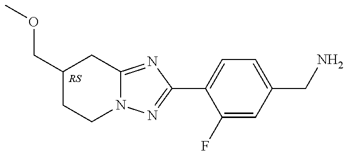 [SMILES: COCC1CCn2nc(-c3ccc(CN)cc3F)nc2C1] | I-124 |
| 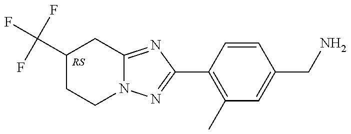 [SMILES: Cc1cc(CN)ccc1-c1nc2n(n1)CCC(C(F)(F)F)C2] | I-125 |
| 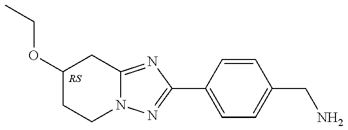 [SMILES: CCOC1CCn2nc(-c3ccc(CN)cc3)nc2C1] | I-126 |
| 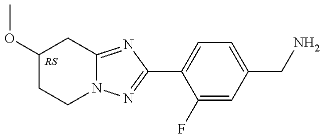 [SMILES: COC1CCn2nc(-c3ccc(CN)cc3F)nc2C1] | I-127 |
| 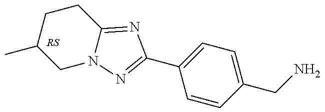 [SMILES: CC1CCc2nc(-c3ccc(CN)cc3)nn2C1] | I-128 |
| 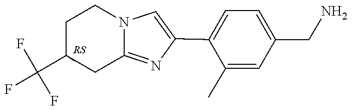 [SMILES: Cc1cc(CN)ccc1-c1cn2c(n1)CC(C)(C(F)(F)F)CC2] | I-129 |
| 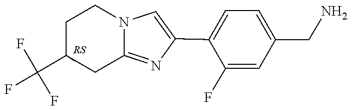 [SMILES: CC1(C(F)(F)F)CCn2cc(-c3ccc(CN)cc3F)nc2C1] | I-130 |
| 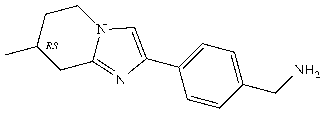 [SMILES: CC1CCn2cc(-c3ccc(CN)cc3)nc2C1] | I-131 |
| 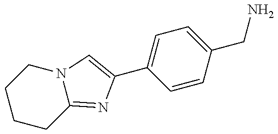 [SMILES: NCc1ccc(-c2cn3c(n2)CCCC3)cc1] | I-132 |
| 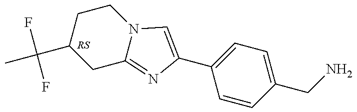 [SMILES: CC(F)(F)C1CCn2cc(-c3ccc(CN)cc3)nc2C1] | I-133 |
| 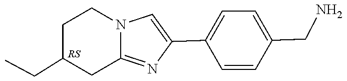 [SMILES: CCC1CCn2cc(-c3ccc(CN)cc3)nc2C1] | I-134 |
| 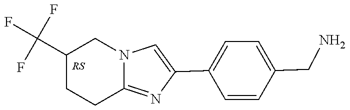 [SMILES: NCc1ccc(-c2cn3c(n2)CCC(C(F)(F)F)C3)cc1] | I-135 |
| 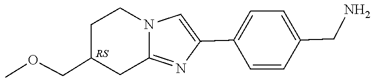 [SMILES: COCC1CCn2cc(-c3ccc(CN)cc3)nc2C1] | I-136 |
| 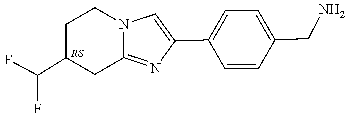 [SMILES: NCc1ccc(-c2cn3c(n2)CC(C(F)F)CC3)cc1] | I-137 |
| 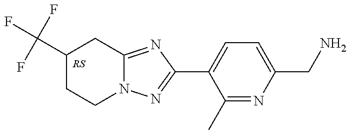 [SMILES: Cc1nc(CN)ccc1-c1nc2n(n1)CCC(C(F)(F)F)C2] | I-138 |
| 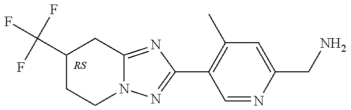 [SMILES: Cc1cc(CN)ncc1-c1nc2n(n1)CCC(C(F)(F)F)C2] | I-139 |
| 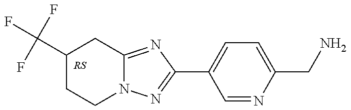 [SMILES: NCc1ccc(-c2nc3n(n2)CCC(C(F)(F)F)C3)cn1] | I-140 |
| 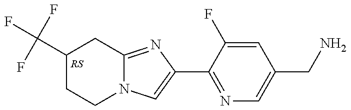 [SMILES: NCc1cnc(-c2cn3c(n2)CC(C(F)(F)F)CC3)c(F)c1] | I-141 |
| 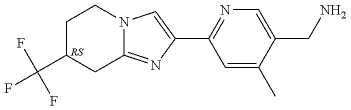 [SMILES: Cc1cc(-c2cn3c(n2)CC(C(F)(F)F)CC3)ncc1CN] | I-142 |
| 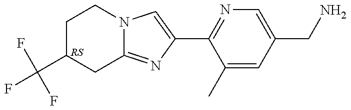 [SMILES: Cc1cc(CN)cnc1-c1cn2c(n1)CC(C(F)(F)F)CC2] | I-143 |
| 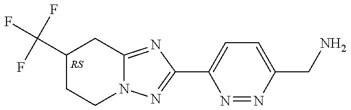 [SMILES: NCc1ccc(-c2nc3n(n2)CCC(C(F)(F)F)C3)nn1] | I-144 |
| 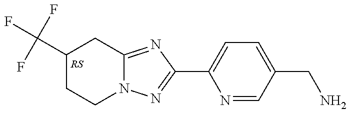 [SMILES: NCc1ccc(-c2nc3n(n2)CCC(C(F)(F)F)C3)nc1] | I-145 |
| 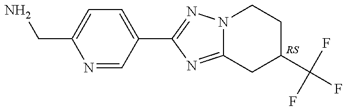 [SMILES: NCc1ccc(-c2nc3n(n2)CCC(C(F)(F)F)C3)cn1] | I-146 |
| 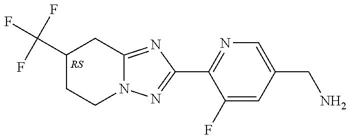 [SMILES: NCc1cnc(-c2nc3n(n2)CCC(C(F)(F)F)C3)c(F)c1] | I-147 |
| 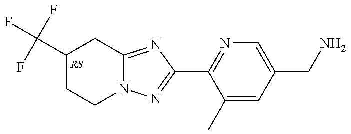 [SMILES: Cc1cc(CN)cnc1-c1nc2n(n1)CCC(C(F)(F)F)C2] | I-148 |
| 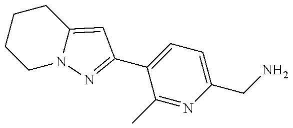 [SMILES: Cc1nc(CN)ccc1-c1cc2n(n1)CCCC2] | I-149 |
| 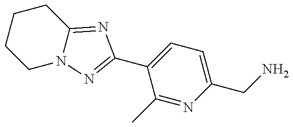 [SMILES: Cc1nc(CN)ccc1-c1nc2n(n1)CCCC2] | I-150 |
| 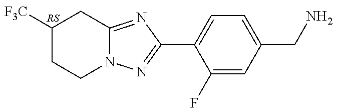 [SMILES: NCc1ccc(-c2nc3n(n2)CCC(C(F)(F)F)C3)c(F)c1] | I-150b |

### Synthesis of Intermediates I-151a and I-151b

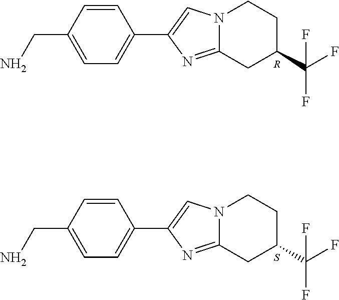

Intermediates I-151a and I-151b were prepared according to an analogous procedure to the one used for the synthesis of intermediates I-6a and I-6b using intermediate I-10 (2.2 g, 7.4 mmol) as starting material. Intermediate I-151a (508 mg, 99800 purity, 22.90%) and intermediate I-151b (415 mg, 92% purity, 17.4%) were obtained as a white solid.

### Synthesis of Intermediates I-152a and I-152b

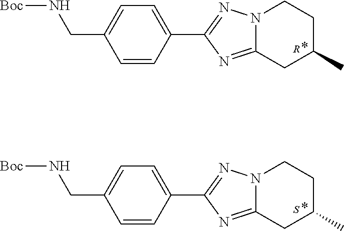

Intermediate I-14 (394 g, 1150.55 mmol) was purified by chiral SFC system using an Phenomenex Lux Cellulose-SZ 50 mm long×30 mm I.D. 3 m particle size, on gradient mode at 100 ml/min from CO₂/Methanol from 10/90 to 50/50+20 mM NH₃, at 35° C., BPR 2200 psi. Acquisition frequency was set to 220 nm for the DAD detector. This resulted to afford intermediate I-152a (144 g, 36.55%, separated two times by SFC) as a white solid and intermediate I-152b (161 g, 40.86%) as a white solid.

### Synthesis of Intermediates I-153

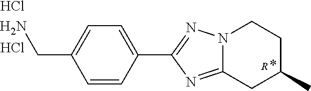

Into a 5 L 4-necked round-bottom flask was added intermediate I-152a (144 g) and DCM (3.2 L) at room temperature. To the above mixture was added HCl (gas) in 1,4-dioxane (705 mL, 4.0 M) dropwise at room temperature. The resulting mixture was stirred for overnight at room temperature under nitrogen atmosphere. The precipitated solids were collected by filtration and washed with DCM (2×1 L). This resulted in I-153 (142.4 g, 96.08%) as a light yellow solid.

### Synthesis of Intermediates I-154

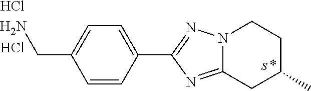

Intermediate I-154 was prepared according to an analogous procedure to the one used for the synthesis of intermediate I-153 using intermediate I-152b (161 g, 470.151 mmol) as starting material. (142.4 g, 96.08%) as a light yellow solid.

### Synthesis of Intermediate I-155

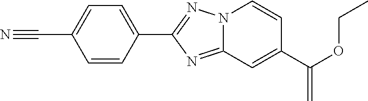 [SMILES: C=C(OCC)c1ccn2nc(-c3ccc(C#N)cc3)nc2c1]

Intermediate I-155 was prepared according to an analogous procedure to the one used for the synthesis of intermediate I-76 using intermediate I-12 (4.5 g, 15 mmol) as starting material. (4.2 g, 95%) as a light brown solid.

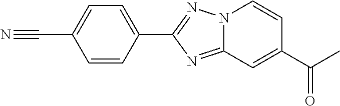 [SMILES: CC(=O)c1ccn2nc(-c3ccc(C#N)cc3)nc2c1]

An aqueous solution 6M of HCl (82 mL, 492 mmol) was added to a solution of I-155 (4.193 g, 14.443 mmol) in Dioxane (15 mL) in a round bottom flask at rt. The mixture was stirred at room temperature for 1 h. The reaction mixture was diluted with water, basified to pH 11 with an aqueous NaOH 6 N solution and extracted with DCM (×3). The combined organic layers were separated, dried (MgSO4), filtered and the solvents evaporated under vacuo to yield intermediate I-156 (3.665 g, 92%) as a brown solid.

### Synthesis of Intermediate I-157

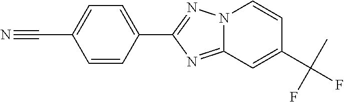 [SMILES: CC(F)(F)c1ccn2nc(-c3ccc(C#N)cc3)nc2c1]

In a round bottom flask, DAST [38078-09-0] (1.755 mL, 13.280 mmol) was added dropwise to a solution of intermediate I-156 (1.161 g, 4.427 mmol) in dichloroethane dry (33 mL) at 0° C. under nitrogen atmosphere. The mixture reaction was stirred at 45° C. for 120 hours. The mixture was diluted with NaHCO3 aq. sat. at 0° C. and extracted with DCM. The combined organic layers were washed with brine and dried over MgSO4, filtered and concentrated. The crude product was purified by flash column chromatography (dry on load, silica 80 g; EtOAc in heptane 0/100 to 0/100). The desired fractions were collected and concentrated to afford intermediate I-157 (337 mg, 26%) as a brown solid.

### Synthesis of Intermediate I-158

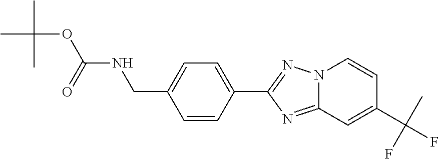 [SMILES: CC(C)(C)OC(=O)NCc1ccc(-c2nc3cc(C(C)(F)F)ccn3n2)cc1]

Intermediate I-158 was prepared according to an analogous procedure to the one used for the synthesis of intermediate I-4 using intermediate I-157 (922 mg, 3.2 mmol) as starting material. (870 mg, 67.7%) as a white solid.

### Synthesis of Intermediate I-159

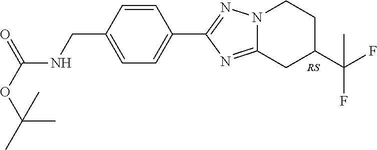 [SMILES: CC(C)(C)OC(=O)NCc1ccc(-c2nc3n(n2)CCC(C(C)(F)F)C3)cc1]

Intermediate I-159 was prepared according to an analogous procedure to the one used for the synthesis of intermediate I-5 using intermediate I-158 (805 mg, 2 mmol) as starting material. (651 mg, 83.4%) as a white solid.

### Synthesis of Intermediate I-160

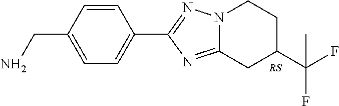 [SMILES: CC(F)(F)C1CCn2nc(-c3ccc(CN)cc3)nc2C1]

Intermediate I-160 was prepared according to an analogous procedure to the one used for the synthesis of intermediate I-6 using intermediate I-159 (651 mg, 1.7 mmol) as starting material. (614 mg, quantitative) as a white solid.

### Synthesis of Intermediates II

### Intermediate II-18

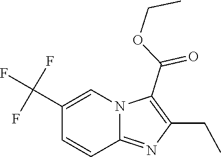 [SMILES: CCOC(=O)c1c(CC)nc2ccc(C(F)(F)F)cn12]

Intermediate II-18 was prepared according to an analogous procedure to the one used for the synthesis of intermediate II-1a, using 2-amino-5-(trifluoromethyl)pyridine [74784-70-6] (3 g, 18.5 mmol) as starting material. 11-18 was obtained as a orange solid (1.7 g, 28%, 88% purity).

### Intermediate II-19—LiCl Salt

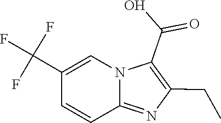 [SMILES: CCc1nc2ccc(C(F)(F)F)cn2c1C(=O)O]

Lithium hydroxide monohydrate [1310-66-3] (77 mg, 1.8 mmol) was added to a stirred solution of Intermediate II-18 (190 mg, 0.6 mmol), in a mixture of ethanol and water (1:1) 10 mL at room temperature. The reaction mixture was stirred at room temperature or at 50° C. for 16 h and then neutralized by a solution of HCl (1M, aq.). The solvents were evaporated in vacuo to yield intermediate II-19 (189 mg, quant.).

### Intermediate II-20

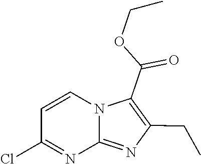 [SMILES: CCOC(=O)c1c(CC)nc2nc(Cl)ccn12]

Intermediate II-20 was prepared according to an analogous procedure to the one used for the synthesis of intermediate II-1a, using 2-amino-4-chloropyrimidine [3993-78-0]. (2 g, 15.4 mmol) as starting material. Intermediate II-20 was obtained as a yellow solid (1.8 g, 44.7%).

### Intermediate II-21

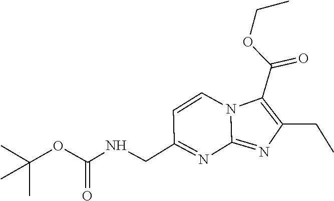 [SMILES: CCOC(=O)c1c(CC)nc2nc(CNC(=O)OC(C)(C)C)ccn12]

Intermediate II-21 was prepared according to an analogous procedure to the one used for the synthesis of intermediate II-12b, using intermediate II-20 (300 mg, 1.2 mmol) as starting material. Intermediate 1-21 was obtained as a yellow solid (90 mg, 85% purity, 18.6%).

### Intermediate II-22—NaCl Salt

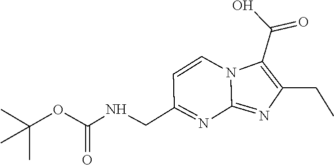 [SMILES: CCc1nc2nc(CNC(=O)OC(C)(C)C)ccn2c1C(=O)O]

Intermediate II-22 was prepared according to an analogous procedure to the one used for the synthesis of intermediate II-8a using II-21 (120 mg, 0.3 mmol) as starting material. Intermediate I-22 was obtained as a yellow solid (120 mg, 90% purity, 97.4%).

### Intermediate II-23

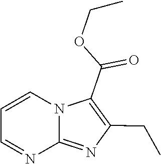 [SMILES: CCOC(=O)c1c(CC)nc2ncccn12]

Intermediate II-23 was prepared according to an analogous procedure to the one used for the synthesis of intermediate II-1a, using 2-aminopyrimidine [109-12-6]. (5 g, 52.6 mmol) as starting material. Intermediate II-23 was obtained as an orange solid (8.7 g, 74.5%).

### Intermediate II-24

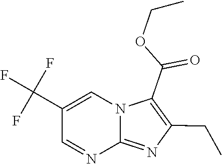 [SMILES: CCOC(=O)c1c(CC)nc2ncc(C(F)(F)F)cn12]

Intermediate II-24 was prepared according to an analogous procedure to the one used for the synthesis of intermediate II-1a, using 5-(Trifluoromethyl) pyrimidin-2-amine [69034-08-8](1.2 g, 7.4 mmol) as starting material. Intermediate II-24 was obtained as a yellow solid (825 mg, 90% purity, 35.1%).

### Intermediate II-25

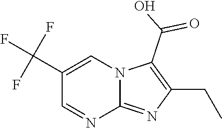 [SMILES: CCc1nc2ncc(C(F)(F)F)cn2c1C(=O)O]

In a round bottom flask, concentrated hydrochloric acid [7647-01-0] (5 mL) was added to Intermediate II-24 (250 mg, 0.870 mmol) and the reaction mixture was stirred at 85° C. for 30 h. Additional concentrated hydrochloric acid [7647-01-0] (2 mL) was added to the reaction mixture and it was stirred at 90° C. for 46 h. The solvent was concentrated in vacuo to yield intermediate II-25 (226 mg, yield: 70%) as a brown solid.

### Synthesis of Intermediate II-26

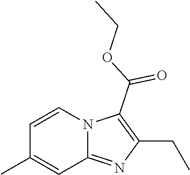 [SMILES: CCOC(=O)c1c(CC)nc2cc(C)ccn12]

Intermediate II-26 was prepared according to an analogous procedure to the one used for the synthesis of intermediate II-7d, using 2-amino-4-picoline [695-34-1] (3 g, 27.7 mmol) as starting material. II-26 was obtained as an orange solid (4.7 g, 71.9%).

### Synthesis of Intermediate II-27

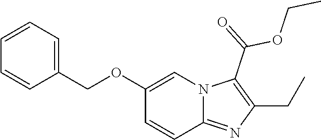 [SMILES: CCOC(=O)c1c(CC)nc2ccc(OCc3ccccc3)cn12]

Intermediate II-27 was prepared according to an analogous procedure to the one used for the synthesis of intermediate II-7d, using 5-(benzyloxy)pyridin-2-amine [96166-00-6] (1 g, 5 mmol) as starting material. Intermediate II-27 was obtained as an orange solid (1.1 g, 62.1%).

### Synthesis of Intermediate II-28

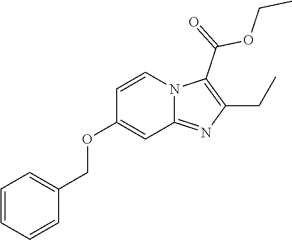 [SMILES: CCOC(=O)c1c(CC)nc2cc(OCc3ccccc3)ccn12]

Intermediate II-28 was prepared according to an analogous procedure to the one used for the synthesis of intermediate II-7d, using 4-(benzyloxy)pyridin-2-amine [85333-26-2] (0.8 g, 4.2 mmol) as starting material. Intermediate II-28 was obtained as a yellow solid (1.1 g, 84.3%).

### Synthesis of Intermediate II-29

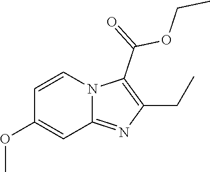 [SMILES: CCOC(=O)c1c(CC)nc2cc(OC)ccn12]

Intermediate II-29 was prepared according to an analogous procedure to the one used for the synthesis of intermediate II-7d, using 2-amino-4-methoxypyridine [10201-73-7] (5 g, 40.3 mmol) as starting material (6.7 g, 67%).

### Synthesis of Intermediate II-30

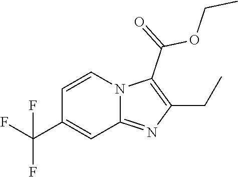 [SMILES: CCOC(=O)c1c(CC)nc2cc(C(F)(F)F)ccn12]

Intermediate II-30 was prepared according to an analogous procedure to the one used for the synthesis of intermediate II-7d, using 4-(trifluoromethyl)-2-pyridinylamine [106447-97-6] (5 g, 30.8 mmol) as starting material. 11-30 was obtained as a yellow solid (3.61 g, 56.8%).

### Synthesis of Intermediate II-31

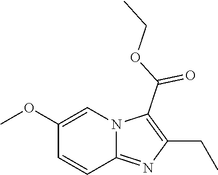 [SMILES: CCOC(=O)c1c(CC)nc2ccc(OC)cn12]

Intermediate II-31 was prepared according to an analogous procedure to the one used for the synthesis of intermediate II-7d, using 2-amino-5-methoxypyridine [10167-97-2] (2 g, 16.1 mmol) as starting material. 11-31 was obtained as an orange solid (2.3 g, 56.4%).

### Synthesis of Intermediate II-32

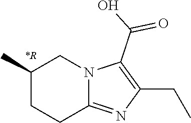

Intermediate II-32 was prepared according to an analogous procedure to the one used for the synthesis of intermediate II-17a using intermediate II-40 (R*) (142 mg, 0.6 mmol) as starting material. Intermediate II-32 was obtained as a white oil (126 mg, quant.).

### Synthesis of Intermediate II-33

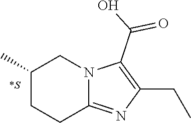

Intermediate II-33 was prepared according to an analogous procedure to the one used for the synthesis of intermediate II-17a, using intermediate II-39 (S*) (142 mg, 0.6 mmol) as starting material. Intermediate II-33 was obtained as a white oil (126 mg, quant.).

### Synthesis of Intermediate II-34—LiCl Salt

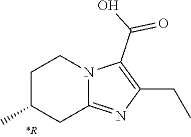

Intermediate II-34 was prepared according to an analogous procedure to the one used for the synthesis of intermediate II-17a, using intermediate II-44 (R*) (124 mg, 0.5 mmol) as starting material. Intermediate II-34 was obtained as a pale yellow solid (131 mg, 98.8%).

### Synthesis of Intermediate II-35—LiCl Salt

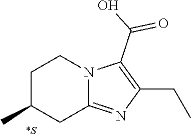

Intermediate II-35 was prepared according to an analogous procedure to the one used for the synthesis of intermediate II-17a, using intermediate II-45 (S*) (121 mg, 0.5 mmol) as starting material. Intermediate II-35 was obtained as a pale yellow solid (127 mg, 99%).

### Synthesis of Intermediate II-36—LiCl Salt

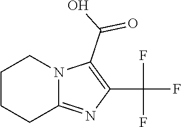 [SMILES: O=C(O)c1c(C(F)(F)F)nc2n1CCCC2]

Intermediate II-36 was prepared according to an analogous procedure to the one used for the synthesis of intermediate II-17a, using intermediate II-48 (160 mg, 0.6 mmol) as starting material. 11-36 was obtained as a white solid (215 mg, 96.5%).

### Synthesis of Intermediate II-37—LiCl Salt

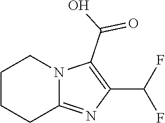 [SMILES: O=C(O)c1c(C(F)F)nc2n1CCCC2]

Intermediate II-37 was prepared according to an analogous procedure to the one used for the synthesis of intermediate II-17a, using intermediate II-98 (130 mg, 0.5 mmol) as starting material. I-37 was obtained as a white solid (144 mg, 99.4%).

### Synthesis of Intermediate II-38

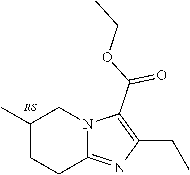 [SMILES: CCOC(=O)c1c(CC)nc2n1CC(C)CC2]

Intermediate II-38 (939 mg, 91%, pale yellow solid) was prepared according to an analogous procedure to the one used for the synthesis of intermediate II-16 from intermediate II-7g (1 g, 4.3 mmol) as starting material.

### Synthesis of Intermediates II-39 (S*) and II-40 (R*)

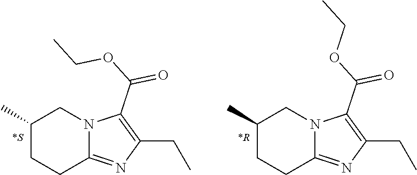 [SMILES: CCOC(=O)c1c(CC)nc2n1C[C@@H](C)CC2 ; CCOC(=O)c1c(CC)nc2n1C[C@H](C)CC2]

A batch of intermediates II-38 (939 mg, 4 mmol) was purified by chiral SFC on a Jasco SFC prep system using an Phenomenex Lux Cellulose-1 250 mm long×30 mm I.D. 5 m particle size, on isocratic mode at 100 ml/min of CO2 (65%)−Isopropanol (35%)+0.1% DEA, at 30° C., BPR 150 Bar. Acquisition frequency was set to 252 nm for the DAD detector. The desired fractions were collected, evaporated, and dried under vacuo to yield intermediate II-39 (S*) (243 mg, 25.6%) and intermediate II-40 (R*) (270 mg, 28.5%) as pale yellow oils.

### Synthesis of Intermediates II-41 (R*) and II-42 (S*)

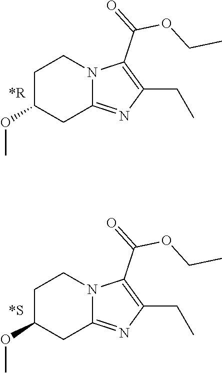 [SMILES: CCOC(=O)c1c(CC)nc2n1CC[C@@H](OC)C2 ; CCOC(=O)c1c(CC)nc2n1CC[C@H](OC)C2]

Intermediates II-41 (R*) and II-42 (S*) were prepared according to an analogous procedure to the one used for the synthesis of intermediate II-39 (S*) and II-40 (R*) using intermediate 11-50 (512 mg, 2 mmol) as starting material. It were obtained intermediate II-41 (R*) (164 mg, 31.7%) and intermediate II-42 (S*) (164 mg, 31.7%) as white solids.

### Synthesis of Intermediates II-43, II-44 (R*) and II-45 (S*)

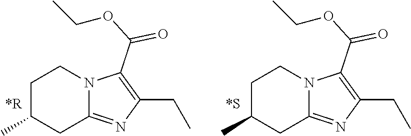

Platinum(IV) oxide [1314-15-4] (177 mg, 0.8 mmol) was added to a stirred solution of intermediate II-7e (1 g, 4.3 mmol) in methanol (9.5 mL) under N₂. Then a concentrated HCl aqueous solution (2.5 μL was added, and the resulting suspension was stirred at 50° C. under H₂ atmosphere for 16 h. The reaction was filtered through a pad of Celite® and the filtrate was evaporated in vacuo to yield intermediate II-43 as light beige solid (1 g, 96%).

Intermediate II-43 was purified by chiral SFC on a Jasco SFC prep system using an Phenomenex Lux Cellulose-1 250 mm long×30 mm I.D. 5 m particle size, on isocratic mode at 100 ml/min of CO₂ (65%)−Isopropanol (10%)+0.1% DEA, at 30° C., BPR 150 Bar.

Acquisition frequency was set to 220 nm for the DAD detector. The desired fractions were collected, evaporated, and dried under vacuo to yield intermediate II-44 (R*) (372 mg, 35.8%) and intermediate II-45 (S*) (364 mg, 35.1%) as light yellow solids.

### Synthesis of Intermediate II-46 (R*) and II-47 (S*)

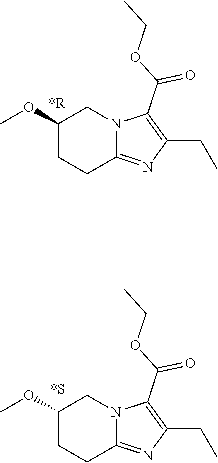

Intermediates II-46 (R*) (161 mg, 31.4%) and II-47 (S*) (103 mg, 20.1%) were prepared according to an analogous procedure to the one used for the synthesis of intermediate II-16 from intermediate II-31 (500 mg, 2 mmol) as starting material.

### Synthesis of Intermediate II-48

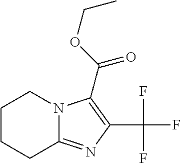 [SMILES: CCOC(=O)c1c(C(F)(F)F)nc2n1CCCC2]

Intermediate II-48 (845 mg, 63.3%, colourless oil) was prepared according to an analogous procedure to the one used for the synthesis of intermediate II-16 from intermediate II-5a (1.3 g, 5.4 mmol) as starting material.

### Synthesis of Intermediate II-49

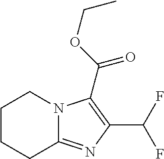 [SMILES: CCOC(=O)c1c(C(F)F)nc2n1CCCC2]

Intermediate II-49 (670 mg, 43.5%, colourless oil) was prepared according to an analogous procedure to the one used for the synthesis of intermediate II-16 from intermediate II-5b (1.5 g, 5.8 mmol) as starting material.

### Synthesis of Intermediate II-50

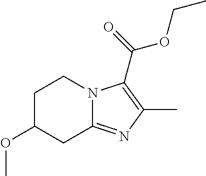 [SMILES: CCOC(=O)c1c(C)nc2n1CCC(OC)C2]

Intermediate II-50 (512 mg, 75%, colourless oil) was prepared according to an analogous procedure to the one used for the synthesis of intermediate II-16 from intermediate II-29 (665 mg, 2.7 mmol) as starting material.

### Synthesis of Intermediate II-51

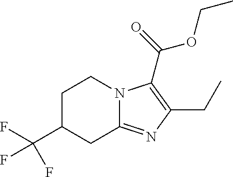 [SMILES: CCOC(=O)c1c(CC)nc2n1CCC(C(F)(F)F)C2]

Intermediate II-51 (698 mg, 73.4%, white solid) was prepared according to an analogous procedure to the one used for the synthesis of intermediate II-16 from intermediate II-30 (938 mg, 3.3 mmol) as starting material.

### Synthesis of Intermediate II-52

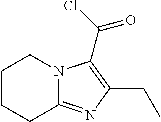

In a round bottom flask, thionyl chloride (120 mL, 1.645 mol) was added dropwise to intermediate II-17a (8 g, 41.188 mmol) at room temperature. The reaction mixture was stirred at 60° C. for 2 hours. Toluene was added and the mixture was concentrated in vacuo to yield intermediate II-52 (8.8 g, 99.5%) as a yellow solid.

### Synthesis of Intermediate II-53

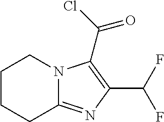 [SMILES: O=C(Cl)c1c(C(F)F)nc2n1CCCC2]

Intermediate II-53 was prepared according to an analogous procedure to the one used for the synthesis of intermediate II-52 from intermediate II-37 (144 mg, 0.5 mmol) as starting material. Intermediate II-53 (154 mg, 99.8%) was obtained as a light orange solid.

### Synthesis of Intermediate II-54

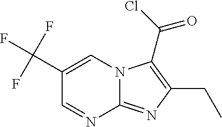 [SMILES: CCc1nc2ncc(C(F)(F)F)cn2c1C(=O)Cl]

Intermediate II-54 (242 mg, 70% purity, 100%, dark brown solid) was prepared according to an analogous procedure to the one used for the synthesis of intermediate II-52 from intermediate II-25 (226 mg, 0.6 mmol) as starting material. Intermediate II-54 (242 mg, 70% purity, 100%) was obtained as a dark brown solid

### Synthesis of Intermediate II-55

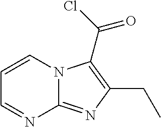

Intermediate II-55) was prepared according to an analogous procedure to the one used for the synthesis of intermediate II-52 from intermediate II-83 (10 g, 0.6 mmol) as starting material.

Intermediate II-55 (11.5 g, 95% purity, 100%) was obtained as a dark brown solid

### Synthesis of Intermediate II-56

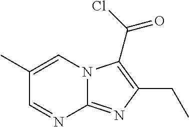

Intermediate II-56 (470 mg, 89.5%, light orange solid) was prepared according to an analogous procedure to the one used for the synthesis of intermediate II-52 from intermediate II-3a (472 mg, 2.3 mmol) as starting material. Intermediate II-56 (470 mg, 89.5%) was obtained as a light orange solid.

### Synthesis of Intermediate II-57

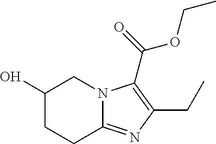 [SMILES: CCOC(=O)c1c(CC)nc2n1CC(O)CC2]

In a vial, a mixture dry EtOH/THF (1:1) (30 mL) was added to intermediate II-7h (1.068 g, 3.292 mmol) and Pd/C 10% (700.8 mg, 0.7 mmol). The vial was placed in a steel vessel, purged 3 times with hydrogen gas and pressurised with 10 bar hydrogen. The mixture was stirred at 30° C. for 19 h and under 20 bar hydrogen at 30° C. for 4 h. The reaction mixture was filtered and the solvent evaporated. The crude material was purified by flash chromatography (silica, MeOH in DCM 0/100 to 10/90). The desired fractions were collected and the solvents removed in vacuo to yield intermediate II-57 (340 mg, 41.2%) as an off-white solid.

### Synthesis of Intermediate II-58

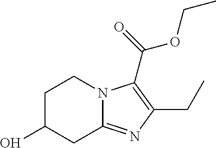 [SMILES: CCOC(=O)c1c(CC)nc2n1CCC(O)C2]

Intermediate II-58 was prepared according to an analogous procedure to the one used for the synthesis of intermediate II-57 using intermediate II-28 (1 g, 3.2 mmol) as starting material.

Intermediate II-58 (540 mg, 69.4%) was obtained as a pale yellow solid.

### Synthesis of Intermediate II-59 (R*) and II-60 (S*)

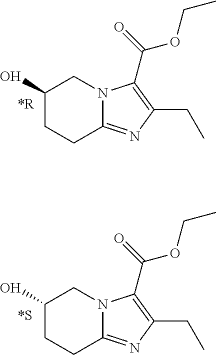 [SMILES: CCOC(=O)c1c(CC)nc2n1C[C@H](O)CC2 ; CCOC(=O)c1c(CC)nc2n1C[C@@H](O)CC2]

Intermediate II-57 (200 mg, 0.8 mmol) was separated by SFC (Jasco SFC prep system using an amylose-1 column, 250*30 mm, 5 μm, isocratic mode 100 ml/min of CO₂ (20%)/MeOH (80%)/DEA (0.1%) at 30° C. 150 bars). Acquisition frequency was set to 220 nm for the DAD detector. Each fraction was diluted with an aqueous saturated NaHCO₃ solution and then extracted with DCM (×2). The combined organic layers were dried over anhydrous MgSO4, filtered and concentrated in vacuo to yield intermediate II-59 (R*) (72 mg, 35.6%) and intermediate II-60 (S*) (73 mg, 36.1%).

### Synthesis of Intermediate II-61 (R*) and II-62 (S*) (VILL_Mcalles_1382_1 and 2)

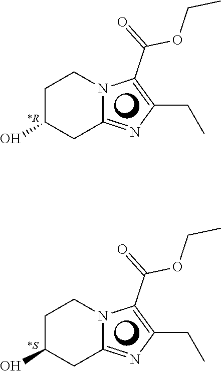 [SMILES: CCOC(=O)c1c(CC)nc2n1CC[C@@H](O)C2 ; CCOC(=O)c1c(CC)nc2n1CC[C@H](O)C2]

Intermediate II-61 (R*) (72 mg, 36.3%) and intermediate II-62 (S*) (59 mg, 29.8%) were prepared according to an analogous procedure to the one used for the synthesis of intermediates II-59 (R*) and II-60 (S*) using intermediate II-58 (200 mg, 0.8 mmol) as starting material.

### Synthesis of Intermediate II-63 (R*) and II-64 (S*)

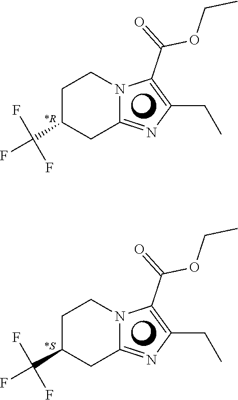 [SMILES: CCOC(=O)c1c(CC)nc2n1CC[C@@H](C(F)(F)F)C2 ; CCOC(=O)c1c(CC)nc2n1CC[C@H](C(F)(F)F)C2]

Intermediates II-63 (R*) (235 mg, 32.9%, white solid) and II-64 (S*) (232 mg, 32.8%, white solid) were prepared according to an analogous procedure to the one used for the synthesis of intermediates II-59 (R*) and II-60 (S*) using intermediate II-51 (700 mg, 2.4 mmol) as starting material.

### Synthesis of Intermediate II-65—NaCl Salt

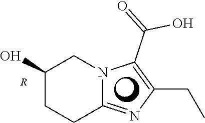

Intermediate II-65 (81.2 mg, quantitative, orange pale solid) was prepared according to an analogous procedure to the one used for the synthesis of intermediate II-8a using intermediate 11-59 (R*) (72 mg, 0.3 mmol) as starting material.

### Synthesis of Intermediate II-66—NaCl Salt

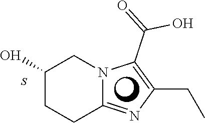

Intermediate II-66 (82 mg, quantitative, yellowish pale solid) was prepared according to an analogous procedure to the one used for the synthesis of intermediate II-8a using intermediate 11-60 (S*) (73 mg, 0.3 mmol) as starting material.

### Synthesis of Intermediate II-67—NaCl Salt

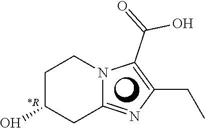

Intermediate II-67 (78.9 mg, 99%, white solid) was prepared according to an analogous procedure to the one used for the synthesis of intermediate II-8a using intermediate II-61 (R*) (70 mg, 0.3 mmol) as starting material.

### Synthesis of Intermediate II-68—NaCl Salt

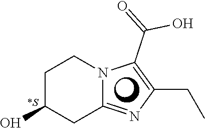

Intermediate II-68 (70 mg, quantitative, white solid) was prepared according to an analogous procedure to the one used for the synthesis of intermediate II-8a using intermediate II-62 (S*) (59 mg, 0.2 mmol) as starting material.

### Synthesis of Intermediate II-69—NaCl Salt

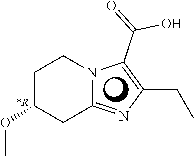 [SMILES: CCc1nc2n(c1C(=O)O)CC[C@@H](OC)C2]

Intermediate II-69 (70 mg, 98.9%, yellow solid) was prepared according to an analogous procedure to the one used for the synthesis of intermediate II-8a using intermediate II-41 (R*) (60 mg, 0.2 mmol) as starting material.

### Synthesis of Intermediate II-70—NaCl Salt

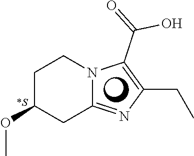 [SMILES: CCc1nc2n(c1C(=O)O)CC[C@H](OC)C2]

Intermediate II-70 (89.6 mg, 99%, yellow solid) was prepared according to an analogous procedure to the one used for the synthesis of intermediate II-8a using intermediate II-42 (S*) (80 mg, 0.3 mmol) as starting material.

### Synthesis of Intermediate II-71—NaCl Salt

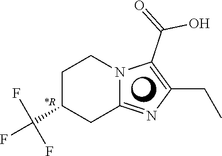 [SMILES: CCc1nc2n(c1C(=O)O)CC[C@@H](C(F)(F)F)C2]

Intermediate II-71 (92 mg, 98.9%, white solid) was prepared according to an analogous procedure to the one used for the synthesis of intermediate II-8a using intermediate II-63 (R*) (80 mg, 0.3 mmol) as starting material.

### Synthesis of Intermediate II-72—NaCl Salt

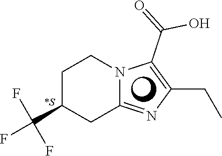 [SMILES: CCc1nc2n(c1C(=O)O)CC[C@H](C(F)(F)F)C2]

Intermediate II-72 (88.4 mg, 99%, white solid) was prepared according to an analogous procedure to the one used for the synthesis of intermediate II-8a using intermediate II-64 (S*) (80 mg, 0.3 mmol) as starting material.

### Synthesis of Intermediate II-73—NaCl Salt

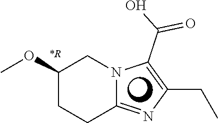

Intermediate II-73 (113 mg, 98.8%, beige solid) was prepared according to an analogous procedure to the one used for the synthesis of intermediate II-8a using intermediate II-46 (R*) (100 mg, 0.4 mmol) as starting material.

### Synthesis of Intermediate II-74—NaCl Salt

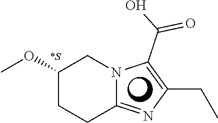 [SMILES: CCc1nc2n(c1C(=O)O)C[C@@H](OC)CC2]

Intermediate II-74 (112 mg, 95%, yellow solid) was prepared according to an analogous procedure to the one used for the synthesis of intermediate II-8a using intermediate II-47 (S*) (100 mg, 0.4 mmol) as starting material.

### Synthesis of Intermediate II-75

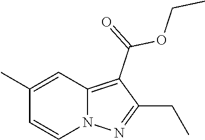 [SMILES: CCOC(=O)c1c(CC)nn2ccc(C)cc12]

4-Methylpyridine [108-89-4] (0.50 ml, 5. mmol) was added to a solution of o-(2,4-dinitrophenyl)hydroxylamine [17508-17-7] (1.00 g, 5.02 mmol) in ACN (25 ml). The reaction mixture was stirred at 40° C. for 24 hours. Solvents were removed in vacuo and the reaction mixture was dissolved in DMF (25 ml). Ethyl 2-pentynoate [55314-57-3] (1.00 ml, 7.36 mmol) and K2CO₃ (2.09 g, 15.12 mmol) were added and the reaction mixture was stirred at room temperature for 24 hours. The mixture was diluted with EtOAc (75 ml), washed with water and brine, dried (anhydrous MgSO4), filtered and concentrated in vacuo. The crude material was purified by flash column chromatography (silica, EtOAc in heptane 0/100 to 50/50). The desired fractions were collected and the solvents removed in vacuo to yield intermediate II-75 (655 mg, 55%) as a yellow solid.

### Synthesis of Intermediate II-76

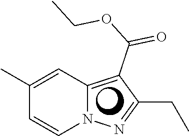 [SMILES: CCOC(=O)c1c(CC)nn2ccc(C)cc12]

Intermediate II-76 (652 mg, 46.9%, yellowish solid) was prepared according to an analogous procedure to the one used for the synthesis of intermediate II-75 using 1-aminopyridin-1-ium iodide [6295-87-0] (1.4 g, 6.3 mmol) as starting material.

### Synthesis of Intermediate II-77

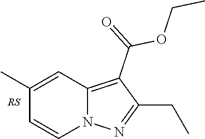 [SMILES: CCOC(=O)c1c(CC)nn2ccc(C)cc12]

Platinum(IV) oxide [1314-15-4] (66 mg, 0.29 mmol) was added to a stirred solution of intermediate II-75 (655 mg, 2.82 mmol) in EtOH (6 mL) under nitrogen atmosphere. Then HCl (concentrated, aq.) (5 μl) was added, and the resulting suspension was stirred at 60° C. under nitrogen atmosphere for 16 hours. Platinum(IV) oxide (66 mg, 0.29 mmol) was added and the reaction mixture was stirred at 60° C. under nitrogen atmosphere for another 18 hours.

The reaction was not complete and platinum (IV) oxide (66 mg, 0.29 mmol) was added four times over 30 hours, the reaction mixture being stirred at 60° C. The reaction mixture was filtered over a pad of Celite® and the solvents were evaporated in vacuo. The crude product was purified by flash column chromatography (silica, EtOAc in heptane 0/100 to 50/50) to afford intermediate II-77 as a pale yellow solid (432 mg, 64%).

### Synthesis of Intermediate II-78

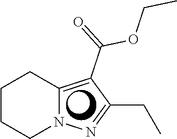 [SMILES: CCOC(=O)c1c(CC)nn2c1CCCC2]

Intermediate II-78 (653 mg, 97.7%, a white solid) was prepared according to an analogous procedure to the one used for the synthesis of intermediate II-77 using intermediate II-76 (650 mg, 3 mmol) as starting material.

### Synthesis of Intermediate II-79 (R*) and II-80 (S*)

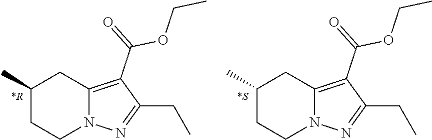 [SMILES: CCOC(=O)c1c(CC)nn2c1C[C@H](C)CC2 ; CCOC(=O)c1c(CC)nn2c1C[C@@H](C)CC2]

Intermediates II-79 (R*) (161 mg, 36.9%) and II-80 (S*) (165 mg, 37.8%) were prepared according to an analogous procedure to the one used for the synthesis of intermediates II-59 (R*) and II-60 (S*) using intermediate II-77 (432 mg, 0.8 mmol) as starting material.

### Synthesis of Intermediate II-81—NaCl Salt

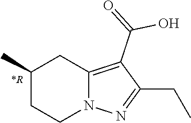

Intermediate II-81 (122 mg, 92%, pale yellow solid) was prepared according to an analogous procedure to the one used for the synthesis of intermediate II-8a using intermediate II-79 (R*) (140 mg, 0.6 mmol) as starting material.

Synthesis of intermediate II-82—NaCl Salt

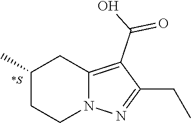

Intermediate II-82 (197 mg, 99.4%, white solid) was prepared according to an analogous procedure to the one used for the synthesis of intermediate II-8a using intermediate II-80 (S*) (158 mg, 0.7 mmol) as starting material.

### Synthesis of Intermediate II-83

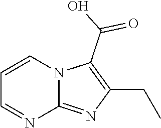

Intermediate II-83 (22 g, 99.9%, pale yellow solid) was prepared according to an analogous procedure to the one used for the synthesis of intermediate II-8a using intermediate II-23 (25 g, 112.9 mmol) as starting material.

### Synthesis of Intermediate II-84—NaCl Salt

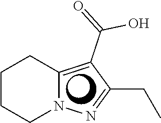

Intermediate II-84 (133 mg, 99%, pale yellow solid) was prepared according to an analogous procedure to the one used for the synthesis of intermediate II-8a using intermediate II-78 (80 mg, 112.9 mmol) as starting material.

### Synthesis of Intermediate II-85

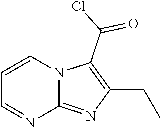

Intermediate II-85 (26.9 g, quantitative, yellow solid) was prepared according to an analogous procedure to the one used for the synthesis of intermediate II-52 from intermediate II-83 (24 g, 125.5 mmol) as starting material.

### Intermediate II-86

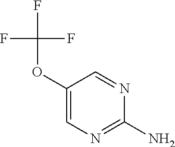 [SMILES: Nc1ncc(OC(F)(F)F)cn1]

In a sealed tube a mixture of 2-Chloro-5-(trifluorometoxy)pyrimidine [1261812-52-5] (300 mg, 1.436 mmol) and ammonium hydroxide [1336-21-6] (13 mL, 83.926 mmol) was stirred at 85° C. for 16 hours. The solvent was evaporated in vacuo to yield intermediate II-86 (295 mg, 94%) as a white solid.

### Intermediate II-87

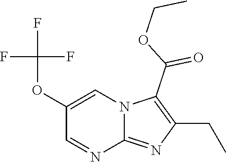 [SMILES: CCOC(=O)c1c(CC)nc2ncc(OC(F)(F)F)cn12]

Intermediate II-87 (69 mg, 72% purity, 12.1%) was prepared according to an analogous procedure to the one used for the synthesis of intermediate II-1a using intermediate II-86 (295 mg, 1.4 mmol) as starting material.

### Intermediate II-88—NaCl Salt

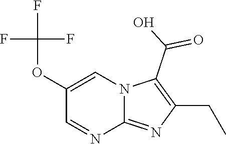 [SMILES: CCc1nc2ncc(OC(F)(F)F)cn2c1C(=O)O]

Intermediate II-88 (53 mg, 97.8%) was prepared according to an analogous procedure to the one used for the synthesis of intermediate II-8a using intermediate II-87 (53 mg, 0.2 mmol) as starting material.

### Intermediate II-89

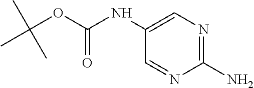

Pd/C [7440-05-3] (0.2 g, 0.188 mmol) was added to a solution of 2-Amino-5-nitropyrimidine [3073-77-6] (1 g, 7.138 mmol) in methanol (16 mL) in a round bottom flask under nitrogen atmosphere at 0° C. The mixture was stirred under H₂ atmosphere [1333-74-0] at rt for 16 h. Then, Di-tertbutyl dicarbonate [24424-99-5] (2.04 mL, 6.786 mmol) wad added at 0° C. and the mixture was stirred at 0° C. for 6 h. The reaction was filtered through pad of celite. The solvent was concentrated in vacuo to give a colorless oil. The crude product was purified by flash column chromatography (silica 80 g; AcOEt in heptane 0/100 to 60/40). The desired fractions were collected and concentrated in vacuo to yield Intermediate II-89 (1.052 g, 69%) as a yellow solid.

### Synthesis of Intermediate II-90

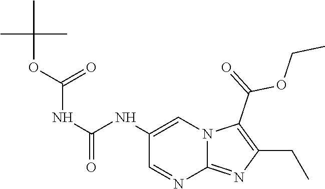 [SMILES: CCOC(=O)c1c(CC)nc2ncc(NC(=O)NC(=O)OC(C)(C)C)cn12]

Intermediate II-90 (127 mg, 20%) was prepared according to an analogous procedure to the one used for the synthesis of intermediate II-89 (400 mg, 1.9 mmol) as starting material.

### Synthesis of Intermediate II-91—NaCl Salt

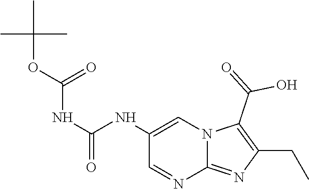 [SMILES: CCc1nc2ncc(NC(=O)NC(=O)OC(C)(C)C)cn2c1C(=O)O]

Intermediate II-91 (183 mg, 99.6%, yellow solid) was prepared according to an analogous procedure to the one used for the synthesis of intermediate II-8a using intermediate II-90 (160 mg, 0.5 mmol) as starting material.

### Synthesis of Intermediate II-92

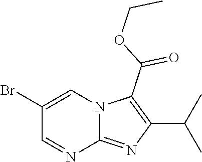 [SMILES: CCOC(=O)c1c(C(C)C)nc2ncc(Br)cn12]

Intermediate II-92 (464 mg, 25%, yellow solid) was prepared according to an analogous procedure to the one used for the synthesis of intermediate 11-la using 2-amino-5-bromopyrimidine [7752-82-1] (1 g, 5.7 mmol) and ethyl isobutyrylacetate [7152-15-0] (1.5 mL, 8.6 mmol) as starting material.

### Synthesis of Intermediate II-93

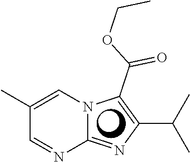 [SMILES: CCOC(=O)c1c(C(C)C)nc2ncc(C)cn12]

Intermediate II-93 (331 mg, 95.9%, yellow solid) was prepared according to an analogous procedure to the one used for the synthesis of intermediate II-1a using intermediate II-92 (454 mg, 1.4 mmol) as starting material.

### Synthesis of Intermediate II-94—KCl Salt

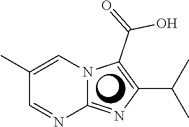 [SMILES: Cc1cnc2nc(C(C)C)c(C(=O)O)n2c1]

Aqueous potassium carbonate (1.1 mL, 1.20 mmol) was added to a solution of intermediate II-93 (130 mg, 0.50 mmol) in Ethanol (3.0 mL) at room temperature. The mixture was stirred at 90° C. for 16 h. Aqueous potassium carbonate (1.4 mL, 1.52 mmol) was added to the mixture, and it was stirred at 90° C. for 1 h. The solution was acidified with HCl 1 M until pH=7 and the solvent was concentrated in vacuo to yield intermediate II-94 (147 mg, 82%) as a yellowish solid.

### Synthesis of Intermediate II-95

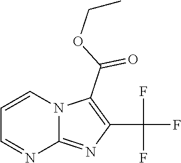 [SMILES: CCOC(=O)c1c(C(F)(F)F)nc2ncccn12]

Intermediate II-95 (1.8 g, 95%) was prepared according to an analogous procedure to the one used for the synthesis of intermediate II-1a using 2-Aminopyrimidine [109-12-6] (2.5 g, 25.5 mmol) and Ethyl 4,4, 4-trifluoroacetoacetate [372-31-6] (4.6 mL, 25.5 mmol) as starting material.

### Synthesis of Intermediate II-96—Li Salt

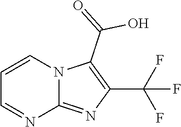 [SMILES: O=C(O)c1c(C(F)(F)F)nc2ncccn12]

Intermediate II-96 (174 mg, 80% purity, 83.4%) was prepared according to an analogous procedure to the one used for the synthesis of intermediate II-17a using intermediate II-95 (158 mg, 0.6 mmol) as starting material.

### Synthesis of Intermediate II-97

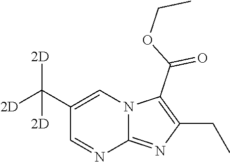

In a round bottom flask, Zn(CD₂)₃ (850 mg, 8.37 mmol) in solution in Et₂O was added to a mixture of Pd(dppf)₂Cl₂ (560 mg, 0.69 mmol) and intermediate II-92 (2.0 g, 6.7 mmol) in anhydrous dioxane (50 ml) under nitrogen atmosphere. The reaction mixture was stirred at 55° C. for 16 hours. A solution of NaHCO₃ (sat., aq.) was added dropwise at 0° C. The solid was filtered off and washed with a solution of DCM and MeOH (9:1). The organic layer was dried (anhydrous MgSO4), filtered and concentrated in vacuo. The crude product was purified by flash column chromatography (silica, 80 g, gradient: EtOAc in heptane from 0/100 to 50/50). The desired fractions were collected and concentrated in vacuo to afford intermediate II-97 (1.2 g, 72%). as a brown solid.

### Synthesis of Intermediate II-98—NaCl Salt

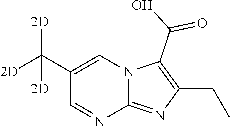

Intermediate II-98 (223 mg, 99.8%) was prepared according to an analogous procedure to the one used for the synthesis of intermediate II-8a using intermediate II-97 (200 mg, 0.8 mmol) as starting material.

### Synthesis of Intermediate II-98

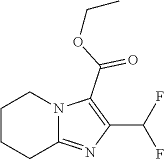 [SMILES: CCOC(=O)c1c(C(F)F)nc2n1CCCC2]

Intermediate II-98 was prepared according to an analogous procedure to the one used for the synthesis of intermediate II-16, using intermediate II-99 (1.3 g, 5.41 mmol) as starting material. I-98 was obtained as a colourless oil (845 mg, 63%).

### Synthesis of Intermediate II-99

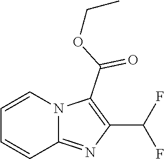 [SMILES: CCOC(=O)c1c(C(F)F)nc2ccccn12]

Intermediate II-99 (0.23 g, 24%) was prepared according to an analogous procedure to the one used for the synthesis of intermediate II-7d, using 2-Aminopyridine [504-29-0] (1.32 g, 14.02 mmol) as starting material.

### Synthesis of Intermediate II-100

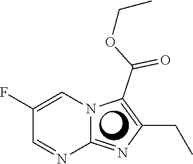 [SMILES: CCOC(=O)c1c(CC)nc2ncc(F)cn12]

Intermediate II-100 was prepared according to an analogous procedure to the one used for the synthesis of intermediate 11-la using Ethyl propionylacetate [4949-44-4] (500 mg, 4.5 mmol) and 2-Amino-5-fluoropyridine [21717-96-4] (983 uL, 6.7 mmol) as starting material. (573 mg, 53.8%) as yellowish solid.

### Synthesis of Intermediate II-101—KCl Salt

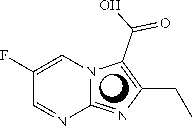

Intermediate II-101 was prepared according to an analogous procedure to the one used for the synthesis of intermediate II-3a using intermediate II-100 (180 mg, 0.8 mmol) as starting material. (215 mg, 98.8%) as yellow solid.

### Synthesis of Intermediate II-102—LiCl Salt

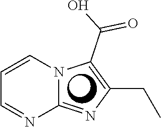

Intermediate II-102 (149 mg, 99.9%, white solid) was prepared according to an analogous procedure to the one used for the synthesis of intermediate II-8e using intermediate II-1d (133 mg, 0.6 mmol) as starting material.

### Synthesis of Intermediate II-103

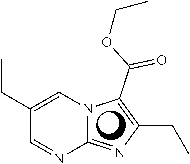 [SMILES: CCOC(=O)c1c(CC)nc2ncc(CC)cn12]

Intermediate II-103 was prepared according to an analogous procedure to the one used for the synthesis of intermediate II-2a using intermediate II-1a (500 mg, 1.6 mmol) as starting material. Intermediate II-103 (135 mg, 34.3%) was obtained as a yellow solid.

### Synthesis of Intermediate II-104—NaCl Salt

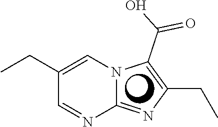 [SMILES: CCc1cnc2nc(CC)c(C(=O)O)n2c1]

Intermediate II-104 was prepared according to an analogous procedure to the one used for the synthesis of intermediate II-8a using intermediate II-103 (110 mg, 0.4 mmol) as starting material. Intermediate II-104 (116 mg, 99%) was obtained as a yellow solid.

### Synthesis of Intermediate II-105—KCl Salt

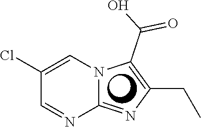

Intermediate II-105 was prepared according to an analogous procedure to the one used for the synthesis of intermediate II-94 using intermediate intermediate II-1c (485 mg, 1.9 mmol) as starting material. Intermediate II-105 (1.2 g, 95.4%) was obtained as a yellow solid.

### Synthesis of Intermediate II-106—KCl Salt

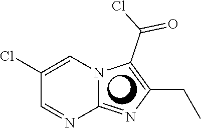

Intermediate II-106 was prepared according to an analogous procedure to the one used for the synthesis of intermediate II-25 using intermediate intermediate II-105 (1.2 g, 1.8 mmol) as starting material. Intermediate II-106 (1.3 g, 99.9%) was obtained as a brown solid.

### Synthesis of Intermediate II-107—LiCl Salt

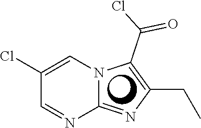

Intermediate II-107 was prepared according to an analogous procedure to the one used for the synthesis of intermediate II-25 using intermediate II-37 (144 mg, 0.5 mmol) as starting material. Intermediate II-107 (154 mg, 99.8%) was obtained as a light orange solid.

### Synthesis of Intermediate II-108—KCl Salt

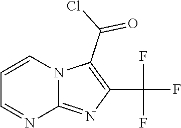 [SMILES: O=C(Cl)c1c(C(F)(F)F)nc2ncccn12]

Intermediate II-108 was prepared according to an analogous procedure to the one used for the synthesis of intermediate II-25 using intermediate intermediate II-96 (207 mg, 0.7 mmol) as starting material. Intermediate II-108 (230 mg, 99.5%) was obtained as a light orange solid.

### Synthesis of Intermediate II-109

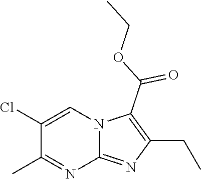 [SMILES: CCOC(=O)c1c(CC)nc2nc(C)c(Cl)cn12]

In a round bottom flask, iron(III) acetylacetonate [14024-18-1] (237 mg, 0.65 mmol) was added to a solution of intermediate II-10 (1.88 g, 6.52 mmol) in anhydrous THE (27 ml) and NMP (2.2 ml) under nitrogen atmosphere at 0° C. Methylmagnesium bromide (3M solution in Et₂O, 4.35 ml, 13.05 mmol) was added. The reaction mixture was stirred at 0° C. for 3 hours. A solution of NH4Cl (sat., aq.) was added and the mixture was extracted with EtOAc. The organic layer was dried (anhydrous MgSO4), filtered and concentrated in vacuo. The crude product was purified by flash column chromatography (silica, 80 g, gradient: EtOAc in heptane from 0/100 to 65/35). The desired fractions were collected and concentrated in vacuo to afford intermediate II-109 as a sticky orange oil (215 mg, 12%).

### Synthesis of Intermediate II-110—KCl Salt

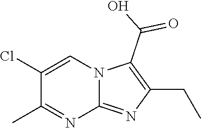 [SMILES: CCc1nc2nc(C)c(Cl)cn2c1C(=O)O]

Intermediate II-110 was prepared according to an analogous procedure to the one used for the synthesis of intermediate II-94 using intermediate II-109 (290 mg, 1 mmol) as starting material. Intermediate II-110 (337 mg, 100%) was obtained as a yellowish solid.

### Synthesis of Final Compounds

### Synthesis of Compound 1ab

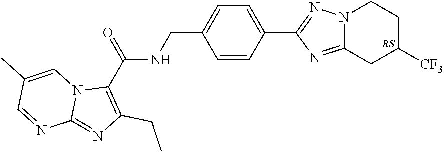 [SMILES: CCc1nc2ncc(C)cn2c1C(=O)NCc1ccc(-c2nc3n(n2)CCC(C(F)(F)F)C3)cc1]

HATU [148893-10-1] (3.84 g, 10.1 mmol) and DIPEA [7087-68-5] (8.21 mL, 41.13 mmol) were added to a solution of intermediate II-3a (2.76 g, 9.43 mmol) in DMF (160 mL) at rt and the mixture was stirred for 10 min. Then, intermediate I-6 (2.49 g, 6.73 mmol) was added, and the mixture reaction was stirred at rt for a further 1 h. Then, a saturated NaHCO3 aqueous solution was added, and the mixture was extracted with DCM (×3). The combined organic extracts were dried (MgSO4), filtered and the solvents were evaporated in vacuo. The crude product was triturated with a saturated NaHCO3 aqueous solution and filtered. The solid was washed with water (×3), DCM (×3) and finally with diethyl ether to yield final compound lab as a white solid (1.59 g, 48%). The mother liquors were concentrated in vacuo and purified by flash column chromatography (silica; DCM/MeOH 9:1 in DCM 0/100 to 30/70). The desired fractions were collected, concentrated in vacuo to yield some two additional fractions of compound lab as a pale brown solid (0.27 g, 8%) and as white solid (0.44 g, 13%).

1H NMR (400 MHz, DMSO) δ 9.16 (d, J=1.0 Hz, 1H), 8.55-8.47 (m, 2H), 7.95 (d, J=8.2 Hz, 2H), 7.45 (d, J=8.2 Hz, 2H), 4.57 (d, J=5.9 Hz, 2H), 4.33 (dd, J=12.8, 3.7 Hz, 1H), 4.17 (td, J=12.1, 4.8 Hz, 1H), 3.27-3.12 (m, 2H), 3.02 (q, J=7.5 Hz, 2H), 2.92 (dd, J=17.4, 11.9 Hz, 1H), 2.34 (s, 3H), 2.30 (d, J=2.3 Hz, 1H), 2.20-2.02 (m, 1H), 1.28 (t, J=7.5 Hz, 3H).

### Synthesis of Compounds 1a and 1b

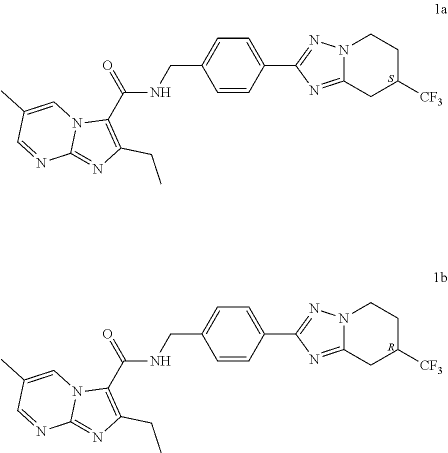

A batch of compound lab (0.32 mg, 0.66 mmol), prepared according to an analogous procedure to the one outlined above, was purified by chiral SFC on a Jasco SFC prep system using an Phenomenex Lux Cellulose-1 250 mm long×30 mm I.D. 5 m particle size, on isocratic mode at 30 ml/min of CO2 (55%)−Methanol (45%)+0.1% diethylamine, at 30° C., BPR 150 Bar. Acquisition frequency was set to 220 nm for the DAD detector. The desired fractions were collected, evaporated, and dried under vacuo. The residues were triturated with diethyl ether to yield compounds 1a (90 mg) and 1b (68 mg) as white solids.

Compound 1a 1H NMR (400 MHz, DMSO) δ 9.16 (s, 1H), 8.51 (d, J=2.4 Hz, 1H), 8.50 (d, J=8.0 Hz, 1H), 7.95 (d, J=8.2 Hz, 2H), 7.45 (d, J=8.2 Hz, 2H), 4.57 (d, J=5.8 Hz, 2H), 4.33 (dd, J=12.6, 3.7 Hz, 1H), 4.17 (td, J=12.1, 4.8 Hz, 1H), 3.23-3.15 (m, 2H), 3.02 (q, J=7.5 Hz, 2H), 2.94-2.85 (m, 1H), 2.34 (s, 3H), 2.31 (s, 1H), 2.11 (qd, J=11.4, 5.7 Hz, 1H), 1.28 (t, J=7.5 Hz, 3H).

Compound 1b 1H NMR (400 MHz, DMSO) δ 9.16 (d, J=1.1 Hz, 1H), 8.52 (d, J=2.4 Hz, 1H), 8.50 (d, J=6.1 Hz, 1H), 7.95 (d, J=8.2 Hz, 2H), 7.45 (d, J=8.2 Hz, 2H), 4.57 (d, J=5.9 Hz, 2H), 4.33 (dd, J=12.9, 3.8 Hz, 1H), 4.17 (td, J=12.0, 4.6 Hz, 1H), 3.24-3.14 (m, 2H), 3.02 (q, J=7.5 Hz, 2H), 2.93 (dd, J=17.5, 12.1 Hz, 1H), 2.34 (s, 3H), 2.31 (s, 1H), 2.18-2.06 (m, 1H), 1.28 (t, J=7.5 Hz, 3H).

### Synthesis of Compound 2ab

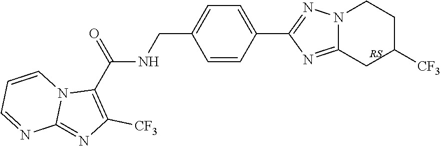 [SMILES: O=C(NCc1ccc(-c2nc3n(n2)CCC(C(F)(F)F)C3)cc1)c1c(C(F)(F)F)nc2ncccn12]

HATU [148893-10-1] (275 mg, 0.72 mmol) was added to a mixture of intermediate I-6 (120 mg, 0.36 mmol), intermediate II-6a (100 mg, 0.43 mmol) and DIPEA [7087-68-5] (0.37 mL, 2.17 mmol) in DMF (5 mL) at rt. The mixture was stirred at rt for 16 h. Then, a 1 M Na2CO3 aqueous solution was added, and the mixture was extracted with EtOAc. The organic layer was dried (MgSO4), filtered and the solvents evaporated in vacuo. The crude product was purified by flash column chromatography (silica; EtOAc in heptane 0/100 to 100/0). The desired fractions were collected, concentrated in vacuo and the residue was repurified by reverse phase HPLC (Phenomenex Gemini C18 100×30 mm 5 μm Column; from 72% [0.1% HCOOH]−28% [ACN:MeOH (1:1)] to 36% [0.1% HCOOH]−64% [ACN:MeOH (1:1)]). The desired fractions were collected and concentrated in vacuo to yield compound 2ab as a brown solid (122 mg, 65%).

1H NMR (400 MHz, DMSO) δ 9.33 (t, J=5.8 Hz, 1H), 8.57 (d, J=7.0 Hz, 1H), 7.97 (d, J=8.2 Hz, 2H), 7.79 (d, J=9.1 Hz, 1H), 7.59-7.50 (m, 1H), 7.45 (d, J=8.2 Hz, 2H), 7.20 (t, J=6.5 Hz, 1H), 4.59 (d, J=5.8 Hz, 2H), 4.33 (dd, J=12.9, 3.5 Hz, 1H), 4.18 (td, J=11.9, 4.7 Hz, 1H), 3.25-3.14 (m, 2H), 2.93 (dd, J=17.7, 12.2 Hz, 1H), 2.37-2.27 (m, 1H), 2.18-2.09 (m, 1H).

### Synthesis of Compounds 2a and 2b

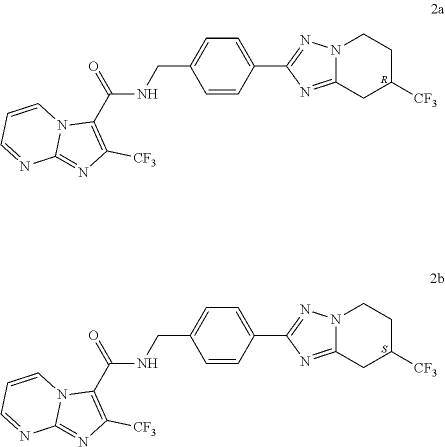

A batch of compound 2ab (350 mg, 0.69 mmol), prepared according to an analogous procedure to the one outlined above, was purified by chiral SFC on a Jasco SFC prep system using an Phenomenex Lux i-Amylose-1 250 mm long×30 mm I.D. 5 m particle size, on isocratic mode at 30 ml/min of CO2 (60%)−Ethanol (40%)+0.1% diethylamine, at 30° C., BPR 120 Bar. Acquisition frequency was set to 220 nm for the DAD detector. The desired fractions were collected, evaporated, and dried under high vacuo. The residues were triturated with diethyl ether to yield compounds 2a (87 mg) and 2b (89 mg) as white-off solids.

Compound 2a 1H NMR (400 MHz, DMSO) δ 9.33 (t, J=5.8 Hz, 1H), 8.57 (d, J=7.0 Hz, 1H), 7.97 (d, J=8.2 Hz, 2H), 7.80 (d, J=9.1 Hz, 1H), 7.61-7.50 (m, 1H), 7.45 (d, J=8.2 Hz, 2H), 7.20 (dd, J=9.9, 3.9 Hz, 1H), 4.59 (d, J=5.4 Hz, 2H), 4.33 (dd, J=12.5, 3.6 Hz, 1H), 4.18 (td, J=12.2, 4.9 Hz, 1H), 3.20 (d, J=13.5 Hz, 2H), 2.93 (dd, J=17.6, 12.1 Hz, 1H), 2.37-2.27 (m, 1H), 2.12 (qd, J=11.6, 6.0 Hz, 1H).

Compound 2b 1H NMR (400 MHz, DMSO) δ 9.34 (d, J=5.4 Hz, 1H), 8.57 (d, J=7.0 Hz, 1H), 7.97 (d, J=8.2 Hz, 2H), 7.79 (d, J=9.2 Hz, 1H), 7.55 (dd, J=12.0, 3.9 Hz, 1H), 7.45 (d, J=8.2 Hz, 2H), 7.20 (t, J=6.9 Hz, 1H), 4.59 (d, J=5.2 Hz, 2H), 4.33 (dd, J=12.7, 3.4 Hz, 1H), 4.24-4.10 (m, 1H), 3.20 (d, J=13.5 Hz, 2H), 2.93 (dd, J=17.6, 12.3 Hz, 1H), 2.33 (dd, J=6.4, 4.4 Hz, 1H), 2.12 (qd, J=11.5, 5.8 Hz, 1H).

### Synthesis of Compound 3ab

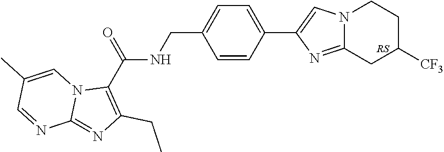 [SMILES: CCc1nc2ncc(C)cn2c1C(=O)NCc1ccc(-c2cn3c(n2)CC(C(F)(F)F)CC3)cc1]

HATU [148893-10-1] (286 mg, 0.52 mmol) was added to a mixture of intermediate I-10 (125 mg, 0.86 mmol), intermediate II-3a (93 mg, 0.45 mmol) and DIPEA [7087-68-5] (0.38 mL, 2.26 mmol) in DMF (5 mL) at rt. The mixture was stirred at rt for 16 h. Then, a 1 M Na2CO3 aqueous solution was added, and the mixture was extracted with EtOAc. The organic layer was dried (MgSO4), filtered and the solvents evaporated in vacuo. The crude product was purified by flash column chromatography (silica; EtOAc in heptane 0/100 to 100/0). The desired fractions were collected, concentrated in vacuo and the residue was repurified by reverse phase HPLC (Phenomenex Gemini C18 100×30 mm 5 μm Column; from 70% [25 mM NH4HCO3]−30% [ACN:MeOH (1:1)] to 27% [25 mM NH4HCO3]−73% [ACN:MeOH (1:1)]). The desired fractions were collected and concentrated in vacuo to yield compound 3ab as a white solid (12 mg, 7%).

1H NMR (400 MHz, DMSO) δ 9.15 (dd, J=2.3, 1.1 Hz, 1H), 8.51 (d, J=2.4 Hz, 1H), 8.47 (t, J=5.9 Hz, 1H), 7.69 (d, J=8.2 Hz, 2H), 7.53 (s, 1H), 7.34 (d, J=8.2 Hz, 2H), 4.52 (d, J=5.9 Hz, 2H), 4.16 (dd, J=12.5, 3.6 Hz, 1H), 3.98 (td, J=12.2, 4.6 Hz, 1H), 3.12-3.05 (m, 2H), 3.01 (q, J=7.5 Hz, 2H), 2.78 (dd, J=17.3, 12.8 Hz, 1H), 2.34 (s, 3H), 2.23 (d, J=11.3 Hz, 1H), 1.95 (ddd, J=24.8, 11.9, 5.6 Hz, 1H), 1.27 (t, J=7.5 Hz, 3H).

### Synthesis of Compounds 3a and 3b

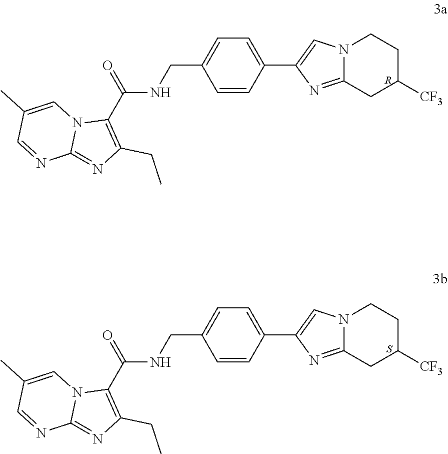

A batch of compound 3ab (350 mg, 0.69 mmol), prepared according to an analogous procedure to the one outlined above, was purified by chiral SFC on a Jasco SFC prep system using an Phenomenex Lux Amylose-1 250 mm long×30 mm I.D. 5 m particle size, on isocratic mode at 30 ml/min of CO2 (50%)−2-propanol (50%)+0.1% diethylamine, at 30° C., BPR 120 Bar. Acquisition frequency was set to 220 nm for the DAD detector. The desired fractions were collected, evaporated, and dried under high vacuo. Both SFC-eluting products were repurified by flash column chromatography (silica; EtOAc in heptane 0/100 to 100/0). The desired fractions were collected and concentrated in vacuo. The first SFC eluting peak product was triturated with a mixture 1:1 of DIPE/DCM and dried under high vacuo at 60° C. and then triturated with DIPE and dried under high vacuo to yield compound 3a as an off-white solid (40 mg). The second SFC eluting peak product was triturated a mixture 1:1 of DIPE/DCM and dried under high vacuo at 60° C. to yield compound 3b as an off-white solid (71 mg).

Compound 3a 1H NMR (400 MHz, DMSO) δ 9.15 (d, J=1.1 Hz, 1H), 8.51 (d, J=2.4 Hz, 1H), 8.47 (t, J=5.9 Hz, 1H), 7.69 (d, J=8.2 Hz, 2H), 7.54 (s, 1H), 7.34 (d, J=8.2 Hz, 2H), 4.52 (d, J=5.9 Hz, 2H), 4.17 (dd, J=12.6, 3.3 Hz, 1H), 3.98 (td, J=12.2, 4.6 Hz, 1H), 3.14-3.05 (m, 2H), 3.01 (q, J=7.5 Hz, 2H), 2.79 (dd, J=17.3, 12.6 Hz, 1H), 2.34 (s, 3H), 2.24 (d, J=11.3 Hz, 1H), 2.02-1.88 (m, 1H), 1.27 (t, J=7.5 Hz, 3H).

Compound 3b 1H NMR (400 MHz, DMSO) δ 9.19-9.11 (m, 1H), 8.51 (d, J=2.4 Hz, 1H), 8.47 (t, J=5.9 Hz, 1H), 7.69 (d, J=8.2 Hz, 2H), 7.55 (s, 1H), 7.35 (d, J=8.2 Hz, 2H), 4.52 (d, J=5.9 Hz, 2H), 4.17 (dd, J=12.6, 3.5 Hz, 1H), 3.98 (td, J=12.1, 4.5 Hz, 1H), 3.15-3.05 (m, 2H), 3.01 (q, J=7.5 Hz, 2H), 2.79 (dd, J=17.4, 12.6 Hz, 1H), 2.34 (s, 3H), 2.28-2.19 (m, 1H), 2.03-1.87 (m, 1H), 1.27 (t, J=7.5 Hz, 3H).

### Synthesis of Compound 4ab

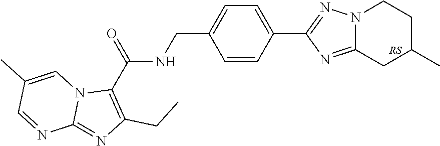

Intermediate I-16 (150 mg, 0.48 mmol) was added to a stirred mixture of intermediate II-3a (189 mg, 0.67 mmol), HATU [148893-10-1] (272 mg, 0.72 mmol), and DIPEA [7087-68-5](0.58 mL, 3.33 mmol) in DMF (10 mL) at rt. The mixture was stirred at rt for 16 h. Then, a saturated NaHCO3 aqueous solution was added, and the mixture was extracted with DCM (3×). The combined organic layers were dried (MgSO4), filtered and the solvents evaporated in vacuo. The crude product was purified by flash column chromatography (silica; DCM/MeOH 9:1 in DCM 0/100 to 100/0). The desired fractions were collected, concentrated in vacuo and the residue triturated with DIPE to yield compound 4ab as a white solid (105 mg, 49%).

1H NMR (400 MHz, DMSO) δ 9.16 (dd, J=2.3, 1.1 Hz, 1H), 8.50 (dd, J=7.1, 4.1 Hz, 2H), 7.94 (d, J=8.3 Hz, 2H), 7.43 (d, J=8.3 Hz, 2H), 4.57 (d, J=5.8 Hz, 2H), 4.22 (ddd, J=12.6, 5.6, 2.8 Hz, 1H), 4.15-4.01 (m, 1H), 3.08-2.93 (m, 3H), 2.47-2.39 (m, 1H), 2.37-2.31 (m, 3H), 2.05 (dt, J=18.1, 13.1 Hz, 2H), 1.74 (dtd, J=13.4, 10.9, 5.8 Hz, 1H), 1.28 (t, J=7.5 Hz, 3H), 1.10 (d, J=6.6 Hz, 3H).

### Synthesis of Compound 5ab

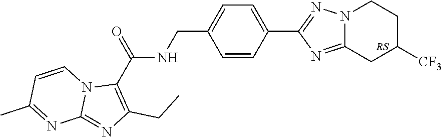 [SMILES: CCc1nc2nc(C)ccn2c1C(=O)NCc1ccc(-c2nc3n(n2)CCC(C(F)(F)F)C3)cc1]

Intermediate I-6 (130 mg, 0.33 mmol) was added to a stirred mixture of intermediate II-8a (167 mg, 0.60 mmol), HATU [148893-10-1] (191 mg, 0.50 mmol), and DIPEA [7087-68-5](0.41 mL, 2.34 mmol) in DMF (5 mL) at rt. The mixture was stirred at rt for 16 h. Then, a saturated NaHCO3 aqueous solution was added, and the mixture was extracted with EtOAc (2×). The combined organic layers were dried (MgSO4), filtered and the solvents evaporated in vacuo. The crude product was purified by flash column chromatography (silica; DCM/MeOH 9:1 in DCM 0/100 to 30/70). The desired fractions were collected, concentrated in vacuo to yield compound 5ab as a white solid (92 mg, 56%).

1H NMR (400 MHz, DMSO) δ 9.20 (d, J=7.0 Hz, 1H), 8.46 (t, J=5.5 Hz, 1H), 7.95 (d, J=7.9 Hz, 2H), 7.44 (d, J=7.9 Hz, 2H), 7.06 (d, J=7.0 Hz, 1H), 4.56 (d, J=5.6 Hz, 2H), 4.39-4.09 (m, 2H), 3.19 (d, J=13.1 Hz, 2H), 3.01 (q, J=7.4 Hz, 2H), 2.97-2.88 (m, 1H), 2.55 (s, 3H), 2.32 (d, J=11.3 Hz, 1H), 2.18-2.05 (m, 1H), 1.27 (t, J=7.4 Hz, 3H).

### Synthesis of Compounds 5a and 5b

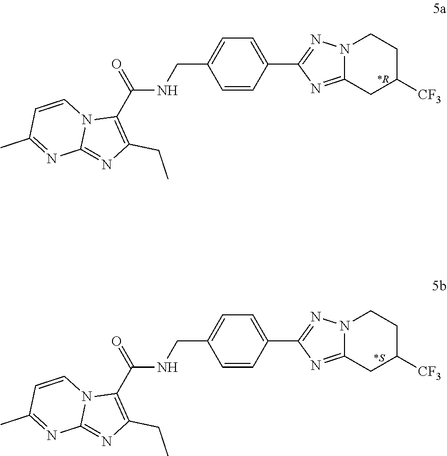

A batch of compound 5ab (1.34 g, 2.87 mmol), prepared according to an analogous procedure to the one outlined above, was purified by chiral SFC method on a Jasco SFC prep system using an i-Cellulose-C column (Regis Technologies) 250 mm long×30 mm I.D. 5 μm particle size, on isocratic mode at 100 ml/min of CO2 (65%)−methanol (45%), at 30° C., BPR 150 Bar. Acquisition frequency was set to 220 nm for the DAD detector. The desired fractions were collected and concentrated in vacuo to yield compound 5a (430 mg, 79%) and compound 5b (420 mg, yield=77%) as white solids.

Compound 5a 1H NMR (400 MHz, DMSO) δ 9.20 (d, J=7.0 Hz, 1H), 8.45 (t, J=6.0 Hz, 1H), 7.95 (d, J=8.2 Hz, 2H), 7.44 (d, J=8.3 Hz, 2H), 7.05 (d, J=7.1 Hz, 1H), 4.56 (d, J=5.9 Hz, 2H), 4.33 (dd, J=12.8, 3.8 Hz, 1H), 4.26-4.07 (m, 1H), 3.24-3.13 (m, 2H), 3.01 (q, J=7.5 Hz, 2H), 2.96-2.85 (m, 1H), 2.55 (s, 3H), 2.32 (d, J=10.8 Hz, 1H), 2.11 (ddd, J=17.3, 12.2, 5.8 Hz, 1H), 1.27 (t, J=7.5 Hz, 3H).

Compound 5b 1H NMR (400 MHz, DMSO) δ 9.20 (d, J=7.0 Hz, 1H), 8.45 (t, J=5.9 Hz, 1H), 7.95 (d, J=8.3 Hz, 2H), 7.44 (d, J=8.3 Hz, 2H), 7.05 (d, J=7.1 Hz, 1H), 4.56 (d, J=5.9 Hz, 2H), 4.37-4.26 (m, 1H), 4.23-4.11 (m, 1H), 3.19 (d, J=13.3 Hz, 2H), 3.01 (q, J=7.5 Hz, 2H), 2.92 (dd, J=17.2, 11.8 Hz, 1H), 2.55 (s, 3H), 2.30 (t, J=10.4 Hz, 1H), 2.20-2.03 (m, 1H), 1.27 (t, J=7.5 Hz, 3H).

### Synthesis of Compound 6ab

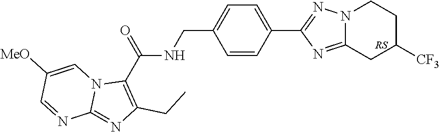 [SMILES: CCc1nc2ncc(OC)cn2c1C(=O)NCc1ccc(-c2nc3n(n2)CCC(C(F)(F)F)C3)cc1]

Intermediate I-6 (130 mg, 0.33 mmol) was added to a stirred mixture of intermediate II-8b (171 mg, 0.60 mmol), HATU [148893-10-1] (176 mg, 0.46 mmol), and DIPEA [7087-68-5](0.38 mL, 2.16 mmol) in DMF (5 mL) at rt. The mixture was stirred at rt for 16 h. Then, a saturated NaHCO3 aqueous solution was added, and the mixture was extracted with EtOAc (2×). The combined organic layers were dried (MgSO4), filtered and the solvents evaporated in vacuo. The crude product was purified by flash column chromatography (silica; DCM/MeOH 9:1 in DCM 0/100 to 30/70). The desired fractions were collected, concentrated in vacuo to yield compound 6ab as a white solid (113 mg, 67%).

1H NMR (400 MHz, DMSO) δ 8.97 (d, J=3.0 Hz, 1H), 8.52 (d, J=3.0 Hz, 1H), 8.47 (t, J=5.8 Hz, 1H), 7.96 (d, J=8.2 Hz, 2H), 7.45 (d, J=8.2 Hz, 2H), 4.58 (d, J=5.8 Hz, 2H), 4.37-4.28 (m, 1H), 4.23-4.11 (m, 1H), 3.86 (s, 3H), 3.21 (s, 1H), 3.17 (d, J=5.2 Hz, 1H), 3.03 (q, J=7.5 Hz, 2H), 2.98-2.87 (m, 1H), 2.32 (d, J=8.7 Hz, 1H), 2.19-2.02 (m, 1H), 1.28 (t, J=7.5 Hz, 3H).

### Synthesis of Compound 7ab

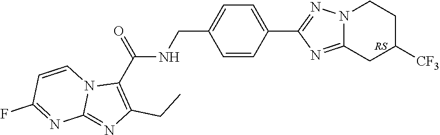 [SMILES: CCc1nc2nc(F)ccn2c1C(=O)NCc1ccc(-c2nc3n(n2)CCC(C(F)(F)F)C3)cc1]

HATU [148893-10-1] (450 mg, 1.18 mmol) was added to a mixture of intermediate I-6 (196 mg, 0.59 mmol), intermediate II-8c (100 mg, 0.43 mmol) and DIPEA [7087-68-5] (0.6 mL, 3.53 mmol) in DMF (5 mL) at rt. The mixture was stirred at rt for 16 h. Then, a 1 M NaHCO3 aqueous solution was added, and the mixture was extracted with DCM. The organic layer was dried (MgSO4), filtered and the solvents evaporated in vacuo. The crude product was purified by flash column chromatography (silica; DCM/MeOH (9:1) in DCM 0/100 to 30/70). The desired fractions were collected and concentrated in vacuo to yield compound 7ab as an off-white solid (97 mg, 34%).

1H NMR (400 MHz, DMSO) δ 9.03 (dd, J=7.5, 6.2 Hz, 1H), 8.46 (t, J=6.0 Hz, 1H), 7.95 (d, J=8.2 Hz, 2H), 7.49 (dd, J=9.8, 2.6 Hz, 1H), 7.44 (d, J=8.3 Hz, 2H), 7.06 (td, J=7.6, 2.7 Hz, 1H), 4.57 (d, J=5.9 Hz, 2H), 4.32 (dd, J=12.8, 3.6 Hz, 1H), 4.17 (td, J=12.1, 4.9 Hz, 1H), 3.26-3.13 (m, 2H), 2.99 (dd, J=15.0, 7.5 Hz, 2H), 2.95-2.87 (m, 1H), 2.37-2.26 (m, 1H), 2.11 (qd, J=11.4, 5.8 Hz, 1H), 1.27 (t, J=7.5 Hz, 3H).

### Synthesis of Compound 8ab

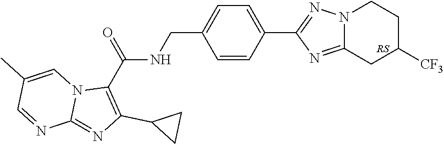 [SMILES: Cc1cnc2nc(C3CC3)c(C(=O)NCc3ccc(-c4nc5n(n4)CCC(C(F)(F)F)C5)cc3)n2c1]

HATU [148893-10-1] (306 mg, 0.81 mmol) was added to a mixture of intermediate I-6 (134 mg, 0.40 mmol), intermediate II-6b (105 mg, 0.48 mmol) and DIPEA [7087-68-5] (0.41 mL, 2.42 mmol) in DMF (5 mL) at rt. The mixture was stirred at rt for 16 h. Then the mixture was washed with a 1 M Na2CO3 aqueous solution and brine and extracted with EtOAc. The organic layer was dried (MgSO4), filtered and the solvents evaporated in vacuo. The crude product was purified by flash column chromatography (silica; EtOAc in heptane 0/100 to 100/0). The desired fractions were collected and concentrated in vacuo and the residue was repurified by reverse phase HPLC (Phenomenex Gemini C18 100×30 mm 5 μm Column; from 59% [0.1% HCOOH]−41% [ACN:MeOH (1:1)] to 17% [0.1% HCOOH]−83% [ACN:MeOH (1:1)]). The desired fractions were collected and concentrated in vacuo and the residue was purified by flash column chromatography (silica; EtOAc in heptane 0/100 to 100/0) to yield compound 8ab as a white solid (13 mg, 6%).

1H NMR (400 MHz, DMSO) δ 9.18 (s, 1H), 8.64 (t, J=5.8 Hz, 1H), 8.48 (d, J=2.1 Hz, 1H), 7.95 (d, J=8.1 Hz, 2H), 7.46 (d, J=8.1 Hz, 2H), 4.59 (d, J=5.8 Hz, 2H), 4.32 (dd, J=12.4, 4.1 Hz, 1H), 4.20-4.11 (m, 1H), 3.19 (d, J=13.3 Hz, 2H), 2.92 (dd, J=17.6, 12.2 Hz, 1H), 2.47-2.40 (m, 1H), 2.32 (s, 3H), 2.31-2.28 (m, 1H), 2.11 (ddd, J=24.4, 11.6, 5.7 Hz, 1H), 1.06 (d, J=6.4 Hz, 4H).

### Synthesis of Compound 9ab

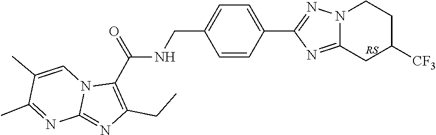 [SMILES: CCc1nc2nc(C)c(C)cn2c1C(=O)NCc1ccc(-c2nc3n(n2)CCC(C(F)(F)F)C3)cc1]

HATU [148893-10-1] (114 mg, 0.30 mmol) was added to a mixture of intermediate I-6 (100 mg, 0.30 mmol), intermediate II-8d (114 mg, 0.52 mmol) and DIPEA [7087-68-5] (0.21 mL, 1.20 mmol) in DMF (1.5 mL) at rt. The mixture was stirred at rt for 19 h. Then a saturated NaHCO3 aqueous solution was added, and the mixture was extracted with DCM. The organic layer was washed with brine, dried (MgSO4), filtered and the solvents evaporated in vacuo. The crude product was purified by flash column chromatography (silica; MeOH in DCM 0/100 to 2/98). The desired fractions were collected and concentrated in vacuo to yield compound 9ab as a white solid (15 mg, 6%).

1H NMR (400 MHz, DMSO) δ 9.06 (s, 1H), 8.42 (t, J=6.0 Hz, 1H), 7.95 (d, J=8.2 Hz, 2H), 7.44 (d, J=8.2 Hz, 2H), 4.56 (d, J=5.9 Hz, 2H), 4.32 (dd, J=12.7, 3.8 Hz, 1H), 4.17 (td, J=12.1, 4.9 Hz, 1H), 3.23-3.15 (m, 2H), 2.99 (q, J=7.6 Hz, 2H), 2.96-2.87 (m, 1H), 2.51 (s, 3H), 2.35-2.29 (m, 1H), 2.27 (s, 3H), 2.17-2.08 (m, 1H), 1.26 (t, J=7.5 Hz, 3H).

### Synthesis of Compound 10ab

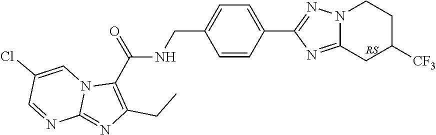 [SMILES: CCc1nc2ncc(Cl)cn2c1C(=O)NCc1ccc(-c2nc3n(n2)CCC(C(F)(F)F)C3)cc1]

A mixture of intermediate II-8e (105 mg, 0.42 mmol), HATU [148893-10-1] (229 mg, 0.60 mmol) and DIPEA [7087-68-5] (0.31 mL, 1.80 mmol) in DMF (7.8 mL) was stirred at rt for 10 min and then intermediate I-6 (100 mg, 0.30 mmol) was added. The mixture was stirred at rt for 16 h. Then a saturated NaHCO3 aqueous solution was added, and the mixture was extracted with EtOAc (3×). The combined organic layers were dried (MgSO4), filtered and the solvents evaporated in vacuo. The crude product was triturated with DIPE, dissolved in DCM, and extracted with a saturated NaHCO3 aqueous solution. The organic layer was dried (MgSO4), filtered and evaporated in vacuo to yield compound 10ab as a white solid (15 mg, 10%).

1H NMR (400 MHz, DMSO) δ 9.43 (d, J=2.6 Hz, 1H), 8.69 (d, J=2.6 Hz, 1H), 8.62 (t, J=5.9 Hz, 1H), 7.95 (d, J=8.2 Hz, 2H), 7.46 (d, J=8.1 Hz, 2H), 4.58 (d, J=5.8 Hz, 2H), 4.33 (dd, J=12.6, 4.2 Hz, 1H), 4.17 (td, J=11.9, 4.8 Hz, 1H), 3.19 (d, J=13.1 Hz, 2H), 3.05 (q, J=7.5 Hz, 2H), 2.93 (dd, J=17.8, 12.3 Hz, 1H), 2.32 (d, J=10.5 Hz, 1H), 2.11 (qd, J=12.0, 5.8 Hz, 1H), 1.29 (t, J=7.5 Hz, 3H).

### Synthesis of Compounds 10a and 10b

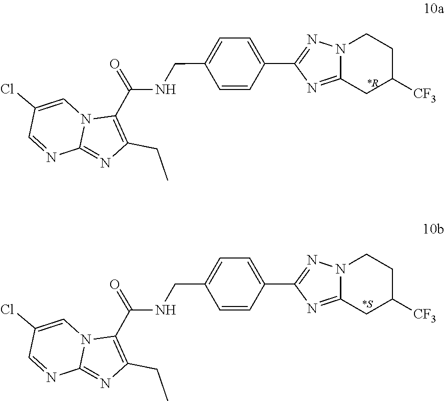 [SMILES: CCc1nc2ncc(Cl)cn2c1C(=O)NCc1ccc(-c2nc3n(n2)CCC(C(F)(F)F)C3)cc1 ; CCc1nc2ncc(Cl)cn2c1C(=O)NCc1ccc(-c2nc3n(n2)CCC(C(F)(F)F)C3)cc1]

A batch of compound 10ab, prepared according to an analogous procedure to the one outlined above starting from intermediate I-6 (0.74 g, 1.89 mmol), was purified by chiral SFC method on a Jasco SFC prep system using an Phenomenex Lux Cellulose-1 250 mm long×30 mm I.D. 5 μm particle size, on isocratic mode at 30 ml/min of CO2 (50%)−methanol (40%)+0.1% diethylamine, at 30° C., BPR 150 Bar. Acquisition frequency was set to 220 nm for the DAD detector. The desired fractions were collected and concentrated in vacuo to yield compound 10a (107 mg, 11%) and compound 10b (118 mg, yield=12%) as beige solids.

Compound 10a 1H NMR (400 MHz, DMSO) δ 9.43 (d, J=2.6 Hz, 1H), 8.68 (d, J=2.6 Hz, 1H), 8.62 (t, J=5.8 Hz, 1H), 7.95 (d, J=8.2 Hz, 2H), 7.46 (d, J=8.2 Hz, 2H), 4.58 (d, J=5.8 Hz, 2H), 4.33 (dd, J=12.9, 3.6 Hz, 1H), 4.17 (td, J=12.2, 4.9 Hz, 1H), 3.23-3.14 (m, 2H), 3.05 (q, J=7.5 Hz, 2H), 2.92 (dd, J=17.7, 12.2 Hz, 1H), 2.32 (dd, J=10.8, 2.1 Hz, 1H), 2.20-2.03 (m, 1H), 1.29 (t, J=7.5 Hz, 3H).

Compound 10b 1H NMR (400 MHz, DMSO) δ 9.43 (d, J=2.6 Hz, 1H), 8.68 (d, J=2.6 Hz, 1H), 8.62 (t, J=5.9 Hz, 1H), 7.94 (t, J=8.3 Hz, 2H), 7.46 (d, J=8.3 Hz, 2H), 4.58 (d, J=5.8 Hz, 2H), 4.33 (dd, J=13.1, 3.4 Hz, 1H), 4.17 (td, J=12.0, 4.8 Hz, 1H), 3.24-3.14 (m, 2H), 3.05 (q, J=7.5 Hz, 2H), 2.92 (dd, J=17.5, 12.1 Hz, 1H), 2.32 (dd, J=10.9, 2.1 Hz, 1H), 2.19-2.04 (m, 1H), 1.29 (t, J=7.5 Hz, 3H).

### Synthesis of Compound 11ab

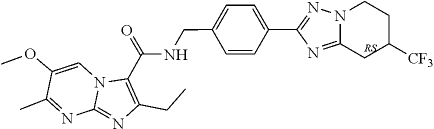 [SMILES: CCc1nc2nc(C)c(OC)cn2c1C(=O)NCc1ccc(-c2nc3n(n2)CCC(C(F)(F)F)C3)cc1]

Intermediate I-6 (110 mg, 0.33 mmol) was added to a solution of intermediate II-8f (106 mg, 0.36 mmol), HATU [148893-10-1] (189 mg, 0.50 mmol) and DIPEA [7087-68-5] (0.40 mL, 2.31 mmol) in DMF (4 mL) at rt. The mixture was stirred at rt for 19 h. Then a saturated NaHCO3 aqueous solution was added, and the mixture was extracted with EtOAc (2×). The combined organic layers were dried (MgSO4), filtered and the solvents evaporated in vacuo. The crude product was purified by flash column chromatography (silica; DCM/MeOH (9:1) in DCM 0/100 to 30/70). The desired fractions were collected and concentrated in vacuo to yield compound 11ab as a pale orange solid (27 mg, 15%).

1H NMR (400 MHz, DMSO) δ 8.88 (s, 1H), 8.36 (t, J=5.6 Hz, 1H), 7.95 (d, J=8.1 Hz, 2H), 7.45 (d, J=8.2 Hz, 2H), 4.57 (d, J=5.6 Hz, 2H), 4.36-4.28 (m, 1H), 4.17 (td, J=12.0, 4.7 Hz, 1H), 3.86 (s, 3H), 3.23-3.14 (m, 2H), 3.00 (q, J=7.4 Hz, 2H), 2.97-2.86 (m, 1H), 2.46 (s, 3H), 2.36-2.27 (m, 1H), 2.19-2.05 (m, 1H), 1.26 (t, J=7.5 Hz, 3H).

### Synthesis of Compound 12ab

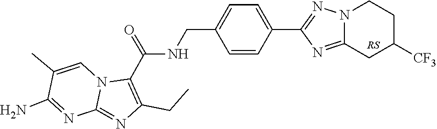 [SMILES: CCc1nc2nc(N)c(C)cn2c1C(=O)NCc1ccc(-c2nc3n(n2)CCC(C(F)(F)F)C3)cc1]

Trifluoroacetic acid [76-05-1] (2.7 mL, 36.35 mmol) was added to intermediate I-17a (179 mg, 0.24 mmol) at 0° C. The mixture was stirred at rt for 1 h. The mixture was neutralized with a saturated NaHCO3 aqueous solution and extracted with DCM (×3). The combined organic layers were dried (MgSO4), filtered and concentrated in vacuo. The crude product was purified by reverse phase HPLC (Phenomenex Gemini C18 30×100 mm 5 μm Column; from 95% [0.1% HCOOH]−5% [ACN:MeOH (1:1)] to 63% [0.1% HCOOH]−37% [ACN:MeOH (1:1)]). The desired fractions were collected, then a saturated NaHCO3 aqueous solution was added, and the mixture was extracted with DCM (×3). The combined organic layers were dried (MgSO4), filtered and concentrated in vacuo to dryness and the solid was triturated with DIPE and n-pentane to yield compound 12ab as a white solid (30 mg, 24%).

1H NMR (400 MHz, DMSO) δ 8.74 (s, 1H), 8.06 (t, J=5.9 Hz, 1H), 7.95 (d, J=8.1 Hz, 2H), 7.42 (d, J=8.2 Hz, 2H), 6.86 (s, 2H), 4.52 (d, J=5.8 Hz, 2H), 4.33 (dd, J=12.5, 3.9 Hz, 1H), 4.17 (td, J=12.1, 4.7 Hz, 1H), 3.23-3.15 (m, 2H), 2.98-2.91 (m, 1H), 2.87 (q, J=7.5 Hz, 2H), 2.33 (d, J=11.5 Hz, 1H), 2.18-2.10 (m, 1H), 2.06 (s, 3H), 1.22 (t, J=7.5 Hz, 3H).

### Synthesis of Compound 13ab

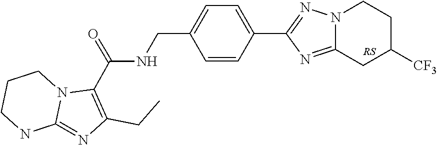 [SMILES: CCc1nc2n(c1C(=O)NCc1ccc(-c3nc4n(n3)CCC(C(F)(F)F)C4)cc1)CCCN2]

HATU [148893-10-1] (570 mg, 1.5 mmol) was added to a mixture of intermediate I-6 (250 mg, 0.75 mmol), intermediate II-17a (248 mg, 1.28 mmol) and DIPEA [7087-68-5] (0.21 mL, 1.20 mmol) in DMF (10 mL) at rt. The mixture was stirred at rt for 16 h. Then a saturated NaHCO3 aqueous solution was added, and the mixture was extracted with DCM. The organic layer was washed with brine, dried (MgSO4), filtered and the solvents evaporated in vacuo. The crude product was purified by flash column chromatography (silica; DCM/MeOH (9:1) in DCM 0/100 to 40/60). The desired fractions were collected and concentrated in vacuo and the residue was repurified by reverse phase HPLC (Phenomenex Gemini C18 100×30 mm 5 μm Column; from 95% [0.1% HCOOH]−5% [ACN:MeOH (1:1)] to 63% [0.1% HCOOH]−37% [ACN:MeOH (1:1)]). The desired fractions were collected and concentrated in vacuo to yield compound 13ab as a white solid (79 mg, 22%)

1H NMR (400 MHz, DMSO) δ 8.25 (t, J=6.0 Hz, 1H), 7.93 (d, J=8.2 Hz, 2H), 7.38 (d, J=8.3 Hz, 2H), 4.45 (d, J=6.0 Hz, 2H), 4.36-4.28 (m, 1H), 4.17 (td, J=12.0, 4.9 Hz, 1H), 3.99 (t, J=5.8 Hz, 2H), 3.18 (dd, J=9.5, 8.3 Hz, 2H), 2.92 (dd, J=17.6, 12.2 Hz, 1H), 2.71 (t, J=6.3 Hz, 2H), 2.64 (q, J=7.5 Hz, 2H), 2.36-2.27 (m, 1H), 2.18-2.03 (m, 1H), 1.91-1.73 (m, 4H), 1.10 (t, J=7.5 Hz, 3H).

### Synthesis of Compound 14ab

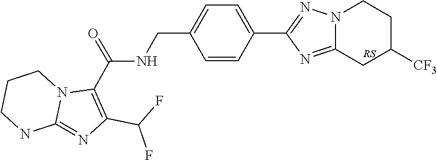 [SMILES: O=C(NCc1ccc(-c2nc3n(n2)CCC(C(F)(F)F)C3)cc1)c1c(C(F)F)nc2n1CCCN2]

Intermediate I-6 (110 mg, 0.33 mmol) was added to a solution of intermediate II-17b (120 mg, 0.46 mmol), HATU [148893-10-1] (189 mg, 0.50 mmol) and DIPEA [7087-68-5] (0.4 mL, 2.31 mmol) in DMF (4 mL) at rt. The mixture was stirred at rt for 72 h. Then a saturated NaHCO3 aqueous solution was added, and the mixture was extracted with EtOAc (×2). The organic layer was washed with brine, separated, dried (MgSO4), filtered and the solvents evaporated in vacuo. The crude product was purified by flash column chromatography (silica; DCM/methanol 9:1 in DCM 0/100 to 30/70). The desired fractions were collected and concentrated in vacuo and the residue was repurified by reverse phase HPLC (Phenomenex Gemini C18 21.2×100 mm 5 μm Column; from 72% [(65 mM NH4OAc)+ACN (90:10) pH 7]−28% (ACN/methanol 1:1) to from 36% [(65 mM NH4OAc)+ACN (90:10) pH 7]−28% (ACN/methanol 1:1). The desired fractions were collected and concentrated in vacuo to yield compound 14ab as a white solid (41 mg, 25%).

1H NMR (400 MHz, DMSO) δ 9.04 (t, J=5.7 Hz, 1H), 8.82 (d, J=7.0 Hz, 1H), 7.96 (d, J=8.2 Hz, 2H), 7.76 (d, J=9.1 Hz, 1H), 7.56-7.50 (m, 1H), 7.48 (s, 1H), 7.45 (s, 1H), 7.35 (t, J=53.8 Hz, 1H), 7.17 (td, J=6.9, 1.1 Hz, 1H), 4.59 (d, J=5.7 Hz, 2H), 4.37-4.29 (m, 1H), 4.23-4.13 (m, 1H), 3.24-3.16 (m, 2H), 2.93 (dd, J=17.8, 12.3 Hz, 1H), 2.36-2.28 (m, 1H), 2.18-2.05 (m, 1H).

### Synthesis of Compound 15ab

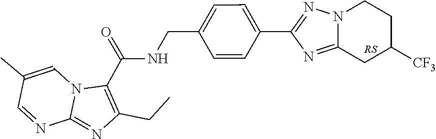 [SMILES: CCc1nc2ncc(C)cn2c1C(=O)NCc1ccc(-c2nc3n(n2)CCC(C(F)(F)F)C3)cc1]

HATU [148893-10-1] (420 mg, 1.10 mmol) was added to a mixture of intermediate I-6 (234 mg, 0.55 mmol), intermediate II-17c (440 mg, 1.06 mmol) and DIPEA [7087-68-5] (0.6 ml, 3.31 mmol) in DMF (4 ml) and the mixture was stirred at rt for 24. The mixture was washed with a saturated NaHCO3 aqueous solution and extracted with DCM. The organic layer was separated, dried (MgSO4), filtered and the solvents evaporated in vacuo. The crude product was washed with toluene twice and then purified by flash column chromatography over silica gel (20; MeOH in DCM 0/100 to 5/95). The desired fractions were collected and concentrated in vacuo and the residue triturated sequentially with DIPE:DCM (9:1), DIPE:DCM (1:9), DIPE:DCM (1:1) and Et2O. The solid was dried in vacuo and repurified by reverse phase HPLC (Phenomenex Gemini C18 30×100 mm 5 μm Column; from 70% [25 mM NH4HCO3]−30% [ACN:MeOH (1:1)] to 27% [25 mM NH4HCO3]−73% [ACN:MeOH (1:1)]). The desired fractions were collected, concentrated, and dried at 60° C. under vacuum to yield compound 15ab as a white solid (69 mg, 26%).

1H NMR (400 MHz, DMSO) δ 8.81 (s, 1H), 8.40 (t, J=6.0 Hz, 1H), 7.96 (d, J=8.3 Hz, 2H), 7.51 (d, J=9.1 Hz, 1H), 7.45 (d, J=8.3 Hz, 2H), 7.25 (dd, J=9.1, 1.7 Hz, 1H), 4.57 (d, J=5.9 Hz, 2H), 4.37-4.28 (m, 1H), 4.17 (td, J=12.1, 4.9 Hz, 1H), 3.24-3.11 (m, 2H), 3.03-2.95 (m, 2H), 2.92 (dd, J=16.0, 10.6 Hz, 1H), 2.36-2.32 (m, 1H), 2.31 (s, 3H), 2.18-2.04 (m, 1H), 1.26 (t, J=7.5 Hz, 3H).

### Synthesis of Compound 16ab and Compound 17ab

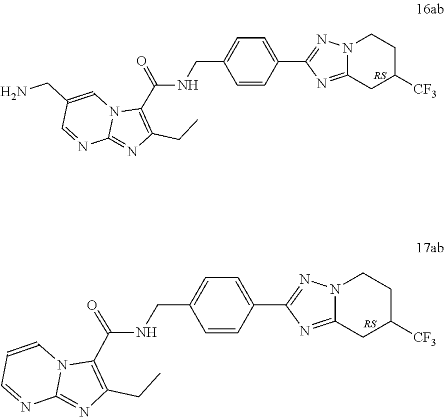 [SMILES: CCc1nc2ncc(CN)cn2c1C(=O)NCc1ccc(-c2nc3n(n2)CCC(C(F)(F)F)C3)cc1 ; CCc1nc2ncccn2c1C(=O)NCc1ccc(-c2nc3n(n2)CCC(C(F)(F)F)C3)cc1]

A 4M HCl dioxane solution (0.42 mL, 1.66 mmol) was added to a stirred solution of intermediate I-17b (165 mg, 0.28 mmol) in DCM (5 mL) at rt. The reaction mixture was stirred at rt for 16 h and then the solvents were evaporated in vacuo. The crude product was purified by flash column chromatography (silica, 12; DCM/MeOH/NH3 9:1:0.1 in DCM 0/100 to 100/0). The desired fractions were collected and concentrated in vacuo to yield compound 16ab as a brown solid (33 mg, 23%) and compound 17ab as a white solid (35 mg, 27%).

Compound 16ab 1H NMR (400 MHz, DMSO) δ 9.24 (d, J=2.4 Hz, 1H), 8.61 (d, J=2.4 Hz, 1H), 8.52 (t, J=5.8 Hz, 1H), 7.95 (d, J=8.2 Hz, 2H), 7.45 (d, J=8.2 Hz, 2H), 4.58 (d, J=5.8 Hz, 2H), 4.32 (dd, J=11.7, 4.4 Hz, 1H), 4.17 (td, J=12.0, 4.9 Hz, 1H), 3.80 (s, 2H), 3.24-3.13 (m, 2H), 3.03 (q, J=7.5 Hz, 2H), 2.92 (dd, J=17.4, 12.0 Hz, 1H), 2.36-2.27 (m, 1H), 2.19-2.02 (m, 1H), 1.28 (t, J=7.5 Hz, 3H).

Compound 17ab 1H NMR (400 MHz, DMSO) δ 9.34 (dd, J=6.9, 2.0 Hz, 1H), 8.63 (dd, J=4.2, 2.0 Hz, 1H), 8.56 (t, J=5.9 Hz, 1H), 7.96 (d, J=8.2 Hz, 2H), 7.46 (d, J=8.3 Hz, 2H), 7.18 (dd, J=6.9, 4.2 Hz, 1H), 4.59 (d, J=5.8 Hz, 2H), 4.33 (dd, J=12.7, 3.8 Hz, 1H), 4.17 (td, J=12.1, 4.9 Hz, 1H), 3.25-3.14 (m, 2H), 3.05 (q, J=7.5 Hz, 2H), 2.93 (dd, J=17.5, 12.0 Hz, 1H), 2.37-2.27 (m, 1H), 2.20-2.05 (m, 1H), 1.30 (t, J=7.5 Hz, 3H).

### Synthesis of Compound 18ab

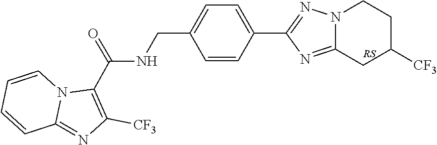 [SMILES: O=C(NCc1ccc(-c2nc3n(n2)CCC(C(F)(F)F)C3)cc1)c1c(C(F)(F)F)nc2ccccn12]

Intermediate I-10 (122 mg, 0.33 mmol) was added to a solution of intermediate II-6a (132 mg, 0.57 mmol), HATU [148893-10-1] (126 mg, 0.33 mmol) and DIPEA [7087-68-5] (0.23 mL, 1.33 mmol) in DMF (1.7 mL) at rt. The mixture was stirred at rt for 3 h. Then a saturated NaHCO3 aqueous solution was added, and the mixture was extracted with DCM. The organic layer was washed with brine, separated, dried (MgSO4), filtered and the solvents evaporated in vacuo. The crude product was purified by flash column chromatography (silica; EtOAc in heptane 0/100 to 80/20). The desired fractions were collected and concentrated in vacuo to yield compound 18ab as a white solid (85 mg, 48%).

1H NMR (400 MHz, DMSO) δ 9.28 (t, J=5.7 Hz, 1H), 8.56 (d, J=7.0 Hz, 1H), 7.80 (d, J=9.2 Hz, 1H), 7.72 (d, J=8.2 Hz, 2H), 7.59-7.53 (m, 2H), 7.35 (d, J=8.2 Hz, 2H), 7.21 (t, J=6.9 Hz, 1H), 4.54 (d, J=5.8 Hz, 2H), 4.17 (dd, J=12.9, 3.5 Hz, 1H), 3.99 (td, J=11.9, 4.4 Hz, 1H), 3.17-3.03 (m, 2H), 2.79 (dd, J=17.3, 12.6 Hz, 1H), 2.24 (d, J=11.5 Hz, 1H), 2.06-1.93 (m, 1H).

### Synthesis of Compound 19ab

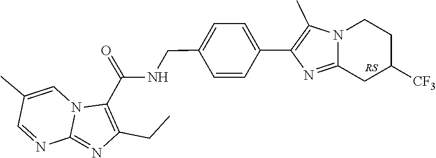 [SMILES: CCc1nc2ncc(C)cn2c1C(=O)NCc1ccc(-c2nc3n(c2C)CCC(C(F)(F)F)C3)cc1]

Intermediate I-22 (200 mg, 0.52 mmol) was added to a solution of intermediate II-3a (140 mg, 0.58 mmol), HATU [148893-10-1] (199 mg, 0.52 mmol) and DIPEA [7087-68-5] (0.37 mL, 2.1 mmol) in DMF (2.9 mL) at 0° C. under N2. The mixture was stirred at rt for 16 h. Then a saturated NaHCO3 aqueous solution was added, and the mixture was extracted with DCM. The organic layer was washed with brine, separated, dried (MgSO4), filtered and the solvents evaporated in vacuo. The crude product was purified by flash column chromatography (silica; MeOH in DCM 0/100 to 2/98). The desired fractions were collected and concentrated in vacuo to yield compound 19ab as a white solid (127 mg, 48%).

1H NMR (400 MHz, DMSO) δ 9.16 (dd, J=2.3, 1.1 Hz, 1H), 8.51 (d, J=2.4 Hz, 1H), 8.48 (t, J=6.0 Hz, 1H), 7.57 (d, J=8.2 Hz, 2H), 7.37 (d, J=8.3 Hz, 2H), 4.54 (d, J=5.8 Hz, 2H), 4.11-4.03 (m, 1H), 3.79 (td, J=12.0, 4.7 Hz, 1H), 3.09-2.98 (m, 4H), 2.75 (dd, J=17.0, 12.5 Hz, 1H), 2.34 (d, J=0.4 Hz, 3H), 2.33-2.23 (m, 4H), 1.94 (ddd, J=24.6, 11.7, 5.6 Hz, 1H), 1.28 (t, J=7.5 Hz, 3H).

### Synthesis of Compound 20ab

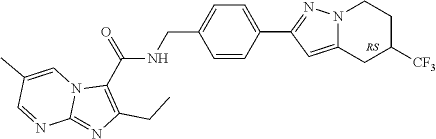 [SMILES: CCc1nc2ncc(C)cn2c1C(=O)NCc1ccc(-c2cc3n(n2)CCC(C(F)(F)F)C3)cc1]

Intermediate I-27 (126 mg, 0.34 mmol) was added to a solution of intermediate II-3a (120 mg, 0.41 mmol), HATU [148893-10-1] (194 mg, 0.51 mmol) and DIPEA [7087-68-5] (0.42 mL, 2.39 mmol) in DMF (5 mL) at rt. The mixture was stirred at rt for 16 h. Then a saturated NaHCO3 aqueous solution was added, and the mixture was extracted with EtOAc (3×). The combined organic layers were, dried (MgSO4), filtered and the solvents evaporated in vacuo. The crude product was purified by flash column chromatography (silica; MeOH in DCM 0/100 to 100/0). The desired fractions were collected and concentrated in vacuo and the residue was repurified by reverse phase (Phenomenex Gemini C18 30×100 mm 5 μm Column; from 59% [25 mM NH4HCO3]−41% [ACN:MeOH (1:1)] to 17% [25 mM NH4HCO3]−83% [ACN:MeOH (1:1)]). The desired fractions were collected and concentrated in vacuo to yield compound 20ab as a white solid (85 mg, 51%).

1H NMR (400 MHz, DMSO) δ 9.18-9.11 (m, 1H), 8.53-8.45 (m, 2H), 7.73 (d, J=8.2 Hz, 2H), 7.38 (d, J=8.2 Hz, 2H), 6.52 (s, 1H), 4.54 (d, J=5.9 Hz, 2H), 4.30 (dd, J=12.9, 3.6 Hz, 1H), 4.11 (td, J=12.3, 4.8 Hz, 1H), 3.13 (dd, J=15.9, 4.0 Hz, 1H), 3.02 (q, J=7.5 Hz, 3H), 2.79 (dd, J=15.8, 11.2 Hz, 1H), 2.34 (s, 3H), 2.27 (dd, J=13.3, 2.2 Hz, 1H), 2.04 (qd, J=11.8, 5.7 Hz, 1H), 1.28 (t, J=7.5 Hz, 3H).

### Synthesis of Compound 21ab

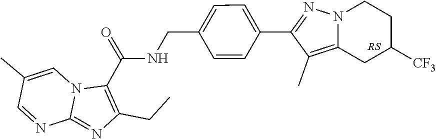 [SMILES: CCc1nc2ncc(C)cn2c1C(=O)NCc1ccc(-c2nn3c(c2C)CC(C(F)(F)F)CC3)cc1]

Intermediate I-32 (59 mg, 0.15 mmol) was added to a solution of intermediate II-3a (63 mg, 0.22), HATU [148893-10-1] (88 mg, 0.23 mmol) and DIPEA [7087-68-5] (0.19 mL, 1.08 mmol) in DMF (3 mL) at rt. The mixture was stirred at rt for 16 h. Then a saturated NaHCO3 aqueous solution was added, and the mixture was extracted with EtOAc (3×). The combined organic layers were, dried (MgSO4), filtered and the solvents evaporated in vacuo. The crude product was purified by flash column chromatography (silica; MeOH in DCM 0/100 to 100/0). The desired fractions were collected and concentrated in vacuo and the residue was repurified by reverse phase (Phenomenex Gemini C18 30×100 mm 5 μm Column; from 70% [0.1% HCOOH]−30% [ACN:MeOH (1:1)] to 27% [0.1% HCOOH]−73% [ACN:MeOH (1:1)]). The desired fractions were collected and concentrated in vacuo to yield compound 21ab as a white solid (25 mg, 32%).

1H NMR (400 MHz, DMSO) δ 9.19-9.13 (m, 1H), 8.51 (d, J=2.4 Hz, 1H), 8.50-8.47 (m, 1H), 7.59 (d, J=8.2 Hz, 2H), 7.41 (d, J=8.2 Hz, 2H), 4.56 (d, J=5.8 Hz, 2H), 4.31-4.22 (m, 1H), 4.07 (td, J=12.3, 4.6 Hz, 1H), 3.08-2.96 (m, 4H), 2.72-2.60 (m, 1H), 2.34 (s, 3H), 2.30-2.22 (m, 1H), 2.08 (s, 3H), 2.06-1.93 (m, 1H), 1.28 (t, J=7.5 Hz, 3H).

### Synthesis of Compound 22ab

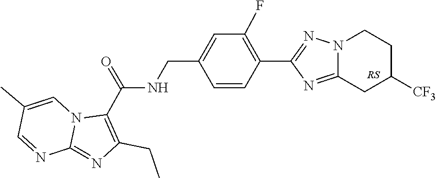 [SMILES: CCc1nc2ncc(C)cn2c1C(=O)NCc1ccc(-c2nc3n(n2)CCC(C(F)(F)F)C3)c(F)c1]

HATU [148893-10-1] (434 mg, 1.14 mmol) was added to a mixture of intermediate I-37 (200 mg, 0.57 mmol), intermediate II-3a (140 mg, 0.68 mmol) and DIPEA [7087-68-5] (0.58 mL, 3.42 mmol) in DMF (10 mL) at rt. The mixture was stirred at rt for 16 h and then a 1M Na2CO3 aqueous solution was added, and the mixture was extracted with EtOAc. The organic layer was separated, washed with brine, dried (MgSO4), filtered and the solvents evaporated in vacuo. The crude product was purified by flash column chromatography (silica; EtOAc in heptane 0/100 to 100/0). The desired fractions were collected and concentrated in vacuo and the residue was repurified by reverse phase (Phenomenex Gemini C18 100×30 mm 5 μm Column; from 81% [0.1% HCOOH]−19% [ACN:MeOH (1:1)] to 45% [0.1% HCOOH]−55% [ACN:MeOH (1:1)]). The desired fractions were collected and concentrated in vacuo to yield compound 22ab as a white solid (122 mg, 42%).

1H NMR (400 MHz, DMSO) δ 9.33 (s, 1H), 8.68 (s, 1H), 8.67 (d, J=8.6 Hz, 1H), 8.11 (t, J=7.8 Hz, 1H), 7.46 (d, J=9.8 Hz, 2H), 4.75 (d, J=5.8 Hz, 2H), 4.51 (dd, J=12.7, 3.9 Hz, 1H), 4.35 (td, J=12.1, 4.8 Hz, 1H), 3.40-3.31 (m, 2H), 3.20 (q, J=7.5 Hz, 2H), 3.10 (dd, J=17.6, 12.2 Hz, 1H), 2.66 (s, 3H), 2.47 (s, 1H), 2.29 (ddd, J=23.7, 11.2, 5.2 Hz, 1H), 1.46 (t, J=7.5 Hz, 3H).

### Synthesis of Compound 23ab

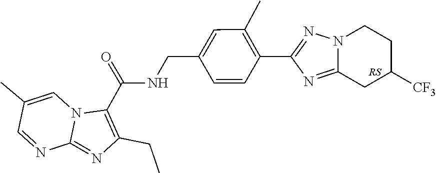 [SMILES: CCc1nc2ncc(C)cn2c1C(=O)NCc1ccc(-c2nc3n(n2)CCC(C(F)(F)F)C3)c(C)c1]

HATU [148893-10-1] (0.44 g, 1.16 mmol) was added to a mixture of intermediate I-42 (0.2 g, 0.58 mmol), intermediate II-3a (0.18 g, 0.89 mmol) and DIPEA [7087-68-5] (0.59 mL, 3.47 mmol) in DMF (5 mL) at rt. The mixture was stirred at rt for 16 h and then a saturated NaHCO3 aqueous solution was added, and the mixture was extracted with DCM. The organic layer was separated, washed with brine, dried (MgSO4), filtered and the solvents evaporated in vacuo. The crude product was washed with toluene and triturated with DCM. The precipitate was collected, and the mother liquor was concentrated in vacuo and the residue was by flash column chromatography (silica; EtOAc in heptane 0/100 to 100/0). The desired fractions were collected and concentrated in vacuo and the residue was repurified by reverse phase (Phenomenex Gemini C18 100×30 mm 5 μm Column; from 70% [25 mM NH4HCO3]−30% [ACN:MeOH (1:1)] to 27% [25 mM NH4HCO3]−73% [ACN:MeOH (1:1)]). The desired fractions were collected and concentrated in vacuo and the solid obtained combined with the solid previously obtained to yield compound 23ab as a white solid (108 mg, 37%).

1H NMR (400 MHz, DMSO) δ 9.15 (s, 1H), 8.51 (s, 2H), 7.87 (d, J=7.8 Hz, 1H), 7.27 (d, J=8.7 Hz, 2H), 4.54 (d, J=5.7 Hz, 2H), 4.34 (dd, J=12.3, 4.2 Hz, 1H), 4.18 (td, J=12.0, 4.6 Hz, 1H), 3.20 (d, J=13.4 Hz, 2H), 3.03 (dd, J=14.9, 7.5 Hz, 2H), 2.93 (dd, J=17.3, 12.1 Hz, 1H), 2.58 (s, 3H), 2.34 (s, 3H), 2.31 (s, 1H), 2.13 (ddd, J=24.9, 12.2, 5.9 Hz, 1H), 1.29 (t, J=7.4 Hz, 3H).

### Synthesis of Compound 24ab

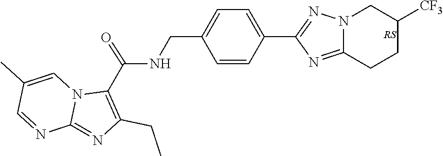 [SMILES: CCc1nc2ncc(C)cn2c1C(=O)NCc1ccc(-c2nc3n(n2)CC(C(F)(F)F)CC3)cc1]

Intermediate II-3a (173 mg, 0.49 mmol) was added to a solution of intermediate I-47 (173 mg, 0.64 mmol), HATU [148893-10-1] (282 mg, 0.74 mmol) and DIPEA [7087-68-5] (0.60 mL, 3.46 mmol) in DMF (5 mL). The reaction mixture was stirred at rt for 16 hours, diluted with a saturated NaHCO3 aqueous solution and extracted with EtOAc (×2). The combined organic layers were dried (MgSO4), filtered and concentrated in vacuo. The crude product was purified by flash column chromatography (silica; DCM/MeOH 9:1 in DCM 0/100 to 30/70). The desired fractions were collected and concentrated in vacuo to yield compound 24ab as a white solid (149 mg, 62%).

1H NMR (400 MHz, DMSO) δ 9.16 (s, 1H), 8.51 (s, 2H), 7.95 (d, J=7.9 Hz, 2H), 7.45 (d, J=7.9 Hz, 2H), 4.57 (s, 2H), 4.51-4.42 (m, 1H), 4.19 (t, J=11.2 Hz, 1H), 3.44-3.35 (m, 1H), 3.17 (s, 1H), 3.08-2.99 (m, 2H), 2.98-2.89 (m, 1H), 2.34 (s, 3H), 2.25 (d, J=11.8 Hz, 1H), 2.09-1.95 (m, 1H), 1.28 (t, J=7.4 Hz, 3H).

### Synthesis of Compound 25ab

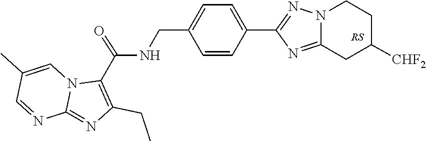 [SMILES: CCc1nc2ncc(C)cn2c1C(=O)NCc1ccc(-c2nc3n(n2)CCC(C(F)F)C3)cc1]

Intermediate I-51 (120 mg, 0.34 mmol) was added to a solution of intermediate II-3a (151 mg, 0.52 mmol), HATU [148893-10-1] (194 mg, 0.51 mmol) and DIPEA [7087-68-5] (0.42 mL, 2.38 mmol) in DMF (3 mL). The reaction mixture was stirred at rt for 16 hours, diluted with a saturated NaHCO3 aqueous solution and extracted with EtOAc (×3). The combined organic layers were dried (MgSO4), filtered and concentrated in vacuo. The crude product was purified by flash column chromatography (silica; DCM/MeOH 9:1 in DCM 0/100 to 100/0). The desired fractions were collected and concentrated in vacuo to yield compound 25ab as a beige solid (89 mg, 55%).

1H NMR (400 MHz, DMSO) δ 9.20-9.12 (m, 1H), 8.56-8.45 (m, 2H), 7.95 (d, J=8.2 Hz, 2H), 7.44 (d, J=8.2 Hz, 2H), 6.20 (td, J=56.2, 4.1 Hz, 1H), 4.57 (d, J=5.9 Hz, 2H), 4.35-4.26 (m, 1H), 4.13 (td, J=12.1, 4.9 Hz, 1H), 3.10-2.97 (m, 3H), 2.85-2.73 (m, 1H), 2.70-2.56 (m, 1H), 2.34 (s, 3H), 2.20 (d, J=13.5 Hz, 1H), 2.02-1.88 (m, 1H), 1.28 (t, J=7.5 Hz, 3H).

### Synthesis of Compound 26ab

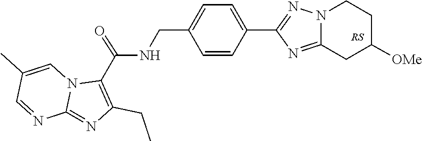 [SMILES: CCc1nc2ncc(C)cn2c1C(=O)NCc1ccc(-c2nc3n(n2)CCC(OC)C3)cc1]

HATU [148893-10-1] (523 mg, 1.37 mmol) was added to a mixture of intermediate I-56 (202 mg, 0.68 mmol), intermediate II-3a (239 mg, 1.16 mmol) and DIPEA [7087-68-5] (0.7 mL, 4.1 mmol) in DMF (5 mL) at rt. The mixture was stirred at rt for 18 h and then a saturated NaHCO3 aqueous solution was added, and the mixture was extracted with DCM. The organic layer was separated, dried (MgSO4), filtered and the solvents evaporated in vacuo. The crude product was purified by flash column chromatography (silica; MeOH in DCM 0/100 to 10/90). The desired fractions were collected and concentrated in vacuo and the residue was dissolved in a mixture 95/5 of DCM and methanol and the solution was extracted with a saturated NaHCO3 aqueous solution. The organic layer was separated, dried (MgSO4), filtered and the solvents evaporated in vacuo. The residue was triturated with DIPE/DCM (9:1) and DIPE/DCM (1:1) and dried in vacuo to yield compound 26ab as a white solid (179 mg, 58%).

1H NMR (300 MHz, DMSO) δ 8.35 (s, 2H), 8.00 (d, J=8.2 Hz, 2H), 7.55 (d, J=8.2 Hz, 2H), 4.28-3.99 (m, 4H), 3.95 (d, J=3.9 Hz, 1H), 3.33 (s, 3H), 3.15-2.91 (m, 2H), 2.34-2.09 (m, 2H).

### Synthesis of Compound 27ab and Compound 28ab

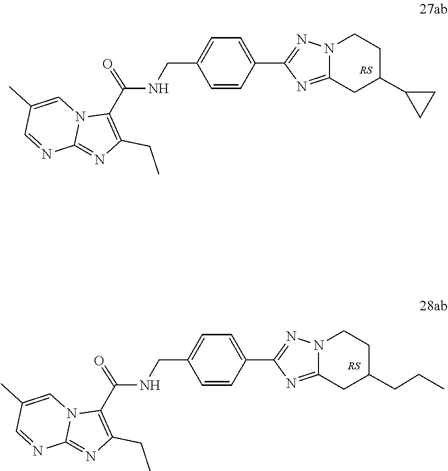 [SMILES: CCc1nc2ncc(C)cn2c1C(=O)NCc1ccc(-c2nc3n(n2)CCC(C2CC2)C3)cc1 ; CCCC1CCn2nc(-c3ccc(CNC(=O)c4c(CC)nc5ncc(C)cn45)cc3)nc2C1]

Intermediate I-60 (154 mg, 0.45 mmol) was added to a solution of intermediate II-3a (575 mg, 1.96 mmol), HATU [148893-10-1] (803 mg, 2.11 mmol) and DIPEA [7087-68-5] (1.71 mL, 9.86 mmol) in DMF (4 mL). The reaction mixture was stirred at rt for 16 hours, diluted with a saturated NaHCO3 aqueous solution and extracted with EtOAc (×3). The combined organic layers were dried (MgSO4), filtered and concentrated in vacuo. The crude product was purified by flash column chromatography (silica; DCM/MeOH 9:1 in DCM 0/100 to 100/0). The desired fractions were collected and concentrated in vacuo and the residue was purified by reverse phase HPLC (Phenomenex Gemini C18 30×100 mm 5 μm Column; from 72% [0.1% HCOOH]−28% [ACN:MeOH (1:1)] to 36% [0.1% HCOOH]−64% [ACN:MeOH (1:1)]). The desired fractions were collected and concentrated to yield compounds 27ab (28 mg, 14%) and 28ab (84 mg, 40%) as white solids.

Compound 27ab 1H NMR (400 MHz, DMSO) δ 9.19-9.12 (m, 1H), 8.55-8.46 (m, 2H), 7.94 (d, J=8.3 Hz, 2H), 7.43 (d, J=8.3 Hz, 2H), 4.57 (d, J=5.9 Hz, 2H), 4.30-4.18 (m, 1H), 4.10-3.99 (m, 1H), 3.08-2.96 (m, 3H), 2.71-2.59 (m, 1H), 2.34 (d, J=0.6 Hz, 3H), 2.20-2.08 (m, 1H), 2.01-1.84 (m, 1H), 1.28 (t, J=7.5 Hz, 4H), 0.81-0.71 (m, 1H), 0.51-0.41 (m, 2H), 0.28-0.15 (m, 2H).

Compound 28ab 1H NMR (400 MHz, DMSO) δ 9.16 (dd, J=2.4, 1.1 Hz, 1H), 8.54-8.46 (m, 2H), 7.94 (d, J=8.3 Hz, 2H), 7.43 (d, J=8.3 Hz, 2H), 4.57 (d, J=5.8 Hz, 2H), 4.27-4.16 (m, 1H), 4.12-4.00 (m, 1H), 3.07-2.95 (m, 3H), 2.46-2.40 (m, 1H), 2.34 (d, J=0.6 Hz, 3H), 2.16-2.05 (m, 1H), 1.97 (s, 1H), 1.80-1.66 (m, 1H), 1.45-1.34 (m, 4H), 1.28 (t, J=7.5 Hz, 3H), 0.98-0.85 (m, 3H).

### Synthesis of Compound 29ab

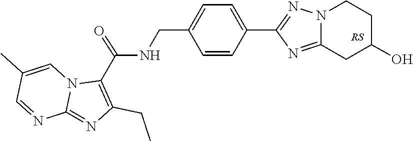 [SMILES: CCc1nc2ncc(C)cn2c1C(=O)NCc1ccc(-c2nc3n(n2)CCC(O)C3)cc1]

Intermediate I-64 (92 mg, 0.32 mmol) was added to a solution of intermediate II-3a (85 mg, 0.27 mmol), HATU [148893-10-1] (103 mg, 0.27 mmol) and DIPEA [7087-68-5] (0.33 mL, 1.88 mmol) in DMF (3 mL). The reaction mixture was stirred at rt for 16 hours, diluted with a saturated NaHCO3 aqueous solution and extracted with DCM (×3). The combined organic layers were dried (MgSO4), filtered and concentrated in vacuo. The crude product was washed with water and triturated with n-pentane and DCM to yield compound 29ab as a pale brown solid (77 mg, 66%).

1H NMR (400 MHz, DMSO) δ 9.16 (s, 1H), 8.50 (dd, J=7.6, 4.1 Hz, 2H), 7.94 (d, J=8.2 Hz, 2H), 7.44 (d, J=8.2 Hz, 2H), 5.21 (d, J=3.4 Hz, 1H), 4.57 (d, J=5.8 Hz, 2H), 4.26 (s, 1H), 4.17 (t, J=6.2 Hz, 2H), 3.09-2.97 (m, 3H), 2.79 (dd, J=17.0, 4.9 Hz, 1H), 2.34 (s, 3H), 2.18-1.98 (m, 2H), 1.27 (q, J=7.8 Hz, 3H).

### Synthesis of Compound 30ab

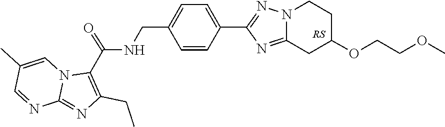

Intermediate I-68 (199 mg, 0.41 mmol) was added to a solution of intermediate II-3a (134 mg, 0.49 mmol), HATU [148893-10-1] (156 mg, 0.41 mmol) and DIPEA [7087-68-5] (0.5 mL, 2.85 mmol) in DMF (3 mL). The reaction mixture was stirred at rt for 16 hours, diluted with a saturated NaHCO3 aqueous solution and extracted with DCM (×3). The combined organic layers were dried (MgSO4), filtered and concentrated in vacuo. The rude product was purified by flash column chromatography (silica; DCM/MeOH 9:1 in DCM 0/100 to 100/0). The desired fractions were collected and concentrated in vacuo to yield compound 30ab as a yellow solid (83 mg, 40%).

1H NMR (400 MHz, DMSO) δ 9.16 (d, J=1.0 Hz, 1H), 8.50 (dd, J=7.1, 4.2 Hz, 2H), 7.94 (d, J=8.2 Hz, 2H), 7.44 (d, J=8.2 Hz, 2H), 4.57 (d, J=5.8 Hz, 2H), 4.22-4.03 (m, 3H), 3.70-3.56 (m, 2H), 3.43 (t, J=4.7 Hz, 2H), 3.22 (s, 3H), 3.10 (dd, J=17.1, 4.2 Hz, 1H), 3.02 (q, J=7.5 Hz, 2H), 2.93 (dd, J=17.1, 4.4 Hz, 1H), 2.34 (s, 3H), 2.29-2.20 (m, 1H), 2.15 (dt, J=13.4, 6.6 Hz, 1H), 1.28 (t, J=7.5 Hz, 3H).

### Synthesis of Compound 31ab

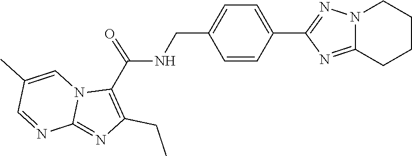 [SMILES: CCc1nc2ncc(C)cn2c1C(=O)NCc1ccc(-c2nc3n(n2)CCCC3)cc1]

DMF (10 mL) was added to intermediate II-3a (0.19 g, 0.67 mmol) at rt and the mixture was stirred at rt for 10 min. Then, DIPEA [7087-68-5] (0.58 ml, 3.33 mmol) was added and the mixture was stirred at rt for a further 10 min. Then, HATU [148893-10-1] (0.26 g, 0.69 mmol) was added to the mixture at rt and the mixture was stirred at rt for 10 min. Finally intermediate I-73a (0.21 g, 0.42 mmol) was added to the mixture and the reaction mixture was stirred for at rt for 18 hours more. The mixture was diluted with an aqueous saturated NaHCO3 solution and then extracted with DCM (×3). The combined organic layers were dried (MgSO4), filtered and concentrated in vacuo. The excess of DMF was co-distilled with toluene (100 ml×3). The residue was dissolved in DCM and the organic phase was washed with a saturated NaHCO3 aqueous solution. The organic layer was dried (MgSO4), filtered and concentrated in vacuo. The crude product was purified by flash column chromatography (silica; MeOH in DCM 0/100 to 5/95). The desired fractions were collected and concentrated in vacuo. The solid was triturated with diethyl ether and drops of DCM and dried in vacuo to yield compound 31ab as a white solid (128 mg, 72%).

1H NMR (400 MHz, DMSO) δ 9.16 (dd, J=2.3, 1.1 Hz, 1H), 8.50 (dd, J=7.4, 4.2 Hz, 2H), 7.94 (d, J=8.2 Hz, 2H), 7.44 (d, J=8.2 Hz, 2H), 4.57 (d, J=5.9 Hz, 2H), 4.13 (t, J=6.0 Hz, 2H), 3.02 (q, J=7.5 Hz, 2H), 2.85 (t, J=6.3 Hz, 2H), 2.34 (s, 3H), 2.05-1.98 (m, 2H), 1.94-1.85 (m, 2H), 1.28 (t, J=7.5 Hz, 3H).

### Synthesis of Compound 32ab

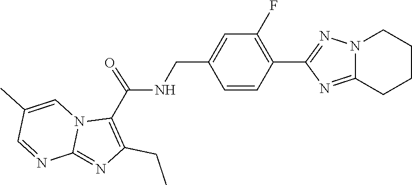 [SMILES: CCc1nc2ncc(C)cn2c1C(=O)NCc1ccc(-c2nc3n(n2)CCCC3)c(F)c1]

Compound 32ab was prepared according to an analogous procedure to the one used for the synthesis of compound 3lab using intermediate I-73b (150 mg, 0.45 mmol) as starting material (116 mg, 59%).

1H NMR (400 MHz, DMSO) δ 9.17 (dd, J=2.3, 1.1 Hz, 1H), 8.51 (dd, J=9.1, 4.2 Hz, 2H), 7.94 (t, J=7.8 Hz, 1H), 7.28 (d, J=9.8 Hz, 2H), 4.57 (d, J=5.9 Hz, 2H), 4.15 (t, J=6.0 Hz, 2H), 3.03 (q, J=7.5 Hz, 2H), 2.86 (t, J=6.3 Hz, 2H), 2.34 (s, 3H), 2.07-1.98 (m, 2H), 1.96-1.85 (m, 2H), 1.29 (t, J=7.5 Hz, 3H).

### Synthesis of Compound 33ab

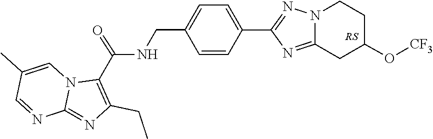 [SMILES: CCc1nc2ncc(C)cn2c1C(=O)NCc1ccc(-c2nc3n(n2)CCC(OC(F)(F)F)C3)cc1]

Intermediate I-75 (100 mg, 0.26 mmol) was added to a solution of intermediate II-3a (110 mg, 0.39 mmol), HATU [148893-10-1] (100 mg, 0.26 mmol) and DIPEA [7087-68-5] (0.32 mL, 1.82 mmol) in DMF (3 mL). The reaction mixture was stirred at rt for 16 hours, diluted with a saturated NaHCO3 aqueous solution and extracted with DCM (×3). The combined organic layers were dried (MgSO4), filtered and concentrated in vacuo. The crude product was purified by flash column chromatography (silica; MeOH in DCM 0/100 to 100/0). The desired fractions were collected and concentrated in vacuo and the residue was repurified by reverse phase HPLC (Phenomenex Gemini C18 30×100 mm 5 μm; from 70% [25 mM NH4HCO3]−30% [ACN:MeOH (1:1)] to 27% [25 mM NH4HCO3]−73% [ACN:MeOH (1:1)]). The desired fractions were collected and concentrated in vacuo to yield compound 33ab as a yellow solid (10 mg, 7%).

1H NMR (400 MHz, DMSO) δ 9.16 (dd, J=2.3, 1.1 Hz, 1H), 8.51 (dd, J=6.6, 4.2 Hz, 2H), 7.95 (d, J=8.3 Hz, 2H), 7.45 (d, J=8.3 Hz, 2H), 5.25-5.19 (m, 1H), 4.57 (d, J=5.9 Hz, 2H), 4.31 (dt, J=11.2, 5.5 Hz, 1H), 4.25-4.15 (m, 1H), 3.36 (dd, J=17.4, 4.4 Hz, 1H), 3.17 (dd, J=17.3, 4.9 Hz, 1H), 3.02 (q, J=7.5 Hz, 2H), 2.40 (dd, J=11.3, 5.6 Hz, 2H), 2.34 (s, 3H), 1.28 (t, J=7.5 Hz, 3H).

### Synthesis of Compound 34ab

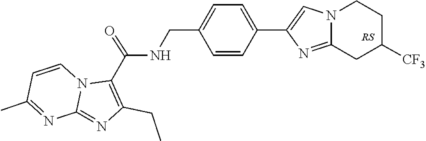 [SMILES: CCc1nc2nc(C)ccn2c1C(=O)NCc1ccc(-c2cn3c(n2)CC(C(F)(F)F)CC3)cc1]

Intermediate I-10 (202 mg, 0.52 mmol) was added to a solution of intermediate II-8a (200 mg, 0.73 mmol), HATU [148893-10-1] (297 mg, 0.78 mmol) and DIPEA [7087-68-5] (0.64 mL, 3.65 mmol) in DMF (10 mL). The reaction mixture was stirred at rt for 16 hours, diluted with a saturated NaHCO3 aqueous solution and extracted with DCM. The combined organic layers were dried (MgSO4), filtered and concentrated in vacuo. The crude product was purified by flash column chromatography (silica; MeOH in DCM 0/100 to 30/70). The desired fractions were collected and concentrated in vacuo to yield compound 34ab as a beige solid (177 mg, 69%).

1H NMR (400 MHz, DMSO) δ 9.18 (d, J=7.0 Hz, 1H), 8.42 (t, J=5.9 Hz, 1H), 7.69 (d, J=8.2 Hz, 2H), 7.52 (s, 1H), 7.33 (d, J=8.2 Hz, 2H), 7.05 (d, J=7.1 Hz, 1H), 4.51 (d, J=5.8 Hz, 2H), 4.20-4.11 (m, 1H), 4.05-3.90 (m, 1H), 3.12-3.04 (m, 2H), 2.99 (q, J=7.5 Hz, 2H), 2.83-2.72 (m, 1H), 2.54 (s, 3H), 2.27-2.18 (m, 1H), 2.01-1.85 (m, 1H), 1.26 (t, J=7.5 Hz, 3H).

### Synthesis of Compound 35ab

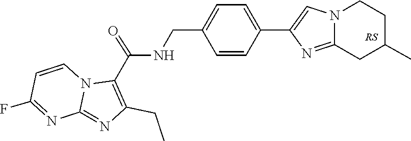 [SMILES: CCc1nc2nc(F)ccn2c1C(=O)NCc1ccc(-c2cn3c(n2)CC(C)CC3)cc1]

HATU [148893-10-1] (440 mg, 1.15 mmol) was added to a mixture of I-16 (160 mg, 0.57 mmol), II-8c (180 mg, 0.86 mmol) and DIPEA [7087-68-5] (0.6 ml, 3.53 mmol) in DMF (6 ml) and the mixture was stirred at rt for 20 hours. The mixture was washed with a 1M NaHCO3 aqueous solution and extracted with DCM. The organic layer was separated, dried (MgSO4), filtered and the solvents evaporated in vacuo. The excess of DMF was co-distilled with toluene (100 ml×3). The residue was dissolved in DCM and the organic phase was washed with a saturated NaHCO3 aqueous solution. The organic layer was separated, dried (MgSO4), filtered and the solvents evaporated in vacuo. The crude product was triturated with DCM:DIPE 1:1 to yield a white solid. The solid was dried in vacuo and purified by flash column chromatography (silica; MeOH in DCM 0/100 to 10/90). The desired fractions were collected and concentrated in vacuo and the residue was triturated with DCM:DIPE 1:1 to yield compound 35ab as a white solid (50 mg, 22%).

1H NMR (400 MHz, DMSO) δ 9.03 (dd, J=7.4, 6.2 Hz, 1H), 8.45 (t, J=5.9 Hz, 1H), 7.94 (d, J=8.2 Hz, 2H), 7.49 (dd, J=9.8, 2.6 Hz, 1H), 7.43 (d, J=8.3 Hz, 2H), 7.06 (td, J=7.7, 2.7 Hz, 1H), 4.56 (d, J=5.9 Hz, 2H), 4.22 (ddd, J=12.6, 5.6, 2.9 Hz, 1H), 4.13-4.02 (m, 1H), 3.29 (s, 1H), 3.05-2.92 (m, 3H), 2.09 (d, J=5.4 Hz, 1H), 2.05 (dd, J=13.7, 2.7 Hz, 1H), 1.74 (dtd, J=13.4, 10.8, 5.8 Hz, 1H), 1.27 (t, J=7.5 Hz, 3H), 1.10 (d, J=6.6 Hz, 3H).

### Synthesis of Compound 36ab

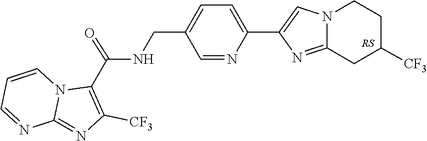 [SMILES: O=C(NCc1ccc(-c2cn3c(n2)CC(C(F)(F)F)CC3)nc1)c1c(C(F)(F)F)nc2ncccn12]

HATU [148893-10-1] (229 mg, 0.6 mmol) and DIPEA [7087-68-5] (0.56 mL, 3.22 mmol) were added to a solution of intermediate II-6a (202 mg, 0.74 mmol) in DMF (5 mL) at rt. The mixture was stirred 10 min and then, intermediate I-81 (171 mg, 0.46 mmol) was added, and the reaction mixture was stirred at rt for 16 h. Then a saturated NaHCO3 aqueous solution was added, and the mixture was extracted with EtOAc (×3). The combined organic layers were dried (MgSO4), filtered and concentrated in vacuo. The excess of DMF was co-distilled with toluene (10 mL×3). The solid was dissolved in DCM and extracted with a saturated NaHCO3 aqueous solution. The organic phase was separated, dried (MgSO4), filtered and concentrated in vacuo. The crude product was purified by flash column chromatography (silica, DCM/MeOH 9:1 in DCM 0/100 to 30/70). The desired fractions were collected and concentrated in vacuo. The residue was repurified by reverse phase HPLC (Phenomenex Gemini C18 30×100 mm 5 μm; from 95% [0.1% HCOOH]−5% [ACN:MeOH (1:1)] to 63% [0.1% HCOOH]−37% [ACN:MeOH (1:1)]). The desired fractions were collected and concentrated in vacuo to yield compound 36ab as white solid (41 mg, yield: 17%).

1H NMR (400 MHz, DMSO) δ 9.30 (t, J=5.8 Hz, 1H), 8.57 (d, J=7.0 Hz, 1H), 8.49 (d, J=1.6 Hz, 1H), 7.85-7.73 (m, 3H), 7.66 (s, 1H), 7.59-7.52 (m, 1H), 7.20 (td, J=6.9, 1.0 Hz, 1H), 4.56 (d, J=5.6 Hz, 2H), 4.27-4.16 (m, 1H), 4.00 (td, J=12.3, 4.5 Hz, 1H), 3.17-3.04 (m, 2H), 2.80 (dd, J=17.4, 12.6 Hz, 1H), 2.29-2.19 (m, 1H), 2.04-1.89 (m, 1H).

### Synthesis of Final Compounds

### Method A: Coupling Reaction Between Carboxylic Acid Derivative and Amine Derivative

### General Procedure

HATU [CAS 148893-10-1] (1.5 to 2.5 eq.) was added to a mixture of carboxylic acid intermediate (1.4 to 4 eq.) and DIPEA (5 to 7 eq.) in DMF. The amine intermediate (1 eq.) was added and the reaction mixture was stirred at room temperature for 18 hours. The mixture was washed with a solution of NaHCO₃ (sat., aq.) and extracted with an appropriate solvent (DCM or EtOAc). The combined organic extracts were dried (anhydrous MgSO₄), filtered and the solvents were removed in vacuo.

The crude product was purified to afford the desired compounds.

Purification techniques used were flash column chromatography (silica, eluent: DCM/MeOH (9:1) in DCM, MeOH in DCM or EtOAc in heptane), reverse phase chromatography or preparative HPLC. Other purification technique may be used if needed, as for example trituration (DIPE, DCM, Et₂O).

It is understood that several purification techniques may be employed in order to get the desired compounds with high purity degree.

The Following Compounds were Obtained Through Method A:

### Synthesis of Compound 37

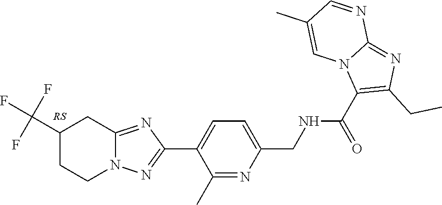 [SMILES: CCc1nc2ncc(C)cn2c1C(=O)NCc1ccc(-c2nc3n(n2)CCC(C(F)(F)F)C3)c(C)n1]

Compound 37 was synthesized from intermediates II-3a and I-138 and obtained as an off-white solid (37.5 mg, 21%). ¹H NMR (400 MHz, DMSO) δ 9.21 (s, 1H), 8.52 (d, J=2.4 Hz, 1H), 8.49 (d, J=5.8 Hz, 1H), 8.23 (d, J=8.1 Hz, 1H), 7.34 (d, J=8.1 Hz, 1H), 4.65 (d, J=5.7 Hz, 2H), 4.37 (dd, J=12.5, 3.9 Hz, 1H), 4.21 (td, J=11.7, 4.5 Hz, 1H), 3.22 (d, J=12.6 Hz, 2H), 3.09 (q, J=7.5 Hz, 2H), 2.95 (dd, J=17.7, 12.3 Hz, 1H), 2.81 (s, 3H), 2.34 (s, 3H), 2.33 (s, 1H), 2.13 (dtd, J=17.2, 11.3, 5.7 Hz, 1H), 1.33 (t, J=7.5 Hz, 3H).

### Synthesis of Compound 38

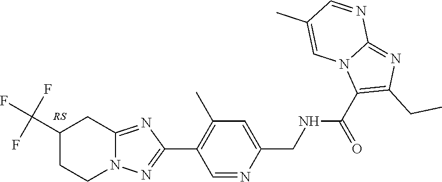 [SMILES: CCc1nc2ncc(C)cn2c1C(=O)NCc1cc(C)c(-c2nc3n(n2)CCC(C(F)(F)F)C3)cn1]

Compound 38 was synthesized from intermediates II-3a and I-139 and obtained as a white solid (41.7 mg, 24%). ¹H NMR (400 MHz, DMSO) δ 9.21 (d, J=1.1 Hz, 1H), 8.96 (s, 1H), 8.52 (d, J=2.5 Hz, 1H), 8.52-8.49 (m, 1H), 7.34 (s, 1H), 4.65 (d, J=5.8 Hz, 2H), 4.37 (dd, J=12.8, 3.7 Hz, 1H), 4.21 (td, J=12.2, 4.8 Hz, 1H), 3.27-3.18 (m, 2H), 3.07 (q, J=7.5 Hz, 2H), 2.96 (dd, J=17.8, 12.3 Hz, 1H), 2.60 (s, 3H), 2.34 (s, 3H), 2.32 (d, J=2.0 Hz, 1H), 2.13 (dtd, J=16.9, 11.3, 5.5 Hz, 1H), 1.32 (t, J=7.5 Hz, 3H).

### Synthesis of Compound 39

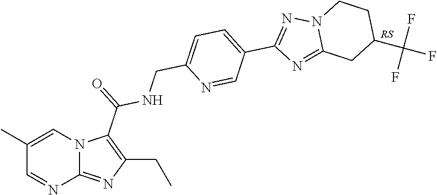 [SMILES: CCc1nc2ncc(C)cn2c1C(=O)NCc1ccc(-c2nc3n(n2)CCC(C(F)(F)F)C3)cn1]

Compound 39 was synthesized from intermediates II-3a and I-146 and obtained as a-white solid (7.6 mg, 11%). ¹H NMR (400 MHz, DMSO) δ 9.21 (dd, J=2.2, 1.1 Hz, 1H), 9.10 (d, J=1.8 Hz, 1H), 8.52 (t, J=4.7 Hz, 2H), 8.29 (dd, J=8.2, 2.2 Hz, 1H), 7.50 (d, J=8.2 Hz, 1H), 4.69 (d, J=5.8 Hz, 2H), 4.42-4.30 (m, 1H), 4.27-4.13 (m, 1H), 3.26-3.16 (m, 2H), 3.07 (q, J=7.5 Hz, 2H), 3.00-2.90 (m, 1H), 2.38-2.31 (m, 4H), 2.22-2.06 (m, 1H), 1.32 (t, J=7.5 Hz, 3H).

### Synthesis of Compound 40

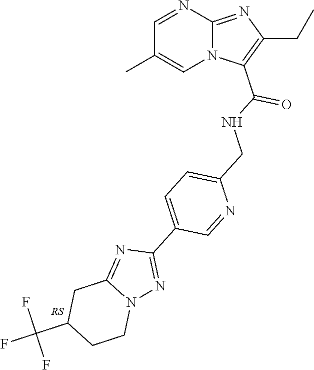 [SMILES: CCc1nc2ncc(C)cn2c1C(=O)NCc1ccc(-c2nc3n(n2)CCC(C(F)(F)F)C3)cn1]

Compound 40 was synthesized from intermediates II-8a and I-146 and obtained as a white solid (48.8 mg, 29%). ¹H NMR (400 MHz, DMSO) δ 9.20 (d, J=7.0 Hz, 1H), 8.46 (t, J=6.0 Hz, 1H), 7.94 (t, J=7.8 Hz, 1H), 7.29 (d, J=9.8 Hz, 2H), 7.06 (d, J=7.1 Hz, 1H), 4.57 (d, J=5.9 Hz, 2H), 4.42-4.26 (m, 1H), 4.18 (td, J=12.0, 4.8 Hz, 1H), 3.18 (d, J=4.1 Hz, 2H), 3.02 (q, J=7.5 Hz, 2H), 2.98-2.88 (m, 1H), 2.55 (s, 3H), 2.41-2.21 (m, 1H), 2.21-1.95 (m, 1H), 1.28 (t, J=7.5 Hz, 3H).

### Synthesis of Compound 41

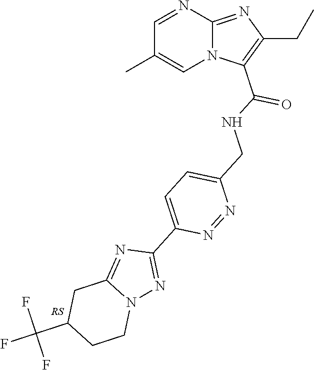 [SMILES: CCc1nc2ncc(C)cn2c1C(=O)NCc1ccc(-c2nc3n(n2)CCC(C(F)(F)F)C3)nn1]

Compound 41 was synthesized from intermediates II-3a and I-144 and obtained as white solid (21.3 mg, 49%). ¹H NMR (400 MHz, DMSO) δ 9.21 (dd, J=2.3, 1.1 Hz, 1H), 8.63 (s, 1H), 8.53 (d, J=2.4 Hz, 1H), 8.20 (d, J=8.8 Hz, 1H), 7.81 (d, J=8.8 Hz, 1H), 4.89 (s, 2H), 4.47-4.20 (m, 2H), 3.29-3.15 (m, 3H), 3.06 (q, J=7.5 Hz, 2H), 3.04-2.94 (m, 1H), 2.34 (d, J=0.7 Hz, 3H), 2.22-2.09 (m, 1H), 1.31 (t, J=7.5 Hz, 3H).

### Synthesis of Compound 42

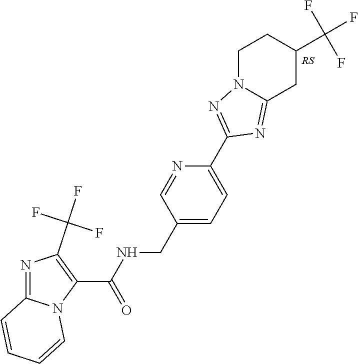 [SMILES: O=C(NCc1ccc(-c2nc3n(n2)CCC(C(F)(F)F)C3)nc1)c1c(C(F)(F)F)nc2ccccn12]

Compound 42 was synthesized from intermediates II-6a and I-145 and obtained as a beige solid (67.2 mg, 33%). ¹H NMR (400 MHz, DMSO) δ 9.36 (t, J=5.7 Hz, 1H), 8.65 (d, J=1.8 Hz, 1H), 8.59 (d, J=7.0 Hz, 1H), 8.02 (d, J=8.1 Hz, 1H), 7.88 (dd, J=8.1, 2.2 Hz, 1H), 7.80 (d, J=9.1 Hz, 1H), 7.60-7.54 (m, 1H), 7.21 (td, J=6.9, 1.0 Hz, 1H), 4.63 (d, J=5.6 Hz, 2H), 4.36 (dd, J=12.8, 3.6 Hz, 1H), 4.21 (td, J=12.2, 4.9 Hz, 1H), 3.25-3.15 (m, 2H), 3.01-2.89 (m, 1H), 2.34-2.30 (m, 1H), 2.13 (qd, J=11.6, 5.8 Hz, 1H).

### Synthesis of Compound 43

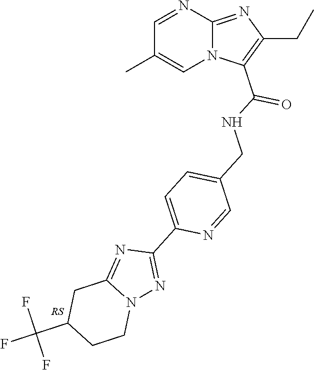 [SMILES: CCc1nc2ncc(C)cn2c1C(=O)NCc1ccc(-c2nc3n(n2)CCC(C(F)(F)F)C3)nc1]

Compound 43 was synthesized from intermediates II-3a and I-145 and obtained as a white solid (66.9 mg, 35%). ¹H NMR (400 MHz, DMSO) δ 9.16 (s, 1H), 8.65 (d, J=1.6 Hz, 1H), 8.54 (d, J=6.0 Hz, 1H), 8.52 (d, J=2.4 Hz, 1H), 8.01 (d, J=8.1 Hz, 1H), 7.88 (dd, J=8.1, 2.2 Hz, 1H), 4.60 (d, J=5.8 Hz, 2H), 4.36 (dd, J=13.1, 3.9 Hz, 1H), 4.25-4.15 (m, 1H), 3.22 (s, 1H), 3.18 (d, J=4.9 Hz, 1H), 3.02 (q, J=7.5 Hz, 2H), 2.94 (dd, J=17.6, 12.2 Hz, 1H), 2.34 (s, 3H), 2.33-2.31 (m, 1H), 2.13 (ddd, J=17.0, 11.0, 5.4 Hz, 1H), 1.28 (t, J=7.5 Hz, 3H).

### Synthesis of Compound 44

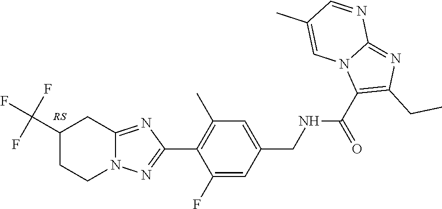 [SMILES: CCc1nc2ncc(C)cn2c1C(=O)NCc1cc(C)c(-c2nc3n(n2)CCC(C(F)(F)F)C3)c(F)c1]

Compound 44 was synthesized from intermediates II-3a and I-122 and obtained as a white solid (117 mg, 47%). ¹H NMR (400 MHz, DMSO) δ 9.16 (dd, J=2.3, 1.1 Hz, 1H), 8.52 (d, J=2.3 Hz, 1H), 8.50 (d, J=5.9 Hz, 1H), 7.15 (s, 1H), 7.10 (d, J=10.7 Hz, 1H), 4.54 (d, J=5.8 Hz, 2H), 4.35 (dd, J=12.7, 3.6 Hz, 1H), 4.20 (td, J=12.0, 4.5 Hz, 1H), 3.23 (s, 1H), 3.18 (d, J=4.1 Hz, 1H), 3.04 (q, J=7.5 Hz, 2H), 2.94 (dd, J=16.5, 10.6 Hz, 1H), 2.34 (s, 3H), 2.33-2.27 (m, 1H), 2.25 (s, 3H), 2.14 (ddd, J=24.6, 11.4, 5.7 Hz, 1H), 1.29 (t, J=7.5 Hz, 3H).

### Synthesis of Compound 45

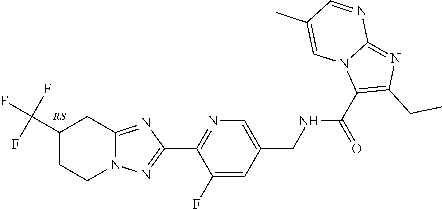 [SMILES: CCc1nc2ncc(C)cn2c1C(=O)NCc1cnc(-c2nc3n(n2)CCC(C(F)(F)F)C3)c(F)c1]

Compound 45 was synthesized from intermediates II-3a and I-141 and obtained as a white solid (50.1 mg, 59%). ¹H NMR (400 MHz, DMSO) δ 9.18 (dd, J=2.3, 1.1 Hz, 1H), 8.55 (s, 1H), 8.52 (q, J=4.7 Hz, 2H), 7.82 (d, J=11.4 Hz, 1H), 4.64 (d, J=5.8 Hz, 2H), 4.37 (dd, J=11.7, 4.7 Hz, 1H), 4.26-4.17 (m, 1H), 3.25-3.17 (m, 2H), 3.03 (q, J=7.5 Hz, 2H), 2.96 (dd, J=17.5, 12.0 Hz, 1H), 2.33 (d, J=5.5 Hz, 4H), 2.15 (tt, J=11.6, 5.8 Hz, 1H), 1.29 (t, J=7.5 Hz, 3H).

### Synthesis of Compound 46

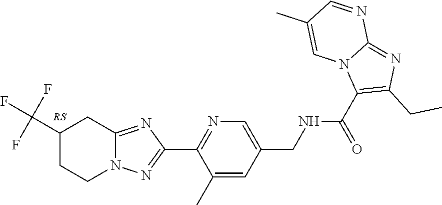 [SMILES: CCc1nc2ncc(C)cn2c1C(=O)NCc1cnc(-c2nc3n(n2)CCC(C(F)(F)F)C3)c(C)c1]

Compound 46 was synthesized from intermediates II-3a and I-143 and obtained as a white solid (125.8 mg, 57%). ¹H NMR (400 MHz, DMSO) δ 9.16 (dd, J=2.4, 1.1 Hz, 1H), 8.56-8.51 (m, J=8.1, 4.1 Hz, 2H), 8.49 (s, 1H), 7.70 (s, 1H), 4.57 (d, J=5.8 Hz, 2H), 4.36 (dd, J=13.0, 3.5 Hz, 1H), 4.20 (tt, J=12.1, 6.1 Hz, 1H), 3.30 (s, 3H), 3.26-3.15 (m, J=12.1 Hz, 2H), 3.03 (q, J=7.5 Hz, 2H), 2.95 (dd, J=17.3, 12.0 Hz, 1H), 2.34 (d, J=0.7 Hz, 3H), 2.33-2.30 (m, 1H), 2.20-2.08 (m, 1H), 1.29 (t, J=7.5 Hz, 3H).

### Synthesis of Compound 47

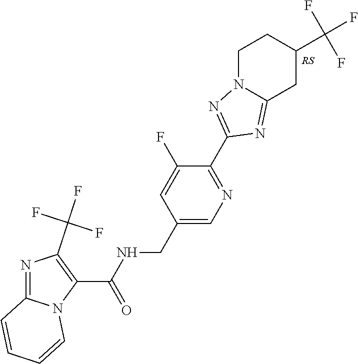 [SMILES: O=C(NCc1cnc(-c2nc3n(n2)CCC(C(F)(F)F)C3)c(F)c1)c1c(C(F)(F)F)nc2ccccn12]

Compound 47 was synthesized from intermediates II-6a and I-147 and obtained as a white solid (20.5 mg, 10%). ¹H NMR (400 MHz, DMSO) δ 9.36 (t, J=5.8 Hz, 1H), 8.62 (d, J=7.0 Hz, 1H), 8.55 (s, 1H), 7.83-7.78 (m, 2H), 7.60-7.55 (m, 1H), 7.21 (td, J=6.9, 1.1 Hz, 1H), 4.67 (d, J=5.4 Hz, 2H), 4.38 (dd, J=12.6, 3.5 Hz, 1H), 4.26-4.18 (m, 1H), 3.26-3.18 (m, 2H), 2.97 (dd, J=17.6, 12.3 Hz, 1H), 2.34 (d, J=10.9 Hz, 1H), 2.15 (qd, J=11.5, 5.8 Hz, 1H).

### Synthesis of Compound 48

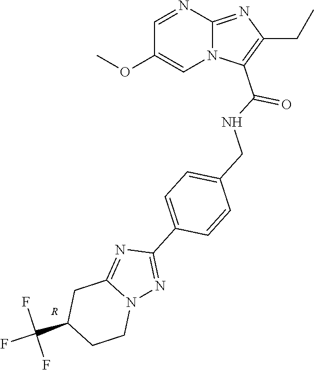

Compound 48 was synthesized from intermediates II-8b and I-88 and obtained as a white solid (1.28 g, 63%). ¹H NMR (400 MHz, DMSO) δ 8.97 (d, J=3.1 Hz, 1H), 8.52 (d, J=3.1 Hz, 1H), 8.47 (t, J=5.9 Hz, 1H), 7.96 (d, J=8.3 Hz, 2H), 7.45 (d, J=8.3 Hz, 2H), 4.58 (d, J=5.7 Hz, 2H), 4.38-4.27 (m, 1H), 4.17 (td, J=12.1, 4.8 Hz, 1H), 3.86 (s, 3H), 3.26-3.13 (m, 2H), 3.03 (q, J=7.5 Hz, 2H), 2.92 (dd, J=17.6, 12.1 Hz, 1H), 2.37-2.25 (m, 1H), 2.19-2.03 (m, 1H), 1.28 (t, J=7.5 Hz, 3H).

### Synthesis of Compound 49

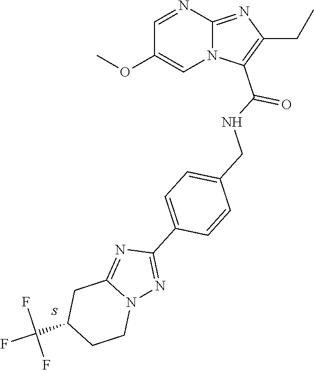

Compound 49 was synthesized from intermediates II-8b and I-87 and obtained as a white solid (1.10 g, 55%). ¹H NMR (400 MHz, DMSO) δ 8.97 (d, J=3.1 Hz, 1H), 8.52 (d, J=3.1 Hz, 1H), 8.48 (t, J=5.9 Hz, 1H), 7.96 (d, J=8.3 Hz, 2H), 7.45 (d, J=8.3 Hz, 2H), 4.58 (d, J=5.9 Hz, 2H), 4.37-4.26 (m, 1H), 4.17 (td, J=12.0, 4.8 Hz, 1H), 3.86 (s, 3H), 3.18 (dd, J=9.5, 8.3 Hz, 2H), 3.03 (q, J=7.5 Hz, 2H), 2.92 (dd, J=17.6, 12.1 Hz, 1H), 2.38-2.26 (m, 1H), 2.17-2.04 (m, 1H), 1.28 (t, J=7.5 Hz, 3H).

### Synthesis of Compound 50

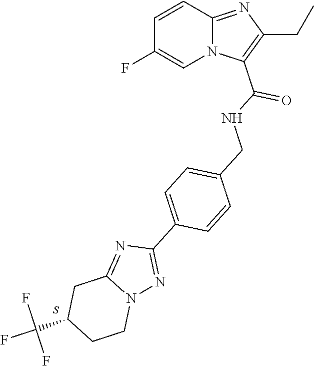

Compound 50 was synthesized from intermediates II-101 and I-87 and obtained as a beige solid (225 mg, 86%). ¹H NMR (400 MHz, DMSO) δ 9.08-9.04 (m, 1H), 8.47 (t, J=5.7 Hz, 1H), 7.95 (d, J=8.2 Hz, 2H), 7.69 (dd, J=9.8, 5.4 Hz, 1H), 7.53-7.47 (m, 1H), 7.45 (d, J=8.3 Hz, 2H), 4.58 (d, J=5.6 Hz, 2H), 4.33 (dd, J=12.7, 3.7 Hz, 1H), 4.17 (td, J=12.0, 4.8 Hz, 1H), 3.23-3.15 (m, 2H), 3.02 (q, J=7.5 Hz, 2H), 2.93 (dd, J=17.5, 12.1 Hz, 1H), 2.32 (dd, J=10.9, 2.1 Hz, 1H), 2.18-2.05 (m, 1H), 1.27 (t, J=7.5 Hz, 3H).

### Synthesis of Compound 51

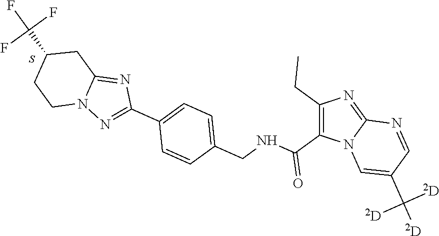

Compound 51 was synthesized from intermediates II-98 and I-87 and obtained as a white solid (264 mg, 97%). ¹H NMR (400 MHz, DMSO) δ 9.16 (d, J=2.4 Hz, 1H), 8.54-8.48 (m, 2H), 7.95 (d, J=8.3 Hz, 2H), 7.45 (d, J=8.3 Hz, 2H), 4.57 (d, J=5.9 Hz, 2H), 4.37-4.28 (m, 1H), 4.17 (td, J=12.0, 4.9 Hz, 1H), 3.23-3.15 (m, 2H), 3.02 (q, J=7.5 Hz, 2H), 2.92 (dd, J=17.6, 12.2 Hz, 1H), 2.32-2.28 (m, 1H), 2.17-2.05 (m, 1H), 1.28 (t, J=7.5 Hz, 3H).

### Synthesis of Compound 52

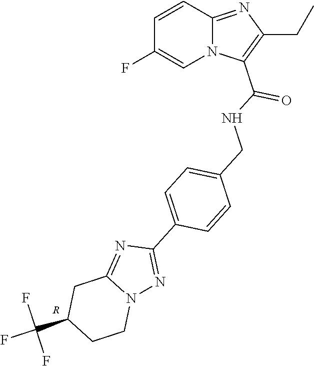

Compound 52 was synthesized from intermediates II-101 and I-88 and obtained as a white solid (239 mg, 92%). ¹H NMR (400 MHz, DMSO) δ 9.07 (dd, J=5.1, 2.3 Hz, 1H), 8.47 (s, 1H), 7.96 (d, J=8.2 Hz, 2H), 7.70 (dd, J=9.8, 5.3 Hz, 1H), 7.53-7.47 (m, 1H), 7.45 (d, J=8.2 Hz, 2H), 4.58 (d, J=3.7 Hz, 2H), 4.33 (dd, J=12.8, 3.5 Hz, 1H), 4.17 (td, J=12.2, 4.7 Hz, 1H), 3.24-3.16 (m, 2H), 3.02 (q, J=7.5 Hz, 2H), 2.93 (dd, J=17.3, 11.9 Hz, 1H), 2.35-2.28 (m, 1H), 2.12 (ddd, J=24.5, 11.5, 5.7 Hz, 1H), 1.28 (t, J=7.5 Hz, 3H).

### Synthesis of Compound 53

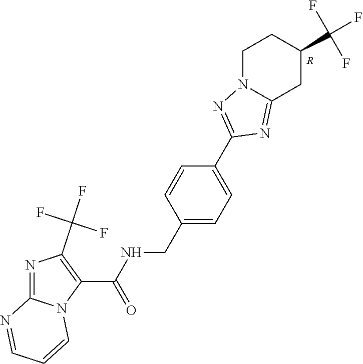

Compound 53 was synthesized from intermediates II-96 and I-88 and obtained as a yellow solid (22.1 mg, 12%). ¹H NMR (400 MHz, DMSO) δ 9.33 (t, J=5.8 Hz, 1H), 9.05 (dd, J=7.0, 2.0 Hz, 1H), 8.85 (dd, J=4.1, 2.0 Hz, 1H), 7.98 (d, J=8.3 Hz, 2H), 7.47 (d, J=8.3 Hz, 2H), 7.36 (dd, J=7.0, 4.1 Hz, 1H), 4.61 (d, J=5.7 Hz, 2H), 4.37-4.31 (m, 1H), 4.23-4.14 (m, 1H), 3.25-3.15 (m, 3H), 2.99-2.89 (m, 1H), 2.19-2.06 (m, 1H). 1H overlapped by solvent signal.

### Synthesis of Compound 54

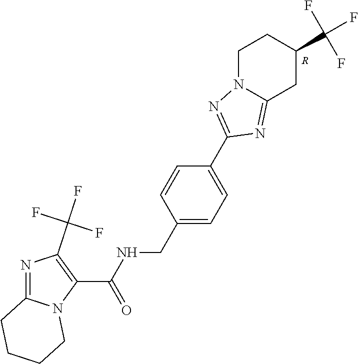

Compound 54 was synthesized from intermediates II-36 and I-88 and obtained as a pale yellow solid (61.9 mg, 32.5%). ¹H NMR (400 MHz, DMSO) δ 9.14 (t, J=6.0 Hz, 1H), 7.94 (d, J=8.3 Hz, 2H), 7.38 (d, J=8.3 Hz, 2H), 4.48 (d, J=5.9 Hz, 2H), 4.33 (dd, J=12.8, 3.5 Hz, 1H), 4.17 (td, J=12.0, 4.8 Hz, 1H), 3.93 (t, J=5.8 Hz, 2H), 3.24-3.14 (m, 2H), 2.93 (dd, J=17.5, 12.1 Hz, 1H), 2.78 (t, J=6.3 Hz, 2H), 2.34-2.28 (m, 1H), 2.12 (qd, J=11.5, 5.8 Hz, 1H), 1.91 (d, J=4.2 Hz, 2H), 1.88-1.79 (m, 2H).

### Synthesis of Compound 55

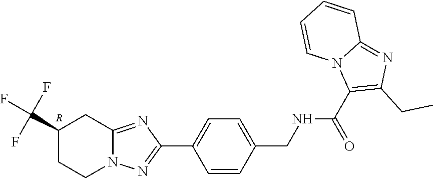

Compound 55 was synthesized from intermediates II-8b and I-88 and obtained as a white solid (126.1 mg, 66%). ¹H NMR (400 MHz, DMSO) δ 8.99 (dt, J=7.0, 1.0 Hz, 1H), 8.43 (t, J=5.9 Hz, 1H), 7.96 (d, J=8.3 Hz, 2H), 7.64-7.57 (m, 1H), 7.45 (d, J=8.3 Hz, 2H), 7.41-7.33 (m, 1H), 7.01 (td, J=6.9, 1.2 Hz, 1H), 4.58 (d, J=5.9 Hz, 2H), 4.37-4.27 (m, 1H), 4.17 (td, J=11.9, 4.7 Hz, 1H), 3.26-3.12 (m, 2H), 3.01 (q, J=7.5 Hz, 2H), 2.93 (dd, J=17.7, 12.2 Hz, 1H), 2.36-2.27 (m, 1H), 2.19-2.05 (m, 1H), 1.28 (t, J=7.5 Hz, 3H).

### Synthesis of Compound 56

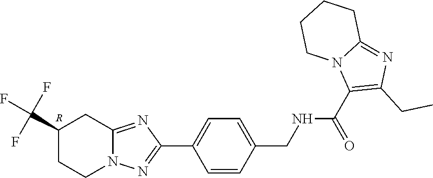

Compound 56 was synthesized from intermediates II-17a and I-88 and obtained as a white solid (130 mg, 66%). ¹H NMR (400 MHz, DMSO) δ 8.33 (t, J=5.6 Hz, 1H), 7.94 (d, J=8.3 Hz, 2H), 7.38 (d, J=8.3 Hz, 2H), 4.46 (d, J=6.0 Hz, 2H), 4.37-4.27 (m, 1H), 4.17 (td, J=12.0, 4.8 Hz, 1H), 4.01 (t, J=5.8 Hz, 2H), 3.26-3.12 (m, 2H), 2.92 (dd, J=17.6, 12.1 Hz, 1H), 2.73 (t, J=6.2 Hz, 2H), 2.65 (q, J=7.5 Hz, 2H), 2.37-2.27 (m, 1H), 2.18-2.05 (m, 1H), 1.93-1.75 (m, 4H), 1.11 (t, J=7.5 Hz, 3H).

### Synthesis of Compound 57

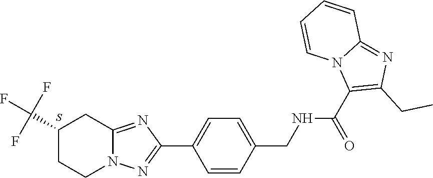

Compound 57 was synthesized from intermediates II-102 and I-87 and obtained as a white solid (158 mg, 81%). ¹H NMR (400 MHz, DMSO) δ 8.99 (d, J=6.9 Hz, 1H), 8.45 (t, J=5.9 Hz, 1H), 7.95 (d, J=8.3 Hz, 2H), 7.60 (d, J=9.0 Hz, 1H), 7.45 (d, J=8.3 Hz, 2H), 7.40-7.35 (m, 1H), 7.01 (td, J=6.9, 1.2 Hz, 1H), 4.57 (d, J=5.8 Hz, 2H), 4.33 (dd, J=13.0, 3.4 Hz, 1H), 4.22-4.11 (m, 1H), 3.25-3.15 (m, 2H), 3.01 (q, J=7.5 Hz, 2H), 2.93 (dd, J=17.6, 12.2 Hz, 1H), 2.36-2.27 (m, 1H), 2.18-2.05 (m, 1H), 1.27 (t, J=7.5 Hz, 3H).

### Synthesis of Compound 58

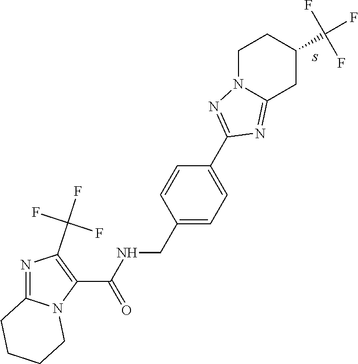

Compound 58 was synthesized from intermediates II-36 and I-87 and obtained as a beige solid (95 mg, 42%). ¹H NMR (400 MHz, DMSO) δ 9.14 (t, J=6.0 Hz, 1H), 7.94 (d, J=8.3 Hz, 2H), 7.38 (d, J=8.3 Hz, 2H), 4.48 (d, J=5.9 Hz, 2H), 4.33 (dd, J=12.9, 3.5 Hz, 1H), 4.17 (td, J=12.1, 4.8 Hz, 1H), 3.93 (t, J=5.8 Hz, 2H), 3.26-3.12 (m, 2H), 2.93 (dd, J=18.0, 12.3 Hz, 1H), 2.78 (t, J=6.3 Hz, 2H), 2.32-2.28 (m, 1H), 2.12 (qd, J=11.6, 5.9 Hz, 1H), 1.91 (d, J=4.4 Hz, 2H), 1.88-1.79 (m, 2H).

### Synthesis of Compound 59

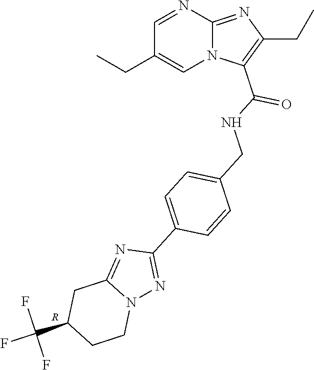

Compound 59 was synthesized from intermediates II-104 and I-88 and obtained as a white solid (85.9 mg, 56%). ¹H NMR (400 MHz, DMSO) δ 9.16 (d, J=2.4 Hz, 1H), 8.59 (d, J=2.5 Hz, 1H), 8.52 (t, J=6.0 Hz, 1H), 7.96 (d, J=8.3 Hz, 2H), 7.46 (d, J=8.3 Hz, 2H), 4.58 (d, J=5.9 Hz, 2H), 4.34 (dd, J=12.8, 4.1 Hz, 1H), 4.23-4.13 (m, 1H), 3.24-3.14 (m, 2H), 3.04 (q, J=7.5 Hz, 2H), 2.93 (dd, J=17.8, 12.5 Hz, 1H), 2.72 (q, J=7.2 Hz, 2H), 2.33-2.29 (m, 1H), 2.18-2.06 (m, 1H), 1.29 (t, J=7.5 Hz, 3H), 1.24 (t, J=7.5 Hz, 3H).

### Synthesis of Compound 60

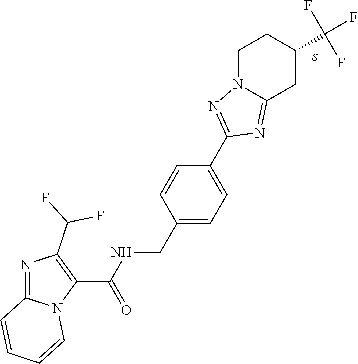

Compound 60 was synthesized from intermediates II-17b and I-87 and obtained as a pale yellow solid (287 mg, 36%). ¹H NMR (400 MHz, DMSO) δ 9.06 (s, 1H), 8.83 (d, J=6.9 Hz, 1H), 7.96 (d, J=8.3 Hz, 2H), 7.76 (d, J=9.1 Hz, 1H), 7.53 (ddd, J=9.1, 6.8, 1.2 Hz, 1H), 7.47 (d, J=8.3 Hz, 2H), 7.36 (t, J=53.9 Hz, 1H), 7.17 (td, J=6.9, 1.1 Hz, 1H), 4.59 (s, 2H), 4.37-4.28 (m, 1H), 4.17 (td, J=12.0, 4.8 Hz, 1H), 3.25-3.15 (m, 2H), 2.93 (dd, J=17.7, 12.2 Hz, 1H), 2.36-2.28 (m, 1H), 2.18-2.04 (m, 1H).

### Synthesis of Compound 61

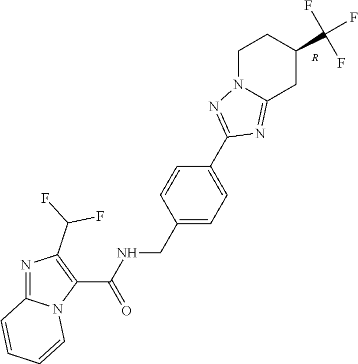

Compound 61 was synthesized from intermediates II-17b and I-88 and obtained as a beige solid (376 mg, 47%). ¹H NMR (400 MHz, DMSO) δ 9.05 (t, J=5.6 Hz, 1H), 8.82 (d, J=7.0 Hz, 1H), 7.96 (d, J=8.3 Hz, 2H), 7.76 (dd, J=9.1, 1.0 Hz, 1H), 7.53 (ddd, J=9.1, 6.8, 1.2 Hz, 1H), 7.47 (d, J=8.3 Hz, 2H), 7.35 (t, J=53.8 Hz, 1H), 7.17 (td, J=6.9, 1.2 Hz, 1H), 4.59 (d, J=5.6 Hz, 2H), 4.40-4.27 (m, 1H), 4.17 (td, J=12.1, 4.9 Hz, 1H), 3.24-3.16 (m, 2H), 2.93 (dd, J=17.7, 12.2 Hz, 1H), 2.38-2.26 (m, 1H), 2.21-2.03 (m, 1H).

### Synthesis of Compound 62

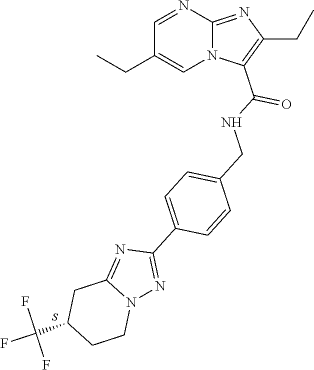

Compound 62 was synthesized from intermediates II-104 and I-87 and obtained as a white solid (69.7 mg, 44%). ¹H NMR (400 MHz, DMSO) δ 9.15 (d, J=2.4 Hz, 1H), 8.58 (d, J=2.5 Hz, 1H), 8.51 (t, J=5.7 Hz, 1H), 7.95 (d, J=8.3 Hz, 2H), 7.45 (d, J=8.3 Hz, 2H), 4.57 (d, J=5.9 Hz, 2H), 4.33 (dd, J=12.8, 3.7 Hz, 1H), 4.21-4.12 (m, 1H), 3.23-3.15 (m, 2H), 3.03 (q, J=7.5 Hz, 2H), 2.93 (dd, J=17.7, 12.1 Hz, 1H), 2.74-2.68 (m, 2H), 2.36-2.27 (m, 1H), 2.11 (qd, J=11.5, 5.8 Hz, 1H), 1.28 (t, J=7.5 Hz, 3H), 1.23 (t, J=7.5 Hz, 3H).

### Synthesis of Compound 63

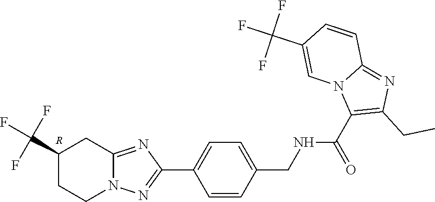

Compound 63 was synthesized from intermediates II-19 and I-88 and obtained as a white solid (115 mg, 51%). ¹H NMR (400 MHz, DMSO) δ 9.44 (s, 1H), 8.65 (t, J=5.8 Hz, 1H), 7.97 (d, J=8.3 Hz, 2H), 7.84 (d, J=9.4 Hz, 1H), 7.66 (dd, J=9.4, 1.9 Hz, 1H), 7.47 (d, J=8.3 Hz, 2H), 4.60 (d, J=5.9 Hz, 2H), 4.37-4.30 (m, 1H), 4.24-4.13 (m, 1H), 3.25-3.16 (m, 2H), 3.06 (q, J=7.5 Hz, 2H), 2.98-2.88 (m, 1H), 2.33-2.28 (m, 1H), 2.17-2.07 (m, 1H), 1.30 (t, J=7.5 Hz, 3H).

### Synthesis of Compound 64

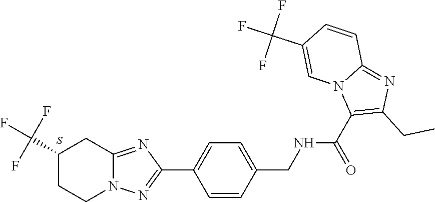

Compound 64 was synthesized from intermediates II-19 and I-87 and obtained as a white solid (82.4 mg, 55%). ¹H NMR (400 MHz, DMSO) δ 9.43 (s, 1H), 8.64 (t, J=5.9 Hz, 1H), 7.96 (d, J=8.3 Hz, 2H), 7.83 (d, J=9.4 Hz, 1H), 7.65 (dd, J=9.4, 1.9 Hz, 1H), 7.46 (d, J=8.3 Hz, 2H), 4.59 (d, J=5.9 Hz, 2H), 4.37-4.29 (m, 1H), 4.17 (td, J=12.1, 4.9 Hz, 1H), 3.24-3.14 (m, 2H), 3.09-3.00 (m, 2H), 2.93 (dd, J=17.7, 12.2 Hz, 1H), 2.36-2.26 (m, 1H), 2.18-2.05 (m, 1H), 1.29 (t, J=7.5 Hz, 3H).

### Synthesis of Compound 65

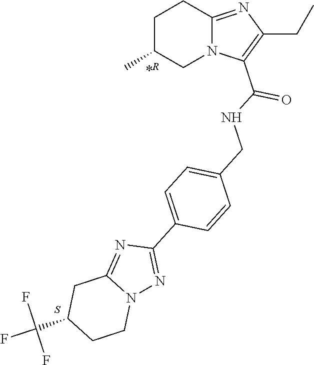

Compound 65 was synthesized from intermediates II-32 and 11-104 and obtained as a white solid (108.3 mg, 54%). ¹H NMR (400 MHz, DMSO) δ 8.25 (t, J=6.1 Hz, 1H), 7.94 (d, J=8.2 Hz, 2H), 7.38 (d, J=8.2 Hz, 2H), 4.45 (d, J=5.9 Hz, 2H), 4.32 (dd, J=12.8, 3.7 Hz, 1H), 4.23-4.11 (m, 2H), 3.46 (dd, J=12.7, 10.7 Hz, 1H), 3.27-3.12 (m, 2H), 2.92 (dd, J=17.6, 12.1 Hz, 1H), 2.79 (ddd, J=17.0, 5.5, 2.8 Hz, 1H), 2.73-2.59 (m, 3H), 2.32 (dd, J=10.8, 2.2 Hz, 1H), 2.18-2.05 (m, 1H), 1.98 (d, J=5.6 Hz, 1H), 1.87 (d, J=12.1 Hz, 1H), 1.46 (qd, J=11.5, 5.8 Hz, 1H), 1.10 (t, J=7.5 Hz, 3H), 1.02 (d, J=6.6 Hz, 3H).

### Synthesis of Compound 66

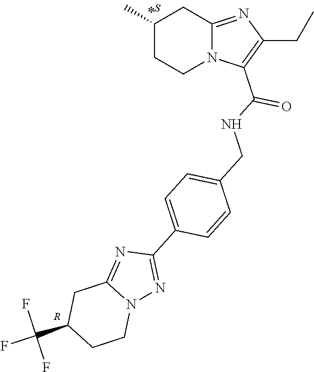

Compound 66 was synthesized from intermediates II-35 and I-88 and obtained as a beige solid (126.7 mg, 69%). ¹H NMR (400 MHz, DMSO) δ 8.26 (t, J=6.0 Hz, 1H), 7.93 (d, J=8.2 Hz, 2H), 7.38 (d, J=8.3 Hz, 2H), 4.45 (d, J=6.0 Hz, 2H), 4.32 (dd, J=12.9, 3.6 Hz, 1H), 4.22-4.11 (m, 2H), 3.91-3.80 (m, 1H), 3.23-3.13 (m, 2H), 2.92 (dd, J=17.6, 12.1 Hz, 1H), 2.84 (dd, J=16.4, 4.9 Hz, 1H), 2.64 (dt, J=8.3, 6.8 Hz, 2H), 2.35-2.23 (m, 2H), 2.18-2.05 (m, 1H), 2.00-1.89 (m, 2H), 1.53 (qd, J=11.3, 5.6 Hz, 1H), 1.10 (t, J=7.5 Hz, 3H), 1.04 (d, J=6.5 Hz, 3H).

### Synthesis of Compound 67

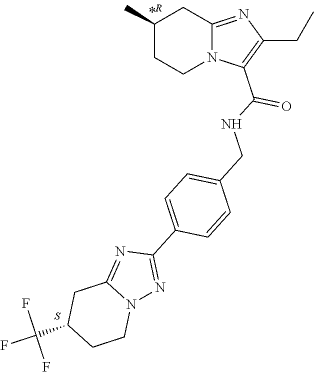

Compound 67 was synthesized from intermediates II-34 and I-87 and obtained as a white solid (123 mg, 64%). ¹H NMR (400 MHz, DMSO) δ 8.25 (t, J=6.0 Hz, 1H), 7.94 (d, J=8.2 Hz, 2H), 7.38 (d, J=8.3 Hz, 2H), 4.46 (d, J=6.0 Hz, 2H), 4.38-4.29 (m, 1H), 4.22-4.13 (m, 2H), 3.93-3.82 (m, 1H), 3.24-3.14 (m, 2H), 2.98-2.88 (m, 1H), 2.88-2.80 (m, 1H), 2.68-2.61 (m, 2H), 2.38-2.24 (m, 2H), 2.19-2.05 (m, 1H), 2.01-1.87 (m, 2H), 1.59-1.46 (m, 1H), 1.11 (t, J=7.5 Hz, 3H), 1.05 (d, J=6.5 Hz, 3H).

### Synthesis of Compound 68

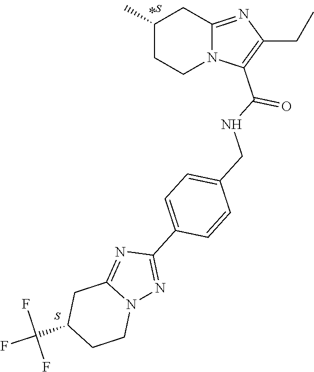

Compound 68 was synthesized from intermediates II-35 and I-87 and obtained as a white solid (121.2 mg, 64%). ¹H NMR (400 MHz, DMSO) δ 8.25 (t, J=6.0 Hz, 1H), 7.93 (d, J=8.3 Hz, 2H), 7.38 (d, J=8.3 Hz, 2H), 4.45 (d, J=6.0 Hz, 2H), 4.36-4.28 (m, 1H), 4.22-4.12 (m, 2H), 3.91-3.82 (m, 1H), 3.25-3.13 (m, 2H), 2.99-2.88 (m, 1H), 2.87-2.79 (m, 1H), 2.69-2.60 (m, 2H), 2.37-2.23 (m, 2H), 2.18-2.04 (m, 1H), 2.01-1.87 (m, 2H), 1.58-1.47 (m, 1H), 1.10 (t, J=7.5 Hz, 3H), 1.04 (d, J=6.5 Hz, 3H).

### Synthesis of Compound 69

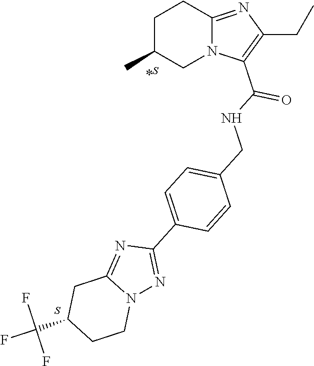

Compound 69 was synthesized from intermediates II-33 and I-87 and obtained as a beige solid (89 mg, 45%). ¹H NMR (400 MHz, DMSO) δ 8.26 (t, J=6.0 Hz, 1H), 7.94 (d, J=8.3 Hz, 2H), 7.38 (d, J=8.3 Hz, 2H), 4.52-4.39 (m, 2H), 4.32 (dd, J=12.8, 3.7 Hz, 1H), 4.23-4.10 (m, 2H), 3.46 (dd, J=12.8, 10.6 Hz, 1H), 3.24-3.12 (m, 2H), 2.98-2.86 (m, 1H), 2.80 (ddd, J=17.0, 5.5, 2.9 Hz, 1H), 2.73-2.60 (m, 3H), 2.32 (dd, J=10.7, 2.1 Hz, 1H), 2.11 (qd, J=11.5, 5.9 Hz, 1H), 2.00 (dd, J=14.2, 6.3 Hz, 1H), 1.87 (d, J=11.8 Hz, 1H), 1.46 (qd, J=11.5, 5.8 Hz, 1H), 1.11 (t, J=7.5 Hz, 3H), 1.02 (d, J=6.6 Hz, 3H).

### Synthesis of Compound 70

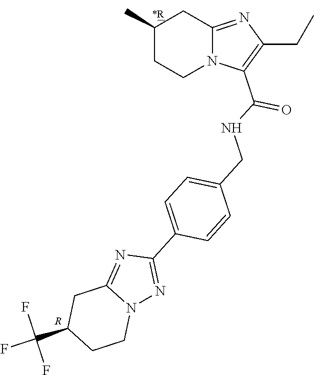

Compound 70 was synthesized from intermediates II-34 and I-88 and obtained as a white solid (118.6 mg, 61%). ¹H NMR (400 MHz, DMSO) δ 8.26 (t, J=5.8 Hz, 1H), 7.93 (d, J=8.3 Hz, 2H), 7.38 (d, J=8.3 Hz, 2H), 4.45 (d, J=6.0 Hz, 2H), 4.36-4.26 (m, 1H), 4.21-4.13 (m, 2H), 3.87 (td, J=13.0, 4.5 Hz, 1H), 3.25-3.14 (m, 2H), 2.92 (dd, J=17.6, 12.1 Hz, 1H), 2.84 (dd, J=16.2, 5.1 Hz, 1H), 2.68-2.60 (m, 2H), 2.36-2.25 (m, 2H), 2.18-2.04 (m, 1H), 2.00-1.90 (m, 2H), 1.59-1.46 (m, 1H), 1.11 (t, J=7.5 Hz, 3H), 1.04 (d, J=6.5 Hz, 3H).

### Synthesis of Compound 71

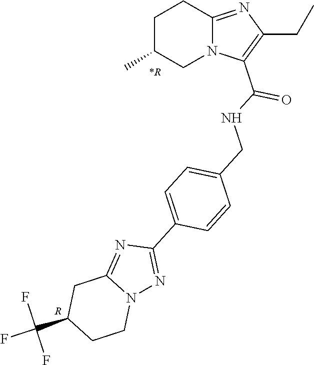

Compound 71 was synthesized from intermediates II-32 and I-88 and obtained as a white solid (79.5 mg, 37%). ¹H NMR (400 MHz, DMSO) δ 8.29-8.24 (m, 1H), 7.94 (d, J=8.2 Hz, 2H), 7.39 (d, J=8.3 Hz, 2H), 4.46 (d, J=5.9 Hz, 2H), 4.37-4.29 (m, 1H), 4.22-4.14 (m, 2H), 3.47 (dd, J=12.8, 10.7 Hz, 1H), 3.24-3.16 (m, 2H), 2.93 (dd, J=17.5, 12.2 Hz, 1H), 2.80 (ddd, J=16.9, 5.5, 2.8 Hz, 1H), 2.74-2.61 (m, 3H), 2.37-2.29 (m, 1H), 2.12 (qd, J=11.4, 5.7 Hz, 1H), 2.03-1.94 (m, 1H), 1.88 (d, J=11.7 Hz, 1H), 1.46 (qd, J=11.6, 5.7 Hz, 1H), 1.11 (t, J=7.5 Hz, 3H), 1.03 (d, J=6.6 Hz, 3H).

### Synthesis of Compound 72

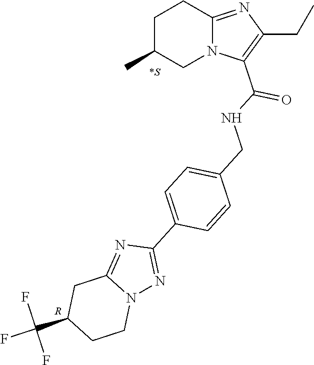

Compound 72 was synthesized from intermediates II-33 and I-88 and obtained as a beige solid (101.4 mg, 59%). ¹H NMR (400 MHz, DMSO) δ 8.27-8.22 (m, 1H), 7.94 (d, J=8.2 Hz, 2H), 7.38 (d, J=8.3 Hz, 2H), 4.45 (d, J=5.9 Hz, 2H), 4.32 (dd, J=12.9, 3.5 Hz, 1H), 4.21-4.12 (m, 2H), 3.46 (dd, J=12.8, 10.7 Hz, 1H), 3.23-3.14 (m, 2H), 2.92 (dd, J=17.6, 12.1 Hz, 1H), 2.80 (ddd, J=17.0, 5.4, 2.8 Hz, 1H), 2.72-2.60 (m, 3H), 2.32 (dd, J=10.8, 2.1 Hz, 1H), 2.18-2.05 (m, 1H), 2.03-1.92 (m, 1H), 1.88 (dd, J=11.3, 2.5 Hz, 1H), 1.52-1.40 (m, 1H), 1.11 (t, J=7.5 Hz, 3H), 1.02 (d, J=6.6 Hz, 3H).

### Synthesis of Compounds 73 and 74

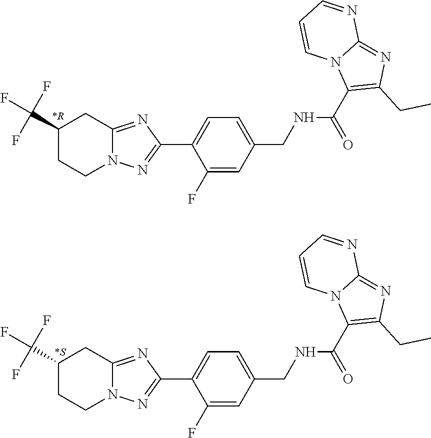 [SMILES: CCc1nc2ncccn2c1C(=O)NCc1ccc(-c2nc3n(n2)CC[C@@H](C(F)(F)F)C3)c(F)c1 ; CCc1nc2ncccn2c1C(=O)NCc1ccc(-c2nc3n(n2)CC[C@H](C(F)(F)F)C3)c(F)c1]

Isomers 73 and 74 were synthesized from intermediates II-83 and I-37, and after SFC separation (Jasco SFC prep system, i-cellulose column (Phenomenex) 250*30 nm, 5 mm particle size, isocratic mode at 100 ml/min of CO2 (50%)/EtOH (50%)/diethylamine (0.1%) at 30° C., 150 bars) it were obtained 73 (R*) (111.4 mg, 35%) and 74 (S*) (109.5 mg, 34%) as beige solids.

73: ¹H NMR (400 MHz, DMSO) δ 9.34 (dd, J=6.9, 2.0 Hz, 1H), 8.62 (dd, J=4.2, 2.0 Hz, 1H), 8.57 (t, J=6.0 Hz, 1H), 7.95 (t, J=7.8 Hz, 1H), 7.30 (d, J=9.3 Hz, 2H), 7.17 (dd, J=6.9, 4.2 Hz, 1H), 4.59 (d, J=5.9 Hz, 2H), 4.40-4.28 (m, 1H), 4.24-4.11 (m, 1H), 3.26-3.14 (m, 2H), 3.06 (q, J=7.5 Hz, 2H), 2.94 (dd, J=17.7, 12.3 Hz, 1H), 2.37-2.27 (m, 1H), 2.19-2.06 (m, 1H), 1.30 (t, J=7.5 Hz, 3H).

74: ¹H NMR (400 MHz, DMSO) δ 9.34 (dd, J=6.9, 2.0 Hz, 1H), 8.62 (dd, J=4.2, 2.0 Hz, 1H), 8.55 (t, J=6.0 Hz, 1H), 7.95 (t, J=7.8 Hz, 1H), 7.30 (d, J=9.4 Hz, 2H), 7.17 (dd, J=6.9, 4.2 Hz, 1H), 4.59 (d, J=5.9 Hz, 2H), 4.42-4.28 (m, 1H), 4.18 (td, J=12.0, 4.7 Hz, 1H), 3.27-3.14 (m, 2H), 3.05 (q, J=7.5 Hz, 2H), 2.94 (dd, J=17.7, 12.3 Hz, 1H), 2.37-2.27 (m, 1H), 2.19-2.05 (m, 1H), 1.30 (t, J=7.5 Hz, 3H).

### Synthesis of Compound 75

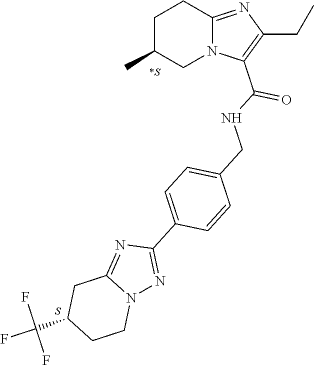

Compound 75 was synthesized from intermediates II-82 and I-87 and obtained as a white solid (150.1 mg, 65.6%). 1H NMR (400 MHz, DMSO) δ 7.93 (d, J=8.2 Hz, 2H), 7.84 (t, J=6.0 Hz, 1H), 7.37 (d, J=8.3 Hz, 2H), 4.48-4.37 (m, 2H), 4.35-4.29 (m, 1H), 4.21-4.05 (m, 2H), 3.96-3.88 (m, 1H), 3.23-3.15 (m, 2H), 3.05 (dd, J=16.6, 4.5 Hz, 1H), 2.97-2.87 (m, 1H), 2.70 (q, J=7.5 Hz, 2H), 2.47-2.41 (m, 1H), 2.35-2.28 (m, 1H), 2.11 (qd, J=11.5, 5.9 Hz, 1H), 2.00-1.86 (m, 2H), 1.68-1.56 (m, 1H), 1.10 (t, J=7.5 Hz, 3H), 1.06 (d, J=6.5 Hz, 3H).

### Synthesis of Compounds 76 and 77

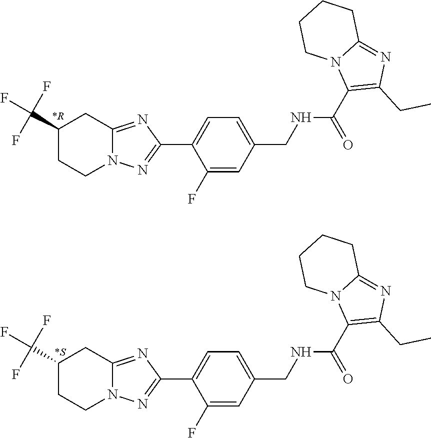 [SMILES: CCc1nc2n(c1C(=O)NCc1ccc(-c3nc4n(n3)CC[C@@H](C(F)(F)F)C4)c(F)c1)CCCC2 ; CCc1nc2n(c1C(=O)NCc1ccc(-c3nc4n(n3)CC[C@H](C(F)(F)F)C4)c(F)c1)CCCC2]

Isomers 76 and 77 were synthesized from intermediates II-17a and I-6, and after SFC separation (Jasco SFC prep system, i-cellulose column (Phenomenex) 250*30 nm, 5 mm particle size, isocratic mode at 100 ml/min of CO2 (50%)/EtOH (50%)/diethylamine (0.1%) at 30° C., 150 bars) it were obtained 76 (R*) (64.8 mg, 24%) and 77 (S*) (70.5 mg, 26%) as white solids.

76: ¹H NMR (400 MHz, DMSO) δ 8.28 (t, J=6.0 Hz, 1H), 7.93 (dd, J=9.8, 6.1 Hz, 1H), 7.22 (dd, J=9.4, 4.8 Hz, 2H), 4.46 (d, J=6.0 Hz, 2H), 4.35 (dd, J=12.8, 3.7 Hz, 1H), 4.18 (td, J=12.1, 4.9 Hz, 1H), 3.99 (t, J=5.8 Hz, 2H), 3.25-3.14 (m, 2H), 2.94 (dd, J=17.6, 12.1 Hz, 1H), 2.71 (dd, J=11.4, 5.3 Hz, 2H), 2.66 (q, J=7.5 Hz, 2H), 2.32 (ddd, J=8.7, 4.8, 2.3 Hz, 1H), 2.19-2.07 (m, 1H), 1.90-1.71 (m, 4H), 1.11 (t, J=7.5 Hz, 3H).

77: ¹H NMR (400 MHz, DMSO) δ 8.28 (t, J=6.0 Hz, 1H), 7.94 (dd, J=10.2, 5.8 Hz, 1H), 7.23 (dd, J=9.4, 4.9 Hz, 2H), 4.47 (d, J=6.0 Hz, 2H), 4.35 (dd, J=12.8, 3.6 Hz, 1H), 4.19 (td, J=12.0, 4.8 Hz, 1H), 4.00 (t, J=5.8 Hz, 2H), 3.28-3.12 (m, 2H), 2.94 (dd, J=17.6, 12.2 Hz, 1H), 2.72 (t, J=6.2 Hz, 2H), 2.66 (q, J=7.5 Hz, 2H), 2.33 (ddd, J=8.8, 4.8, 2.5 Hz, 1H), 2.13 (ddd, J=24.9, 11.5, 5.8 Hz, 1H), 1.83 (dd, J=25.1, 4.6 Hz, 4H), 1.12 (t, J=7.5 Hz, 3H).

### Synthesis of Compound 78 and 79

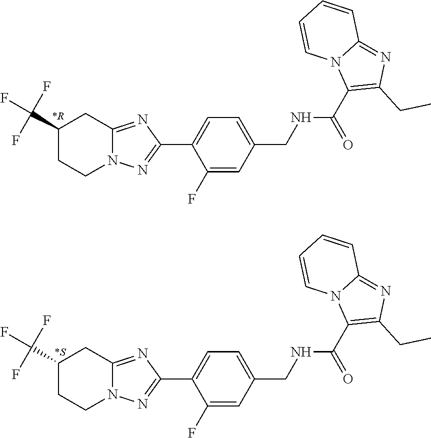 [SMILES: CCc1nc2ccccn2c1C(=O)NCc1ccc(-c2nc3n(n2)CC[C@@H](C(F)(F)F)C3)c(F)c1 ; CCc1nc2ccccn2c1C(=O)NCc1ccc(-c2nc3n(n2)CC[C@H](C(F)(F)F)C3)c(F)c1]

Isomers 78 and 79 were synthesized from intermediates I-37 and 11-85, and after SFC separation (Jasco SFC prep system, amylose column (Regis Technologies) 250*30 mm, 5 mm particle size, isocratic mode at 40 ml/min of CO2 (35%)/EtOH (65%)/diethylamine (0.1%) at 30° C., 120 bars) it were obtained 78 (R*) (76 mg, 20%) and 79 (S*) (62.6 mg, 16%) as white solids.

78: ¹H NMR (400 MHz, DMSO) δ 9.00 (d, J=6.9 Hz, 1H), 8.45 (t, J=5.9 Hz, 1H), 7.95 (t, J=8.0 Hz, 1H), 7.61 (d, J=9.0 Hz, 1H), 7.41-7.36 (m, 1H), 7.31 (s, 1H), 7.28 (d, J=3.1 Hz, 1H), 7.02 (td, J=6.9, 1.2 Hz, 1H), 4.58 (d, J=5.9 Hz, 2H), 4.39-4.30 (m, 1H), 4.18 (td, J=11.8, 4.5 Hz, 1H), 3.25-3.15 (m, 2H), 3.02 (q, J=7.5 Hz, 2H), 2.98-2.89 (m, 1H), 2.36-2.29 (m, 1H), 2.18-2.07 (m, 1H), 1.28 (t, J=7.5 Hz, 3H).

79: ¹H NMR (400 MHz, DMSO) δ 9.00 (d, J=7.0 Hz, 1H), 8.45 (t, J=6.0 Hz, 1H), 7.95 (t, J=8.0 Hz, 1H), 7.61 (d, J=9.0 Hz, 1H), 7.39 (dd, J=7.9, 6.6 Hz, 1H), 7.31 (s, 1H), 7.28 (d, J=3.4 Hz, 1H), 7.03-6.99 (m, 1H), 4.58 (d, J=6.2 Hz, 2H), 4.34 (d, J=11.6 Hz, 1H), 4.23-4.14 (m, 1H), 3.24-3.17 (m, 2H), 3.03 (t, J=7.5 Hz, 2H), 2.98-2.88 (m, 1H), 2.35-2.28 (m, 1H), 2.20-2.05 (m, 1H), 1.28 (t, J=7.5 Hz, 3H).

### Synthesis of Compound 80

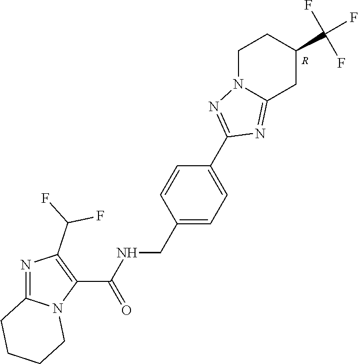

Compound 80 was synthesized from intermediates I-87 (R) and II-37 and obtained as a white solid (123.3 mg, 60%). ¹H NMR (400 MHz, DMSO) δ 8.82 (t, J=5.8 Hz, 1H), 7.94 (d, J=8.2 Hz, 2H), 7.39 (d, J=8.2 Hz, 2H), 6.95 (t, J=54.3 Hz, 1H), 4.48 (d, J=5.9 Hz, 2H), 4.36-4.29 (m, 1H), 4.23-4.12 (m, 1H), 4.04 (t, J=5.5 Hz, 2H), 3.23-3.14 (m, 2H), 2.93 (dd, J=17.7, 12.0 Hz, 1H), 2.79 (t, J=6.3 Hz, 2H), 2.32 (d, J=7.6 Hz, 1H), 2.19-2.05 (m, 1H), 1.90 (d, J=4.9 Hz, 2H), 1.83 (d, J=4.7 Hz, 2H).

### Synthesis of Compound 81

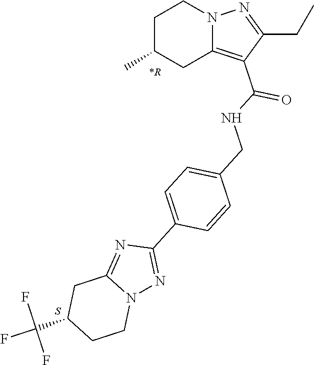

Compound 81 was synthesized from intermediates I-87 (S) and II-81 and obtained as a pale yellow oil (134.1 mg, 73%). ¹H NMR (400 MHz, DMSO) δ 7.93 (d, J=8.3 Hz, 2H), 7.83 (t, J=6.0 Hz, 1H), 7.37 (d, J=8.3 Hz, 2H), 4.49-4.37 (m, 2H), 4.36-4.27 (m, 1H), 4.18 (dd, J=11.6, 4.8 Hz, 1H), 4.10 (dtd, J=10.7, 8.2, 5.2 Hz, 1H), 3.97-3.86 (m, 1H), 3.26-3.13 (m, 2H), 3.05 (dd, J=16.5, 4.4 Hz, 1H), 2.97-2.87 (m, 1H), 2.74-2.66 (m, 2H), 2.48-2.40 (m, 1H), 2.37-2.27 (m, 1H), 2.19-2.05 (m, 1H), 1.98 (t, J=12.7 Hz, 1H), 1.93-1.83 (m, 1H), 1.62 (ddd, J=24.1, 11.3, 5.7 Hz, 1H), 1.10 (t, J=7.5 Hz, 3H), 1.06 (d, J=6.5 Hz, 3H).

### Synthesis of Compound 82

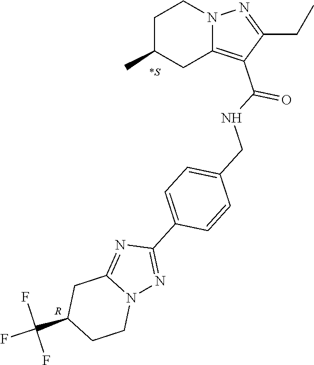

Compound 82 was synthesized from intermediates I-87 (R) and II-82 and obtained as a pale yellow solid (134.1 mg, 73%). ¹H NMR (400 MHz, DMSO) δ 7.93 (d, J=8.2 Hz, 2H), 7.83 (t, J=6.0 Hz, 1H), 7.37 (d, J=8.3 Hz, 2H), 4.43 (d, J=4.4 Hz, 2H), 4.36-4.28 (m, 1H), 4.22-4.06 (m, 2H), 3.98-3.87 (m, 1H), 3.24-3.14 (m, 2H), 3.05 (dd, J=16.7, 4.2 Hz, 1H), 2.92 (dd, J=17.6, 12.1 Hz, 1H), 2.70 (q, J=7.5 Hz, 2H), 2.48-2.41 (m, 1H), 2.32 (dd, J=11.1, 2.1 Hz, 1H), 2.19-2.05 (m, 1H), 2.01-1.84 (m, 2H), 1.69-1.55 (m, 1H), 1.13-1.02 (m, 6H).

### Synthesis of Compound 83

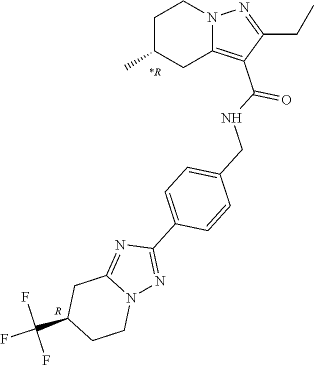

Compound 83 was synthesized from intermediates I-87 (R) and II-81 and obtained as a pale yellow solid (134.1 mg, 73%). ¹H NMR (400 MHz, DMSO) δ 7.93 (d, J=8.3 Hz, 2H), 7.83 (t, J=6.0 Hz, 1H), 7.37 (d, J=8.3 Hz, 2H), 4.49-4.37 (m, 2H), 4.32 (dd, J=12.8, 3.7 Hz, 1H), 4.17 (td, J=12.1, 5.0 Hz, 1H), 4.09 (ddd, J=12.8, 5.4, 2.7 Hz, 1H), 3.97-3.87 (m, 1H), 3.25-3.13 (m, 2H), 3.05 (dd, J=16.6, 4.4 Hz, 1H), 2.97-2.86 (m, 1H), 2.70 (q, J=7.5 Hz, 2H), 2.48-2.40 (m, 1H), 2.32 (dd, J=11.1, 2.1 Hz, 1H), 2.11 (qd, J=11.5, 5.9 Hz, 1H), 1.97 (d, J=16.3 Hz, 1H), 1.91 (d, J=5.6 Hz, 1H), 1.62 (qd, J=11.3, 5.7 Hz, 1H), 1.10 (t, J=7.5 Hz, 3H), 1.06 (d, J=6.5 Hz, 3H).

### Synthesis of Compound 84

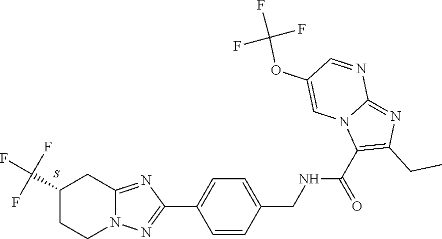

Compound 84 was synthesized from intermediates I-87 (S) and II-88 and obtained as a white solid (17.1 mg, 27%). ¹H NMR (400 MHz, DMSO) δ 9.54 (d, J=2.3 Hz, 1H), 8.83 (d, J=2.6 Hz, 1H), 8.65 (t, J=5.8 Hz, 1H), 7.95 (d, J=8.3 Hz, 2H), 7.46 (d, J=8.3 Hz, 2H), 4.59 (d, J=5.8 Hz, 2H), 4.33 (dd, J=12.9, 3.6 Hz, 1H), 4.17 (td, J=11.9, 4.8 Hz, 1H), 3.24-3.15 (m, 2H), 3.08 (q, J=7.5 Hz, 2H), 2.92 (dd, J=17.7, 12.3 Hz, 1H), 2.32 (dd, J=10.7, 2.0 Hz, 1H), 2.17-2.07 (m, 1H), 1.30 (t, J=7.5 Hz, 3H).

### Synthesis of Compound 85

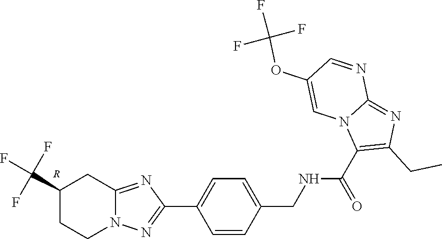

Compound 85 was synthesized from intermediates I-87 (R) and II-88 and obtained as a white solid (20.7 mg, 26%). ¹H NMR (400 MHz, DMSO) δ 9.54 (d, J=2.7 Hz, 1H), 8.83 (d, J=2.8 Hz, 1H), 8.65 (t, J=5.9 Hz, 1H), 7.95 (d, J=8.2 Hz, 2H), 7.46 (d, J=8.2 Hz, 2H), 4.59 (d, J=5.9 Hz, 2H), 4.36-4.29 (m, 1H), 4.17 (td, J=12.1, 4.7 Hz, 1H), 3.23-3.15 (m, 2H), 3.08 (q, J=7.5 Hz, 2H), 2.92 (dd, J=17.7, 12.2 Hz, 1H), 2.36-2.28 (m, 1H), 2.18-2.05 (m, 1H), 1.30 (t, J=7.5 Hz, 3H).

### Synthesis of Compounds 86 and 87

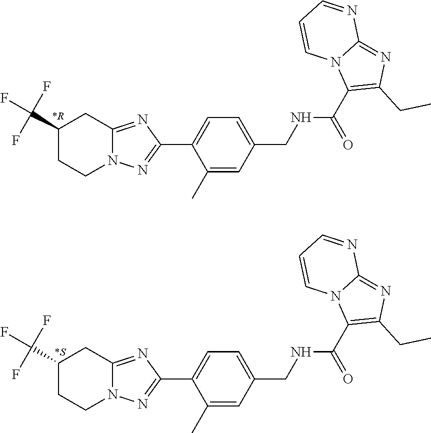 [SMILES: CCc1nc2ncccn2c1C(=O)NCc1ccc(-c2nc3n(n2)CC[C@@H](C(F)(F)F)C3)c(C)c1 ; CCc1nc2ncccn2c1C(=O)NCc1ccc(-c2nc3n(n2)CC[C@H](C(F)(F)F)C3)c(C)c1]

Isomers 86 and 87 were synthesized from intermediates I-37 and 11-85, and after SFC separation (Jasco SFC prep system, i-cellulose column (Regis Technologies) 250*30 mm, 5 mm particle size, isocratic mode at 40 ml/min of CO2 (50%)/EtOH (50%)/diethylamine (0.1%) at 30° C., 120 bars) it were obtained 86 (R) (83.9 mg, 33%) and 87 (S) (82.4 mg, 33%) as beige solids.

86: ¹H NMR (400 MHz, DMSO) δ 9.32 (dd, J=6.9, 2.0 Hz, 1H), 8.62 (dd, J=4.2, 2.0 Hz, 1H), 8.54 (t, J=5.9 Hz, 1H), 7.87 (d, J=7.8 Hz, 1H), 7.27 (d, J=8.8 Hz, 2H), 7.16 (dd, J=6.9, 4.2 Hz, 1H), 4.55 (d, J=5.9 Hz, 2H), 4.41-4.29 (m, 1H), 4.18 (td, J=12.1, 4.9 Hz, 1H), 3.26-3.14 (m, 2H), 3.05 (q, J=7.5 Hz, 2H), 2.93 (dd, J=17.4, 12.0 Hz, 1H), 2.58 (s, 3H), 2.36-2.27 (m, 1H), 2.20-2.06 (m, 1H), 1.30 (t, J=7.5 Hz, 3H).

87: ¹H NMR (400 MHz, DMSO) δ 9.32 (dd, J=6.9, 2.0 Hz, 1H), 8.62 (dd, J=4.2, 2.0 Hz, 1H), 8.54 (t, J=6.0 Hz, 1H), 7.87 (d, J=7.8 Hz, 1H), 7.27 (d, J=8.8 Hz, 2H), 7.17 (dd, J=6.9, 4.2 Hz, 1H), 4.55 (d, J=5.9 Hz, 2H), 4.39-4.28 (m, 1H), 4.18 (td, J=12.0, 4.8 Hz, 1H), 3.26-3.14 (m, 2H), 3.05 (q, J=7.5 Hz, 2H), 2.93 (dd, J=17.4, 12.0 Hz, 1H), 2.58 (s, 3H), 2.38-2.28 (m, 1H), 2.20-2.07 (m, 1H), 1.30 (t, J=7.5 Hz, 3H).

### Synthesis of Compound 88

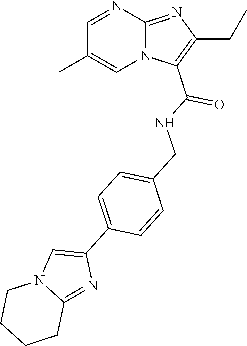 [SMILES: CCc1nc2ncc(C)cn2c1C(=O)NCc1ccc(-c2cn3c(n2)CCCC3)cc1]

Compound 88 was synthesized from intermediates I-132 and II-3a and obtained as a white solid (75.4 mg, 39%). ¹H NMR (400 MHz, DMSO) δ 9.14 (s, 1H), 8.51 (d, J=2.3 Hz, 1H), 8.46 (t, J=5.9 Hz, 1H), 7.68 (d, J=8.1 Hz, 2H), 7.42 (s, 1H), 7.32 (d, J=8.1 Hz, 2H), 4.51 (d, J=5.8 Hz, 2H), 3.95 (t, J=5.7 Hz, 2H), 3.00 (q, J=7.5 Hz, 2H), 2.74 (t, J=6.2 Hz, 2H), 2.34 (s, 3H), 1.95-1.80 (m, 4H), 1.27 (t, J=7.5 Hz, 3H).

### Synthesis of Compound 89

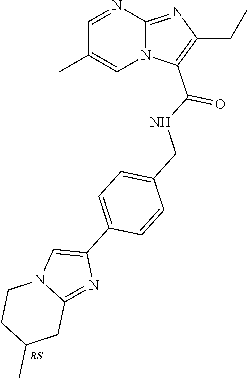 [SMILES: CCc1nc2ncc(C)cn2c1C(=O)NCc1ccc(-c2cn3c(n2)CC(C)CC3)cc1]

Compound 89 was synthesized from intermediates I-131 and II-3a and obtained as a white solid (64.1 mg, 37%). ¹H NMR (400 MHz, DMSO-d6) δ 9.15 (dd, J=2.1, 1.0 Hz, 1H), 8.51 (d, J=2.4 Hz, 1H), 8.47 (t, J=6.0 Hz, 1H), 7.68 (d, J=8.2 Hz, 2H), 7.44 (s, 1H), 7.32 (d, J=8.2 Hz, 2H), 4.51 (d, J=5.9 Hz, 2H), 4.05 (ddd, J=12.5, 5.4, 2.7 Hz, 1H), 3.89 (td, J=11.7, 4.6 Hz, 1H), 3.01 (q, J=7.5 Hz, 2H), 2.88 (dd, J=17.2, 5.6 Hz, 1H), 2.33 (d, J=5.0 Hz, 4H), 2.07-1.90 (m, 2H), 1.60 (dtd, J=13.7, 11.3, 5.8 Hz, 1H), 1.27 (t, J=7.5 Hz, 3H), 1.07 (d, J=6.5 Hz, 3H).

### Synthesis of Compound 90

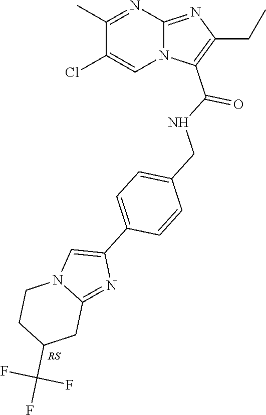 [SMILES: CCc1nc2nc(C)c(Cl)cn2c1C(=O)NCc1ccc(-c2cn3c(n2)CC(C(F)(F)F)CC3)cc1]

Compound 90 was synthesized from intermediates II-110 and II-8e and obtained as a white solid (94.5 mg, 21%). ¹H NMR (400 MHz, DMSO) δ 9.35 (s, 1H), 8.49 (t, J=5.9 Hz, 1H), 7.69 (d, J=8.2 Hz, 2H), 7.52 (s, 1H), 7.33 (d, J=8.2 Hz, 2H), 4.51 (d, J=5.8 Hz, 2H), 4.23-4.10 (m, 1H), 3.98 (td, J=12.1, 4.4 Hz, 1H), 3.11-3.04 (m, 2H), 3.01 (q, J=7.5 Hz, 2H), 2.77 (dd, J=17.3, 12.7 Hz, 1H), 2.61 (s, 3H), 2.23 (d, J=11.2 Hz, 1H), 2.01-1.88 (m, 1H), 1.27 (t, J=7.5 Hz, 3H).

### Synthesis of Compound 91

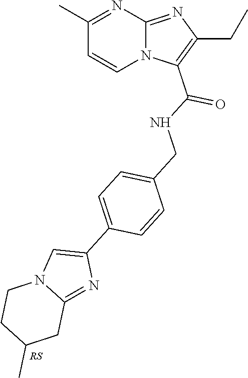 [SMILES: CCc1nc2nc(C)ccn2c1C(=O)NCc1ccc(-c2cn3c(n2)CC(C)CC3)cc1]

Compound 91 was synthesized from intermediates I-131 and II-8a and obtained as a white solid (120 mg, 53%). ¹H NMR (400 MHz, DMSO) δ 9.18 (d, J=7.0 Hz, 1H), 8.41 (t, J=5.9 Hz, 1H), 7.67 (d, J=8.2 Hz, 2H), 7.43 (s, 1H), 7.31 (d, J=8.2 Hz, 2H), 7.05 (d, J=7.1 Hz, 1H), 4.50 (d, J=5.8 Hz, 2H), 4.05 (ddd, J=12.4, 5.4, 2.8 Hz, 1H), 3.88 (td, J=11.9, 4.7 Hz, 1H), 2.99 (q, J=7.5 Hz, 2H), 2.87 (dd, J=16.4, 4.9 Hz, 1H), 2.54 (s, 3H), 2.32 (dd, J=16.3, 10.3 Hz, 1H), 2.05-1.90 (m, 2H), 1.67-1.52 (m, 1H), 1.26 (t, J=7.5 Hz, 3H), 1.07 (d, J=6.5 Hz, 3H).

### Synthesis of Compound 92

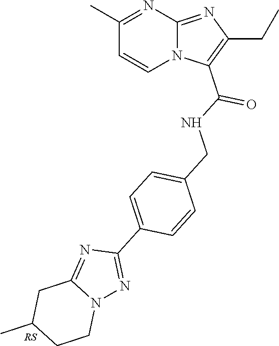 [SMILES: CCc1nc2nc(C)ccn2c1C(=O)NCc1ccc(-c2nc3n(n2)CCC(C)C3)cc1]

Compound 92 was synthesized from intermediates I-16 and II-8a and obtained as a white solid (90.1 mg, 41%). ¹H NMR (400 MHz, DMSO) δ 9.20 (d, J=7.0 Hz, 1H), 8.45 (t, J=5.8 Hz, 1H), 7.93 (d, J=8.2 Hz, 2H), 7.43 (d, J=8.2 Hz, 2H), 7.05 (d, J=7.1 Hz, 1H), 4.56 (d, J=5.8 Hz, 2H), 4.27-4.17 (m, 1H), 4.15-3.96 (m, 1H), 3.05-2.93 (m, 3H), 2.55 (s, 3H), 2.47-2.40 (m, 1H), 2.15-1.99 (m, 2H), 1.81-1.67 (m, 1H), 1.27 (t, J=7.5 Hz, 3H), 1.10 (d, J=6.6 Hz, 3H).

### Synthesis of Compound 93 and 94

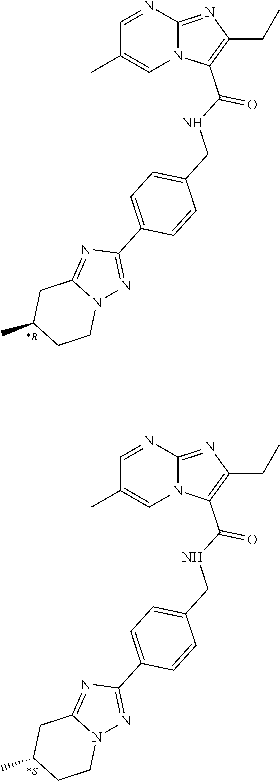

Isomers 93 and 94 were synthesized from intermediates I-16 and II-3a, and after SFC separation (SFC prep system, amylose column (Phenomenex) 250*30 mm, 5 mm particle size, isocratic mode of CO2 (500%)/EtOH (500%)/diethylamine (0.10%) it were obtained compound 93 (R*) (542 mg, 390%) and intermediate 94 (S*) (543 mg, 400%) as beige solids.

93: ¹H NMR (400 MHz, DMSO) δ 9.16 (s, 1H), 8.51 (d, J=2.3 Hz, 1H), 8.51-8.48 (m, 1H), 7.94 (d, J=8.2 Hz, 2H), 7.43 (d, J=8.2 Hz, 2H), 4.57 (d, J=5.6 Hz, 2H), 4.22 (ddd, J=12.5, 5.6, 2.8 Hz, 1H), 4.15-4.02 (m, 1H), 3.06-2.99 (m, 2H), 2.99-2.94 (m, 1H), 2.48-2.40 (m, 1H), 2.34 (s, 3H), 2.14-1.96 (m, 2H), 1.82-1.66 (m, 1H), 1.28 (t, J=7.5 Hz, 3H), 1.10 (d, J=6.6 Hz, 3H).

94: ¹H NMR (400 MHz, DMSO) δ 9.16 (d, J=1.2 Hz, 1H), 8.51 (d, J=2.4 Hz, 1H), 8.51-8.48 (m, 1H), 7.94 (d, J=8.2 Hz, 2H), 7.43 (d, J=8.2 Hz, 2H), 4.57 (d, J=5.6 Hz, 2H), 4.22 (ddd, J=12.7, 5.9, 3.0 Hz, 1H), 4.13-4.02 (m, 1H), 3.02 (q, J=7.5 Hz, 2H), 3.00-2.93 (m, 1H), 2.45-2.39 (m, 1H), 2.34 (s, 3H), 2.12-1.93 (m, 2H), 1.82-1.67 (m, 1H), 1.28 (t, J=7.5 Hz, 3H). 1.10 (d. J=6.5 Hz, 3H).

### Synthesis of Compound 95

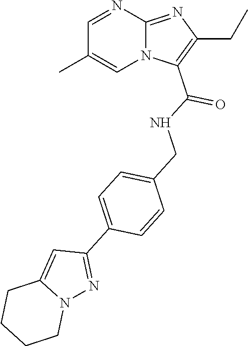 [SMILES: CCc1nc2ncc(C)cn2c1C(=O)NCc1ccc(-c2cc3n(n2)CCCC3)cc1]

Compound 95 was synthesized from intermediates I-86 and II-3a and obtained as a white solid (63.7 mg, 51%). ¹H NMR (400 MHz, DMSO) δ 9.15 (d, J=1.2 Hz, 1H), 8.51 (d, J=2.4 Hz, 1H), 8.47 (t, J=5.9 Hz, 1H), 7.72 (d, J=8.2 Hz, 2H), 7.37 (d, J=8.2 Hz, 2H), 6.41 (s, 1H), 4.54 (d, J=5.9 Hz, 2H), 4.07 (t, J=6.1 Hz, 2H), 3.01 (q, J=7.5 Hz, 2H), 2.76 (t, J=6.3 Hz, 2H), 2.34 (s, 3H), 2.01-1.94 (m, 2H), 1.84-1.74 (m, 2H), 1.28 (t, J=7.5 Hz, 3H).

### Synthesis of Compound 96

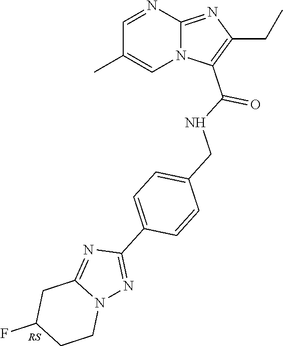 [SMILES: CCc1nc2ncc(C)cn2c1C(=O)NCc1ccc(-c2nc3n(n2)CCC(F)C3)cc1]

Compound 96 was synthesized from intermediates I-89 and II-3a and obtained as a white solid (21.4 mg, 20%). ¹H NMR (400 MHz, DMSO) δ 9.16 (dd, J=2.2, 1.1 Hz, 1H), 8.50 (dd, J=9.8, 4.2 Hz, 2H), 7.95 (d, J=8.2 Hz, 2H), 7.45 (d, J=8.2 Hz, 2H), 5.49-5.28 (m, 1H), 4.57 (d, J=5.9 Hz, 2H), 4.38-4.09 (m, 2H), 3.29-3.15 (m, 2H), 3.02 (q, J=7.5 Hz, 2H), 2.46-2.22 (m, 2H), 2.34 (s, 3H), 1.28 (t, J=7.5 Hz, 3H).

### Synthesis of Compound 97

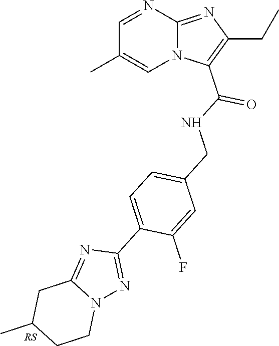 [SMILES: CCc1nc2ncc(C)cn2c1C(=O)NCc1ccc(-c2nc3n(n2)CCC(C)C3)c(F)c1]

Compound 97 was synthesized from intermediates I-16 and II-3a and obtained as a white solid (115 mg, 41%). ¹H NMR (400 MHz, DMSO) δ 9.17 (d, J=1.1 Hz, 1H), 8.52 (d, J=2.4 Hz, 1H), 8.49 (t, J=5.9 Hz, 1H), 7.94 (t, J=7.8 Hz, 1H), 7.28 (d, J=6.5 Hz, 1H), 7.27 (s, 1H), 4.58 (d, J=5.9 Hz, 2H), 4.24 (ddd, J=12.6, 5.6, 2.9 Hz, 1H), 4.16-4.03 (m, 1H), 3.08-2.93 (m, 3H), 2.48-2.42 (m, 1H), 2.34 (s, 3H), 2.20-2.01 (m, 2H), 1.75 (dtd, J=13.4, 10.8, 5.8 Hz, 1H), 1.29 (t, J=7.5 Hz, 3H), 1.10 (d, J=6.6 Hz, 3H).

### Synthesis of Compound 98

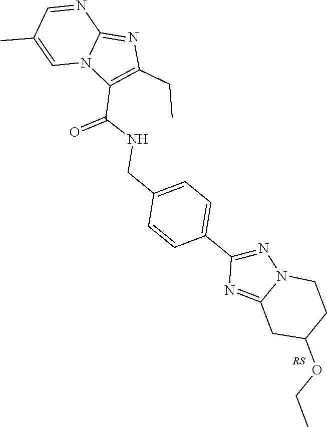 [SMILES: CCOC1CCn2nc(-c3ccc(CNC(=O)c4c(CC)nc5ncc(C)cn45)cc3)nc2C1]

Compound 98 was synthesized from intermediates I-126 and II-3a and obtained as a white solid (213 mg, 58%). ¹H NMR (400 MHz, DMSO) δ 9.16 (s, 1H), 8.50 (dd, J=10.7, 4.2 Hz, 2H), 7.94 (d, J=8.2 Hz, 2H), 7.44 (d, J=8.2 Hz, 2H), 4.57 (d, J=5.9 Hz, 2H), 4.22-4.07 (m, 2H), 4.07-4.00 (m, 1H), 3.64-3.47 (m, 2H), 3.10 (dd, J=17.1, 4.3 Hz, 1H), 3.02 (q, J=7.5 Hz, 2H), 2.92 (dd, J=17.1, 4.6 Hz, 1H), 2.34 (s, 3H), 2.27-2.11 (m, 2H), 1.28 (t, J=7.5 Hz, 3H), 1.11 (t, J=7.0 Hz, 3H).

### Synthesis of Compound 99

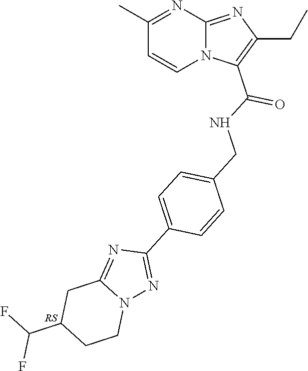 [SMILES: CCc1nc2nc(C)ccn2c1C(=O)NCc1ccc(-c2nc3n(n2)CCC(C(F)F)C3)cc1]

Compound 99 was synthesized from intermediates I-51 and II-8a and obtained as a beige solid (85 mg, 66%). ¹H NMR (400 MHz, DMSO) δ 9.20 (d, J=7.0 Hz, 1H), 8.46 (t, J=5.8 Hz, 1H), 7.93 (s, 2H), 7.44 (d, J=8.1 Hz, 2H), 7.05 (d, J=7.1 Hz, 1H), 6.20 (td, J=56.3, 3.9 Hz, 1H), 4.56 (d, J=5.7 Hz, 2H), 4.40-4.24 (m, 1H), 4.13 (td, J=12.1, 4.8 Hz, 1H), 3.09-2.95 (m, 3H), 2.79 (dd, J=16.7, 10.8 Hz, 1H), 2.71-2.58 (m, 1H), 2.55 (s, 3H), 2.20 (d, J=11.9 Hz, 1H), 2.03-1.83 (m, 1H), 1.27 (t, J=7.5 Hz, 3H).

### Synthesis of Compound 100

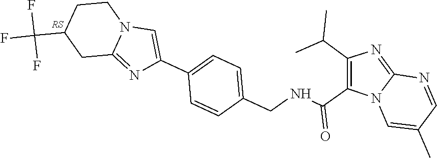 [SMILES: Cc1cnc2nc(C(C)C)c(C(=O)NCc3ccc(-c4cn5c(n4)CC(C(F)(F)F)CC5)cc3)n2c1]

Compound 100 was synthesized from intermediates I-81 and 11-94 and obtained as a yellow solid (78.3 mg, 46%). ¹H NMR (400 MHz, DMSO) δ 9.06 (dd, J=2.3, 1.1 Hz, 1H), 8.56 (t, J=5.9 Hz, 1H), 8.50 (d, J=2.4 Hz, 1H), 7.70 (d, J=8.2 Hz, 2H), 7.54 (s, 1H), 7.34 (d, J=8.3 Hz, 2H), 4.52 (d, J=5.9 Hz, 2H), 4.16 (dd, J=12.6, 3.5 Hz, 1H), 3.98 (td, J=12.2, 4.6 Hz, 1H), 3.65-3.50 (m, 1H), 3.19-3.01 (m, 2H), 2.78 (dd, J=17.3, 12.6 Hz, 1H), 2.33 (s, 3H), 2.28-2.19 (m, 1H), 2.06-1.86 (m, 1H), 1.28 (d, J=6.7 Hz, 6H).

### Synthesis of Compound 101

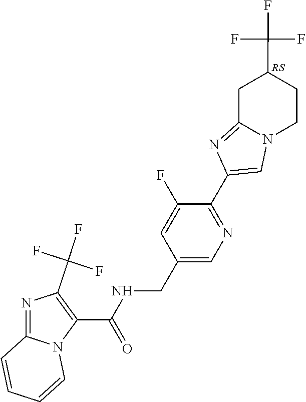 [SMILES: O=C(NCc1cnc(-c2cn3c(n2)CC(C(F)(F)F)CC3)c(F)c1)c1c(C(F)(F)F)nc2ccccn12]

Compound 101 was synthesized from intermediates I-135 and II-6a and obtained as a beige solid (47.2 mg, 23%). ¹H NMR (400 MHz, DMSO) δ 9.33 (t, J=5.6 Hz, 1H), 8.60 (d, J=7.0 Hz, 1H), 8.43 (s, 1H), 7.80 (d, J=9.1 Hz, 1H), 7.69-7.63 (m, 2H), 7.57 (ddd, J=9.1, 6.8, 1.2 Hz, 1H), 7.21 (td, J=6.9, 1.1 Hz, 1H), 4.61 (d, J=5.5 Hz, 2H), 4.29-4.19 (m, 1H), 4.06-3.95 (m, 1H), 3.15-3.06 (m, 2H), 2.81 (dd, J=17.4, 12.8 Hz, 1H), 2.30-2.20 (m, 1H), 2.06-1.89 (m, 1H).

### Synthesis of Compound 102

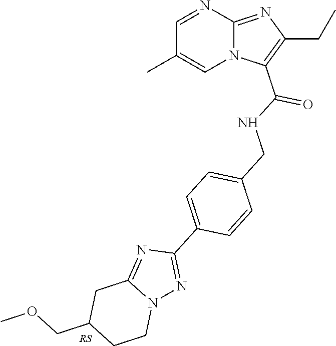 [SMILES: CCc1nc2ncc(C)cn2c1C(=O)NCc1ccc(-c2nc3n(n2)CCC(COC)C3)cc1]

Compound 102 was synthesized from intermediates I-120 and II-3a and obtained as a pale orange solid (105.1 mg, 57%). ¹H NMR (400 MHz, DMSO) δ 9.16 (dd, J=2.4, 1.1 Hz, 1H), 8.54-8.47 (m, 2H), 7.94 (d, J=8.3 Hz, 2H), 7.43 (d, J=8.3 Hz, 2H), 4.57 (d, J=5.9 Hz, 2H), 4.23 (ddd, J=12.7, 5.5, 3.0 Hz, 1H), 4.08 (td, J=11.7, 4.7 Hz, 1H), 3.38 (dq, J=6.0, 2.9 Hz, 2H), 3.29 (s, 3H), 3.06-2.93 (m, 3H), 2.60-2.53 (m, 1H), 2.33 (dd, J=6.1, 1.3 Hz, 3H), 2.31-2.24 (m, 1H), 2.13 (d, J=13.4 Hz, 1H), 1.85-1.72 (m, 1H), 1.27 (q, J=7.1 Hz, 3H).

### Synthesis of Compound 103, 104 and 105

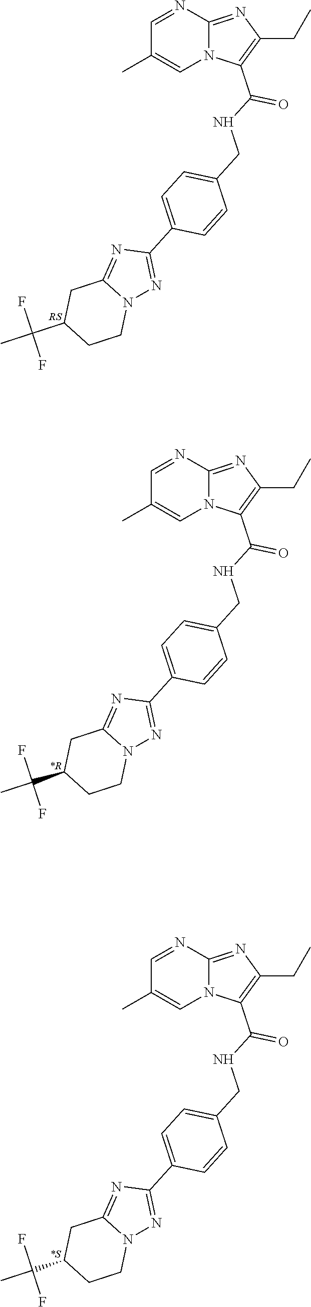

Racemic compound 103 was synthesized from intermediates I-133 and II-3a and obtained as a white solid (128 mg, 80%).

Isomers were separated by SFC (Jasco SFC prep system, i-cellulose column (Phenomenex) 250*30 mm, 5 mm particle size, isocratic mode at 100 ml/min of CO2 (40%)/MeOH (60%)/diethylamine (0.1%) at 30° C., 120 bars) to obtain compound 104 (*R) and compound 105 (*S) as white solids.

103: ¹H NMR (400 MHz, DMSO) δ 9.16 (dd, J=2.3, 1.1 Hz, 1H), 8.54-8.46 (m, 2H), 7.95 (d, J=8.3 Hz, 2H), 7.44 (d, J=8.3 Hz, 2H), 4.57 (d, J=5.8 Hz, 2H), 4.30 (dd, J=12.7, 3.5 Hz, 1H), 4.12 (td, J=12.0, 4.6 Hz, 1H), 3.09 (dd, J=16.6, 4.0 Hz, 1H), 3.02 (q, J=7.5 Hz, 2H), 2.79 (dd, J=16.5, 11.5 Hz, 1H), 2.69-2.64 (m, 1H), 2.34 (s, 3H), 2.29-2.21 (m, 1H), 2.02-1.90 (m, 1H), 1.71 (t, J=19.5 Hz, 3H), 1.28 (t, J=7.5 Hz, 3H).

104: ¹H NMR (400 MHz, DMSO) δ 9.16 (dd, J=2.3, 1.1 Hz, 1H), 8.51 (dd, J=7.0, 4.2 Hz, 2H), 7.95 (d, J=8.3 Hz, 2H), 7.44 (d, J=8.3 Hz, 2H), 4.57 (d, J=5.9 Hz, 2H), 4.30 (dd, J=12.6, 3.8 Hz, 1H), 4.12 (td, J=12.1, 4.6 Hz, 1H), 3.09 (dd, J=16.5, 4.1 Hz, 1H), 3.01 (t, J=7.5 Hz, 2H), 2.79 (dd, J=16.5, 11.4 Hz, 1H), 2.71-2.60 (m, 1H), 2.33 (d, J=5.5 Hz, 3H), 2.25 (dd, J=13.5, 2.1 Hz, 1H), 1.96 (ddd, J=25.1, 11.7, 5.7 Hz, 1H), 1.71 (t, J=19.5 Hz, 3H), 1.28 (t, J=7.5 Hz, 3H).

105: ¹H NMR (400 MHz, DMSO) δ 9.16 (dd, J=2.4, 1.1 Hz, 1H), 8.51 (dd, J=6.9, 4.2 Hz, 2H), 7.98-7.91 (m, 2H), 7.44 (d, J=8.3 Hz, 2H), 4.57 (d, J=5.9 Hz, 2H), 4.34-4.25 (m, 1H), 4.12 (td, J=12.1, 4.6 Hz, 1H), 3.09 (dd, J=16.6, 3.9 Hz, 1H), 3.01 (t, J=7.5 Hz, 2H), 2.79 (dd, J=16.5, 11.3 Hz, 1H), 2.71-2.59 (m, 1H), 2.34 (t, J=2.7 Hz, 3H), 2.25 (dd, J=13.5, 2.3 Hz, 1H), 1.96 (ddd, J=25.2, 11.7, 5.7 Hz, 1H), 1.71 (t, J=19.5 Hz, 3H), 1.28 (t, J=7.5 Hz, 3H).

### Synthesis of Compound 106

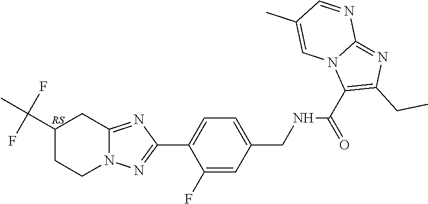 [SMILES: CCc1nc2ncc(C)cn2c1C(=O)NCc1ccc(-c2nc3n(n2)CCC(C(C)(F)F)C3)c(F)c1]

Compound 106 was synthesized from intermediates I-121 and II-3a and obtained as a beige solid (96.2 mg, 48%). ¹H NMR (400 MHz, DMSO) δ 9.17 (s, 1H), 8.52 (s, 2H), 7.94 (t, J=7.7 Hz, 1H), 7.29 (d, J=9.6 Hz, 2H), 4.58 (s, 2H), 4.37-4.28 (m, 1H), 4.19-4.07 (m, 1H), 3.15-3.07 (m, 1H), 3.03 (dd, J=14.8, 7.4 Hz, 2H), 2.87-2.74 (m, 1H), 2.74-2.60 (m, 1H), 2.34 (s, 3H), 2.26 (d, J=11.6 Hz, 1H), 2.06-1.91 (m, 1H), 1.71 (t, J=19.5 Hz, 3H), 1.29 (t, J=7.4 Hz, 3H).

### Synthesis of Compound 107

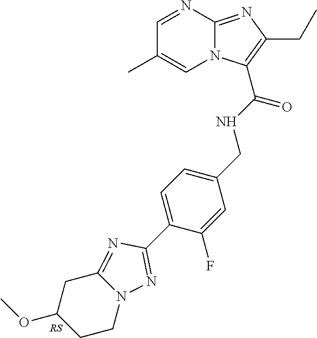 [SMILES: CCc1nc2ncc(C)cn2c1C(=O)NCc1ccc(-c2nc3n(n2)CCC(OC)C3)c(F)c1]

Compound 107 was synthesized from intermediates I-56 and II-3a and obtained as an orange solid (87.2 mg, 43%). ¹H NMR (400 MHz, DMSO) δ 9.21-9.10 (m, 1H), 8.54-8.48 (m, 2H), 7.93 (t, J=7.8 Hz, 1H), 7.28 (d, J=9.8 Hz, 2H), 4.58 (d, J=5.5 Hz, 2H), 4.25-4.06 (m, 2H), 3.99-3.90 (m, 1H), 3.32 (s, 3H), 3.10 (dd, J=17.2, 4.2 Hz, 1H), 3.03 (dd, J=15.0, 7.5 Hz, 2H), 3.00-2.94 (m, 1H), 2.34 (s, 3H), 2.30-2.12 (m, 2H), 1.29 (t, J=7.5 Hz, 3H).

### Synthesis of Compounds 108 and 109

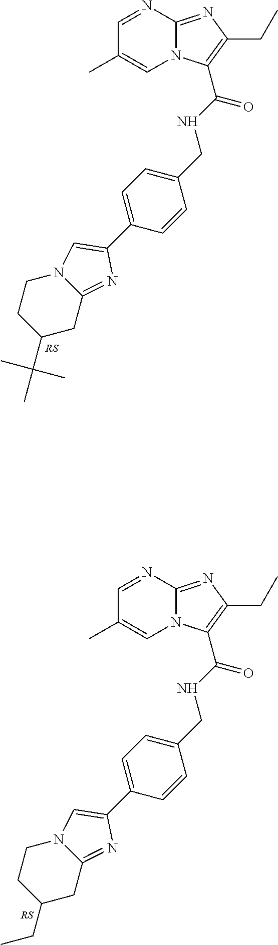

Compounds 108 and 109 were synthesized from intermediates I-133 and II-3a. Purification of the crude mixture delivered compound 108 (CF2) as a white solid (21.4 mg, 40%) and compound 109 (Et) as a yellow solid (12.1 mg, 25%).

108: ¹H NMR (400 MHz, DMSO) δ 9.16 (s, 1H), 8.52 (d, J=2.2 Hz, 1H), 8.47 (t, J=5.8 Hz, 1H), 7.70 (d, J=8.1 Hz, 2H), 7.49 (s, 1H), 7.34 (d, J=8.1 Hz, 2H), 4.52 (d, J=5.7 Hz, 2H), 4.15 (dd, J=12.4, 3.9 Hz, 1H), 3.93 (dt, J=12.2, 6.1 Hz, 1H), 3.01 (dt, J=12.5, 6.2 Hz, 3H), 2.71-2.60 (m, 1H), 2.58-2.54 (m, 1H), 2.35 (s, 3H), 2.18 (d, J=12.0 Hz, 1H), 1.87-1.76 (m, 1H), 1.70 (t, J=19.5 Hz, 3H), 1.34-1.26 (m, 3H).

109: ¹H NMR (400 MHz, DMSO) δ 9.15 (dd, J=2.4, 1.1 Hz, 1H), 8.51 (d, J=2.4 Hz, 1H), 8.46 (t, J=6.0 Hz, 1H), 7.68 (d, J=8.2 Hz, 2H), 7.43 (s, 1H), 7.32 (d, J=8.3 Hz, 2H), 4.51 (d, J=5.8 Hz, 2H), 4.06 (ddd, J=12.4, 5.5, 3.0 Hz, 1H), 3.87 (td, J=11.9, 4.6 Hz, 1H), 3.01 (q, J=7.5 Hz, 2H), 2.91 (dd, J=16.5, 3.9 Hz, 1H), 2.34 (t, J=1.8 Hz, 4H), 1.77 (s, 1H), 1.58 (ddd, J=23.9, 11.0, 5.4 Hz, 1H), 1.46-1.36 (m, 2H), 1.27 (t, J=7.5 Hz, 3H), 1.04 (d, J=6.1 Hz, 1H), 0.95 (t, J=7.4 Hz, 3H).

### Synthesis of Compound 110

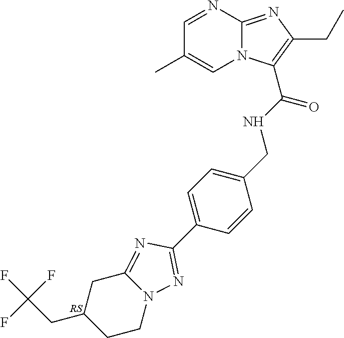 [SMILES: CCc1nc2ncc(C)cn2c1C(=O)NCc1ccc(-c2nc3n(n2)CCC(CC(F)(F)F)C3)cc1]

Compound 110 was synthesized from intermediates I-100b and II-3a and obtained as a white solid (76.2 mg, 39%). ¹H NMR (400 MHz, DMSO) δ 9.17 (s, 1H), 8.55-8.48 (m, 2H), 7.95 (d, J=8.2 Hz, 2H), 7.45 (d, J=8.2 Hz, 2H), 4.58 (d, J=5.8 Hz, 2H), 4.31-4.22 (m, 1H), 4.18-4.08 (m, 1H), 3.13-2.99 (m, 3H), 2.74-2.65 (m, 1H), 2.40 (s, 1H), 2.35 (s, 3H), 2.19 (d, J=12.9 Hz, 1H), 1.98-1.85 (m, 1H), 1.29 (t, J=7.5 Hz, 3H). CH2 signal overlapped by DMSO signal. Confirmed by HSQC.

### Synthesis of Compound 111

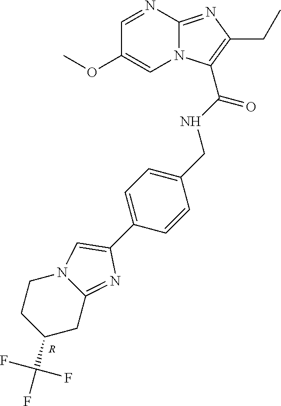

Compound 111 was synthesized from intermediates I-151a (R) and II-8b and obtained as a white solid (90.2 mg, 65%). ¹H NMR (400 MHz, DMSO) δ 8.95 (d, J=2.8 Hz, 1H), 8.52 (d, J=2.8 Hz, 1H), 8.44 (t, J=5.6 Hz, 1H), 7.70 (d, J=8.0 Hz, 2H), 7.52 (s, 1H), 7.34 (d, J=8.0 Hz, 2H), 4.53 (d, J=5.4 Hz, 2H), 4.21-4.12 (m, 1H), 3.98 (td, J=12.1, 4.3 Hz, 1H), 3.86 (s, 3H), 3.12-3.06 (m, 2H), 3.05-2.97 (m, 2H), 2.77 (dd, J=17.2, 12.7 Hz, 1H), 2.23 (d, J=12.0 Hz, 1H), 2.02-1.88 (m, 1H), 1.27 (t, J=7.4 Hz, 3H).

### Synthesis of Compound 112

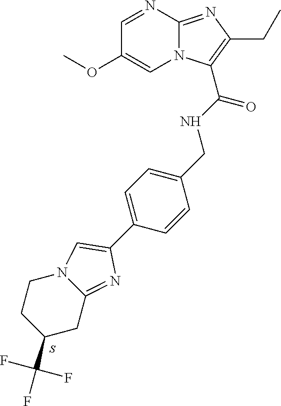

Compound 112 was synthesized from intermediates II-8b and I-151b (S) and obtained as a white solid (45.1 mg, 33%). ¹H NMR (400 MHz, DMSO) δ 8.95 (d, J=3.1 Hz, 1H), 8.52 (d, J=3.1 Hz, 1H), 8.44 (t, J=6.0 Hz, 1H), 7.69 (d, J=8.2 Hz, 2H), 7.52 (s, 1H), 7.34 (d, J=8.3 Hz, 2H), 4.53 (d, J=5.9 Hz, 2H), 4.19-4.13 (m, 1H), 3.98 (td, J=12.4, 4.9 Hz, 1H), 3.86 (s, 3H), 3.12-2.95 (m, 4H), 2.77 (dd, J=17.4, 12.9 Hz, 1H), 2.23 (d, J=11.1 Hz, 1H), 2.00-1.88 (m, 1H), 1.27 (t, J=7.5 Hz, 3H).

### Synthesis of Compound 113

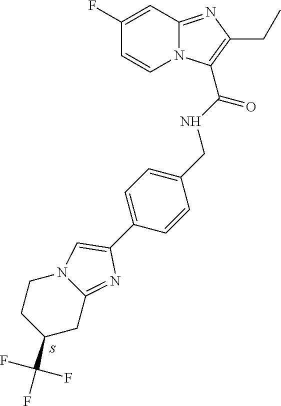

Compound 113 was synthesized from intermediates I-151b (S) and II-8c and obtained as an a pink solid (71.8 mg, 49%). ¹H NMR (400 MHz, DMSO) δ 9.06-8.98 (m, 1H), 8.42 (t, J=6.0 Hz, 1H), 7.69 (d, J=8.3 Hz, 2H), 7.52 (d, J=7.9 Hz, 1H), 7.49 (dd, J=9.8, 2.3 Hz, 1H), 7.33 (d, J=8.3 Hz, 2H), 7.06 (td, J=7.6, 2.7 Hz, 1H), 4.51 (d, J=5.9 Hz, 2H), 4.21-4.11 (m, 1H), 3.98 (td, J=12.1, 4.6 Hz, 1H), 3.14-3.03 (m, 2H), 3.01-2.93 (m, 2H), 2.78 (dd, J=17.4, 12.7 Hz, 1H), 2.23 (dd, J=13.2, 2.2 Hz, 1H), 1.95 (qd, J=11.8, 5.5 Hz, 1H), 1.26 (td, J=7.5, 3.1 Hz, 3H).

### Synthesis of Compound 114

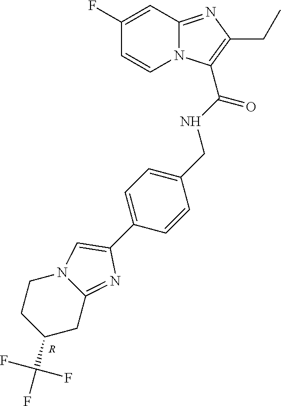

Compound 114 was synthesized from intermediates I-151a (R) and II-8c and obtained as a beige solid (63.9 mg, 48%). ¹H NMR (400 MHz, DMSO) δ 9.02 (dd, J=7.4, 6.2 Hz, 1H), 8.42 (t, J=5.9 Hz, 1H), 7.69 (d, J=8.2 Hz, 2H), 7.52 (s, 1H), 7.49 (dd, J=9.9, 2.4 Hz, 1H), 7.33 (d, J=8.3 Hz, 2H), 7.06 (td, J=7.6, 2.7 Hz, 1H), 4.51 (d, J=5.7 Hz, 2H), 4.21-4.10 (m, 1H), 3.98 (td, J=12.1, 4.6 Hz, 1H), 3.15-3.02 (m, 2H), 2.97 (q, J=7.5 Hz, 2H), 2.77 (dd, J=17.3, 12.6 Hz, 1H), 2.28-2.19 (m, 1H), 2.03-1.88 (m, 1H), 1.26 (t, J=7.5 Hz, 3H).

### Synthesis of Compound 115

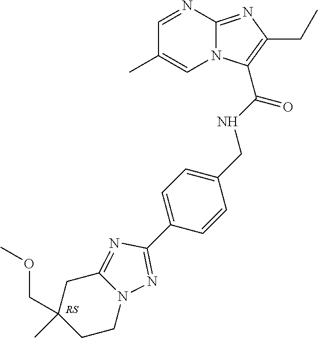

Compound 115 was synthesized from intermediates I-111 and II-3a and obtained as a pale orange solid (98.9 mg, 57%). ¹H NMR (400 MHz, DMSO) δ 9.16 (dd, J=2.4, 1.1 Hz, 1H), 8.54-8.47 (m, 2H), 7.94 (d, J=8.3 Hz, 2H), 7.44 (d, J=8.3 Hz, 2H), 4.57 (d, J=5.8 Hz, 2H), 4.21-4.07 (m, 2H), 3.29 (s, 3H), 3.22 (s, 2H), 3.02 (q, J=7.5 Hz, 2H), 2.78 (d, J=17.1 Hz, 1H), 2.63 (d, J=17.0 Hz, 1H), 2.34 (s, 3H), 1.99 (dt, J=13.9, 6.8 Hz, 1H), 1.89-1.80 (m, 1H), 1.28 (t, J=7.5 Hz, 3H), 1.00 (s, 3H).

### Synthesis of Compound 116

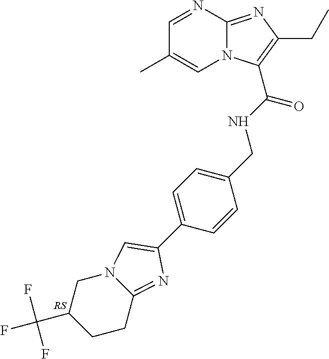

Compound 116 was synthesized from intermediates I-135 and II-3a and obtained as a white solid (61.3 mg, 33%). ¹H NMR (400 MHz, DMSO) δ 9.15 (dd, J=2.3, 1.1 Hz, 1H), 8.51 (d, J=2.4 Hz, 1H), 8.47 (t, J=6.0 Hz, 1H), 7.68 (d, J=8.2 Hz, 2H), 7.50 (s, 1H), 7.34 (d, J=8.3 Hz, 2H), 4.52 (d, J=5.9 Hz, 2H), 4.30 (dd, J=12.4, 5.2 Hz, 1H), 4.03-3.94 (m, 1H), 3.20 (s, 1H), 3.01 (q, J=7.5 Hz, 2H), 2.97-2.88 (m, 1H), 2.83 (ddd, J=16.8, 11.1, 6.0 Hz, 1H), 2.34 (s, 3H), 2.24-2.14 (m, 1H), 1.92 (ddd, J=24.3, 11.3, 5.8 Hz, 1H), 1.27 (t, J=7.5 Hz, 3H).

### Synthesis of Compound 117

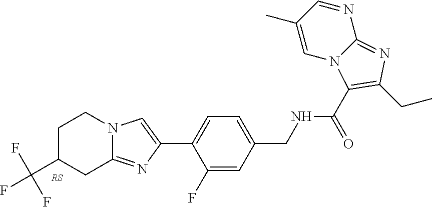 [SMILES: CCc1nc2ncc(C)cn2c1C(=O)NCc1ccc(-c2cn3c(n2)CC(C(F)(F)F)CC3)c(F)c1]

Compound 117 was synthesized from intermediates I-130 and II-3a and obtained as a white solid (102 mg, 65%). ¹H NMR (400 MHz, DMSO) δ 9.16 (dd, J=2.3, 1.1 Hz, 1H), 8.51 (d, J=2.4 Hz, 1H), 8.48 (s, 1H), 7.98 (t, J=8.2 Hz, 1H), 7.43 (d, J=4.3 Hz, 1H), 7.26-7.18 (m, 2H), 4.53 (s, 2H), 4.27-4.17 (m, 1H), 3.99 (td, J=12.2, 4.5 Hz, 1H), 3.17-3.06 (m, 2H), 3.02 (q, J=7.5 Hz, 2H), 2.80 (dd, J=17.4, 12.7 Hz, 1H), 2.34 (s, 3H), 2.28-2.20 (m, 1H), 1.95 (qd, J=11.9, 5.6 Hz, 1H), 1.28 (t, J=7.5 Hz, 3H).

### Synthesis of Compound 118

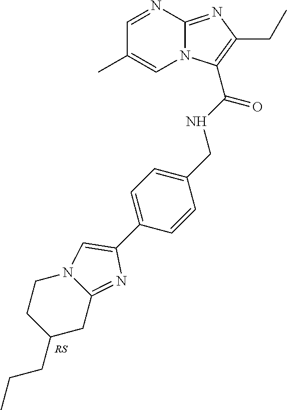

Compound 118 was synthesized from intermediates I-136 and II-3a and obtained as a white solid (78.8 mg, 24%). ¹H NMR (400 MHz, DMSO) δ 9.15 (dd, J=2.3, 1.1 Hz, 1H), 8.51 (d, J=2.4 Hz, 1H), 8.47 (t, J=5.9 Hz, 1H), 7.68 (d, J=8.2 Hz, 2H), 7.44 (s, 1H), 7.32 (d, J=8.3 Hz, 2H), 4.51 (d, J=5.7 Hz, 2H), 4.11-4.02 (m, 1H), 3.95-3.83 (m, 1H), 3.37-3.33 (m, 2H), 3.29 (s, J=1.9 Hz, 3H), 3.01 (q, J=7.5 Hz, 2H), 2.86 (dd, J=16.5, 4.3 Hz, 1H), 2.41 (dd, J=16.5, 10.6 Hz, 1H), 2.34 (d, J=0.6 Hz, 3H), 2.24-2.13 (m, 1H), 2.08-1.97 (m, 1H), 1.71-1.57 (m, 1H), 1.27 (t, J=7.5 Hz, 3H).

### Synthesis of Compound 119

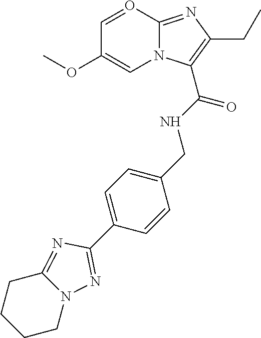

Compound 119 was synthesized from intermediates I-73a and II-8b and obtained as a beige solid (96.6 mg, 48%). ¹H NMR (400 MHz, DMSO) δ 8.97 (d, J=2.9 Hz, 1H), 8.52 (d, J=2.9 Hz, 1H), 8.47 (s, 1H), 7.94 (d, J=8.1 Hz, 2H), 7.44 (d, J=8.0 Hz, 2H), 4.58 (d, J=4.8 Hz, 2H), 4.13 (t, J=5.8 Hz, 2H), 3.86 (s, 3H), 3.03 (q, J=7.5 Hz, 2H), 2.85 (t, J=6.2 Hz, 2H), 2.06-1.97 (m, 2H), 1.95-1.86 (m, 2H), 1.28 (t, J=7.5 Hz, 3H).

### Synthesis of Compound 120

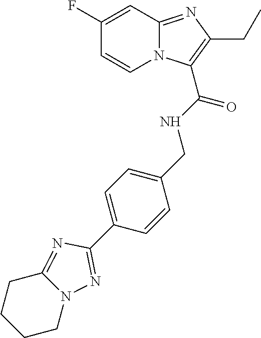 [SMILES: CCc1nc2cc(F)ccn2c1C(=O)NCc1ccc(-c2nc3n(n2)CCCC3)cc1]

Compound 120 was synthesized from intermediates I-73a and 11-101 and obtained as a white solid (100.5 mg, 51%). ¹H NMR (400 MHz, DMSO) δ 9.04 (dd, J=7.5, 6.2 Hz, 1H), 8.46 (t, J=5.9 Hz, 1H), 7.95 (d, J=8.3 Hz, 2H), 7.50 (dd, J=9.8, 2.6 Hz, 1H), 7.44 (d, J=8.3 Hz, 2H), 7.07 (td, J=7.6, 2.7 Hz, 1H), 4.57 (d, J=5.7 Hz, 2H), 4.14 (t, J=6.0 Hz, 2H), 2.99 (q, J=7.5 Hz, 2H), 2.86 (t, J=6.3 Hz, 2H), 2.06-1.99 (m, 2H), 1.95-1.87 (m, 2H), 1.27 (t, J=7.5 Hz, 3H).

### Synthesis of Compound 121

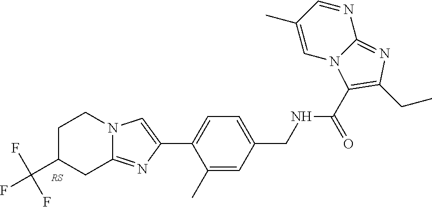 [SMILES: CCc1nc2ncc(C)cn2c1C(=O)NCc1ccc(-c2cn3c(n2)CC(C(F)(F)F)CC3)c(C)c1]

Compound 121 was synthesized from intermediates I-129 and II-3a and obtained as a white solid (87.1 mg, 38%). ¹H NMR (400 MHz, DMSO) δ 9.14 (s, 1H), 8.51 (d, J=2.3 Hz, 1H), 8.47 (t, J=5.2 Hz, 1H), 7.77 (d, J=8.5 Hz, 1H), 7.31 (s, 1H), 7.19 (d, J=4.5 Hz, 2H), 4.50 (d, J=4.4 Hz, 2H), 4.20 (dd, J=12.5, 3.8 Hz, 1H), 4.00 (td, J=12.2, 4.5 Hz, 1H), 3.12-3.05 (m, 2H), 3.02 (q, J=7.5 Hz, 2H), 2.79 (dd, J=17.2, 12.6 Hz, 1H), 2.43 (s, 3H), 2.34 (s, 3H), 2.24 (d, J=13.4 Hz, 1H), 1.96 (qd, J=11.9, 5.4 Hz, 1H), 1.28 (t, J=7.5 Hz, 3H).

### Synthesis of Compound 122

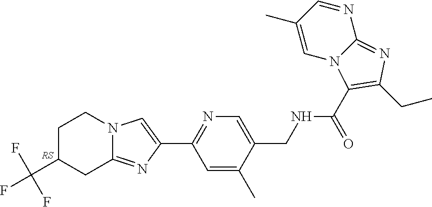 [SMILES: CCc1nc2ncc(C)cn2c1C(=O)NCc1cnc(-c2cn3c(n2)CC(C(F)(F)F)CC3)cc1C]

Compound 122 was synthesized from intermediates I-142 and II-3a and obtained as a white solid (78.6 mg, 42%). ¹H NMR (400 MHz, DMSO) δ 9.21 (s, 1H), 8.84 (s, 1H), 8.52 (d, J=2.3 Hz, 1H), 8.48 (t, J=5.7 Hz, 1H), 7.46 (s, 1H), 7.24 (s, 1H), 4.61 (d, J=5.7 Hz, 2H), 4.22 (dd, J=12.8, 3.5 Hz, 1H), 4.02 (td, J=12.3, 4.5 Hz, 1H), 3.21-2.98 (m, 4H), 2.81 (dd, J=17.2, 12.6 Hz, 1H), 2.44 (s, 3H), 2.34 (s, 3H), 2.25 (d, J=11.3 Hz, 1H), 2.05-1.90 (m, 1H), 1.32 (t, J=7.5 Hz, 3H).

### Synthesis of Compound 123

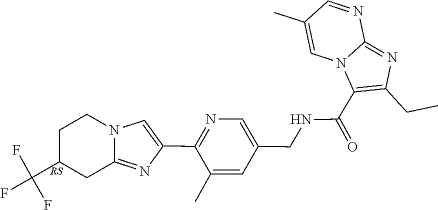 [SMILES: CCc1nc2ncc(C)cn2c1C(=O)NCc1cnc(-c2cn3c(n2)CC(C(F)(F)F)CC3)c(C)c1]

Compound 123 was synthesized from intermediates I-143 and II-3a and obtained as a beige solid (47.2 mg, 26%). ¹H NMR (400 MHz, DMSO) δ 9.15 (dd, J=2.4, 1.1 Hz, 1H), 8.51 (d, J=2.4 Hz, 1H), 8.48 (t, J=5.9 Hz, 1H), 8.36 (d, J=1.9 Hz, 1H), 7.58 (s, 1H), 7.55 (d, J=1.6 Hz, 1H), 4.51 (d, J=5.8 Hz, 2H), 4.27-4.18 (m, 1H), 4.00 (td, J=12.2, 4.6 Hz, 1H), 3.18-3.05 (m, 2H), 3.01 (q, J=7.5 Hz, 2H), 2.80 (dd, J=17.2, 12.5 Hz, 1H), 2.61 (s, 3H), 2.34 (d, J=0.6 Hz, 3H), 2.29-2.19 (m, 1H), 2.03-1.88 (m, 1H), 1.28 (t, J=7.5 Hz, 3H).

### Synthesis of Compound 124

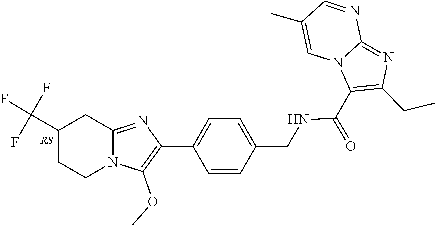 [SMILES: CCc1nc2ncc(C)cn2c1C(=O)NCc1ccc(-c2nc3n(c2OC)CCC(C(F)(F)F)C3)cc1]

Compound 124 was synthesized from intermediates I-96 and II-3a and obtained as a white solid (60.3 mg, 48%). ¹H NMR (400 MHz, DMSO) δ 9.16 (dd, J=2.3, 1.1 Hz, 1H), 8.51 (d, J=2.4 Hz, 1H), 8.47 (t, J=5.9 Hz, 1H), 7.72 (d, J=8.3 Hz, 2H), 7.37 (d, J=8.3 Hz, 2H), 4.52 (d, J=5.9 Hz, 2H), 4.10-3.98 (m, 1H), 3.85 (dd, J=12.0, 4.6 Hz, 1H), 3.81 (s, 3H), 3.06-2.94 (m, 4H), 2.73 (dd, J=15.9, 11.4 Hz, 1H), 2.34 (s, J=6.1 Hz, 3H), 2.26 (d, J=11.4 Hz, 1H), 1.99-1.84 (m, 1H), 1.28 (t, J=7.5 Hz, 3H).

### Synthesis of Compound 125

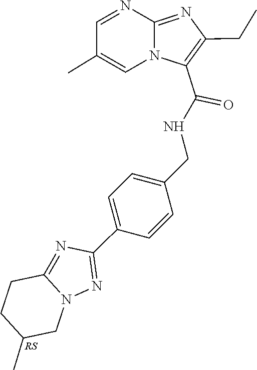 [SMILES: CCc1nc2ncc(C)cn2c1C(=O)NCc1ccc(-c2nc3n(n2)CC(C)CC3)cc1]

Compound 125 was synthesized from intermediates I-16 and II-3a and obtained as a white solid (136.6 mg, 58%). ¹H NMR (400 MHz, DMSO) δ 9.16 (s, 1H), 8.51 (s, 2H), 7.94 (d, J=7.6 Hz, 2H), 7.43 (d, J=7.6 Hz, 2H), 4.57 (d, J=5.0 Hz, 2H), 4.28-4.19 (m, 1H), 3.69 (t, J=11.0 Hz, 1H), 3.02 (dd, J=14.6, 7.2 Hz, 2H), 2.88 (dd, J=40.3, 13.9 Hz, 2H), 2.34 (s, 3H), 2.20 (s, 1H), 1.96 (s, 1H), 1.60 (d, J=6.8 Hz, 1H), 1.28 (t, J=7.3 Hz, 3H), 1.08 (d, J=6.4 Hz, 3H).

### Synthesis of Compound 126 and 127

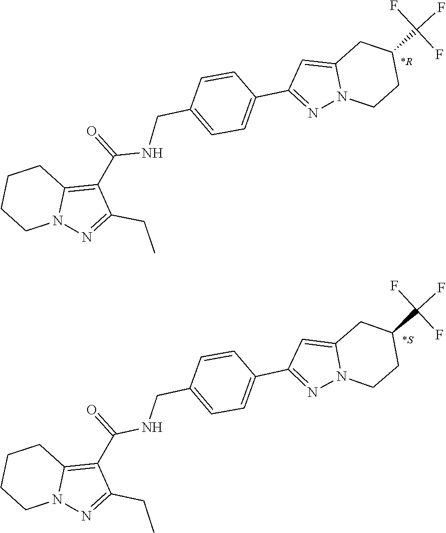 [SMILES: CCc1nn2c(c1C(=O)NCc1ccc(-c3cc4n(n3)CC[C@@H](C(F)(F)F)C4)cc1)CCCC2 ; CCc1nn2c(c1C(=O)NCc1ccc(-c3cc4n(n3)CC[C@H](C(F)(F)F)C4)cc1)CCCC2]

Isomers 126 and 127 were synthesized from intermediates I-27 and 11-84, after SFC separation (Jasco SFC prep system, i-cellulose column (Phenomenex) 250*30 mm, 5 mm particle size, isocratic mode at 100 ml/min of CO₂ (40%)/MeOH (60%)/diethylamine (0.1%) at 30° C., 120 bars) it were obtained 126 (R*) (37.4 mg, yield: 24%) and compound 127 (S*) (40.3 mg, yield: 26%) as white solids.

126: ¹H NMR (400 MHz, DMSO) δ 7.82 (t, J=6.0 Hz, 1H), 7.70 (d, J=8.2 Hz, 2H), 7.30 (d, J=8.3 Hz, 2H), 6.51 (s, 1H), 4.40 (d, J=6.0 Hz, 2H), 4.34-4.25 (m, 1H), 4.11 (td, J=12.3, 4.7 Hz, 1H), 3.98 (t, J=6.0 Hz, 2H), 3.18-3.08 (m, 1H), 3.09-2.95 (m, 1H), 2.89 (t, J=6.3 Hz, 2H), 2.79 (dd, J=15.8, 11.1 Hz, 1H), 2.70 (q, J=7.5 Hz, 2H), 2.31-2.22 (m, 1H), 2.04 (qd, J=11.8, 5.7 Hz, 1H), 1.96-1.86 (m, 2H), 1.81-1.67 (m, 2H), 1.11 (t, J=7.5 Hz, 3H). 127: ¹H NMR (400 MHz, DMSO) δ 7.82 (t, J=6.0 Hz, 1H), 7.70 (d, J=8.2 Hz, 2H), 7.30 (d, J=8.3 Hz, 2H), 6.51 (s, 1H), 4.40 (d, J=6.0 Hz, 2H), 4.33-4.25 (m, 1H), 4.11 (td, J=12.3, 4.8 Hz, 1H), 3.98 (t, J=6.0 Hz, 2H), 3.13 (dd, J=15.8, 3.7 Hz, 1H), 3.08-2.96 (m, 1H), 2.88 (t, J=6.3 Hz, 2H), 2.79 (dd, J=15.8, 11.2 Hz, 1H), 2.70 (q, J=7.5 Hz, 2H), 2.27 (dd, J=13.2, 2.3 Hz, 1H), 2.03 (ddd, J=25.0, 11.8, 5.7 Hz, 1H), 1.95-1.88 (m, 2H), 1.80-1.72 (m, 2H), 1.11 (t, J=7.5 Hz, 3H).

### Synthesis of Compound 128

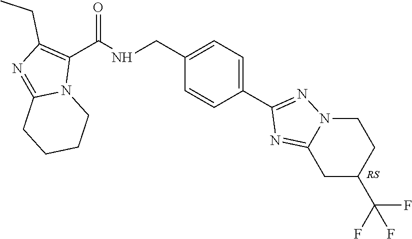 [SMILES: CCc1nc2n(c1C(=O)NCc1ccc(-c3nc4n(n3)CCC(C(F)(F)F)C4)cc1)CCCC2]

Compound 128 was synthesized from intermediates I-47 and II-17a and obtained as a white solid (78.9 mg, 22%). ¹H NMR (400 MHz, DMSO) δ 8.25 (t, J=6.0 Hz, 1H), 7.93 (d, J=8.2 Hz, 2H), 7.38 (d, J=8.3 Hz, 2H), 4.45 (d, J=6.0 Hz, 2H), 4.36-4.28 (m, 1H), 4.17 (td, J=12.0, 4.9 Hz, 1H), 3.99 (t, J=5.8 Hz, 2H), 3.18 (dd, J=9.5, 8.3 Hz, 2H), 2.92 (dd, J=17.6, 12.2 Hz, 1H), 2.71 (t, J=6.3 Hz, 2H), 2.64 (q, J=7.5 Hz, 2H), 2.36-2.27 (m, 1H), 2.18-2.03 (m, 1H), 1.91-1.73 (m, 4H), 1.10 (t, J=7.5 Hz, 3H).

### Synthesis of Compound 129

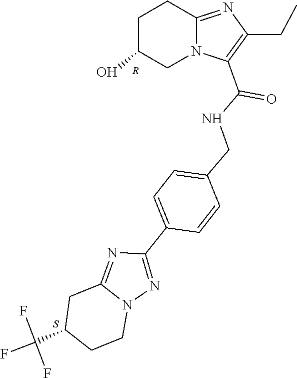

Compound 129 was synthesized from intermediates I-87 (S) and II-65 and obtained as a white solid (22.4 mg, 18%). ¹H NMR (400 MHz, DMSO) δ 8.25 (t, J=5.8 Hz, 1H), 7.94 (d, J=8.2 Hz, 2H), 7.39 (d, J=8.2 Hz, 2H), 5.12 (d, J=3.2 Hz, 1H), 4.46 (d, J=6.0 Hz, 2H), 4.37-4.30 (m, 1H), 4.19 (dd, J=11.9, 4.9 Hz, 1H), 4.15-4.05 (m, 2H), 3.87 (dd, J=13.1, 4.5 Hz, 1H), 3.20 (d, J=12.9 Hz, 1H), 2.93 (dd, J=17.5, 12.1 Hz, 1H), 2.81 (dd, J=15.7, 8.9 Hz, 1H), 2.74-2.61 (m, 4H), 2.34 (dt, J=3.8, 1.9 Hz, 1H), 2.18-2.06 (m, 1H), 1.86 (d, J=4.8 Hz, 2H), 1.12 (t, J=7.5 Hz, 3H).

### Synthesis of Compound 130

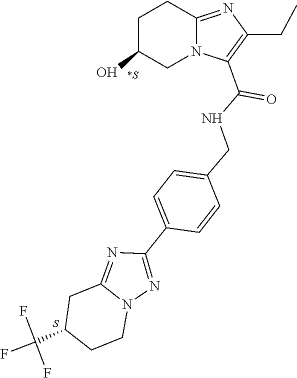

Compound 130 was synthesized from intermediates I-87 (S) and II-66 and obtained as a white solid (69.2 mg, 65%). ¹H NMR (400 MHz, DMSO) δ 8.36 (s, 1H), 7.94 (d, J=8.3 Hz, 2H), 7.38 (d, J=8.3 Hz, 2H), 5.16 (d, J=3.2 Hz, 1H), 4.46 (d, J=6.0 Hz, 2H), 4.32 (dd, J=11.3, 4.2 Hz, 1H), 4.21-4.05 (m, 3H), 3.89 (dd, J=13.2, 4.1 Hz, 1H), 3.24-3.14 (m, 2H), 2.97-2.63 (m, 5H), 2.36-2.28 (m, 1H), 2.18-2.05 (m, 1H), 1.87 (dd, J=11.4, 5.9 Hz, 2H), 1.12 (t, J=7.5 Hz, 3H).

### Synthesis of Compound 131

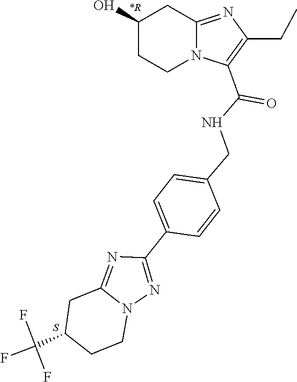

Compound 131 was synthesized from intermediates I-87 (S) and II-67 and obtained as a white solid (79.4 mg, 63%). ¹H NMR (400 MHz, DMSO) δ 8.28 (t, J=5.9 Hz, 1H), 7.93 (d, J=8.3 Hz, 2H), 7.38 (d, J=8.3 Hz, 2H), 5.06 (d, J=3.4 Hz, 1H), 4.46 (d, J=6.0 Hz, 2H), 4.38-4.27 (m, 1H), 4.18 (dd, J=11.7, 4.9 Hz, 1H), 4.15-4.07 (m, 1H), 4.07-4.00 (m, 2H), 3.24-3.12 (m, 2H), 3.00-2.85 (m, 2H), 2.69-2.59 (m, 3H), 2.37-2.25 (m, 1H), 2.18-2.05 (m, 1H), 2.00-1.91 (m, 1H), 1.92-1.81 (m, 1H), 1.11 (t, J=7.5 Hz, 3H).

### Synthesis of Compound 132

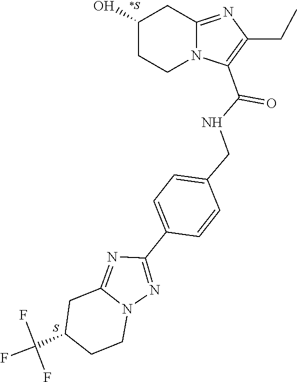

Compound 132 was synthesized from intermediates I-87 (S) and II-68 and obtained as an orange solid (43 mg, 43%). ¹H NMR (400 MHz, DMSO) δ 8.31 (s, 1H), 7.94 (d, J=8.3 Hz, 2H), 7.38 (d, J=8.3 Hz, 2H), 5.08 (d, J=3.3 Hz, 1H), 4.46 (d, J=5.9 Hz, 2H), 4.32 (dd, J=12.8, 3.6 Hz, 1H), 4.22-4.13 (m, 1H), 4.13-3.97 (m, 3H), 3.24-3.14 (m, 2H), 2.99-2.86 (m, 2H), 2.71-2.56 (m, 3H), 2.38-2.27 (m, 1H), 2.11 (ddd, J=24.7, 11.5, 5.8 Hz, 1H), 2.01-1.82 (m, 2H), 1.11 (t, J=7.5 Hz, 3H).

### Synthesis of Compound 133

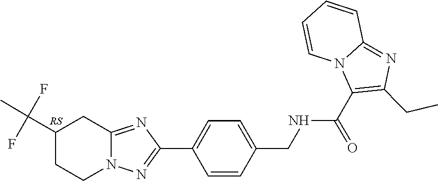 [SMILES: CCc1nc2ccccn2c1C(=O)NCc1ccc(-c2nc3n(n2)CCC(C(C)(F)F)C3)cc1]

Compound 133 was synthesized from intermediates I-160 and 11-102 and obtained as a white solid (53.4 mg, 52%). ¹H NMR (400 MHz, DMSO) δ 8.99 (dt, J=7.0, 1.1 Hz, 1H), 8.45 (t, J=6.0 Hz, 1H), 7.96 (d, J=8.3 Hz, 2H), 7.61 (dt, J=9.0, 1.1 Hz, 1H), 7.45 (d, J=8.4 Hz, 2H), 7.39 (ddd, J=8.9, 6.8, 1.3 Hz, 1H), 4.58 (d, J=5.9 Hz, 2H), 4.38-4.21 (m, 1H), 4.12 (td, J=12.3, 4.8 Hz, 1H), 3.10 (dd, J=16.6, 3.9 Hz, 1H), 3.01 (q, J=7.5 Hz, 2H), 2.80 (dd, J=16.5, 11.4 Hz, 1H), 2.73-2.57 (m, 1H), 2.26 (dd, J=13.4, 2.0 Hz, 1H), 1.96 (tt, J=18.9, 6.5 Hz, 1H), 1.72 (t, J=19.5 Hz, 3H), 1.28 (t, J=7.5 Hz, 3H).

### Synthesis of Compound 134

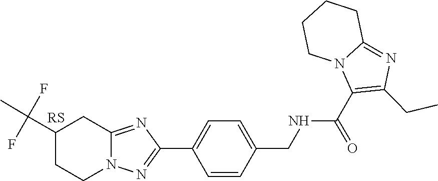 [SMILES: CCc1nc2n(c1C(=O)NCc1ccc(-c3nc4n(n3)CCC(C(C)(F)F)C4)cc1)CCCC2]

Compound 134 was synthesized from intermediates I-160 and II-17a and obtained as a white solid (50 mg, 49%). ¹H NMR (400 MHz, DMSO) δ 8.25 (t, J=6.0 Hz, 1H), 7.93 (d, J=8.3 Hz, 2H), 7.37 (d, J=8.3 Hz, 2H), 4.45 (d, J=6.0 Hz, 2H), 4.34-4.27 (m, 1H), 4.11 (td, J=12.1, 4.7 Hz, 1H), 3.99 (t, J=5.8 Hz, 2H), 3.09 (dd, J=16.6, 3.9 Hz, 1H), 2.79 (dd, J=16.4, 11.4 Hz, 1H), 2.71 (t, J=6.3 Hz, 2H), 2.64 (q, J=7.5 Hz, 3H), 2.25 (dd, J=13.4, 2.1 Hz, 1H), 1.96 (ddd, J=25.2, 11.8, 5.8 Hz, 1H), 1.89-1.77 (m, 4H), 1.71 (t, J=19.5 Hz, 3H), 1.10 (t, J=7.5 Hz, 3H).

### Synthesis of Compound 135

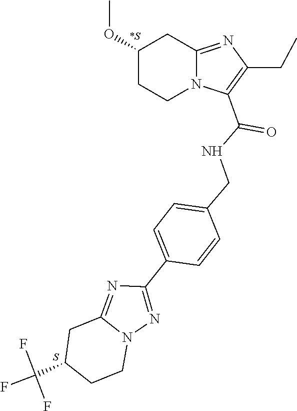

Compound 135 was synthesized from intermediates I-87 (S) and II-70 and obtained as a white solid (39 mg, 29%). ¹H NMR (400 MHz, DMSO) δ 8.28 (t, J=6.0 Hz, 1H), 7.98-7.89 (m, 2H), 7.38 (d, J=8.3 Hz, 2H), 4.45 (d, J=6.0 Hz, 2H), 4.37-4.25 (m, 1H), 4.17 (td, J=11.9, 4.7 Hz, 1H), 4.10-4.01 (m, 1H), 4.01-3.90 (m, 1H), 3.84-3.76 (m, 1H), 3.29 (s, 3H), 3.23-3.14 (m, 2H), 2.99-2.87 (m, 2H), 2.78 (dd, J=16.9, 4.9 Hz, 1H), 2.64 (q, J=7.5 Hz, 2H), 2.36-2.26 (m, 1H), 2.19-2.07 (m, 1H), 2.07-1.98 (m, 2H), 1.11 (t, J=7.5 Hz, 3H).

### Synthesis of Compound 136

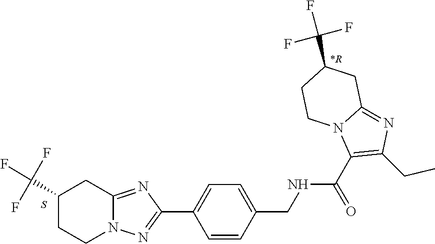

Compound 136 was synthesized from intermediates I-87 (S) and II-71 and obtained as a white solid (65 mg, 61%). ¹H NMR (400 MHz, DMSO) δ 8.32 (t, J=5.9 Hz, 1H), 7.94 (d, J=8.3 Hz, 2H), 7.38 (d, J=8.3 Hz, 2H), 4.46 (d, J=6.1 Hz, 2H), 4.35-4.26 (m, 2H), 4.17 (td, J=11.9, 4.9 Hz, 1H), 3.95 (td, J=12.7, 4.4 Hz, 1H), 3.22-3.15 (m, 2H), 3.06-2.99 (m, 2H), 2.92 (dd, J=17.5, 11.9 Hz, 1H), 2.78-2.63 (m, 3H), 2.31 (ddd, J=6.9, 4.2, 2.1 Hz, 1H), 2.23-2.18 (m, 1H), 2.17-2.05 (m, 1H), 1.92-1.81 (m, 1H), 1.12 (t, J=7.5 Hz, 3H).

### Synthesis of Compound 137

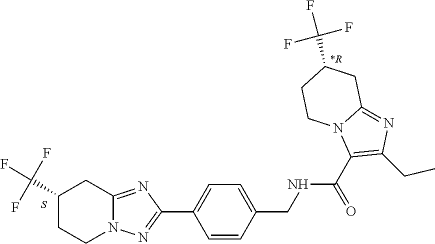

Compound 137 was synthesized from intermediates I-87 (S) and II-72 and obtained as a white solid (85 mg, 79%). 1H NMR (400 MHz, DMSO) δ 8.33 (t, J=5.8 Hz, 1H), 7.94 (d, J=8.1 Hz, 2H), 7.39 (d, J=8.0 Hz, 2H), 4.47 (d, J=5.7 Hz, 2H), 4.40-4.25 (m, 2H), 4.24-4.10 (m, 1H), 4.05-3.87 (m, 1H), 3.26-3.14 (m, 2H), 3.11-2.99 (m, 2H), 2.99-2.87 (m, 1H), 2.82-2.60 (m, 3H), 2.39-2.26 (m, 1H), 2.26-2.05 (m, 2H), 1.93-1.80 (m, 1H), 1.13 (t, J=7.5 Hz, 3H).

### Synthesis of Compound 139

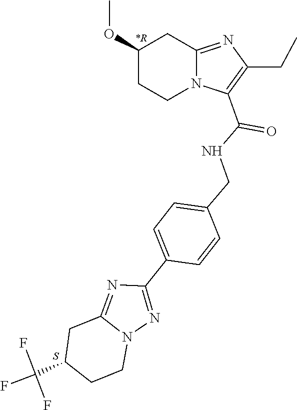

Compound 139 was synthesized from intermediates I-87 (S) and II-69 and obtained as a white solid (49 mg, 68%). ¹H NMR (400 MHz, DMSO) δ 8.28 (t, J=6.0 Hz, 1H), 7.93 (d, J=8.3 Hz, 2H), 7.38 (d, J=8.3 Hz, 2H), 4.45 (d, J=6.0 Hz, 2H), 4.36-4.28 (m, 1H), 4.17 (td, J=12.1, 4.9 Hz, 1H), 4.11-4.02 (m, 1H), 4.00-3.90 (m, 1H), 3.83-3.76 (m, 1H), 3.29 (s, 3H), 3.22-3.15 (m, 2H), 2.99-2.87 (m, 2H), 2.78 (dd, J=16.9, 4.8 Hz, 1H), 2.64 (q, J=7.5 Hz, 2H), 2.37-2.27 (m, 1H), 2.18-2.06 (m, 1H), 2.06-1.97 (m, 2H), 1.11 (t, J=7.5 Hz, 3H).

### Synthesis of Compound 140

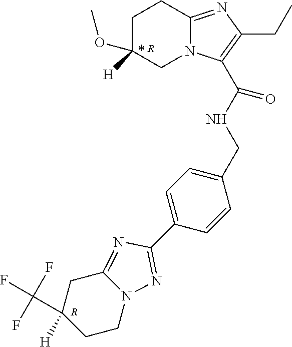

Compound 140 was synthesized from intermediates I-88 (R) and II-73 and obtained as a white solid (37 mg, 23%). ¹H NMR (400 MHz, DMSO) δ 8.27-8.23 (m, 1H), 7.93 (d, J=8.2 Hz, 2H), 7.38 (d, J=8.2 Hz, 2H), 4.46 (d, J=6.0 Hz, 2H), 4.36-4.28 (m, 1H), 4.21-4.00 (m, 3H), 3.84-3.80 (m, 1H), 3.28 (s, 3H), 3.22-3.15 (m, 2H), 2.92 (dd, J=17.8, 12.3 Hz, 1H), 2.74-2.60 (m, 4H), 2.32 (d, J=7.8 Hz, 1H), 2.18-1.84 (m, 3H), 1.11 (t, J=7.5 Hz, 3H).

### Synthesis of Compound 141

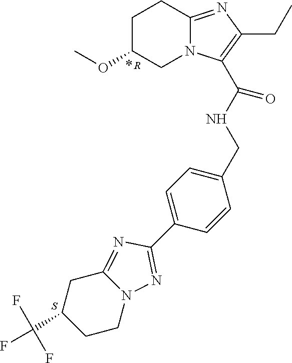

Compound 141 was synthesized from intermediates I-87 (S) and II-73 and obtained as a white solid (71 mg, 44%). ¹H NMR (400 MHz, DMSO) δ 8.26 (t, J=6.0 Hz, 1H), 7.94 (d, J=8.2 Hz, 2H), 7.38 (d, J=8.2 Hz, 2H), 4.46 (d, J=6.0 Hz, 2H), 4.32 (dd, J=12.5, 3.8 Hz, 1H), 4.22-4.00 (m, 3H), 3.85-3.79 (m, 1H), 3.28 (s, 3H), 3.22-3.14 (m, 2H), 2.92 (dd, J=17.7, 12.2 Hz, 1H), 2.73-2.60 (m, 4H), 2.36-2.28 (m, 1H), 2.18-2.03 (m, 2H), 1.94-1.84 (m, 1H), 1.11 (t, J=7.5 Hz, 3H).

### Synthesis of Compound 142

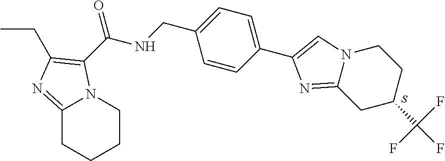

Compound 142 was synthesized from intermediates I-151b and II-17a and obtained as a white solid (60.7 mg, 33%). ¹H NMR (400 MHz, DMSO) δ 8.21 (t, J=6.0 Hz, 1H), 7.67 (d, J=8.2 Hz, 2H), 7.52 (s, 1H), 7.26 (d, J=8.2 Hz, 2H), 4.40 (d, J=6.0 Hz, 2H), 4.16 (dd, J=12.6, 3.3 Hz, 1H), 4.02-3.92 (m, 3H), 3.13-3.03 (m, 2H), 2.77 (dd, J=17.5, 12.6 Hz, 1H), 2.70 (t, J=6.3 Hz, 2H), 2.63 (q, J=7.5 Hz, 2H), 2.27-2.19 (m, 1H), 2.01-1.89 (m, 1H), 1.89-1.82 (m, 2H), 1.81-1.74 (m, 2H), 1.10 (t, J=7.5 Hz, 3H).

### Synthesis of Compound 143

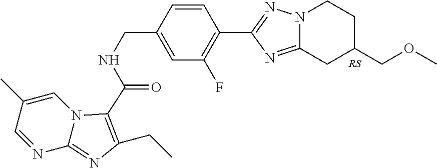

Compound 143 was synthesized from intermediates I-124 and II-3a and obtained as a white solid (122.6 mg, 66%). ¹H NMR (400 MHz, DMSO) δ 9.20-9.15 (m, 1H), 8.55-8.48 (m, 2H), 7.94 (t, J=7.8 Hz, 1H), 7.28 (d, J=9.7 Hz, 2H), 4.57 (s, 2H), 4.31-4.19 (m, 1H), 4.15-4.03 (m, 1H), 3.41-3.36 (m, 2H), 3.32 (s, 3H), 3.03 (q, J=7.5 Hz, 2H), 3.00-2.88 (m, 1H), 2.60-2.54 (m, 1H), 2.36-2.31 (m, 3H), 2.31-2.25 (m, 1H), 2.17-2.09 (m, 1H), 1.86-1.73 (m, 1H), 1.29 (t, J=7.5 Hz, 3H).

### Synthesis of Compound 144

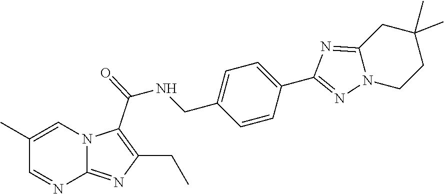

Compound 144 was synthesized from intermediates I-116 and II-3a and obtained as a white solid (155.8 mg, 64%). ¹H NMR (400 MHz, DMSO) δ 9.16 (dd, J=2.3, 1.1 Hz, 1H), 8.51 (t, J=5.0 Hz, 2H), 7.94 (d, J=8.3 Hz, 2H), 7.44 (d, J=8.3 Hz, 2H), 4.57 (d, J=5.9 Hz, 2H), 4.14 (t, J=6.3 Hz, 2H), 3.02 (q, J=7.5 Hz, 2H), 2.67 (s, 2H), 2.34 (s, 3H), 1.86 (t, J=6.3 Hz, 2H), 1.30-1.25 (t, J=7.5 Hz, 3H), 1.04 (s, 6H).

### Synthesis of Compound 145

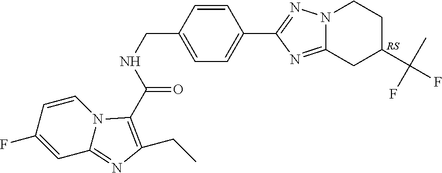 [SMILES: CCc1nc2cc(F)ccn2c1C(=O)NCc1ccc(-c2nc3n(n2)CCC(C(C)(F)F)C3)cc1]

Compound 145 was synthesized from intermediates I-73a and I-160 and obtained as a beige solid (146.1 mg, 73%). ¹H NMR (400 MHz, DMSO) δ 9.04 (dd, J=7.4, 6.2 Hz, 1H), 8.48 (t, J=5.9 Hz, 1H), 7.95 (d, J=8.2 Hz, 2H), 7.50 (dd, J=9.8, 2.6 Hz, 1H), 7.45 (d, J=8.3 Hz, 2H), 7.07 (td, J=7.6, 2.7 Hz, 1H), 4.57 (d, J=5.8 Hz, 2H), 4.35-4.27 (m, 1H), 4.12 (td, J=12.1, 4.6 Hz, 1H), 3.10 (dd, J=16.6, 4.1 Hz, 1H), 3.00 (q, J=7.5 Hz, 2H), 2.80 (dd, J=16.5, 11.4 Hz, 1H), 2.71-2.57 (m, 1H), 2.26 (dd, J=13.4, 2.2 Hz, 1H), 1.96 (qd, J=11.8, 5.7 Hz, 1H), 1.72 (t, J=19.5 Hz, 3H), 1.27 (t, J=7.5 Hz, 3H).

### Synthesis of Compound 146

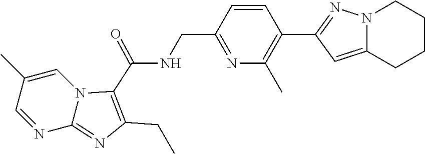 [SMILES: CCc1nc2ncc(C)cn2c1C(=O)NCc1ccc(-c2cc3n(n2)CCCC3)c(C)n1]

Compound 146 was synthesized from intermediates I-149 and II-3a and obtained as a beige solid (15.1 mg, 7%). ¹H NMR (400 MHz, DMSO) δ 9.21 (s, 1H), 8.52 (t, J=3.9 Hz, 1H), 8.49 (t, J=5.5 Hz, 1H), 7.89 (d, J=8.0 Hz, 1H), 7.26 (d, J=7.9 Hz, 1H), 6.33 (s, 1H), 4.62 (d, J=5.6 Hz, 2H), 4.11 (t, J=6.1 Hz, 2H), 3.09 (q, J=7.5 Hz, 2H), 2.80 (t, J=6.2 Hz, 2H), 2.66 (s, 3H), 2.34 (s, 3H), 1.99 (d, J=5.3 Hz, 2H), 1.85-1.76 (m, 2H), 1.33 (t, J=7.5 Hz, 3H).

### Synthesis of Compound 147

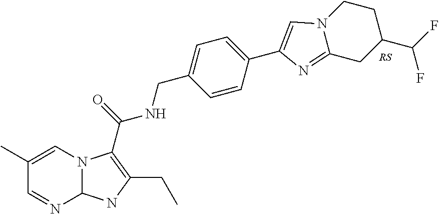 [SMILES: CCC1=C(C(=O)NCc2ccc(-c3cn4c(n3)CC(C(F)F)CC4)cc2)N2C=C(C)C=NC2N1]

Compound 147 was synthesized from intermediates I-137 and II-3a and obtained as a white solid (68 mg, 31%). ¹H NMR (400 MHz, DMSO) δ 9.15 (s, 1H), 8.51 (d, J=2.3 Hz, 1H), 8.47 (t, J=5.8 Hz, 1H), 7.69 (d, J=8.2 Hz, 2H), 7.49 (s, 1H), 7.33 (d, J=8.2 Hz, 2H), 6.15 (td, J=56.1, 4.1 Hz, 1H), 4.52 (d, J=5.8 Hz, 2H), 4.18-4.09 (m, 1H), 4.00-3.87 (m, 1H), 3.01 (q, J=7.5 Hz, 2H), 2.94 (dd, J=16.5, 4.5 Hz, 1H), 2.66 (dd, J=10.4, 5.7 Hz, 2H), 2.34 (s, 3H), 2.14-2.07 (m, 1H), 1.88-1.74 (m, 1H), 1.27 (t, J=7.5 Hz, 3H).

### Synthesis of Compound 148

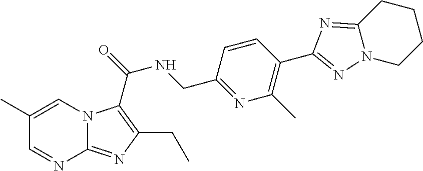 [SMILES: CCc1nc2ncc(C)cn2c1C(=O)NCc1ccc(-c2nc3n(n2)CCCC3)c(C)n1]

Compound 148 was synthesized from intermediates I-150 and II-3a and obtained as a white solid (86.6 mg, 46%). ¹H NMR (400 MHz, DMSO) δ 9.21 (s, 1H), 8.54-8.45 (m, 2H), 8.22 (d, J=8.0 Hz, 1H), 7.32 (d, J=8.1 Hz, 1H), 4.65 (d, J=5.6 Hz, 2H), 4.17 (t, J=5.9 Hz, 2H), 3.09 (q, J=7.4 Hz, 2H), 2.88 (t, J=6.2 Hz, 2H), 2.80 (s, 3H), 2.34 (s, 3H), 2.09-1.97 (m, 2H), 1.92 (dd, J=11.1, 5.4 Hz, 2H), 1.37-1.28 (m, 3H).

### Synthesis of Compound 149

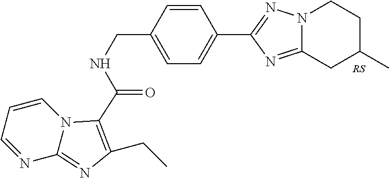 [SMILES: CCc1nc2ncccn2c1C(=O)NCc1ccc(-c2nc3n(n2)CCC(C)C3)cc1]

Compound 149 was synthesized from intermediates I-16 and 11-102 and obtained as a white solid (52 mg, 21%). ¹H NMR (400 MHz, DMSO) δ 9.33 (dd, J=6.9, 2.0 Hz, 1H), 8.62 (dd, J=4.2, 2.0 Hz, 1H), 8.54 (s, 1H), 7.94 (d, J=8.2 Hz, 2H), 7.44 (d, J=8.2 Hz, 2H), 7.17 (dd, J=6.9, 4.2 Hz, 1H), 4.57 (d, J=5.9 Hz, 2H), 4.22 (ddd, J=12.6, 5.6, 2.9 Hz, 1H), 4.16-4.00 (m, 1H), 3.04 (q, J=7.5 Hz, 2H), 2.98 (dd, J=17.2, 5.4 Hz, 1H), 2.49-2.40 (m, 1H), 2.18-2.00 (m, 2H), 1.82-1.67 (m, 1H), 1.29 (t, J=7.5 Hz, 3H), 1.10 (d, J=6.6 Hz, 3H).

### Synthesis of Compound 150

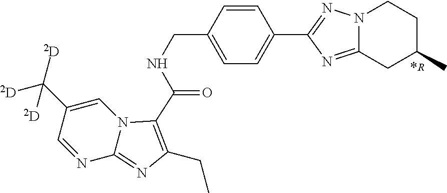

Compound 150 was synthesized from intermediates I-153 and 11-98 and obtained as an off-white solid (136 mg, 75%). ¹H NMR (400 MHz, DMSO) δ 9.16 (d, J=2.4 Hz, 1H), 8.50 (t, J=5.3 Hz, 2H), 7.94 (d, J=8.2 Hz, 2H), 7.43 (d, J=8.2 Hz, 2H), 4.57 (d, J=5.4 Hz, 2H), 4.22 (ddd, J=12.5, 5.5, 2.8 Hz, 1H), 4.13-4.04 (m, 1H), 3.06-2.94 (m, 3H), 2.47-2.39 (m, 1H), 2.13-2.00 (m, 2H), 1.80-1.68 (m, 1H), 1.28 (t, J=7.5 Hz, 3H), 1.10 (d, J=6.6 Hz, 3H).

### Synthesis of Compound 151

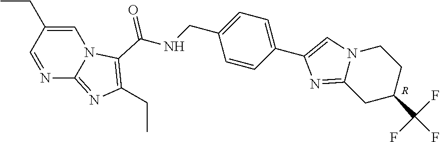

Compound 151 was synthesized from intermediates I-151a and II-3a and obtained as a beige solid (63.8 mg, 35%). ¹H NMR (400 MHz, DMSO) δ 9.15 (d, J=2.4 Hz, 1H), 8.58 (d, J=2.5 Hz, 1H), 8.49 (t, J=5.9 Hz, 1H), 7.70 (d, J=8.2 Hz, 2H), 7.53 (s, 1H), 7.35 (d, J=8.3 Hz, 2H), 4.53 (d, J=5.9 Hz, 2H), 4.21-4.13 (m, 1H), 3.99 (td, J=12.1, 4.5 Hz, 1H), 3.12-3.06 (m, 2H), 3.02 (q, J=7.5 Hz, 2H), 2.83-2.66 (m, 3H), 2.28-2.19 (m, 1H), 2.02-1.90 (m, 1H), 1.28 (t, J=7.5 Hz, 3H), 1.24 (t, J=7.5 Hz, 3H).

### Synthesis of Compound 152

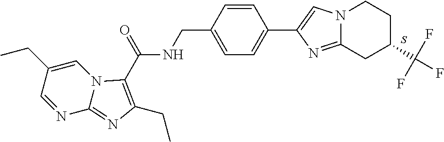

Compound 152 was synthesized from intermediates I-151b and 11-104 and obtained as a white solid (68.8 mg, 38%). ¹H NMR (400 MHz, DMSO) δ 9.14 (d, J=2.4 Hz, 1H), 8.57 (d, J=2.5 Hz, 1H), 8.48 (t, J=5.9 Hz, 1H), 7.69 (d, J=8.3 Hz, 2H), 7.53 (s, 1H), 7.34 (d, J=8.3 Hz, 2H), 4.52 (d, J=5.9 Hz, 2H), 4.16 (dd, J=12.8, 3.4 Hz, 1H), 3.98 (td, J=12.2, 4.7 Hz, 1H), 3.13-2.96 (m, 4H), 2.84-2.64 (m, 3H), 2.23 (d, J=11.1 Hz, 1H), 2.02-1.88 (m, 1H), 1.27 (t, J=7.5 Hz, 3H), 1.23 (t, J=7.5 Hz, 3H).

### Synthesis of Compound 153

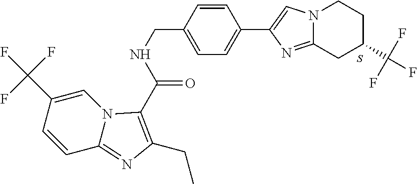

Compound 153 was synthesized from intermediates I-151b and 11-19 and obtained as an off white solid (110.9 mg, 50%). ¹H NMR (400 MHz, DMSO) δ 9.42 (s, 1H), 8.61 (s, 1H), 7.82 (d, J=9.4 Hz, 1H), 7.70 (d, J=8.1 Hz, 2H), 7.64 (d, J=8.1 Hz, 1H), 7.53 (s, 1H), 7.35 (d, J=8.1 Hz, 2H), 4.54 (s, 2H), 4.22-4.11 (m, 1H), 3.98 (td, J=12.1, 4.3 Hz, 1H), 3.15-2.99 (m, 4H), 2.78 (dd, J=17.3, 12.6 Hz, 1H), 2.23 (d, J=11.6 Hz, 1H), 2.03-1.88 (m, 1H), 1.28 (t, J=7.5 Hz, 3H).

### Synthesis of Compound 154

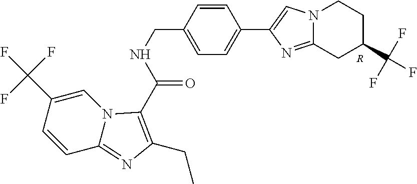

Compound 154 was synthesized from intermediates I-151a and II-19 and obtained as an off white solid (70.5 mg, 32%). ¹H NMR (400 MHz, DMSO) δ 9.43 (s, 1H), 8.62 (t, J=6.0 Hz, 1H), 7.83 (d, J=9.4 Hz, 1H), 7.71 (d, J=8.2 Hz, 2H), 7.65 (dd, J=9.4, 1.9 Hz, 1H), 7.61 (s, 1H), 7.38 (d, J=8.0 Hz, 2H), 4.55 (d, J=5.8 Hz, 2H), 4.19 (d, J=13.4 Hz, 1H), 4.01 (td, J=12.5, 4.8 Hz, 1H), 3.15-3.09 (m, 2H), 3.05 (q, J=7.5 Hz, 2H), 2.89-2.78 (m, 1H), 2.30-2.19 (m, 1H), 1.99 (dt, J=18.6, 6.4 Hz, 1H), 1.29 (t, J=7.5 Hz, 3H).

### Synthesis of Compound 155

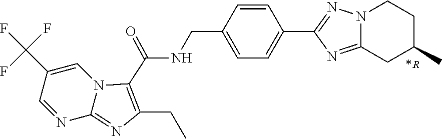

Compound 155 was synthesized from intermediates 1-153 and 11-19 and obtained as a white solid (15 mg, 10%). ¹H NMR (400 MHz, DMSO) δ 9.68 (s, 1H), 8.97 (d, J=2.4 Hz, 1H), 8.73 (t, J=5.7 Hz, 1H), 7.94 (d, J=8.1 Hz, 2H), 7.45 (d, J=8.2 Hz, 2H), 4.59 (d, J=5.8 Hz, 2H), 4.22 (ddd, J=12.7, 5.3, 2.6 Hz, 1H), 4.14-4.03 (m, 1H), 3.09 (q, J=7.5 Hz, 2H), 2.98 (dd, J=16.7, 4.6 Hz, 1H), 2.46-2.39 (m, 1H), 2.16-1.97 (m, 2H), 1.83-1.67 (m, 1H), 1.31 (t, J=7.5 Hz, 3H), 1.10 (d, J=6.6 Hz, 3H).

### Synthesis of Compound 156

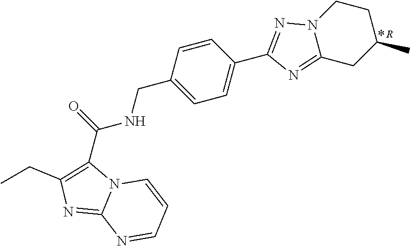

Compound 156 was synthesized from intermediates I-153 and 11-102 and obtained as a white solid (139.5 mg, 59%). ¹H NMR (400 MHz, DMSO) δ 9.34 (dd, J=6.9, 2.0 Hz, 1H), 8.63 (dd, J=4.2, 2.0 Hz, 1H), 8.56 (t, J=5.9 Hz, 1H), 7.94 (d, J=8.3 Hz, 2H), 7.44 (d, J=8.3 Hz, 2H), 7.18 (dd, J=6.9, 4.2 Hz, 1H), 4.57 (d, J=5.9 Hz, 2H), 4.24-4.17 (m, 1H), 4.13-4.03 (m, 1H), 3.04 (q, J=7.5 Hz, 2H), 3.01-2.94 (m, 1H), 2.46-2.40 (m, 1H), 2.15-2.00 (m, 2H), 1.81-1.68 (m, 1H), 1.29 (t, J=7.5 Hz, 3H), 1.10 (d, J=6.6 Hz, 3H).

### Synthesis of Compound 157

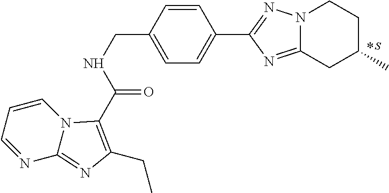

Compound 157 was synthesized from intermediates I-154 and 11-102 and obtained as a white solid (153.5 mg, 71%). ¹H NMR (400 MHz, DMSO) δ 9.33 (dd, J=6.9, 2.0 Hz, 1H), 8.62 (dd, J=4.2, 2.0 Hz, 1H), 8.55 (t, J=5.9 Hz, 1H), 7.94 (d, J=8.3 Hz, 2H), 7.44 (d, J=8.3 Hz, 2H), 7.17 (dd, J=6.9, 4.2 Hz, 1H), 4.57 (d, J=5.9 Hz, 2H), 4.22 (ddd, J=12.6, 5.5, 2.8 Hz, 1H), 4.13-4.03 (m, 1H), 3.04 (q, J=7.5 Hz, 2H), 3.01-2.94 (m, 1H), 2.48-2.39 (m, 1H), 2.09 (d, J=7.7 Hz, 1H), 2.05 (dd, J=14.3, 3.6 Hz, 1H), 1.74 (dtd, J=13.4, 10.8, 5.8 Hz, 1H), 1.29 (t, J=7.5 Hz, 3H), 1.10 (d, J=6.6 Hz, 3H).

### Synthesis of Compound 158

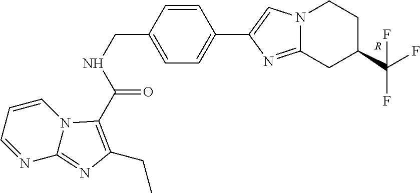

Compound 158 was synthesized from intermediates I-151a and 11-102 and obtained as a white solid (64 mg, 33%). ¹H NMR (400 MHz, DMSO) δ 9.32 (dd, J=6.9, 2.0 Hz, 1H), 8.62 (dd, J=4.2, 2.0 Hz, 1H), 8.51 (t, J=5.9 Hz, 1H), 7.69 (d, J=8.3 Hz, 2H), 7.52 (s, 1H), 7.34 (d, J=8.3 Hz, 2H), 7.16 (dd, J=6.9, 4.2 Hz, 1H), 4.52 (d, J=5.9 Hz, 2H), 4.16 (dd, J=12.5, 3.3 Hz, 1H), 3.98 (td, J=12.1, 4.6 Hz, 1H), 3.13-3.05 (m, 2H), 3.02 (t, J=7.5 Hz, 2H), 2.78 (dd, J=17.3, 12.6 Hz, 1H), 2.29-2.16 (m, 1H), 2.04-1.89 (m, 1H), 1.29 (t, J=7.5 Hz, 3H).

### Synthesis of Compound 159

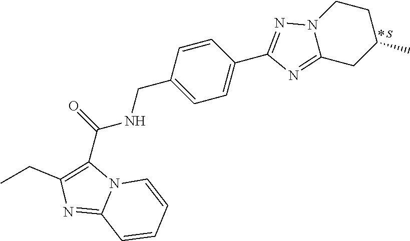

Compound 159 was synthesized from intermediates I-154 and II-102 and obtained as a white solid (41.5 mg, 21%). ¹H NMR (400 MHz, DMSO) δ 8.98 (d, J=6.9 Hz, 1H), 8.44 (t, J=6.0 Hz, 1H), 7.94 (d, J=8.3 Hz, 2H), 7.60 (dd, J=9.0, 1.0 Hz, 1H), 7.43 (d, J=8.3 Hz, 2H), 7.40-7.35 (m, 1H), 7.01 (td, J=6.9, 1.2 Hz, 1H), 4.57 (d, J=5.9 Hz, 2H), 4.26-4.18 (m, 1H), 4.12-4.04 (m, 1H), 3.00 (q, J=7.5 Hz, 2H), 2.97-2.94 (m, 1H), 2.46-2.40 (m, 1H), 2.15-2.00 (m. 2H). 1.81-1.68 (m. 1H). 1.27 (t. J=7.5 Hz. 3H). 1.10 (d. J=6.6 Hz. 3H).

### Synthesis of Compound 160

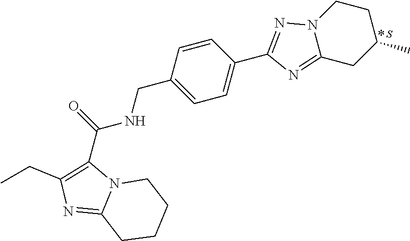

Compound 160 was synthesized from intermediates II-17a and I-154 and obtained as a white solid (126.8 mg, 53%). ¹H NMR (400 MHz, DMSO) δ 8.25 (t, J=6.0 Hz, 1H), 7.93 (d, J=8.3 Hz, 2H), 7.37 (d, J=8.3 Hz, 2H), 4.46 (d, J=6.0 Hz, 2H), 4.26-4.19 (m, 1H), 4.14-4.04 (m, 1H), 4.00 (t, J=5.8 Hz, 2H), 3.03-2.94 (m, 1H), 2.72 (t, J=6.3 Hz, 2H), 2.65 (q, J=7.5 Hz, 2H), 2.48-2.40 (m, 1H), 2.17-2.03 (m, 2H), 1.89-1.66 (m, 5H), 1.11 (t, J=7.5 Hz, 3H), 1.12-1.10 (m, 3H).

### Synthesis of Compound 161

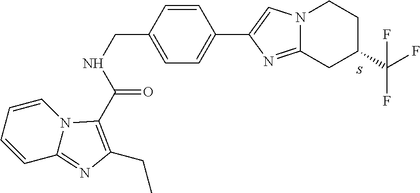

Compound 161 was synthesized from intermediates II-102 and I-151b and obtained as a white solid (112.1 mg, 58%). ¹H NMR (400 MHz, DMSO) δ 8.99-8.90 (m, 1H), 8.40 (t, J=5.9 Hz, 1H), 7.69 (d, J=8.3 Hz, 2H), 7.62-7.56 (m, 1H), 7.52 (s, 1H), 7.40-7.36 (m, 1H), 7.34 (dd, J=6.8, 4.8 Hz, 2H), 7.01 (td, J=6.9, 1.2 Hz, 1H), 4.52 (d, J=5.9 Hz, 2H), 4.20-4.12 (m, 1H), 3.98 (td, J=12.1, 4.6 Hz, 1H), 3.12-3.03 (m, 2H), 2.99 (q, J=7.5 Hz, 2H), 2.78 (dd, J=17.4, 12.7 Hz, 1H), 2.23 (dd, J=13.2, 2.2 Hz, 1H), 2.04-1.88 (m, 1H), 1.27 (t, J=7.5 Hz, 3H).

### Synthesis of Compound 162

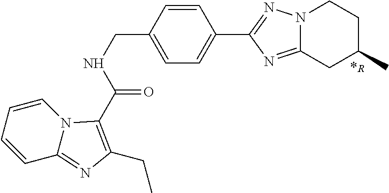

Compound 162 was synthesized from intermediates II-102 and I-153 and obtained as a white solid (78.9 mg, 46%). ¹H NMR (400 MHz, DMSO) δ 9.01-8.95 (m, 1H), 8.44 (t, J=6.0 Hz, 1H), 7.94 (d, J=8.3 Hz, 2H), 7.64-7.55 (m, 1H), 7.43 (d, J=8.3 Hz, 2H), 7.41-7.34 (m, 1H), 7.01 (td, J=6.9, 1.2 Hz, 1H), 4.57 (d, J=5.9 Hz, 2H), 4.27-4.17 (m, 1H), 4.13-4.01 (m, 1H), 3.04-2.94 (m, 3H), 2.46-2.40 (m, 1H), 2.15-2.00 (m, 2H), 1.82-1.68 (m, 1H), 1.27 (t, J=7.5 Hz, 3H), 1.10 (d, J=6.6 Hz, 3H).

### Synthesis of Compound 163

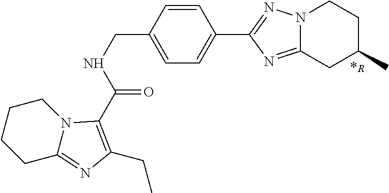

Compound 163 was synthesized from intermediates II-17a and I-153 and obtained as a white solid (50.6 mg, 29%). ¹H NMR (400 MHz, DMSO) δ 8.25 (t, J=6.0 Hz, 1H), 7.92 (d, J=8.3 Hz, 2H), 7.36 (d, J=8.3 Hz, 2H), 4.45 (d, J=6.0 Hz, 2H), 4.25-4.17 (m, 1H), 4.13-4.03 (m, 1H), 3.99 (t, J=5.8 Hz, 2H), 2.98 (dd, J=17.0, 5.2 Hz, 1H), 2.71 (t, J=6.3 Hz, 2H), 2.64 (q, J=7.5 Hz, 2H), 2.48-2.40 (m, 1H), 2.17-2.00 (m, 2H), 1.91-1.69 (m, 5H), 1.13-1.07 (m, 6H).

### Synthesis of Compound 164

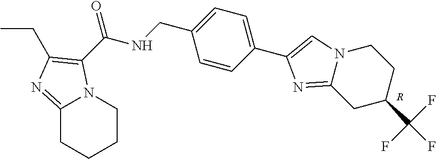

Compound 164 was synthesized from intermediates II-17a and I-151a and obtained as a white solid (114.8 mg, 61%). ¹H NMR (400 MHz, DMSO) δ 8.21 (t, J=6.0 Hz, 1H), 7.67 (d, J=8.2 Hz, 2H), 7.52 (s, 1H), 7.26 (d, J=8.2 Hz, 2H), 4.40 (d, J=6.0 Hz, 2H), 4.19-4.12 (m, 1H), 4.03-3.92 (m, 3H), 3.16-3.00 (m, 2H), 2.77 (dd, J=17.4, 12.6 Hz, 1H), 2.70 (t, J=6.3 Hz, 2H), 2.63 (q, J=7.5 Hz, 2H), 2.28-2.18 (m, 1H), 2.02-1.89 (m, 1H), 1.88-1.73 (m, 4H), 1.10 (t, J=7.5 Hz, 3H).

### Synthesis of Compound 165

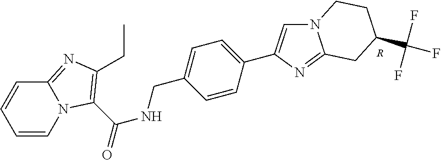

Compound 165 was synthesized from intermediates II-102 and I-151a and obtained as a white solid (91.6 mg, 38%).

¹H NMR (400 MHz, DMSO) δ 8.97 (d, J=6.9 Hz, 1H), 8.40 (t, J=6.0 Hz, 1H), 7.69 (d, J=8.2 Hz, 2H), 7.60 (d, J=9.0 Hz, 1H), 7.52 (s, 1H), 7.39-7.35 (m, 1H), 7.34 (d, J=8.3 Hz, 2H), 7.01 (td, J=6.9, 1.2 Hz, 1H), 4.52 (d, J=5.9 Hz, 2H), 4.21-4.12 (m, 1H), 3.98 (td, J=12.1, 4.7 Hz, 1H), 3.13-3.03 (m, 2H), 2.99 (q, J=7.5 Hz, 2H), 2.78 (dd, J=17.3, 12.5 Hz, 1H), 2.28-2.19 (m, 1H), 2.01-1.89 (m, 1H), 1.27 (t, J=7.5 Hz, 3H).

### Synthesis of Compound 166

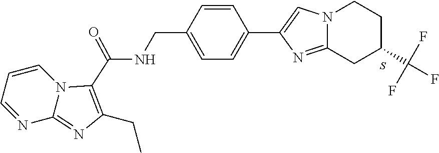

Compound 166 was synthesized from intermediates II-83 and I-151b and obtained as a white solid (155.9 mg, 61%).

¹H NMR (400 MHz, DMSO) δ 9.32 (dd, J=6.9, 2.0 Hz, 1H), 8.61 (dd, J=4.2, 2.0 Hz, 1H), 8.51 (t, J=5.8 Hz, 1H), 7.69 (d, J=8.2 Hz, 2H), 7.52 (s, 1H), 7.34 (d, J=8.3 Hz, 2H), 7.16 (dd, J=6.9, 4.2 Hz, 1H), 4.52 (d, J=5.9 Hz, 2H), 4.20-4.11 (m, 1H), 3.98 (td, J=12.1, 4.6 Hz, 1H), 3.14-2.98 (m, 4H), 2.77 (dd, J=17.4, 12.7 Hz, 1H), 2.23 (dd, J=13.2, 2.1 Hz, 1H), 2.03-1.86 (m, 1H), 1.29 (t, J=7.5 Hz, 3H).

### Synthesis of Compound 167

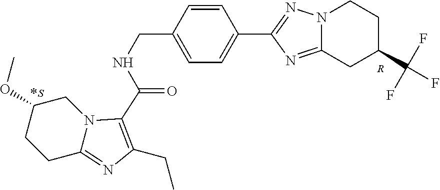

Compound 167 was synthesized from intermediates II-74 and I-88 and obtained as a white solid (78.4 mg, 47%). ¹H NMR (400 MHz, DMSO) δ 8.26 (t, J=5.9 Hz, 1H), 7.94 (d, J=8.2 Hz, 2H), 7.38 (d, J=8.2 Hz, 2H), 4.46 (d, J=6.0 Hz, 2H), 4.36-4.28 (m, 1H), 4.22-4.09 (m, 2H), 4.04 (dd, J=13.8, 3.5 Hz, 1H), 3.87-3.79 (m, 1H), 3.29 (s, 3H), 3.23-3.13 (m, 2H), 2.92 (dd, J=17.8, 12.3 Hz, 1H), 2.71 (dd, J=7.9, 4.9 Hz, 2H), 2.65 (q, J=7.5 Hz, 2H), 2.37-2.27 (m, 1H), 2.18-2.02 (m, 2H), 1.95-1.85 (m, 1H), 1.11 (t, J=7.5 Hz, 3H).

### Synthesis of Compound 168

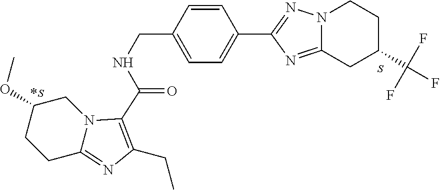

Compound 168 was synthesized from intermediates II-74 and I-87 obtained as a white solid (132.3 mg, 83%). ¹H NMR (400 MHz, DMSO) δ 8.33 (t, J=5.6 Hz, 1H), 7.94 (d, J=8.2 Hz, 2H), 7.38 (d, J=8.2 Hz, 2H), 4.46 (d, J=6.0 Hz, 2H), 4.36-4.27 (m, 1H), 4.22-4.10 (m, 2H), 4.05 (dd, J=13.8, 3.5 Hz, 1H), 3.84 (dd, J=5.5, 2.0 Hz, 1H), 3.29 (s, 3H), 3.25-3.14 (m, 2H), 2.92 (dd, J=17.6, 12.1 Hz, 1H), 2.74 (dd, J=8.2, 5.1 Hz, 2H), 2.66 (q, J=7.5 Hz, 2H), 2.37-2.27 (m, 1H), 2.18-2.02 (m, 2H), 1.96-1.85 (m, 1H), 1.12 (t, J=7.5 Hz, 3H).

### Method B: Coupling Reaction Between Acyl Chloride Derivatives and Amine Derivatives

### General Procedure

Method A: In a round bottom flask, amine derivative (1 eq.) was added to a solution of acyl chloride derivative (1.6 eq.) in anhydrous dioxane at room temperature under nitrogen atmosphere. The mixture was stirred for 10 min and DIPEA (1.6 eq.) was added. The reaction mixture was stirred at room temperature for 16 hours. The mixture was diluted with a solution of NaHCO₃ (sat., aq.) and water.

Method B: In a round bottom flask, DIPEA (3 to 6 eq.) was added to a solution of amine derivative (1 eq.) in anhydrous dioxane. The reaction mixture was stirred for 10 min and the mixture was added to a solution of acyl chloride derivative (1.7 to 3 eq.) in anhydrous dioxane. The reaction mixture was stirred at room temperature for 16 hours.

The mixture was worked up by dilution of the reaction mixture with a solution of NaHCO₃ (sat., aq.) and water, and extracted with DCM. The combined organic extracts were dried (anhydrous MgSO₄), filtered and concentrated in vacuo.

In an alternative, after dilution, the mixture was filtered and the solid was washed before being dried under vacuum.

The crude product may be purified by flash column chromatography (silica) and/or triturated with DIPE, Et₂O, DCM/MeOH (9:1).

The Following Compounds were Obtained Through Method B:

### Synthesis of Compound 169

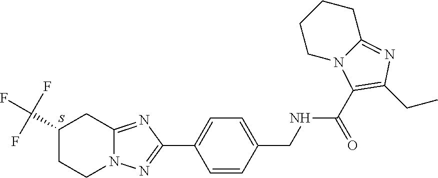

Compound 169 was synthesized from intermediates II-52 and I-88 and obtained as a white solid (117.7 mg, 72%). ¹H NMR (400 MHz, DMSO) δ 8.24 (t, J=5.9 Hz, 1H), 7.93 (d, J=8.2 Hz, 2H), 7.38 (d, J=8.2 Hz, 2H), 4.45 (d, J=5.9 Hz, 2H), 4.32 (dd, J=12.8, 3.6 Hz, 1H), 4.17 (td, J=12.0, 4.8 Hz, 1H), 3.99 (t, J=5.7 Hz, 2H), 3.25-3.12 (m, 2H), 2.92 (dd, J=17.7, 12.2 Hz, 1H), 2.71 (t, J=6.2 Hz, 2H), 2.64 (dd, J=15.0, 7.5 Hz, 2H), 2.36-2.28 (m, 1H), 2.19-2.04 (m, 1H), 1.90-1.72 (m, 4H), 1.11 (t, J=7.5 Hz, 3H).

### Synthesis of Compound 170

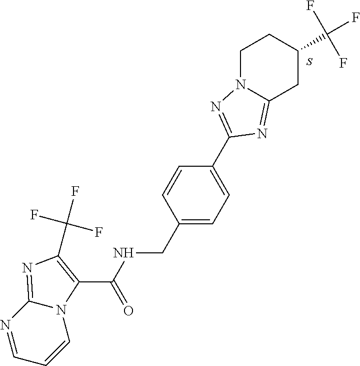

Compound 170 was synthesized from intermediates II-108 and I-87 and obtained as a pale brown solid (146.7 mg, 70%). ¹H NMR (400 MHz, DMSO) δ 9.00 (dd, J=6.9, 1.8 Hz, 1H), 8.90 (s, 1H), 8.81 (dd, J=3.9, 1.9 Hz, 1H), 7.98 (d, J=8.1 Hz, 2H), 7.46 (d, J=8.0 Hz, 2H), 7.31 (dd, J=6.9, 4.1 Hz, 1H), 4.62 (d, J=5.7 Hz, 2H), 4.40-4.12 (m, 2H), 3.31-3.10 (m, 2H), 2.91 (s, 1H), 2.42-2.29 (m, 1H), 2.25-2.05 (m, 1H).

### Synthesis of Compound 171

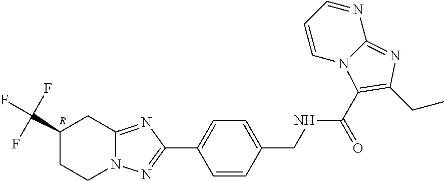

Compound 171 was synthesized from intermediates II-55 and I-88 and obtained as a beige solid (12.1 g, 94%). ¹H NMR (400 MHz, DMSO) δ 9.34 (dd, J=6.9, 1.9 Hz, 1H), 8.62 (dd, J=4.1, 2.0 Hz, 1H), 8.55 (t, J=5.9 Hz, 1H), 7.95 (d, J=8.2 Hz, 2H), 7.45 (d, J=8.2 Hz, 2H), 7.17 (dd, J=6.9, 4.2 Hz, 1H), 4.58 (d, J=5.9 Hz, 2H), 4.33 (dd, J=12.9, 3.6 Hz, 1H), 4.17 (td, J=12.1, 4.9 Hz, 1H), 3.25-3.14 (m, 2H), 3.04 (q, J=7.5 Hz, 2H), 2.92 (dd, J=17.7, 12.2 Hz, 1H), 2.38-2.26 (m, 1H), 2.11 (qd, J=11.6, 5.8 Hz, 1H), 1.29 (t, J=7.5 Hz, 3H).

### Synthesis of Compound 172

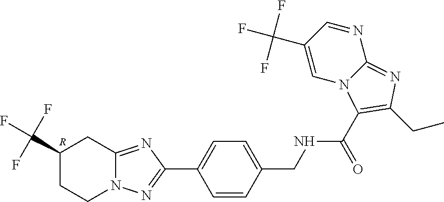

Compound 172 was synthesized from intermediates II-54 and I-88 obtained as a white solid (121.4 mg, 63%). ¹H NMR (400 MHz, DMSO) δ 9.69 (dd, J=2.4, 1.1 Hz, 1H), 8.97 (d, J=2.6 Hz, 1H), 8.73 (t, J=6.0 Hz, 1H), 7.96 (d, J=8.3 Hz, 2H), 7.47 (d, J=8.3 Hz, 2H), 4.60 (d, J=5.9 Hz, 2H), 4.33 (dd, J=12.8, 3.7 Hz, 1H), 4.17 (td, J=12.2, 4.9 Hz, 1H), 3.25-3.15 (m, 2H), 3.09 (q, J=7.5 Hz, 2H), 2.93 (dd, J=17.7, 12.2 Hz, 1H), 2.32 (dd, J=10.8, 2.2 Hz, 1H), 2.11 (qd, J=11.4, 5.9 Hz, 1H), 1.31 (t, J=7.5 Hz, 3H).

### Synthesis of Compound 173

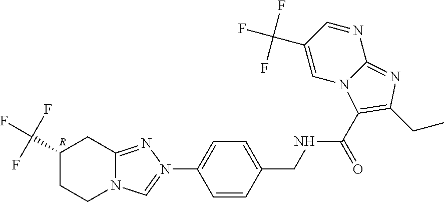

Compound 173 was synthesized from intermediates II-54 and I-87 obtained as a light beige solid (66 mg, 33%). ¹H NMR (400 MHz, DMSO) δ 9.70-9.68 (m, 1H), 8.97 (d, J=2.5 Hz, 1H), 8.73 (t, J=5.9 Hz, 1H), 7.96 (d, J=8.3 Hz, 2H), 7.47 (d, J=8.3 Hz, 2H), 4.60 (d, J=5.8 Hz, 2H), 4.33 (dd, J=12.9, 3.7 Hz, 1H), 4.17 (td, J=12.0, 4.7 Hz, 1H), 3.23-3.15 (m, 2H), 3.09 (q, J=7.5 Hz, 2H), 2.93 (dd, J=17.6, 12.2 Hz, 1H), 2.35-2.29 (m, 1H), 2.17-2.05 (m, 1H), 1.31 (t, J=7.5 Hz, 3H).

### Synthesis of Compound 174

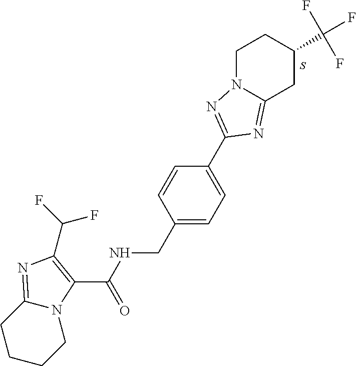

Compound 174 was synthesized from intermediates II-107 and I-87 obtained as a beige solid (66.8 mg, 34.2%). ¹H NMR (400 MHz, DMSO) δ 8.83 (t, J=5.9 Hz, 1H), 7.94 (d, J=8.2 Hz, 2H), 7.39 (d, J=8.2 Hz, 2H), 6.95 (t, J=54.3 Hz, 1H), 4.48 (d, J=5.8 Hz, 2H), 4.37-4.29 (m, 1H), 4.17 (td, J=12.0, 4.9 Hz, 1H), 4.04 (t, J=5.7 Hz, 2H), 3.24-3.13 (m, 2H), 2.93 (dd, J=17.6, 12.1 Hz, 1H), 2.79 (t, J=6.3 Hz, 2H), 2.37-2.27 (m, 1H), 2.21-2.05 (m, 1H), 1.96-1.78 (m, 4H).

### Synthesis of Compound 175

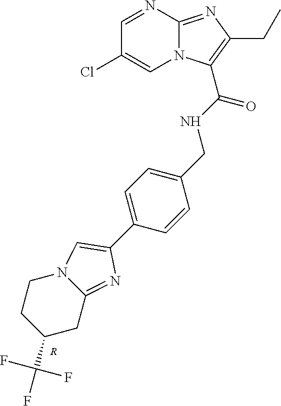

Compound 175 was synthesized from intermediates II-106 and I-151a obtained as a pale orange solid (45.4 mg, 29%). ¹H NMR (400 MHz, DMSO) δ 9.41 (d, J=2.6 Hz, 1H), 8.68 (d, J=2.6 Hz, 1H), 8.59 (t, J=5.9 Hz, 1H), 7.69 (d, J=8.2 Hz, 2H), 7.53 (s, 1H), 7.34 (d, J=8.3 Hz, 2H), 4.53 (d, J=5.9 Hz, 2H), 4.16 (dd, J=12.7, 3.5 Hz, 1H), 3.98 (td, J=12.1, 4.5 Hz, 1H), 3.12-3.08 (m, 2H), 3.07-3.00 (m, 2H), 2.78 (dd, J=17.4, 12.7 Hz, 1H), 2.28-2.17 (m, 1H), 2.03-1.87 (m, 1H), 1.28 (t, J=7.5 Hz, 3H).

### Synthesis of Compound 176

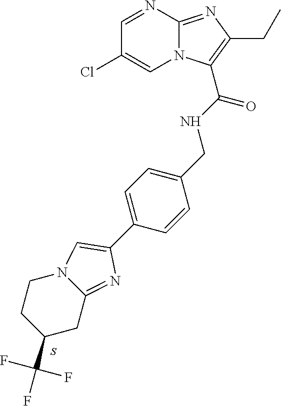

Compound 176 was synthesized from intermediates II-106 and I-151b obtained as a yellow solid (85.3 mg, 30%). ¹H NMR (400 MHz, DMSO) δ 9.41 (d, J=2.6 Hz, 1H), 8.68 (d, J=2.6 Hz, 1H), 8.59 (t, J=5.9 Hz, 1H), 7.69 (d, J=8.3 Hz, 2H), 7.53 (s, 1H), 7.34 (d, J=8.3 Hz, 2H), 4.53 (d, J=5.8 Hz, 2H), 4.16 (dd, J=12.5, 3.4 Hz, 1H), 4.01-3.93 (m, 1H), 3.04 (dd, J=15.0, 7.5 Hz, 4H), 2.78 (dd, J=17.5, 12.7 Hz, 1H), 2.23 (dd, J=13.2, 2.1 Hz, 1H), 1.95 (dq, J=11.6, 5.3 Hz, 1H), 1.28 (t, J=7.5 Hz, 3H).

### Synthesis of Compounds 177 and 178

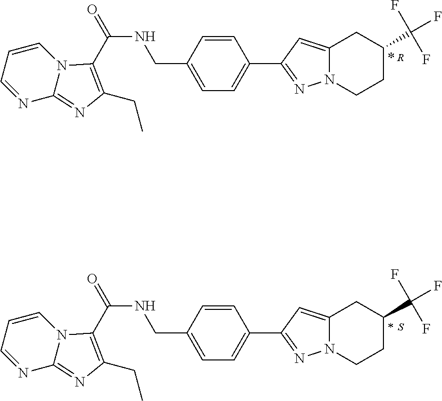

Isomers 177 and 178 were synthesized from intermediates II-55 and I-47, after SFC separation (Jasco SFC prep system, i-cellulose column (Phenomenex) 250*30 mm, 5 mm particle size, isocratic mode at 100 ml/min of CO₂ (40%)/MeOH (60%)/diethylamine (0.1%) at 30° C., 120 bars), to yield 177 (386.1 mg, 48% yield) and 178 (335.8 mg, 42% yield) as white solids.

177: ¹H NMR (400 MHz, DMSO) δ 9.33 (dd, J=6.9, 2.0 Hz, 1H), 8.63 (dd, J=4.2, 2.0 Hz, 1H), 8.54 (t, J=5.9 Hz, 1H), 7.74 (d, J=8.3 Hz, 2H), 7.39 (d, J=8.3 Hz, 2H), 7.17 (dt, J=12.4, 6.2 Hz, 1H), 6.53 (s, 1H), 4.55 (d, J=5.9 Hz, 2H), 4.31 (dd, J=12.9, 3.5 Hz, 1H), 4.12 (td, J=12.2, 4.8 Hz, 1H), 3.14 (dd, J=15.8, 3.8 Hz, 1H), 3.04 (q, J=7.5 Hz, 2H), 2.80 (dd, J=15.8, 11.f1 Hz, 1H), 2.28 (dd, J=13.3, 2.3 Hz, 1H), 2.04 (ddd, J=25.0, 11.9, 5.8 Hz, 1H), 1.30 (t, J=7.5 Hz, 3H).

178: ¹H NMR (400 MHz, DMSO) δ 9.33 (dd, J=6.9, 2.0 Hz, 1H), 8.63 (dd, J=4.2, 2.0 Hz, 1H), 8.54 (t, J=5.9 Hz, 1H), 7.74 (d, J=8.3 Hz, 2H), 7.39 (d, J=8.3 Hz, 2H), 7.17 (dd, J=6.9, 4.2 Hz, 1H), 6.53 (s, 1H), 4.55 (d, J=5.9 Hz, 2H), 4.31 (dd, J=12.7, 3.4 Hz, 1H), 4.12 (td, J=12.3, 4.8 Hz, 1H), 3.13 (dt, J=16.0, 7.9 Hz, 1H), 3.04 (q, J=7.5 Hz, 2H), 2.80 (dd, J=15.8, 11.2 Hz, 1H), 2.28 (dd, J=13.4, 2.3 Hz, 1H), 2.04 (ddd, J=24.9, 11.8, 5.7 Hz, 1H), 1.30 (t, J=7.5 Hz, 3H).

### Synthesis of Compound 179

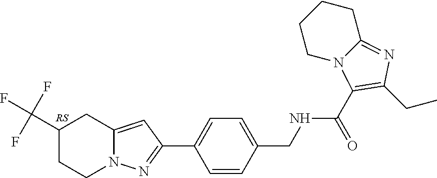 [SMILES: CCc1nc2n(c1C(=O)NCc1ccc(-c3cc4n(n3)CCC(C(F)(F)F)C4)cc1)CCCC2]

Compound 179 was synthesized from intermediates II-52 and I-27 and obtained as a white solid (49.4 mg, yield: 51%). ¹H NMR (400 MHz, DMSO) δ 8.23 (t, J=6.0 Hz, 1H), 7.71 (d, J=8.3 Hz, 2H), 7.31 (d, J=8.3 Hz, 2H), 6.52 (s, 1H), 4.42 (d, J=5.9 Hz, 2H), 4.30 (dd, J=12.9, 3.5 Hz, 1H), 4.11 (td, J=12.3, 4.8 Hz, 1H), 3.99 (t, J=5.8 Hz, 2H), 3.13 (dd, J=15.9, 3.6 Hz, 1H), 3.07-2.93 (m, 1H), 2.78 (dd, 1H), 2.70 (t, J=6.3 Hz, 2H), 2.64 (q, 2H), 2.28 (dd, J=13.4, 2.3 Hz, 1H), 2.04 (qd, J=11.8, 5.7 Hz, 1H), 1.92-1.81 (m, J=7.8 Hz, 2H), 1.81-1.71 (m, 2H), 1.10 (t, J=7.5 Hz, 3H).

### Synthesis of Compound 180

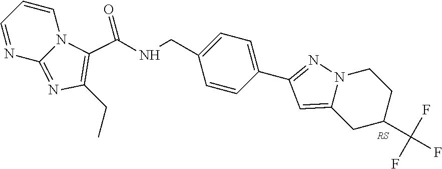 [SMILES: CCc1nc2ncccn2c1C(=O)NCc1ccc(-c2cc3n(n2)CCC(C(F)(F)F)C3)cc1]

Compound 180 was synthesized from intermediates II-55 and I-10 and obtained as a pale orange solid (54.5 mg, yield: 49%). ¹H NMR (400 MHz, DMSO) δ 9.33 (dd, J=6.9, 2.0 Hz, 1H), 8.63 (dd, J=4.2, 2.0 Hz, 1H), 8.54 (t, J=5.9 Hz, 1H), 7.74 (d, J=8.3 Hz, 2H), 7.39 (d, J=8.3 Hz, 2H), 7.17 (dd, J=6.9, 4.2 Hz, 1H), 6.53 (s, 1H), 4.55 (d, J=5.9 Hz, 2H), 4.31 (dd, J=12.6, 3.6 Hz, 1H), 4.12 (td, J=12.4, 4.8 Hz, 1H), 3.24-3.08 (m, 1H), 3.04 (q, J=7.5 Hz, 3H), 2.95-2.68 (m, 1H), 2.42-2.20 (m, 1H), 2.04 (qd, J=11.8, 5.7 Hz, 1H), 1.30 (t, J=7.5 Hz, 3H).

### Synthesis of Compound 181

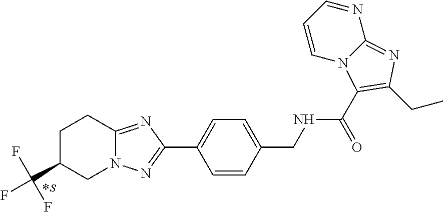

Compound 181 was synthesized from intermediates II-55 and I-47. Isomers were separated by SFC (Jasco SFC prep system, i-cellulose column (Phenomenex) 250*30 mm, 5 mm particle size, isocratic mode at 100 ml/min of CO₂ (40%)/MeOH (60%)/diethylamine (0.1%) at 30° C., 120 bars) to yield a pale orange solid (35 mg, yield: 23%). ¹H NMR (400 MHz, DMSO) δ 9.34 (dd, J=6.9, 2.0 Hz, 1H), 8.63 (dd, J=4.2, 2.0 Hz, 1H), 8.56 (t, J=6.0 Hz, 1H), 7.96 (d, J=8.3 Hz, 2H), 7.46 (d, J=8.3 Hz, 2H), 7.18 (dd, J=6.9, 4.2 Hz, 1H), 4.59 (d, J=5.9 Hz, 2H), 4.47 (dd, J=12.4, 5.6 Hz, 1H), 4.24-4.16 (m, 1H), 3.30 (s, 1H), 3.05 (q, J=7.5 Hz, 2H), 2.99-2.88 (m, 1H), 2.29-2.19 (m, 1H), 2.09-1.92 (m, 1H), 1.30 (t, J=7.5 Hz, 3H).

### Synthesis of Compound 182

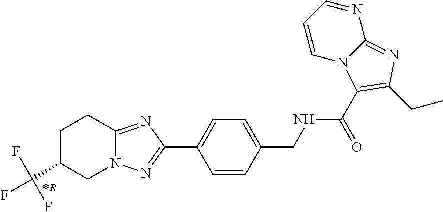

Compound 182 was synthesized from intermediates II-55 and I-47. Isomers were separated by SFC (Jasco SFC prep system, i-cellulose column (Phenomenex) 250*30 mm, 5 mm particle size, isocratic mode at 100 ml/min of CO₂ (40%)/MeOH (60%)/diethylamine (0.1%) at 30° C., 120 bars) to yield a pale orange solid (30.5 mg, yield: 20%). ¹H NMR (400 MHz, DMSO) δ 9.34 (dd, J=6.9, 2.0 Hz, 1H), 8.63 (dd, J=4.2, 2.0 Hz, 1H), 8.56 (t, J=5.9 Hz, 1H), 7.96 (d, J=8.3 Hz, 2H), 7.46 (d, J=8.3 Hz, 2H), 7.18 (dd, J=6.9, 4.2 Hz, 1H), 4.59 (d, J=5.9 Hz, 2H), 4.47 (dd, J=12.4, 5.6 Hz, 1H), 4.25-4.15 (m, 1H), 3.30 (s, 1H), 3.05 (q, J=7.5 Hz, 2H), 2.94 (ddd, J=17.1, 10.5, 6.2 Hz, 1H), 2.25 (dd, J=8.6, 5.0 Hz, 1H), 2.10-1.95 (m, 1H), 1.30 (t, J=7.5 Hz, 3H).

### Synthesis of Compound 183

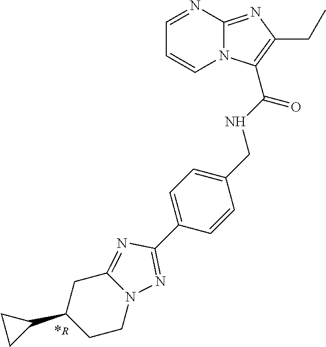

Compound 183 was synthesized from intermediates II-55 and I-60. Isomers were separated by SFC (Jasco SFC prep system, i-cellulose column (Phenomenex) 250*30 mm, 5 mm particle size, isocratic mode at 100 ml/min of CO₂ (40%)/MeOH (60%)/diethylamine (0.1%) at 30° C., 120 bars) to yield as a pale white solid (45 mg, yield: 14%). ¹H NMR (400 MHz, DMSO) δ 9.33 (dd, J=6.9, 2.0 Hz, 1H), 8.62 (dd, J=4.2, 2.0 Hz, 1H), 8.55 (t, J=5.9 Hz, 1H), 7.96-7.91 (m, 2H), 7.44 (d, J=8.4 Hz, 2H), 7.17 (dd, J=6.9, 4.2 Hz, 1H), 4.57 (d, J=5.9 Hz, 2H), 4.25 (ddd, J=12.7, 5.7, 3.3 Hz, 1H), 4.09-4.01 (m, 1H), 3.07-2.97 (m, 3H), 2.68-2.60 (m, 1H), 2.17-2.10 (m, 1H), 1.96-1.85 (m, 1H), 1.29 (t, J=7.5 Hz, 4H), 0.81-0.71 (m, 1H), 0.50-0.43 (m, 2H), 0.27-0.18 (m, 2H).

### Synthesis of Compound 184

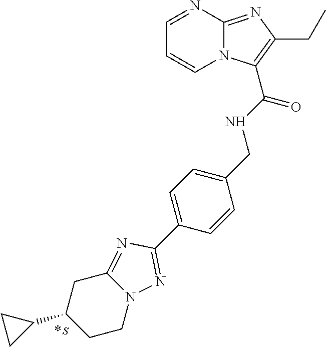

Compound 184 was synthesized from intermediates II-55 and I-60. Isomers were separated by SFC (Jasco SFC prep system, i-cellulose column (Phenomenex) 250*30 mm, 5 mm particle size, isocratic mode at 100 ml/min of CO₂ (40%)/MeOH (60%)/diethylamine (0.1%) at 30° C., 120 bars) to yield a pale white solid (61 mg, yield: 19%). ¹H NMR (400 MHz, DMSO) δ 9.33 (dd, J=6.9, 2.0 Hz, 1H), 8.62 (dd, J=4.2, 2.0 Hz, 1H), 8.55 (t, J=6.0 Hz, 1H), 7.94 (d, J=8.2 Hz, 2H), 7.44 (d, J=8.2 Hz, 2H), 7.17 (dd, J=6.9, 4.2 Hz, 1H), 4.57 (d, J=5.9 Hz, 2H), 4.25 (ddd, J=12.6, 5.6, 3.4 Hz, 1H), 4.10-4.00 (m, 1H), 3.08-2.97 (m, 3H), 2.64 (dd, J=16.9, 10.0 Hz, 1H), 2.19-2.09 (m, 1H), 1.96-1.85 (m, 1H), 1.29 (t, J=7.5 Hz, 4H), 0.81-0.71 (m, 1H), 0.49-0.44 (m, 2H), 0.26-0.18 (m, 2H).

### Synthesis of Compound 185

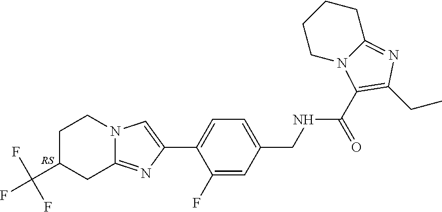 [SMILES: CCc1nc2n(c1C(=O)NCc1ccc(-c3cn4c(n3)CC(C(F)(F)F)CC4)c(F)c1)CCCC2]

Compound 185 was synthesized from intermediates II-52 and 1-130 and obtained as a white solid (87 mg, yield: 68%). ¹H NMR (400 MHz, DMSO) δ 8.24 (t, J=5.9 Hz, 1H), 7.96 (t, J=8.1 Hz, 1H), 7.43 (d, J=4.2 Hz, 1H), 7.14 (t, J=9.9 Hz, 2H), 4.42 (d, J=5.9 Hz, 2H), 4.21 (dd, J=12.7, 3.3 Hz, 1H), 4.03-3.93 (m, 3H), 3.14-3.05 (m, 2H), 2.80 (dd, J=17.3, 12.6 Hz, 1H), 2.71 (t, J=6.1 Hz, 2H), 2.64 (q, J=7.5 Hz, 2H), 2.24 (d, J=13.2 Hz, 1H), 1.95 (qd, J=11.9, 5.6 Hz, 1H), 1.85 (d, J=4.2 Hz, 2H), 1.78 (d, J=5.0 Hz, 2H), 1.11 (t, J=7.5 Hz, 3H).

### Synthesis of Compound 186

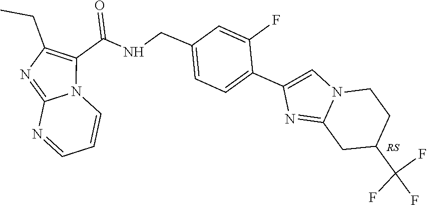 [SMILES: CCc1nc2ncccn2c1C(=O)NCc1ccc(-c2cn3c(n2)CC(C(F)(F)F)CC3)c(F)c1]

Compound 186 was synthesized from intermediates II-55 and I-130 and obtained as a white solid (43 mg, yield: 34%). ¹H NMR (400 MHz, DMSO) δ 9.34 (dd, J=6.9, 2.0 Hz, 1H), 8.63 (dd, J=4.2, 2.0 Hz, 1H), 8.54 (t, J=6.0 Hz, 1H), 7.98 (t, J=8.1 Hz, 1H), 7.44 (d, J=4.3 Hz, 1H), 7.24 (s, 1H), 7.21 (d, J=4.7 Hz, 1H), 7.17 (dd, J=6.9, 4.2 Hz, 1H), 4.55 (d, J=5.8 Hz, 2H), 4.29-4.13 (m, 1H), 3.99 (td, J=12.3, 4.7 Hz, 1H), 3.14-3.09 (m, 1H), 3.05 (q, J=7.5 Hz, 2H), 2.80 (dd, J=17.4, 12.5 Hz, 1H), 2.25 (d, J=11.1 Hz, 1H), 1.96 (qd, J=12.0, 5.4 Hz, 1H), 1.30 (t, J=7.5 Hz, 3H).

### Synthesis of Compound 187

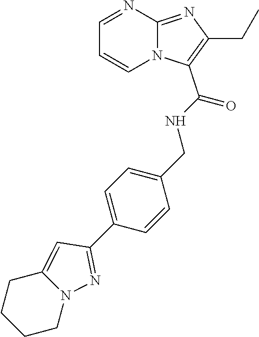 [SMILES: CCc1nc2ncccn2c1C(=O)NCc1ccc(-c2cc3n(n2)CCCC3)cc1]

Compound 187 was synthesized from intermediates II-55 and I-86 and obtained as a yellowish solid (62 mg, yield: 71%). ¹H NMR (400 MHz, DMSO) δ 9.32 (dd, J=6.9, 2.0 Hz, 1H), 8.62 (dd, J=4.2, 2.0 Hz, 1H), 8.51 (t, J=5.8 Hz, 1H), 7.72 (d, J=8.2 Hz, 2H), 7.37 (d, J=8.2 Hz, 2H), 7.16 (dd, J=6.9, 4.2 Hz, 1H), 6.40 (s, 1H), 4.54 (d, J=5.9 Hz, 2H), 4.08 (t, J=6.1 Hz, 2H), 3.03 (q, J=7.5 Hz, 2H), 2.77 (t, J=6.3 Hz, 2H), 2.02-1.94 (m, 2H), 1.83-1.75 (m, 2H), 1.29 (t, J=7.5 Hz, 3H).

### Synthesis of Compound 188

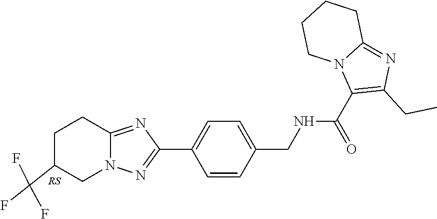 [SMILES: CCc1nc2n(c1C(=O)NCc1ccc(-c3nc4n(n3)CC(C(F)(F)F)CC4)cc1)CCCC2]

Compound 188 was synthesized from intermediates II-52 and I-47 and obtained as a yellowish solid (51 mg, yield: 53%). ¹H NMR (400 MHz, DMSO) δ 8.25 (t, J=6.0 Hz, 1H), 7.93 (d, J=8.3 Hz, 2H), 7.38 (d, J=8.3 Hz, 2H), 4.46 (t, J=7.2 Hz, 3H), 4.23-4.16 (m, 1H), 3.99 (t, J=5.8 Hz, 2H), 3.29 (s, 1H), 3.06-2.98 (m, 1H), 2.95 (dt, J=12.8, 6.3 Hz, 1H), 2.71 (t, J=6.3 Hz, 2H), 2.64 (q, J=7.5 Hz, 2H), 2.28-2.20 (m, 1H), 2.02 (dtd, J=13.2, 10.8, 6.0 Hz, 1H), 1.87-1.76 (m, 4H), 1.10 (t, J=7.5 Hz, 3H).

### Synthesis of Compound 189

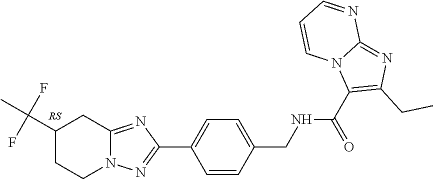 [SMILES: CCc1nc2ncccn2c1C(=O)NCc1ccc(-c2nc3n(n2)CCC(C(C)(F)F)C3)cc1]

Compound 189 was synthesized from intermediates II-55 and I-160 and obtained as a palid beige solid (66.9 mg, yield: 74%). ¹H NMR (400 MHz, DMSO) δ 8.55 (dd, J=6.9, 1.9 Hz, 1H), 7.83 (dd, J=4.2, 1.9 Hz, 1H), 7.18 (d, J=8.2 Hz, 2H), 6.69 (d, J=8.1 Hz, 2H), 6.35 (dd, J=6.9, 4.2 Hz, 1H), 3.87 (s, 2H), 3.77 (s, 1H), 3.58 (dd, J=12.7, 3.4 Hz, 1H), 3.36 (td, J=12.3, 5.1 Hz, 1H), 2.39 (dd, J=16.5, 4.8 Hz, 1H), 2.26 (q, J=7.6 Hz, 2H), 2.08 (dd, J=16.9, 11.3 Hz, 1H), 1.82 (s, 1H), 1.59 (d, J=13.8 Hz, 1H), 1.34-1.22 (m, 1H), 0.92 (t, J=19.1 Hz, 3H), 0.54 (t, J=7.6 Hz, 3H).

### Synthesis of Compound 190

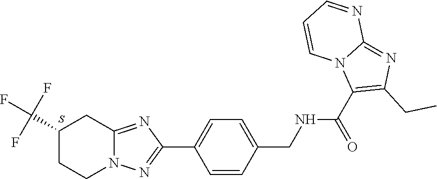

Compound 190 was synthesized from intermediates II-55 and I-87 and obtained as a beige solid (13.2 g, 98%). ¹H NMR (400 MHz, DMSO) δ 9.34 (dd, J=6.9, 2.0 Hz, 1H), 8.62 (dd, J=4.2, 2.0 Hz, 1H), 8.54 (t, J=5.9 Hz, 1H), 7.95 (d, J=8.3 Hz, 2H), 7.45 (d, J=8.3 Hz, 2H), 7.17 (dd, J=6.9, 4.2 Hz, 1H), 4.58 (d, J=5.9 Hz, 2H), 4.33 (dd, J=12.9, 3.4 Hz, 1H), 4.17 (td, J=12.2, 4.9 Hz, 1H), 3.23-3.14 (m, 2H), 3.04 (q, J=7.5 Hz, 2H), 2.92 (dd, J=17.7, 12.2 Hz, 1H), 2.36-2.27 (m, 1H), 2.11 (qd, J=11.4, 5.8 Hz, 1H), 1.29 (t, J=7.5 Hz, 3H).

### Synthesis of Compound 191

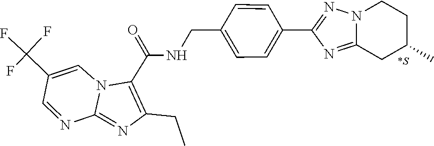 [SMILES: CCc1nc2ncc(C(F)(F)F)cn2c1C(=O)NCc1ccc(-c2nc3n(n2)CC[C@H](C)C3)cc1]

Compound 191 was synthesized from intermediates II-54 and I-154 and obtained as a beige solid (45.8 mg, 23%). ¹H NMR (400 MHz, DMSO) δ 9.68 (d, J=1.3 Hz, 1H), 8.97 (d, J=2.5 Hz, 1H), 8.73 (t, J=5.8 Hz, 1H), 7.94 (d, J=8.2 Hz, 2H), 7.45 (d, J=8.3 Hz, 2H), 4.59 (d, J=5.8 Hz, 2H), 4.22 (ddd, J=12.6, 5.5, 2.8 Hz, 1H), 4.12-4.03 (m, 1H), 3.09 (q, J=7.5 Hz, 2H), 2.98 (dd, J=17.1, 5.3 Hz, 1H), 2.46-2.39 (m, 1H), 2.14-2.01 (m, 2H), 1.74 (dtd, J=13.3, 10.8, 5.7 Hz, 1H), 1.31 (t, J=7.5 Hz, 3H), 1.10 (d, J=6.6 Hz, 3H).

### Synthesis of Compound 192

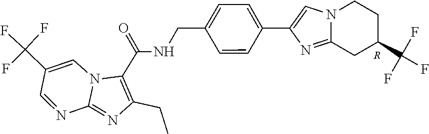

Compound 192 was synthesized from intermediates II-54 and I-151a and obtained as a light beige solid (41.1 mg, 25%). ¹H NMR (400 MHz, DMSO) 9.67 (d, J=1.3 Hz, 1H), 8.96 (d, J=2.5 Hz, 1H), 8.69 (t, J=5.8 Hz, 1H), 7.70 (d, J=8.2 Hz, 2H), 7.53 (s, 1H), 7.35 (d, J=8.3 Hz, 2H), 4.54 (d, J=5.8 Hz, 2H), 4.16 (dd, J=12.5, 3.4 Hz, 1H), 3.98 (td, J=12.1, 4.5 Hz, 1H), 3.08 (dd, J=15.0, 7.4 Hz, 4H), 2.77 (dd, J=17.4, 12.6 Hz, 1H), 2.23 (d, J=11.3 Hz, 1H), 1.95, (qd, J=11.8, 5.4 Hz, 1H), 1.30 (t, J=7.5 Hz, 3H).

### Synthesis of Compound 193

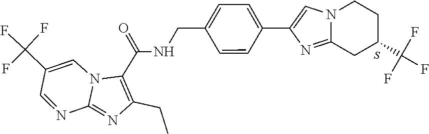

Compound 193 was synthesized from intermediates II-54 and I-151b and obtained as a pale orange solid (17.8 mg, 11%). ¹H NMR (400 MHz, DMSO) δ 9.67 (d, J=1.4 Hz, 1H), 8.96 (d, J=2.6 Hz, 1H), 8.69 (t, J=5.8 Hz, 1H), 7.70 (d, J=8.2 Hz, 2H), 7.53 (s, 1H), 7.35 (d, J=8.3 Hz, 2H), 4.54 (d, J=5.8 Hz, 2H), 4.16 (dd, J=12.6, 3.4 Hz, 1H), 3.98 (td, J=12.1, 4.6 Hz, 1H), 3.12-3.01 (m, 4H), 2.77 (dd, J=17.4, 12.7 Hz, 1H), 2.23 (d, J=11.0 Hz, 1H), 2.02-1.87 (m, 1H), 1.30 (t, J=7.5 Hz, 3H).

### Method C: Coupling Reaction Followed by Protecting Group Cleavage

### General Procedure

### a) Coupling Reaction

In a round bottom flask, carboxylic acid derivative (1.1 to 1.3 eq.) was added to a solution of amine derivative (1 eq.) in DMF. DIPEA (7 eq.) and HATU [CAS 148893-10-1] (1.5 eq.) were added and the reaction mixture was stirred at room temperature for 16 hours. The mixture was diluted with a solution of NaHCO₃ (sat., aq.) and extracted with DCM. The combined organic extracts were dried (anhydrous MgSO₄), filtered and concentrated in vacuo. The crude product was purified by flash column chromatography (silica, gradient: DCM/MeOH (9:1) in DCM). The desired fractions were collected and concentrated in vacuo to afford the desired intermediate XX.

### b) Protecting Group Cleavage

Method CA: HCl in dioxane (4M, 6 eq.) was added to a solution of the obtained product from step a) (1 eq.) in DCM in a round bottom flask at room temperature. The reaction mixture was stirred at room temperature for 16 hours. The mixture was concentrated in vacuo. An extraction step prior to the concentration may be used. The crude product was then purified by flash column chromatography (silica) and/or trituration (with DIPE, DCM) to afford the desired compound.

Method CB: TFA was added to a solution of the obtained product from step a) (1 eq.) in a round bottom flask at 0° C. The reaction mixture was stirred at room temperature for 1 hours. The mixture was neutralised with a solution of NaHCO₃ (sat., aq.) and extracted with an appropriate solvent. The combined organic extracts were dried (anhydrous MgSO₄), filtered and concentrated in vacuo. The crude product was purified by flash column chromatography, reverse phase or triturated (DCM) to afford the desired compound.

The Following Compounds were Obtained Through Method C:

### Synthesis of Compound 138

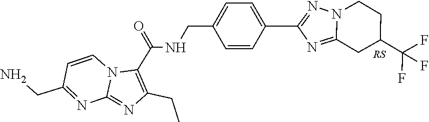

The coupling reaction (step a) between intermediates II-22 and I-6 delivered a yellow solid (200 mg, 80%, 85% purity).

Treatment with HCl in an analogous procedure to the one described for method A afforded compound 138 as a yellow solid (33 mg, 16%). ¹H NMR (400 MHz, DMSO) δ 9.26 (d, J=7.1 Hz, 1H), 8.46 (t, J=5.9 Hz, 1H), 7.95 (d, J=8.2 Hz, 2H), 7.45 (d, J=8.2 Hz, 2H), 7.25 (d, J=7.1 Hz, 1H), 4.57 (d, J=5.8 Hz, 2H), 4.32 (dd, J=12.5, 3.7 Hz, 1H), 4.22-4.12 (m, 1H), 3.86 (s, 1H), 3.19 (d, J=13.1 Hz, 2H), 3.01 (q, J=7.5 Hz, 2H), 2.92 (dd, J=17.7, 12.2 Hz, 1H), 2.69 (d, J=13.7 Hz, 1H), 2.32 (d, J=8.4 Hz, 2H), 2.17-2.03 (m, 2H), 1.27 (dd, J=15.9, 8.4 Hz, 3H).

### Synthesis of Compound 194

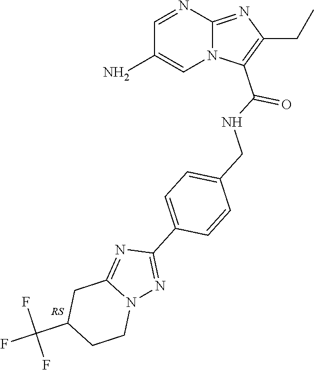 [SMILES: CCc1nc2ncc(N)cn2c1C(=O)NCc1ccc(-c2nc3n(n2)CCC(C(F)(F)F)C3)cc1]

The coupling reaction (step a) was performed between intermediates II-91 and I-6. Treatment with HCl in an analogous procedure to the one described for method A afforded compound 194 as a beige solid (158 mg, 72%). ¹H NMR (400 MHz, DMSO) δ 9.78 (d, J=10.7 Hz, 2H), 8.53 (d, J=2.7 Hz, 1H), 8.48 (t, J=5.9 Hz, 1H), 7.96 (d, J=8.2 Hz, 2H), 7.45 (d, J=8.2 Hz, 2H), 4.57 (d, J=5.8 Hz, 2H), 4.33 (dd, J=12.9, 3.9 Hz, 1H), 4.17 (td, J=12.1, 4.7 Hz, 1H), 3.19 (d, J=12.6 Hz, 1H), 3.02 (q, J=7.5 Hz, 2H), 2.96-2.86 (m, 2H), 2.36-2.27 (m, 1H), 2.11 (qd, J=11.5, 5.8 Hz, 1H), 1.49 (s, 9H), 1.27 (t, J=7.5 Hz, 3H).

### Synthesis of Compound 195

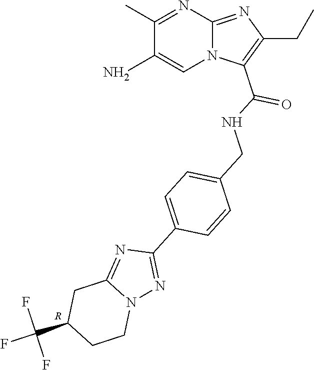

The coupling reaction (step a) between intermediates II-15 and I-88 delivered a beige solid (191 mg, 68%).

Treatment with TFA in an analogous procedure to the one described for method B afforded compound 195 (45.8 mg, 37%). ¹H NMR (400 MHz, DMSO) δ 8.97 (d, J=27.8 Hz, 1H), 8.45 (s, 1H), 7.96 (d, J=8.3 Hz, 2H), 7.44 (d, J=8.3 Hz, 2H), 7.11 (s, 1H), 6.42 (d, J=8.2 Hz, 2H), 4.69-4.58 (m, 2H), 4.55 (d, J=5.8 Hz, 2H), 4.37-4.27 (m, 1H), 4.17 (td, J=12.2, 4.9 Hz, 1H), 3.69 (s, 3H), 3.47 (d, J=21.5 Hz, 3H), 3.26-3.13 (m, 2H), 3.05-2.97 (m, 2H), 2.93 (dd, J=17.9, 12.5 Hz, 1H), 2.39-2.27 (m, 1H), 2.23 (s, 3H), 2.17-2.05 (m, 1H), 1.56-1.21 (m, 12H).

### Synthesis of Compound 196

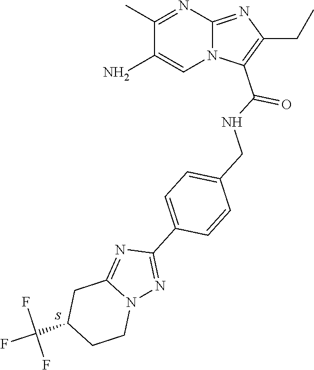

The coupling reaction (step a) between intermediates II-15 and I-87 delivered a beige solid (203 mg, 73%).

Treatment with TFA in an analogous procedure to the one described for method B afforded compound 196 (19.1 mg, 14%). ¹H NMR (300 MHz, DMSO) δ 8.94 (s, 1H), 8.46 (s, 1H), 7.96 (d, J=8.3 Hz, 2H), 7.44 (d, J=8.3 Hz, 2H), 7.11 (s, 1H), 6.42 (d, J=8.2 Hz, 2H), 4.64 (s, 2H), 4.55 (d, J=5.7 Hz, 2H), 4.31 (dd, J=12.5, 8.1 Hz, 1H), 4.17 (td, J=11.8, 4.7 Hz, 1H), 3.69 (s, 3H), 3.44 (s, 3H), 3.25-3.12 (m, 2H), 3.07-2.98 (m, 2H), 2.98-2.85 (m, 1H), 2.37-2.26 (m, 1H), 2.22 (s, 3H), 2.18-2.02 (m, 1H), 1.56-1.19 (m, 12H).

### Synthesis of Compound 197

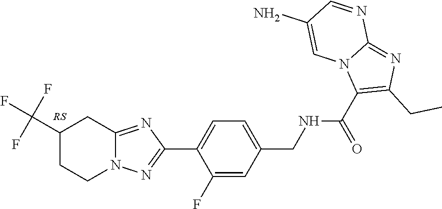 [SMILES: CCc1nc2ncc(N)cn2c1C(=O)NCc1ccc(-c2nc3n(n2)CCC(C(F)(F)F)C3)c(F)c1]

The coupling reaction (step a) between intermediates II-91 and I-150b delivered a brown solid (186 mg, 73%).

Treatment with HCl in an analogous procedure to the one described for method A afforded compound 197 (126 mg, 80%). ¹H NMR (400 MHz, DMSO) δ 8.74 (d, J=2.8 Hz, 1H), 8.31 (t, J=6.0 Hz, 1H), 8.24 (d, J=2.9 Hz, 1H), 7.94 (t, J=7.9 Hz, 1H), 7.29 (s, 1H), 7.27 (d, J=1.9 Hz, 1H), 5.28 (d, J=8.2 Hz, 2H), 4.55 (d, J=5.5 Hz, 2H), 4.41-4.29 (m, 1H), 4.18 (td, J=12.1, 4.8 Hz, 1H), 3.26-3.14 (m, 2H), 2.98 (q, J=7.5 Hz, 2H), 2.95-2.89 (m, 1H), 2.37-2.28 (m, 1H), 2.21-2.05 (m, 1H), 1.26 (t, J=7.5 Hz, 3H).

### Synthesis of Compound 198

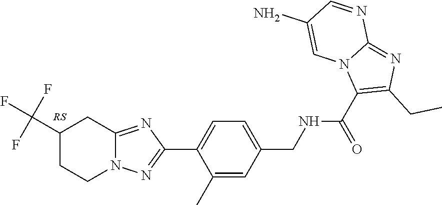 [SMILES: CCc1nc2ncc(N)cn2c1C(=O)NCc1ccc(-c2nc3n(n2)CCC(C(F)(F)F)C3)c(C)c1]

The coupling reaction (step a) between intermediates II-91 and I-125 delivered a brown solid (193 mg, 74%).

Treatment with HCl in an analogous procedure to the one described for method A afforded compound 198 as a white solid (79.4 mg, 49%). ¹H NMR (400 MHz, DMSO) δ 8.73 (d, J=2.9 Hz, 1H), 8.30 (t, J=6.0 Hz, 1H), 8.24 (d, J=2.9 Hz, 1H), 7.86 (d, J=7.8 Hz, 1H), 7.25 (d, J=8.8 Hz, 2H), 5.28 (s, 2H), 4.52 (d, J=5.9 Hz, 2H), 4.38-4.29 (m, 1H), 4.18 (td, J=12.0, 4.8 Hz, 1H), 3.26-3.14 (m, 2H), 3.02-2.88 (m, 3H), 2.57 (s, 3H), 2.36-2.28 (m, 1H), 2.20-2.06 (m, 1H), 1.26 (t, J=7.5 Hz, 3H).

### Synthesis of Compound 199

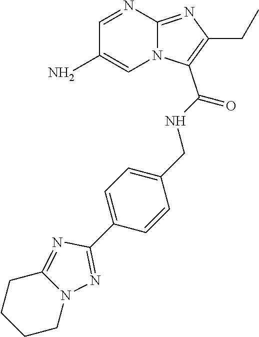 [SMILES: CCc1nc2ncc(N)cn2c1C(=O)NCc1ccc(-c2nc3n(n2)CCCC3)cc1]

The coupling reaction (step a) between intermediates II-91 and I-72a delivered a brown solid (193 mg, 74%).

Treatment with HCl in an analogous procedure to the one described for method A afforded compound 199 as a white solid (187 mg, 77%). ¹H NMR (400 MHz, DMSO) δ 8.74 (d, J=2.9 Hz, 1H), 8.27-8.33 (m, 1H), 8.22-8.25 (m, 1H), 7.90-7.96 (m, 2H), 7.40-7.44 (m, 2H), 5.23-5.31 (m, 2H), 4.51-4.57 (m, 2H), 4.10-4.16 (m, 2H), 2.93-3.00 (m, 2H), 2.85 (t, J=6.3 Hz, 2H), 1.98-2.05 (m, 2H), 1.86-1.94 (m, 2H), 1.25 ppm (t, J=7.5 Hz, 3H).

### Synthesis of Compound 200

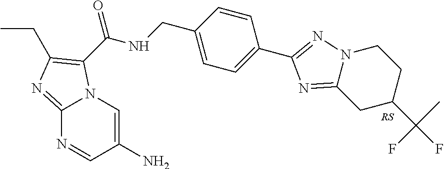 [SMILES: CCc1nc2ncc(N)cn2c1C(=O)NCc1ccc(-c2nc3n(n2)CCC(C(C)(F)F)C3)cc1]

The coupling reaction (step a) between intermediates II-91 and I-160 delivered a yellow solid (200 mg, 81%).

Treatment with HCl in an analogous procedure to the one described for method A afforded compound 200 as a beige solid (121.4 mg, 72%).

¹H NMR (400 MHz, DMSO) δ 8.74 (d, J=2.8 Hz, 1H), 8.30 (t, J=6.0 Hz, 1H), 8.24 (d, J=2.9 Hz, 1H), 7.94 (d, J=8.2 Hz, 2H), 7.43 (d, J=8.3 Hz, 2H), 5.27 (d, J=8.2 Hz, 2H), 4.55 (d, J=5.8 Hz, 2H), 4.37-4.25 (m, 1H), 4.11 (td, J=12.2, 4.9 Hz, 1H), 3.09 (dd, J=16.5, 4.3 Hz, 1H), 2.96 (t, J=7.5 Hz, 2H), 2.79 (dd, J=16.5, 11.4 Hz, 1H), 2.71-2.60 (m, 1H), 2.29-2.20 (m, 1H), 2.04-1.88 (m, 1H), 1.71 (t, J=19.5 Hz, 3H), 1.25 (t, J=7.5 Hz, 3H).

The Following Compounds were Also Prepared in Accordance with the Procedures Disclosed Herein:

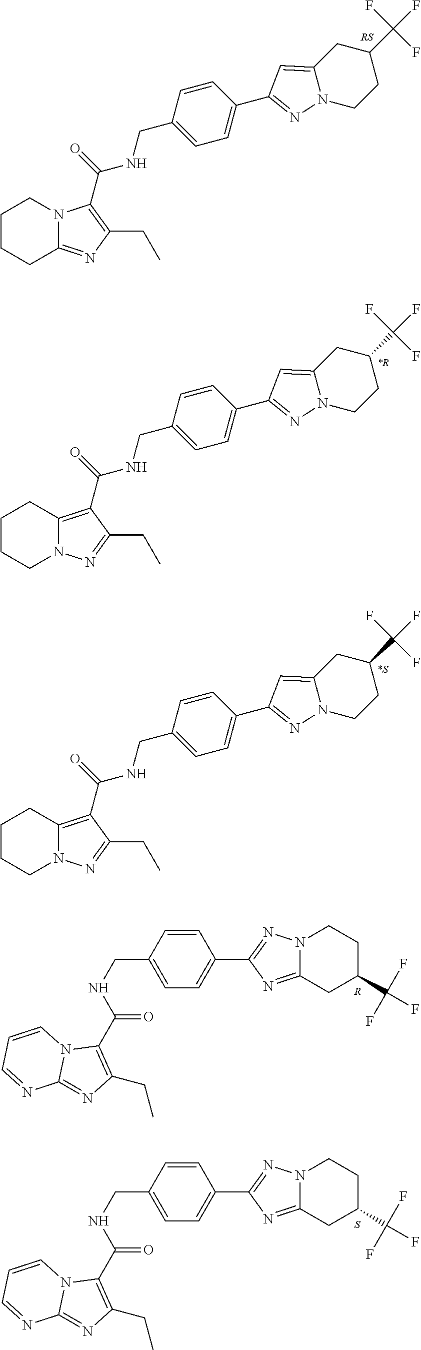

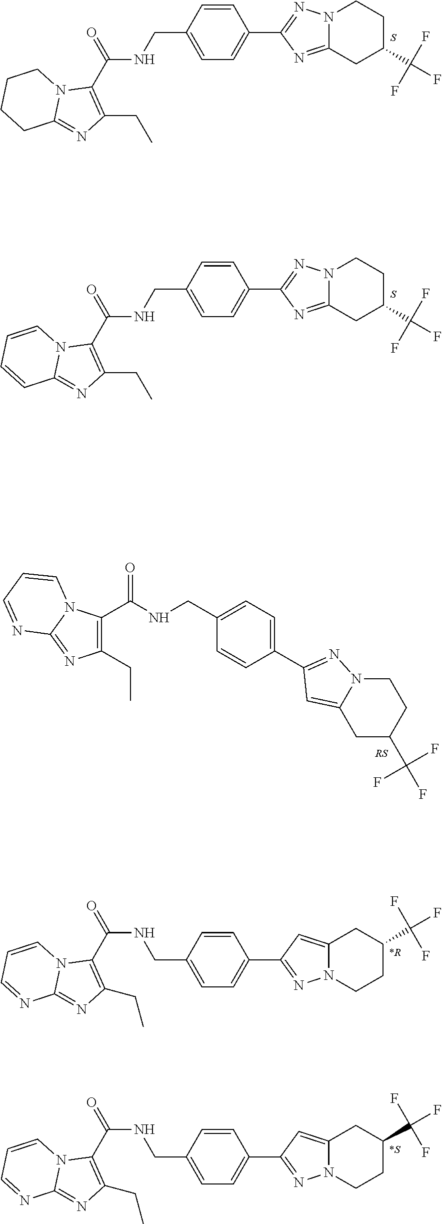

### 3—Analytical Data

### HPLC-LCMS

The mass of some compounds was recorded with LCMS (liquid chromatography mass spectrometry). The methods used are described below.

### General Procedure LCMS Methods

The High-Performance Liquid Chromatography (HPLC) measurement was performed using a LC pump, a diode-array (DAD) or a UV detector and a column as specified in the respective methods. If necessary, additional detectors were included (see table of methods below). Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rt) and ions. If not specified differently in the table of data, the reported molecular ion corresponds to the [M+H]+ (protonated molecule) and/or [M−H]− (deprotonated molecule). In case the compound was not directly ionizable the type of adduct is specified (i.e. [M+NH4]+, [M+HCOO]−, etc. . . . ). For molecules with multiple isotopic patterns (Br, Cl . . . ), the reported value is the one obtained for the lowest isotope mass. All results were obtained with experimental uncertainties that are commonly associated with the method used.

Hereinafter, “SQD” means Single Quadrupole Detector, “RT” room temperature, “BEH” bridged ethylsiloxane/silica hybrid, “HSS” High Strength Silica, “DAD” Diode Array Detector, “MSD” Mass Selective Detector.

TABLE 1
LCMS Method codes (Flow expressed in mL/min; column
temperature (T) in ° C.; Run time in minutes).

| Method | | | Mobile | | Flow | Run |
|---|---|---|---|---|---|---|
| code | Instrument | Column | phase | gradient | Column T | time |
| A | Agilent 1100 | YMC-pack | A: 0.1% | From 95% A to 5% | 2.6 | 6.2 |
| | HPLC DAD | ODS-AQ C18 | HCOOH | A in 4.8 min, held | 35 | |
| | LC/MS | (50 × 4.6 mm, | in H₂O | for 1.0 min, to 95% | | |
| | G1956A | 3 μm) | B: CH₃CN | A in 0.2 min. | | |
| B | Agilent 1290 | Phenomenex | A: 0.1% | From 90% A to 10% | 1.2 | 2.2 |
| | Infinity II | Kinetex C18 | HCOOH | A in 1.6 min, held | 60 | |
| | HPLC DAD | (50 × 2.1 mm, | in H₂O | for 0.4 min, to 90% | | |
| | LC/MSD iQ | 1.7 μm) | B: CH₃CN | A in 0.2 min. | | |
| | G6160A | | | | | |
| C | Agilent 1260 | YMC-pack | A: 0.1% | From 95% A to 5% | 2.6 | 6.8 |
| | Infinity DAD | ODS-AQ C18 | HCOOH | A in 4.8 min, held | 35 | |
| | TOF-LC/MS | (50 × 4.6 mm, | in H₂O | for 1.0 min, to 95% | | |
| | G6224A | 3 μm) | B: CH₃CN | A in 0.2 min. | | |
| D | Agilent 1290 | Thermo | A: 0.1% | From 95% A to 5% | 1.5 | 2.0 |
| | Infinity DAD | Scientific | HCOOH | A in 1.3 min, held | 35 | |
| | LC/MS 6120 | Accucore C18 | in H2O | for 0.5 min, to 95% | | |
| | (G1948B) | (50 × 2.1 mm, | B: CH3CN | A in 0.1 min. | | |
| | | 2.6 μm) | | | | |

TABLE 2
Characterizing data of the compounds. Rt means retention time
(in minutes), MW means the molecular weight of the compound,
[M + H]+ means the protonated mass of the compound,
method refers to the method used for LCMS analysis of compounds.

| | Melting point | | UV | | | |
|---|---|---|---|---|---|---|
| Compound | (° C.) (DSC | | Area | | | |
| number | or MT) | Rₜ | (%) | MW | [M + H]⁺ | Method |
| 1ab | N.D. | 2.57 | 99 | 483.5 | 484.2 | A |
| 1a | 255.1 (MT) | 2.57 | 99 | 483.5 | 484.2 | A |
| 1b | 263.1 (MT) | 2.57 | 99 | 483.5 | 484.2 | A |
| 2ab | 213.3 (MT) | 3.09 | 98 | 508.4 | 509.1. | A |
| 2a | 226.1 (DSC) | 3.08 | 99 | 508.4 | 509.1 | A |
| 2b | 226.5 (DSC) | 3.08 | 99 | 508.4 | 509.1 | A |
| 3ab | 220.0 (MT) | 1.86 | 99 | 482.5 | 483.2 | A |
| 3a | 223.3 (MT) | 1.82 | 99 | 482.5 | 483.2 | A |
| 3b | 224.7 (MT) | 1.83 | 98 | 482.5 | 483.2 | A |
| 4ab | 236.7 (MT) | 2.37 | 97 | 429.5 | 430.2 | A |
| 5ab | 255.0 (MT) | 2.43 | 99 | 483.5 | 484.2 | A |
| 5a | nd | 0.78 | 99 | 483.5 | 484.2 | B |
| 5b | nd | 0.78 | 99 | 483.5 | 484.2 | B |
| 6ab | 224.9 (MT) | 2.64 | 99 | 499.5 | 500.2 | A |
| 7ab | 228.3 (MT) | 2.44 | 99 | 486.5 | 487.1 | A |
| 8ab | 271.8 (MT) | 2.81 | 99 | 495.5 | 496.2 | A |
| 9ab | 256.8 (MT) | 2.50 | 99 | 497.5 | 498.2 | A |
| 10ab | 229.9 (MT) | 3.02 | 97 | 503.9 | 504.1 | A |
| 10a | nd | 2.86 | 99 | 503.9 | 504.1 | A |
| 10b | nd | 2.99 | 99 | 503.9 | 504.1 | A |
| 11ab | 271.1 (MT) | 2.53 | 98 | 513.5 | 514.2 | A |
| 12ab | 272.5 (MT) | 2.24 | 99 | 498.5 | 499.2 | A |
| 13ab | 181.9 (MT) | 2.17 | 99 | 472.5 | 473.2 | A |
| 14ab | 206.9 (MT) | 3.10 | 99 | 490.4 | 491.2 | A |
| 15ab | 226.8 (MT) | 2.29 | 99 | 482.5 | 483.2 | A |
| 16ab | 172.9 (MP) | 1.81 | 96 | 498.5 | 499.2 | A |
| 17ab | 251.1 (MT) | 2.34 | 99 | 469.5 | 470.1 | A |
| 18ab | 183.1 (MT) | 2.85 | 99 | 507.4 | 508.2 | C |
| 19ab | 252.4 (MT) | 1.92 | 99 | 496.5 | 497.2 | A |
| 20ab | 225 (MT) | 3.05 | 99 | 482.5 | 483.2 | A |
| 21ab | 236.7 (MT) | 3.18 | 99 | 496.5 | 497.2 | A |
| 22ab | 251.7 (MT) | 2.56 | 99 | 501.5 | 502.2 | A |
| 23ab | 211.6 (MT) | 2.73 | 99 | 497.5 | 498.2 | A |
| 24ab | 257.4 (MT) | 2.63 | 99 | 483.5 | 484.2 | A |
| 25ab | 246.7 (MT) | 2.35 | 99 | 465.5 | 466.2 | A |
| 26ab | 216.7 (MP) | 2.18 | 99 | 445.5 | 446.2 | A |
| 27ab | 221.7 (MP) | 2.70 | 99 | 455.6 | 456.2 | A |
| 28ab | 213.3 (MP) | 2.90 | 99 | 457.6 | 458.3 | A |
| 29ab | 274.2 (DSC) | 1.82 | 99 | 431.5 | 432.2 | A |
| 30ab | 137.9 (DSC) | 2.3 | 97 | 489.6 | 491.2 | A |
| 31ab | 252.1 (MT) | 2.06 | 99 | 415.5 | 416.2 | A |
| 32ab | 243.9 (DSC) | 1.99 | 99 | 433.5 | 434.2 | A |
| 33ab | 226.6 (MT) | 2.63 | 99 | 499.5 | 500.2 | A |
| 34ab | 265.6 (DSC) | 1.68 | 99 | 482.5 | 483.3 | A |
| 35ab | 235.2 (MT) | 2.16 | 98 | 432.5 | 433.2 | A |
| 36ab | 210.4 (MT) | 1.94 | 99 | 508.5 | 509.1 | A |
| 37 | 245.0 (MT) | 2.32 | 98 | 498.5 | 499.1 | A |
| 38 | 233.3 (MT) | 2.37 | 99 | 498.5 | 499.2 | A |
| 39 | 225.68 (DSC) | 2.17 | 99 | 484.5 | 485.1 | A |
| 138 | 193.44 (DSC) | 1.90 | 95 | 498.5 | 499.2 | A |
| 40 | 216.31 (DSC) | 2.31 | 99 | 501.5 | 502.2 | A |
| 41 | 226.6 (MT) | 1.97 | 97 | 485.5 | 486.2 | A |
| 42 | 265.23 (DSC) | 2.71 | 98 | 409.4 | 409.7 | A |
| 45 | 242.15 (DSC) | 2.23 | 99 | 502.5 | 503.1 | A |
| 43 | 247.11 (DSC) | 2.16 | 99 | 484.5 | 485.2 | A |
| 44 | 180.31 (DSC) | 2.67 | 99 | 515.5 | 516.2 | A |
| 194 | N.D. | 0.75 | 93 | 584.6 | 585.2 | D |
| 46 | 233.39 (DSC) | 2.17 | 99 | 498.5 | 499.2 | A |
| 47 | 223.3 (MT) | 2.77 | 99 | 527.4 | 528.0 | A |
| 195 | N.D. | 3.86 | 97 | 748.9 | 749.3 | A |
| 196 | N.D. | 0.919 | 96 | 748.8 | 749.4 | D |
| 197 | 263.38 (DSC) | 2.12 | 99 | 502.5 | 503.1 | A |
| 198 | 222.92 (DSC) | 2.252 | 99 | 498.5 | 499.1 | A |
| 48 | 223.58 (DSC) | 2.693 | 99 | 499.5 | 500.2 | A |
| 49 | 221.73 (DSC) | 2.686 | 99 | 499.5 | 500.2 | A |
| 50 | 220.16 (DSC) | 2.627 | 99 | 486.5 | 486.9 | A |
| 51 | 247.37 (DSC) | 2.621 | 99 | 486.5 | 487.2 | A |
| 52 | 223.56 (DSC) | 2.676 | 99 | 486.5 | 487.1 | A |
| 61 | 203.08 (DSC) | 3.063 | 99 | 490.4 | 491.1 | A |
| 53 | 237.89 (DSC) | 2.932 | 98 | 509.4 | 510.1 | A |
| 54 | 260.2 (DSC) | 3.039 | 97 | 512.5 | 513.2 | A |
| 55 | 219.18 (DSC) | 2.200 | 99 | 468.5 | 469.1 | A |
| 56 | 194.23 (DSC) | 2.121 | 98 | 472.5 | 473.2 | A |
| 57 | 217.2 (DSC) | 2.206 | 98 | 468.5 | 469.1 | A |
| 58 | 258.9 (DSC) | 3.00 | 99 | 512.5 | 513.1 | A |
| 169 | 195.66 (DSC) | 2.09 | 98 | 472.5 | 473.2 | A |
| 170 | 235.43 (DSC) | 2.93 | 96 | 509.4 | 510.1 | A |
| 59 | 227.07 (DSC) | 2.820 | 99 | 497.5 | 498.2 | A |
| 60 | 201.06 (DSC) | 3.063 | 99 | 490.4 | 491.1 | A |
| 62 | 224.33 (DSC) | 2.819 | 98 | 497.5 | 498.2 | A |
| 63 | 199.74 (DSC) | 3.472 | 98 | 536.5 | 537.0 | A |
| 64 | 202.03 (DSC) | 3.470 | 97 | 536.5 | 537.1 | A |
| 171 | 246.61 (DSC) | 2.553 | 99 | 469.5 | 470.1 | A |
| 190 | 244.92 (DSC) | 2.573 | 99 | 469.5 | 470.1 | A |
| 172 | 182.0 (DSC) | 3.338 | 99 | 537.5 | 538.1 | A |
| 173 | 180.1 (DSC) | 3.330 | 99 | 537.5 | 538.1 | A |
| 65 | 194.29 (DSC) | 2.257 | 98 | 486.5 | 487.2 | A |
| 66 | 228.63 (DSC) | 2.241 | 99 | 486.5 | 487.2 | A |
| 67 | 232.0 (DSC) | 2.251 | 99 | 486.5 | 487.0 | A |
| 68 | 229.4 (DSC) | 2.251 | 98 | 486.5 | 487.0 | A |
| 69 | 192.78 (DSC) | 2.269 | 99 | 486.5 | 487.2 | A |
| 70 | 226 (DSC) | 2.236 | 97 | 486.5 | 487.2 | A |
| 71 | 192.7 (DSC) | 2.259 | 99 | 486.5 | 487.2 | A |
| 72 | 180.1 (DSC) | 2.263 | 99 | 486.5 | 487.2 | A |
| 74 | 279.11 (DSC) | 2.545 | 98 | 487.5 | 488.1 | A |
| 73 | 267.75 (DSC) | 2.544 | 98 | 487.5 | 488.1 | A |
| 174 | 249.75 (DSC) | 2.793 | 99 | 494.5 | 495.1 | A |
| 75 | 196.7 (DSC) | 3.110 | 99 | 486.5 | 487.1 | A |
| 76 | 237.55 (DSC) | 2.124 | 99 | 490.5 | 491.0 | A |
| 77 | 238.17 (DSC) | 2.113 | 99 | 490.5 | 491.0 | A |
| 78 | 214.5 (DSC) | 2.203 | 99 | 486.5 | 487.0 | A |
| 79 | 214.9 (DSC) | 2.192 | 99 | 486.5 | 487.1 | A |
| 80 | 249.79 (DSC) | 2.793 | 99 | 494.5 | 495.0 | A |
| 81 | 172.36 (DSC) | 3.111 | 98 | 486.5 | 487.2 | A |
| 82 | 172.03 (DSC) | 3.113 | 99 | 486.5 | 487.2 | A |
| 83 | 189.73 (DSC) | 3.114 | 99 | 486.5 | 487.1 | A |
| 84 | 230.9 (DSC) | 3.373 | 98 | 553.5 | 554.1 | A |
| 85 | 224.8 (DSC) | 3.369 | 99 | 553.5 | 554.1 | A |
| 86 | 184.93 (DSC) | 2.708 | 99 | 483.5 | 484.1 | A |
| 87 | 202.06 (DSC) | 2.707 | 98 | 483.5 | 484.1 | A |
| 88 | N.D. | 1.630 | 95 | 414.5 | 415.1 | A |
| 103 | >300 (MT) | 2.533 | 99 | 479.5 | 480.1 | A |
| 89 | 231.05 (DSC) | 1.65 | 99 | 428.5 | 429.2 | A |
| 90 | N.D. | 2.073 | 99 | 516.9 | 517.1 | A |
| 91 | 232.55 (DSC) | 1.59 | 99 | 428.5 | 429.3 | A |
| 93 | 235.98 (DSC) | 2.18 | 99 | 429.5 | 430.2 | A |
| 94 | 235.34 (DSC) | 2.33 | 99 | 429.5 | 430.2 | A |
| 92 | 235.39 (DSC) | 2.11 | 99 | 429.5 | 430.2 | A |
| 95 | 228.81 (DSC) | 2.52 | 99 | 414.5 | 415.2 | A |
| 96 | 244.49 (DSC) | 2.02 | 99 | 433.5 | 434.2 | A |
| 97 | 229.91 (DSC) | 2.255 | 99 | 447.5 | 448.2 | A |
| 98 | 198.05 (DSC) | 2.14 | 99 | 459.5 | 460.2 | A |
| 99 | 236.12 (DSC) | 2.227 | 99 | 465.5 | 466.3 | A |
| 100 | 202.36 (DSC) | 1.849 | 98 | 496.5 | 497.2 | A |
| 101 | 232.4 (DSC) | 2.076 | 98 | 526.4 | 527.1 | A |
| 102 | 197.52 (DSC) | 2.224 | 96 | 459.5 | 460.2 | A |
| 104 | 223.22 (DSC) | 2.563 | 99 | 479.5 | 480.2 | A |
| 105 | 223.57 (DSC) | 2.557 | 99 | 479.5 | 480.2 | A |
| 106 | 217.83 (DSC) | 2.562 | 97 | 497.5 | 498.2 | A |
| 107 | 202.43 (DSC) | 2.107 | 99 | 463.5 | 464.2 | A |
| 108 | 202.18 (DSC) | 1.797 | 98 | 478.5 | 479.2 | A |
| 109 | 190.70 (DSC) | 1.839 | 96 | 442.6 | 443.2 | A |
| 110 | 227.98 (DSC) | 2.676 | 96 | 497.5 | 498.1 | A |
| 111 | 232.63 (DSC) | 1.816 | 98 | 498.5 | 499.2 | A |
| 112 | 220.51 (DSC) | 1.827 | 98 | 498.5 | 499.1 | A |
| 113 | 181.84 (DSC) | 1.682 | 97 | 485.5 | 486.1 | A |
| 114 | 183.44 (DSC) | 1.687 | 98 | 485.5 | 486.2 | A |
| 115 | 151.4 (MT) | 2.45 | 97 | 473.6 | 474.2 | A |
| 116 | 240.81 (DSC) | 1.803 | 98 | 482.5 | 483.2 | A |
| 176 | 252.23 (DSC) | 2.075 | 98 | 502.9 | 503.1 | A |
| 117 | 260.7 (DSC) | 1.919 | 99 | 500.5 | 501.1 | A |
| 175 | 258.12 (DSC) | 2.076 | 99 | 502.9 | 503.1 | A |
| 143 | 230.41 (DSC) | 2.229 | 99 | 477.5 | 478.2 | A |
| 118 | 184.47 (DSC) | 1.698 | 99 | 458.6 | 459.2 | A |
| 119 | 238.18 (DSC) | 2.209 | 99 | 431.5 | 432.2 | A |
| 120 | 2.055 (DSC) | 2.055 | 99 | 418.5 | 419.2 | A |
| 121 | 200.95 (DSC) | 1.851 | 98 | 496.5 | 497.2 | A |
| 122 | N.D. | 1.681 | 99 | 497.5 | 498.2 | A |
| 123 | 205.16 (DSC) | 1.753 | 99 | 497.5 | 498.2 | A |
| 199 | 267.18 (DSC) | 1.754 | 99 | 416.5 | 417.0 | A |
| 124 | 222.36 (DSC) | 1.950 | 99 | 512.5 | 513.2 | A |
| 144 | 206.86 (DSC) | 2.537 | 97 | 443.5 | 444.2 | A |
| 200 | 252.02 (DSC) | 2.111 | 98 | 480.5 | 481.2 | A |
| 145 | 216.15 (DSC) | 2.445 | 96 | 482.5 | 483.2 | A |
| 146 | 220.44 (DSC) | 2.084 | 99 | 429.5 | 430.2 | A |
| 125 | 230.42 (DSC) | 2.384 | 98 | 429.5 | 430.2 | A |
| 147 | 179.96 (DSC) | 1.673 | 98 | 464.5 | 465.1 | A |
| 148 | 221.24 (DSC) | 1.892 | 99 | 430.5 | 431.0 | A |
| 149 | 219.76 (DSC) | 2.302 | 99 | 415.5 | 416.0 | A |
| 150 | 235.14 (DSC) | 2.38 | 98 | 432.5 | 433.2 | A |
| 151 | 248.67 (DSC) | 1.966 | 98 | 496.5 | 497.1 | A |
| 152 | 247.79 (DSC) | 1.978 | 98 | 496.5 | 497.2 | A |
| 153 | 188.18 (DSC) | 2.464 | 98 | 535.5 | 536.1 | A |
| 154 | 191.6 (DSC) | 2.48 | 98 | 535.5 | 536.1 | A |
| 155 | 203.8 (DSC) | 3.142 | 98 | 483.5 | 484.2 | A |
| 156 | 216.42 (DSC) | 2.334 | 97 | 415.5 | 416.1 | A |
| 157 | 219.59 (DSC) | 2.342 | 98 | 415.5 | 416.2 | A |
| 191 | 212.4 (DSC) | 3.145 | 99 | 483.5 | 484.2 | A |
| 158 | 206.97 (DSC) | 1.768 | 98 | 468.5 | 469.2 | A |
| 159 | 222.55 (DSC) | 2.05 | 99 | 414.5 | 415.2 | A |
| 160 | 214.62 (DSC) | 1.970 | 99 | 418.5 | 419.2 | A |
| 161 | 202.04 (DSC) | 1.550 | 99 | 467.5 | 468.1 | A |
| 162 | 224.44 (DSC) | 2.055 | 99 | 414.5 | 415.2 | A |
| 163 | 214.18 (DSC) | 1.988 | 99 | 418.5 | 419.2 | A |
| 192 | 176.9 (DSC) | 2.360 | 98 | 536.5 | 537.1 | A |
| 164 | 216.09 (DSC) | 1.469 | 99 | 471.5 | 472.2 | A |
| 165 | 201.39 (DSC) | 1.564 | 99 | 467.5 | 468.0 | A |
| 166 | 210.32 (DSC) | 1.75 | 98 | 468.5 | 469.1 | A |
| 193 | 177.52 (DSC) | 2.348 | 96 | 536.5 | 537.1 | A |
| 142 | 219.04 (DSC) | 1.456 | 99 | 471.5 | 472.2 | A |
| 129 | 258.41 (DSC) | 2.01 | 99 | 488.5 | 489.2 | C |
| 178 | 220.99 (DSC) | 2.96 | 99 | 468.5 | 469.2 | C |
| 177 | 219.28 (DSC) | 2.99 | 98 | 468.5 | 469.2 | C |
| 181 | 241.83 (DSC) | 2.57 | 99 | 469.5 | 470.2 | C |
| 182 | 243.26 (DSC) | 2.51 | 99 | 469.5 | 470.2 | C |
| 130 | 216.8 (MT) | 2.04 | 98 | 488.5 | 489.2 | C |
| 131 | 192.55 (DSC) | 1.99 | 98 | 488.5 | 489.2 | A |
| 183 | 191.5 (DSC) | 2.59 | 98 | 441.5 | 442.2 | C |
| 184 | 192.0 (DSC) | 2.72 | 98 | 441.5 | 442.2 | C |
| 185 | 224 (DSC) | 1.66 | 99 | 489.5 | 490.2 | C |
| 132 | 221.6 (MT) | 1.97 | 98 | 488.5 | 489.2 | A |
| 179 | 197 (DSC) | 2.51 | 99 | 471.5 | 472.3 | C |
| 186 | 238.11 (DSC) | 1.97 | 99 | 486.5 | 487.2 | C |
| 187 | 190.8 (DSC) | 2.63 | 99 | 400.5 | 401.2 | C |
| 180 | 219.4 (DSC) | 2.93 | 99 | 468.5 | 469.2 | C |
| 188 | 197 (DSC) | 2.26 | 99 | 472.5 | 473.1 | A |
| 133 | 205.04 (DSC) | 2.26 | 99 | 464.5 | 465.2 | C |
| 134 | 204 (DSC) | 2.11 | 99 | 468.5 | 469.1 | A |
| 135 | 214.62 (DSC) | 2.12 | 99 | 502.5 | 503.2 | A |
| 189 | 205.21 (DSC) | 2.49 | 99 | 465.5 | 466.1 | C |
| 136 | 259.3 (DSC) | 2.46 | 99 | 540.5 | 541.2 | A |
| 137 | 257.94 (DSC) | 2.45 | 98 | 540.5 | 541.2 | A |
| 139 | 213.23 (DSC) | 2.23 | 98 | 502.5 | 503.2 | A |
| 140 | 222.6 (DSC) | 2.15 | 99 | 502.5 | 503.2 | A |
| 141 | 200.7 (DSC) | 2.15 | 99 | 502.5 | 503.1 | A |
| 167 | 174.94 (DSC) | 2.15 | 99 | 502.5 | 503.2 | A |
| 168 | 208.74 (DSC) | 2.15 | 99 | 502.5 | 503.2 | A |
| 126 | 168 (DSC) | 3.18 | 99 | 471.5 | 472.2 | C |
| 127 | 168 (DSC) | 3.18 | 98 | 471.5 | 472.2 | C |
| 128 | 181.09 (DSC) | 2.17 | 99 | 472.5 | 473.2 | A |

### SFCMS—Methods

### General Procedure for SFC-MS Methods

The SFC measurement was performed using an Analytical Supercritical fluid chromatography (SFC) system composed by a binary pump for delivering carbon dioxide (CO₂) and modifier, an autosampler, a column oven, an UV detector equipped with a high-pressure flow cell standing up to 400 bars. Data acquisition was performed with appropriate software.

TABLE 3
Analytical SFC-MS Methods (Flow expressed in mL/min; column temperature
(T) in ° C.; run time in minutes; backpressure (BPR) in bars.

| | | | | | Run |
|---|---|---|---|---|---|
| | | | | Flow | time |
| | | Mobile | | — | — |
| Method | Column | Phase | Gradient | T | BPR |
| 1 | Phenomenex Lux | A: CO₂ | From 5% B to | 3 | 20 |
| | Cellulose-1 | B: MeOH | 60% B in 10 | — | — |
| | 150 × 4.6 mm, 5 um | (+0.1% DEA) | min, hold 3 min. | 35 | 100 |
| | | | To 5% B in 1.9 | | |
| | | | min. | | |
| 2 | Phenomenex Lux | A: CO₂ | From 5% B to | 3 | 20 |
| | i-Amylose-1 | B: EtOH (+0.1% | 60% B in 10 | — | — |
| | 150 × 4.6 mm, 5 um | DEA) | min, hold 3 min. | 35 | 100 |
| | | | To 5% B in 1.9 | | |
| | | | min. | | |
| 3 | Phenomenex Lux | A: CO₂ | From 5% B to | 3 | 20 |
| | i-Amylose-1 | B: iPrOH (+0.1% | 60% B in 10 | — | — |
| | 150 × 4.6 mm, 5 um | DEA) | min, hold 3 min. | 35 | 100 |
| | | | To 5% B in 1.9 | | |
| | | | min. | | |
| 4 | Regis | A: CO₂ | From 5% B to | 3 | 13 |
| | Technologies | B: EtOH (+0.1% | 60% B in 7 min, | — | — |
| | Reflect i- | DEA) | hold 2 min. To | 35 | 100 |
| | Cellulose-C | | 5% B in 1 min. | | |
| | 100 × 4.6 mm, 3 um | | | | |
| 5 | Phenomenex Lux | A: CO2 | From 5% B to | 3 | 20 |
| | Amylose-2 | B: Ethanol + 0.1% | 60% B in 10 | — | — |
| | 150 × 4.6 mm, 5 um | Diethylamine | min, hold 3 min. | 35 | 100 |
| | | | To 5% B in 1.9 | | |
| | | | min. | | |
| 6 | Phenomenex Lux | A: CO2 | From 5% B to | 3 | 20 |
| | Amylose-1 | B: iPROH + 0.1% | 60% B in 10 | — | — |
| | 150 × 4.6 mm, 5 um | Diethylamine | min, hold 3 min. | 35 | 100 |
| | | | To 5% B in 1.9 | | |
| | | | min. | | |
| 7 | Phenomenex Lux | A: CO2 | From 5% B to | 3 | 20 |
| | Amylose-1 | B: Ethanol + 0.1% | 60% B in 10 | — | — |
| | 150 × 4.6 mm, 5 um | Diethylamine | min, hold 3 min. | 35 | 100 |
| | | | To 5% B in 1.9 | | |
| | | | min. | | |
| 8 | Phenomenex Lux | A: CO2 | From 5% B to | 3 | 20 |
| | Amylose-1 | B: Methanol + | 60% B in 10 | — | — |
| | 150 × 4.6 mm, 5 um | 0.1% | min, hold 3 min. | 35 | 100 |
| | | Diethylamine | To 5% B in 1.9 | | |
| | | | min. | | |
| 9 | Phenomenex Lux | A: CO2 | From 5% B to | 3 | 20 |
| | Cellulose-1 | B: Ethanol + 0.1% | 60% B in 10 | — | — |
| | 150 × 4.6 mm, 5 um | Diethylamine | min, hold 3 min. | 35 | 100 |
| | | | To 5% B in 1.9 | | |
| | | | min. | | |
| 10 | Regis | A: CO₂ | From 5% B to | 2.5 | 13 |
| | Technologies | B: Methanol | 60% B in 7 min, | — | — |
| | Reflect i- | (+0.1% DEA) | hold 2 min. To | 35 | 100 |
| | Cellulose-C | | 5% B in 1 min. | | |
| | 100 × 4.6 mm, 3 um | | | | |
| 11 | Regis | A: CO2 | From 5% B to | 3 | 20 |
| | Technologies | B: Ethanol + 0.1% | 60% B in 10 | — | — |
| | Reflect i- | Diethylamine | min, hold 3 min. | 35 | 100 |
| | Amylose-A | | To 5% B in 1.9 | | |
| | 150 × 4.6 mm, 5 um | | min. | | |

TABLE 4
Analytical SFC data - Rt means retention time (in minutes),
MW the molecular weight of the compound, [M +
H]+ means the protonated mass of the compound,
method refers to the method used for (SFC)MS analysis
of enantiomerically pure compounds.

| | | | | | | Isomer |
|---|---|---|---|---|---|---|
| Compound | | UV | | | | Elution |
| number | Rₜ | Area % | MW | [M + H]⁺ | Method | Order |
| 1a | 8.8 | 99 | 483.5 | 484.2 | 1 | A |
| 1b | 9.51 | 99 | 483.5 | 484.2 | 1 | B |
| 2a | 5.41 | 99 | 508.4 | 509.1 | 2 | A |
| 2b | 5.82 | 99 | 508.4 | 509.1 | 2 | B |
| 3a | 7.76 | 99 | 482.5 | 483.2 | 3 | A |
| 3b | 8.54 | 99 | 482.5 | 483.2 | 3 | B |
| 5a | 8.30 | 99 | 483.5 | 484.2 | 4 | A |
| 5a | 8.64 | 99 | 483.5 | 484.2 | 1 | A |
| 5b | 9.84 | 99 | 483.5 | 484.2 | 4 | B |
| 5b | 9.24 | 99 | 483.5 | 484.2 | 1 | A |
| 10a | 9.18 | 99 | 503.9 | 504.1 | 1 | A |
| 10a | 9.03 | 99 | 503.9 | 504.1 | 1 | A |
| 10b | 9.95 | 99 | 503.9 | 504.1 | 1 | B |
| 10b | 9.90 | 99 | 503.9 | 504.1 | 1 | B |
| 48 | 7.685 | 99 | 499.5 | — | 5 | A |
| 49 | 8.070 | 99 | 499.5 | — | 5 | B |
| 50 | 9.327 | 99 | 486.5 | — | 6 | B |
| 51 | 9.900 | 99 | 486.5 | — | 6 | — |
| 52 | 9.205 | 95 | 486.5 | — | 6 | A |
| 61 | 9.773 | 99 | 490.4 | — | 6 | A |
| 55 | 10.182 | 91 | 468.5 | — | 6 | A |
| 56 | 8.943 | 99 | 472.5 | — | 6 | A |
| 57 | 10.833 | 91 | 468.5 | — | 6 | B |
| 58 | 5.543 | 98 | 512.5 | — | 1 | — |
| 169 | 9.28 | 99 | 472.5 | — | 6 | B |
| 170 | 9.833 | 99 | 509.4 | — | 6 | — |
| 60 | 10.398 | 99 | 409.4 | — | 6 | B |
| 62 | 9.733 | 99 | 497.5 | — | 6 | B |
| 63 | 8.058 | 98 | 536.5 | — | 6 | A |
| 64 | 8.307 | 99 | 536.5 | — | 6 | B |
| 171 | 10.122 | 99 | 469.5 | — | 1 | B |
| 190 | 8.317 | 99 | 469.5 | — | 1 | A |
| 172 | 7.843 | 99 | 537.5 | — | 6 | A |
| 173 | 8.097 | 99 | 537.5 | — | 6 | B |
| 65 | 9.927 | 97 | 486.5 | — | 7 | — |
| 66 | 10.095 | 99 | 486.5 | — | 6 | A |
| 67 | 10.378 | 98 | 486.5 | — | 6 | B |
| 68 | 10.410 | 98 | 486.5 | — | 6 | B |
| 69 | 9.103 | 99 | 486.5 | — | 6 | B |
| 70 | 10.032 | 99 | 486.5 | — | 6 | A |
| 71 | 8.880 | 99 | 486.5 | — | 6 | A |
| 72 | 8.788 | 99 | 486.5 | — | 6 | — |
| 74 | 7.883 | 99 | 487.5 | — | 4 | A |
| 73 | 8.708 | 99 | 487.5 | — | 4 | B |
| 174 | 10.888 | 99 | 494.5 | — | 7 | — |
| 75 | 6.787 | 99 | 486.5 | — | 8 | — |
| 76 | 6.678 | 99 | 490.5 | — | 9 | B |
| 77 | 6.257 | 99 | 490.5 | — | 9 | A |
| 78 | 9.628 | 99 | 486.5 | — | 6 | A |
| 79 | 9.938 | 99 | 486.5 | — | 6 | B |
| 80 | 9.917 | 98 | 494.5 | — | 7 | — |
| 81 | 8.975 | 99 | 486.5 | — | 6 | — |
| 82 | 9.480 | 99 | 486.5 | — | 6 | — |
| 83 | 8.603 | 99 | 486.5 | — | 6 | — |
| 84 | 7.370 | 99 | 553.5 | — | 6 | B |
| 85 | 7.302 | 99 | 553.5 | — | 6 | A |
| 86 | 2.558 | 99 | 483.5 | — | 4 | A |
| 87 | 3.267 | 99 | 483.5 | — | 4 | B |
| 93 | 9.233 | 99 | 429.5 | — | 7 | A |
| 94 | 10.047 | 99 | 429.5 | — | 7 | B |
| 104 | 9.785 | 99 | 479.5 | — | 1 | A |
| 105 | 10.815 | 98 | 479.5 | — | 1 | B |
| 111 | 10.343 | 99 | 498.5 | — | 7 | A |
| 112 | 11.368 | 99 | 498.5 | — | 7 | B |
| 113 | 11.30 | 97 | 485.5 | — | 8 | B |
| 114 | 10.515 | 99 | 485.5 | — | 8 | A |
| 176 | 10.972 | 98 | 502.9 | — | 6 | B |
| 175 | 10.218 | 97 | 502.9 | — | 6 | A |
| 150 | 9.388 | 99 | 432.5 | — | 9 | — |
| 151 | 9.707 | 99 | 496.5 | — | 6 | A |
| 152 | 10.733 | 99 | 496.5 | — | 6 | B |
| 153 | 9.222 | 99 | 535.5 | — | 6 | B |
| 154 | 8.442 | 99 | 535.5 | — | 6 | A |
| 155 | 7.657 | 99 | 483.5 | — | 6 | A |
| 156 | 10.128 | 99 | 415.5 | — | 6 | A |
| 157 | 10.465 | 99 | 415.5 | — | 6 | B |
| 191 | 7.868 | 99 | 483.5 | — | 6 | B |
| 158 | 11.477 | 96 | 468.5 | — | 6 | A |
| 159 | 10.7 | 99 | 414.5 | — | 6 | B |
| 160 | 9.387 | 99 | 418.5 | — | 6 | B |
| 161 | 11.418 | 99 | 468.5 | — | 6 | B |
| 162 | 10.582 | 99 | 414.5 | — | 6 | A |
| 163 | 9.192 | 99 | 418.5 | — | 6 | A |
| 192 | 8.455 | 99 | 536.5 | — | 6 | A |
| 164 | 10.053 | 99 | 471.5 | — | 7 | A |
| 165 | 10.678 | 99 | 467.5 | — | 6 | A |
| 166 | 12.587 | 99 | 468.5 | — | 6 | B |
| 193 | 9.040 | 96 | 536.5 | — | 6 | B |
| 142 | 11.072 | 99 | 471.5 | — | 7 | B |
| 129 | 7.52 | 98 | 488.5 | — | 4 | — |
| 178 | 11.51 | 98 | 468.5 | — | 6 | B |
| 177 | 10.78 | 98 | 468.5 | — | 6 | A |
| 181 | 9.27 | 99 | 469.6 | — | 10 | B |
| 182 | 8.12 | 99 | 469.6 | — | 10 | A |
| 130 | 5.12 | 96 | 488.5 | — | 11 | — |
| 131 | 9.26 | 99 | 488.5 | — | 6 | B |
| 183 | 8.03 | 99 | 441.5 | — | 1 | A |
| 184 | 8.47 | 99 | 441.5 | — | 1 | B |
| 132 | 7.78 | 98 | 488.5 | — | 6 | A |
| 135 | 8.47 | 99 | 502.5 | — | 7 | — |
| 136 | 5.64 | 99 | 540.5 | — | 5 | — |
| 137 | 10.85 | 99 | 540.5 | — | 6 | — |
| 139 | 10.54 | 97 | 502.5 | — | 7 | — |
| 140 | 6.748 | 99 | 502.5 | — | 6 | A |
| 141 | 8.47 | 97 | 502.5 | — | 6 | B |
| 167 | 8.23 | 99 | 502.5 | — | 6 | A |
| 168 | 9.07 | 99 | 502.5 | — | 6 | B |
| 126 | 5.22 | 99 | 471.5 | — | 1 | B |
| 127 | 2.84 | 99 | 471.5 | — | 1 | A |

### 4—Chemical Stability Testing

HTChem stab assay was used to assess the chemical stability of the compounds. 72 h stirring at rt in a buffer pH2, pH4 and pH7 vs a reference DMSO solution. HTChem stab assay is based on the handbook of Pharmaceutical Stress Testing, Predicting Drug Degradation, second edition, authors Baertschi, Alsante, Reed ISBN 978-143980179-6.

Compounds disclosed herein may have the advantage that they are chemically more stable than other compounds (e.g. than other known compounds), for instance as tested in the chemical stability assay described below.

### General Procedure HTChem Stab Assay

Compounds were plated (1 μl 10 mM DMSO stock in 96 well plate (DMSO control+3 pH conditions), one plate per condition).

200 μL of respectively DMSO, Buffer/DMSO mix pH2, Buffer/DMSO mix pH4 and Buffer/DMSO mix pH7 was added.

After 72 h shaking (300 rpm) at room temperature the samples were analysed by LC/UV/MS. Chromatograms of samples (stability conditions) were compared with the chromatograms of control sample (DMSO). Extra peaks were flagged and identified by mass spectrometry. Sample purity (reference included) were reported.

### Buffer Preparation

pH3 buffer: For 11; weigh 4.14 g NaH2PO4·H2O, add 600 ml H2O, add H3PO4 to pH3, add H2O to 1000 ml. Filter over 0.2 μm and store at 4° C. Expires after 30 days.
pH6 buffer: For 1 l; weigh 4.14 g NaH2PO4·H2O, add 600 ml H2O, add 1N NaOH to pH6, add H2O to 1000 ml. Filter over 0.2 m and store at 4° C. Expires after 30 days.

### Buffer/DMSO Mixture Preparation:

pH2 Buffer/DMSO (StabSolvent): mix 2 parts of HCl 0.1N with 1 part of DMSO
pH4 Buffer/DMSO (StabSolvent): mix 2 parts of buffer pH3 with 1 part of DMSO
pH7 Buffer/DMSO (StabSolvent): mix 2 parts of buffer pH6 with 1 part of DMSO measure pH prior to use.

### UPLC Method HT Chemical Stability:

Instrument: Acquity UPLC; Column: Acquity HSS T3, 50 mm length×2.1 mm i.d., 1.8 μm particle size. Detectors: PDA (250-350 nm) and SQD2 (90-1500m/z). Mobile phase A: 0.1% Formic acid in water; Mobile phase B: 0.1% Formic acid in Acetonitrile.

Injection volume: 5 μL, flow rate 0.6 mL/min.

### Gradient

| Time(min) | % A | % B |
|---|---|---|
| 0 | 100 | 0 |
| 0.3 | 95 | 5 |
| 1.8 | 0 | 100 |
| 2.6 | 0 | 100 |
| 2.9 | 100 | 0 |

Data processing including peak integration and identification was done with Waters OpenLynx software.

TABLE 5
Chemical stability data

| Compound | Vehicle | | | |
|---|---|---|---|---|
| number | DMSO | Buffer pH2 | Buffer pH4 | Buffer pH7 |
| 1a | 100 | 100 | 100 | 100 |
| 1ab | 100 | 100 | 100 | 100 |
| 1b | 100 | 99 | 96 | 96 |
| 2a | 100 | 100 | 100 | 100 |
| 2ab | 100 | 100 | 100 | 98 |
| 2b | 100 | 100 | 100 | 100 |
| 3a | 100 | 100 | 100 | 100 |
| 3b | 98 | 93 | 99 | 93 |
| 4ab | 98 | 98 | 98 | 98 |
| 5a | 100 | 100 | 100 | 100 |
| 5ab | 94 | 95 | 96 | 96 |
| 5b | 100 | 100 | 100 | 100 |
| 6ab | 93 | 100 | 100 | 100 |
| 7ab | 100 | 100 | 100 | 100 |
| 8ab | 100 | 100 | 100 | 100 |
| 9ab | 100 | 100 | 100 | 100 |
| 10a | 100 | 84 | 76 | 97 |
| 10ab | 90 | 72 | 64 | 90 |
| 10b | 96 | 81 | 71 | 92 |
| 12ab | 99 | 100 | 100 | 100 |
| 13ab | 100 | 100 | 100 | 100 |
| 14ab | 100 | 100 | 100 | 100 |
| 15ab | 98 | 98 | 100 | 99 |
| 16ab | 77 | 74 | 74 | 80 |
| 17ab | 100 | 100 | 97 | 100 |
| 18ab | 98 | 98 | 100 | 100 |
| 19ab | 98 | 98 | 98 | 98 |
| 20ab | 100 | 100 | 100 | 100 |
| 21ab | 100 | 100 | 100 | 100 |
| 22ab | 98 | 97 | 97 | 98 |
| 23ab | 100 | 100 | 100 | 100 |
| 24ab | 100 | 100 | 100 | 100 |
| 25ab | 100 | 100 | 100 | 100 |
| 26ab | 95 | 95 | 95 | 95 |
| 27ab | 100 | 100 | 100 | 100 |
| 28ab | 100 | 100 | 100 | 100 |
| 29ab | 100 | 100 | 100 | 100 |
| 30ab | 98 | 98 | 98 | 98 |
| 31ab | 100 | 100 | 100 | 100 |
| 32ab | 100 | 100 | 100 | 100 |
| 33ab | 100 | 64 | 100 | 100 |
| 34ab | 94 | 94 | 95 | 93 |
| 35ab | 100 | 100 | 99 | 98 |
| 36ab | 100 | 100 | 100 | 100 |

These chemical stability data % depend on the purity of the compounds, their solubility and the solubility of their potential degradation products. Nevertheless, this showed that, under the tested conditions, the compounds were stable, and mostly not susceptible to unwanted degradation in acidic media.

### 5—Biological Assays/Pharmacological Examples

MIC Determination for Testing Compounds Against M. tuberculosis.

### Test 1

Test compounds and reference compounds are dissolved in DMSO and 1 μl of solution is spotted per well in 96 well plates at 200× the final concentration. Column 1 and column 12 are left compound-free, and from column 2 to 11 compound concentration is diluted 3-fold.

Frozen stocks of Mycobacterium tuberculosis strain EH4.0 expressing green-fluorescent protein (GFP) are previously prepared and titrated. To prepare the inoculum, 1 vial of frozen bacterial stock is thawed to room temperature and diluted to 5×10 exp5 colony forming units per ml in 7H9 broth. 200 μl of inoculum, which corresponds to 1×10 exp5 colony forming units, are transferred per well to the whole plate, except column 12. 200 μl 7H9 broth are transferred to wells of column 12. Plates are incubated at 37° C. in plastic bags to prevent evaporation. After 7 days, fluorescence is measured on a Gemini EM Microplate Reader with 485 excitation and 538 nm emission wavelengths and IC₅₀ and/or pIC₅₀ values (or the like, e.g. IC₅₀, IC₉₀, pIC₉₀, etc) are (or may be) calculated.

### Test 2

Appropriate solutions of experimental/test and reference compounds were made in 96 well plates with 7H9 medium. Samples of Mycobacterium tuberculosis strain H37Rv were taken from cultures in logarithmic growth phase. These were first diluted to obtain an optical density of 0.3 at 600 nm wavelength and then diluted 1/100, resulting in an inoculum of approximately 5×10 exp5 colony forming units per ml. 100 μl of inoculum, which corresponds to 5×10 exp4 colony forming units, were transferred per well to the whole plate, except column 12. Plates were incubated at 37° C. in plastic bags to prevent evaporation. After 7 days, resazurin was added to all wells. Two days later, fluorescence was measured on a Gemini EM Microplate Reader with 543 excitation and 590 nm emission wavelengths and MIC₅₀ and/or pIC₅₀ values (or the like, e.g. IC₅₀, IC₉₀, pIC₉₀, etc) were (or may be) calculated.

### Test 3: Time Kill Assays

Bactericidal or bacteriostatic activity of the compounds can be determined in a time kill kinetic assay using the broth dilution method. In this assay, the starting inoculum of M. tuberculosis (strain H37Rv and H37Ra) is 10⁶ CFU/ml in Middlebrook (lx) 7H9 broth. The test compounds are tested alone or in combination with another compound (e.g. a compound with a different mode of action, such as with a cytochrome bd inhibitor) at a concentration ranging from 10-30 μM to 0.9-0.3 μM respectively. Tubes receiving no antibacterial agent constitute the culture growth control. The tubes containing the microorganism and the test compounds are incubated at 37° C. After 0, 1, 4, 7, 14 and 21 days of incubation samples are removed for determination of viable counts by serial dilution (10⁰ to 10⁻⁶) in Middlebrook 7H9 medium and plating (100 μl) on Middlebrook 7H11 agar. The plates are incubated at 37° C. for 21 days and the number of colonies are determined. Killing curves can be constructed by plotting the log₁₀ CFU per ml versus time. A bactericidal effect of a test compound (either alone or in combination) is commonly defined as 2-log₁₀ decrease (decrease in CFU per ml) compared to Day 0. The potential carryover effect of the drugs is limited by using 0.4% charcoal in the agar plates, and by serial dilutions and counting the colonies at highest dilution possible used for plating.

### Results

Compounds of the invention/examples, for example when tested in Test 2 described above, may typically have a pMIC₅₀ from 3 to 10 (e.g. from 4.0 to 9.0, such as from 5.0 to 8.5)

### Further Data

The compounds of the invention/examples may have advantages associated with in vitro potency, kill kinetics (i.e. bactericidal effect) in vitro, PK properties, food effect, safety/toxicity (including liver toxicity, coagulation, 5-LO oxygenase), metabolic stability, Ames II negativity, MNT negativity, aqueous based solubility (and ability to formulate) and/or cardiovascular effect e.g. on animals (e.g. anesthetized guinea pig). The data below that was generated/calculated may be obtained using standard methods/assays, for instance that are available in the literature or which may be performed by a supplier (e.g. Microsomal Stability Assay—Cyprotex, Mitochondrial toxicity (Glu/Gal) assay—Cyprotex, as well as literature CYP cocktail inhibition assays).

### Metabolic Stability in Liver Microsomes

The test compound was incubated under the generic condition (0.5 mg/ml microsomal protein, 1 mM NADPH, 1 mM MgCl2, and 0.1 M phosphate buffer, pH 7.4, 37° C.) at a defined substrate concentration (typically 1 μM) in liver microsomes of selected species across a time course (typically 0, 5, 10, 20, 40 and 60 minutes) at Cyprotex (Cheshire, UK). The liver microsomes, buffer and test compound will be pre-incubated for 5 minutes. The addition of the co-factor NADPH will initiate the reaction and the reaction was terminated by addition of acetonitrile. The samples were centrifuged prior to analysis by LC-MS/MS analysis. The relative amount of parent compound remaining in the active incubations vs. the control incubations (t=0 mins) for each compound was measured by peak area comparison. The in vitro metabolic half-life (t_{1/2}) was calculated using the slope of the log-linear regression from the percentage parent compound remaining versus time relationship (κ), The in vitro intrinsic clearance (Cl^{int}) (μl/min/mg microsomal protein) was calculated using the in vitro metabolic half-life, incubation volume and the weight of microsomal protein in the incubation.

### Genotoxicity Assay (AmesIIb Mutagenicity Test)

The aim is to evaluate compounds (and/or their metabolites) for their ability to induce reverse mutations in a gene of histidine-requiring Salmonella typhimurium strains to produce histidine-independent strains of these bacteria, in the absence and in the presence of a mammalian metabolic activation system.

Point mutations were made in the histidine (His) operon in Salmonella typhimurium, rendering the bacteria incapable of producing histidine. These single base substitutions are positioned at strategic points within the His gene, resulting in his-organisms that cannot grow unless histidine is supplied. When a mutagenic event occurs, base substitutions within the His gene then cause the reversion of the his-Ames II™ strains to His.

A chemical's mutagenic potential is assessed by exposing these his-organisms to varying concentrations of chemical and selecting for the reversion event. Medium lacking histidine is used for this selection which allows only those cells that have undergone the his- to His reversion to survive and grow.

The assay may be performed using a kit and instructions from Xenometric. The two strains provided in the Xenometric kit are the Ames II™ Mixed Strains and TA98. The Ames II™ Mixed Strains contains an equimolar mixture of the Ames II™ TA7001-TA7006 strains. Individually, these strains are designed to revert by only one specific base-pair substitution out of six possible changes. Thus, when mixed, all six base substitution mutations can be represented in one culture. This kit also contains TA98 for the detection of frameshift mutations. For the avoidance of doubt, an AmesIIb score of 1 shows no genotoxicity signals.

### Biological Results

Compounds of the examples were tested for bacterial activity (Test 2 described above in section “MIC determination for testing compounds against M. tuberculosis.”) and Metabolic Stability in liver microsome; the results presented in Table 6 were obtained:

TABLE 6
Biological data table

| | | | Metabolic Stability | | | | |
|---|---|---|---|---|---|---|---|
| | | | (liver microsome intrinsic clearance) | | | | |
| | | | Clearance | | Clearance | | |
| | Bacterial activity | | (mouse, | Half life | (human, | Half life | GeneTox |
| Compound | | MIC₉₀ | μl/min./mg | (mouse, | μl/min./mg | (human, | AmesIIb |
| number | pMIC₅₀ | (in μM) | protein) | min.) | protein) | min.) | Score |
| 1ab | 8.020 | 0.028 | <7.7 | >180 | <7.7 | >180 | 1 |
| 1a | 8.505 | 0.009 | <7.7 | >180 | <7.7 | >180 | |
| 1b | 8.576 | 0.007 | 7.8 | 178 | 7.81 | 178 | |
| 2ab | 7.627 | 0.089 | 10.7 | 129 | <7.7 | >180 | 1 |
| 2a | 7.648 | 0.086 | 11.4 | 121 | <7.7 | >180 | 1 |
| 2b | 7.502 | 0.098 | 12.3 | 112 | <7.7 | >180 | 1 |
| 3ab | 8.450 | 0.009 | 13.6 | 102 | 15 | 92.5 | |
| 3a | 8.532 | 0.008 | 7.78 | 178 | 13.4 | 103 | 1 |
| 3b | 8.483 | 0.010 | 23.8 | 58.3 | 26.1 | 53.2 | 1 |
| 4ab | 8.152 | 0.017 | 8.38 | 165 | | | 1 |
| 5ab | 8.082 | 0.023 | <7.7 | >180 | | | 1 |
| 5a | 7.665 | 0.060 | <7.7 | >180 | | | 1 |
| 5b | 7.873 | 0.044 | <7.7 | >180 | | | 1 |
| 6ab | 8.459 | 0.007 | 14.2 | 97.4 | | | 1 |
| 7ab | 8.362 | 0.014 | 20.5 | 67.7 | | | |
| 8ab | 8.148 | 0.018 | | | | | 1 |
| 9ab | 8.306 | 0.011 | 13.9 | 99.7 | | | 1 |
| 10ab | 8.260 | 0.016 | <7.7 | >180 | | | 1 |
| 10a | <6.301 | | <7.7 | >180 | | | 1 |
| 10b | 8.207 | 0.021 | 11.1 | 125 | | | 1 |
| 11ab | | | | | | | |
| 12ab | 7.631 | 0.064 | <7.7 | >180 | <7.7 | >180 | 1 |
| 13ab | 8.067 | 0.024 | 15.5 | 89.4 | | | 1 |
| 14ab | 7.877 | 0.036 | <7.7 | >180 | | | 1 |
| 15ab | 8.512 | 0.006 | | | | | 1 |
| 16ab | <6.301 | | | | | | |
| 17ab | 7.526 | 0.105 | <7.7 | >180 | | | 1 |
| 18ab | 7.431 | 0.142 | 11.1 | 125 | | | 1 |
| 19ab | 7.940 | 0.047 | | | | | |
| 20ab | 9.031 | 0.003 | 21.3 | 65 | 36.3 | 38.2 | 1 |
| 21ab | 8.457 | 0.011 | | | | | |
| 22ab | 8.641 | 0.007 | 13 | 107 | | | 1 |
| 23ab | 8.081 | 0.022 | 14.6 | 94.9 | | | 1 |
| 24ab | 8.471 | 0.008 | <7.7 | >180 | | | 1 |
| 25ab | 8.127 | 0.014 | 9.97 | 139 | | | 1 |
| 26ab | 7.306 | 0.143 | 10.9 | 127 | | | 1 |
| 27ab | 8.336 | 0.014 | | | | | |
| 28ab | 8.436 | 0.011 | | | | | |
| 29ab | <6.301 | 3.188 | <7.7 | >180 | <7.7 | >180 | 1 |
| 30ab | 6.964 | 0.332 | 9.08 | 153 | | | 1 |
| 31ab | 7.797 | 0.052 | <7.7 | >180 | | | 1 |
| 32ab | 7.098 | 0.248 | | | | | |
| 33ab | 7.895 | 0.039 | | | | | |
| 34ab | 7.024 | 0.387 | <7.7 | >180 | | | 1 |
| 35ab | 7.873 | 0.041 | 46.4 | 29.9 | | | 1 |
| 36ab | <6.301 | | | | | | |
| 37 | 7.9 | 0.0035 | <7.7 | >168 | | | 1 |
| 38 | 7.77 | 0.052 | <7.7 | >180 | | | 1 |
| 39 | 7.31 | 0.16 | <7.7 | >180 | | | 1 |
| 138 | <6.3 | >0.5 | | | | | |
| 40 | 7.47 | 0.11 | <7.7 | >180 | | | 1 |
| 41 | <6.3 | >0.5 | | | | | |
| 42 | <6.3 | >0.5 | | | | | |
| 45 | 6.61 | 1.05 | | | | | |
| 43 | 6.81 | 0.37 | | | | | |
| 44 | 7.34 | 0.21 | | | | | |
| 194 | 7.43 | 0.1 | <7.7 | >180 | <7.7 | >180 | 1 |
| 46 | <6.3 | >0.5 | | | | | |
| 47 | <6.3 | >0.5 | | | | | |
| 196 | 7.56 | 0.067 | <7.7 | >180 | | | 1 |
| 197 | 7.16 | 0.21 | | | | | |
| 198 | 7.34 | 0.15 | | | | | |
| 48 | 8.08 | 0.026 | | | | | |
| 49 | 8.11 | 0.023 | 12.9 | 108 | | | |
| 50 | 7.55 | 0.11 | 11.7 | 118 | | | 1 |
| 51 | 8.5 | 0.008 | <7.7 | >180 | | | 1 |
| 52 | 7.38 | 0.16 | | | | | |
| 61 | 7.96 | 0.036 | | | | | |
| 53 | 7.96 | 0.036 | | | | | |
| 54 | 7.14 | 0.22 | | | | | |
| 55 | 8.53 | 0.0086 | | | | | |
| 57 | 8.16 | 0.022 | | | | | |
| 58 | 7.02 | 0.39 | | | | | |
| 170 | <6.56 | >0.45 | | | | | |
| 59 | 7.75 | 0.061 | 59.4 | 23.4 | 347 | 4 | 1 |
| 60 | 7.76 | 0.067 | | | | | |
| 62 | 7.96 | 0.033 | 57.8 | 24 | 294 | 4.7 | 1 |
| 63 | <6.65 | >0.46 | | | | | |
| 64 | <6.3 | >0.5 | | | | | |
| 171 | 7.33 | 0.18 | | | | | |
| 190 | 7.32 | 0.19 | | | | | |
| 172 | <6.53 | >0.46 | | | | | |
| 173 | <6.49 | >0.47 | | | | | |
| 65 | 7.87 | 0.039 | | | | | |
| 66 | 7.29 | 0.24 | | | | | |
| 67 | 7.39 | 0.18 | | | | | |
| 68 | 7.32 | 0.23 | | | | | |
| 69 | 7.95 | 0.031 | | | | | |
| 70 | 7.25 | 0.23 | | | | | |
| 71 | 8.02 | 0.024 | | | | | |
| 72 | 7.86 | 0.042 | | | | | |
| 74 | 6.94 | >0.5 | <7.7 | >180 | | | |
| 73 | 7.18 | 0.35 | <7.7 | >180 | | | |
| 174 | 7.17 | 0.25 | 17.3 | 80.3 | | | |
| 75 | 7.48 | 0.086 | | | | | |
| 76 | 7.55 | 0.09 | | | | | |
| 77 | 7.6 | 0.098 | 26.1 | 53.1 | | | |
| 78 | 8.08 | 0.028 | | | | | |
| 79 | 7.99 | 0.035 | | | | | |
| 80 | 7.25 | 0.2 | 16.3 | 84.9 | | | |
| 81 | 7.24 | 0.19 | | | | | |
| 82 | 7.51 | 0.087 | | | | | |
| 83 | 7.2 | 0.23 | | | | | |
| 84 | <7.03 | >0.38 | | | | | |
| 85 | <6.3 | >0.5 | | | | | |
| 86 | 6.98 | 0.44 | | | | | |
| 87 | 6.97 | 0.47 | | | | | |
| 88 | 7.43 | 0.099 | | | | | |
| 103 | 8.12 | 0.02 | <7.7 | >180 | 119 | 11.6 | |
| 89 | 7.85 | 0.046 | <7.7 | >180 | | | 1 |
| 90 | 7.22 | 0.15 | | | | | |
| 91 | 6.72 | 0.48 | <7.7 | >180 | | | 1 |
| 94 | 7.86 | 0.039 | | | | | |
| 92 | 7.21 | 0.2 | | | | | |
| 95 | 8.44 | 0.011 | 0.012 | 58.7 | <0.0012 | >560 | 1 |
| 96 | 7.47 | 0.094 | | | | | |
| 97 | 7.72 | 0.057 | | | | | |
| 98 | 7.56 | 0.11 | | | | | |
| 99 | 7.15 | 0.22 | | | | | |
| 100 | 7.21 | 0.2 | | | | | |
| 101 | 5.7 | 3.95 | | | | | |
| 102 | 7.59 | 0.058 | <7.7 | >180 | | | 1 |
| 104 | 8.25 | 0.015 | | | | | |
| 105 | 8.24 | 0.014 | | | | | |
| 106 | 7.92 | 0.03 | 12.1 | 115 | | | 1 |
| 107 | 6.87 | 0.33 | | | | | |
| 108 | 8.12 | 0.018 | | | | | |
| 109 | 8.04 | 0.024 | | | | | |
| 110 | 8.23 | 0.016 | | | | | |
| 111 | 8.33 | 0.012 | | | | | |
| 112 | 8.07 | 0.027 | | | | | |
| 113 | 7.89 | 0.034 | | | | | |
| 114 | 8.12 | 0.032 | | | | | |
| 115 | 7.61 | 0.096 | | | | | |
| 116 | 8.34 | 0.0097 | | | | | |
| 176 | 7.9 | 0.039 | | | | | |
| 117 | 8.51 | 0.077 | | | | | |
| 175 | 7.66 | 0.059 | | | | | |
| 143 | 7.48 | 0.092 | | | | | |
| 118 | 7.48 | 0.083 | | | | | |
| 119 | 7.72 | 0.052 | | | | | |
| 120 | 7.84 | 0.042 | 14.4 | 96.6 | | | 1 |
| 121 | 8.06 | 0.035 | | | | | |
| 122 | 7.36 | 0.21 | | | | | |
| 123 | <6.3 | >0.5 | | | | | |
| 199 | <6.3 | >0.5 | | | | | |
| 144 | 7.9 | 0.05 | <7.7 | >180 | | | 1 |
| 200 | 7.21 | 0.22 | | | | | |
| 145 | 7.94 | 0.041 | | | | | |
| 146 | 7.37 | 0.15 | | | | | |
| 125 | 7.95 | 0.04 | | | | | |
| 147 | 7.82 | 0.05 | | | | | |
| 148 | 6.48 | >0.5 | | | | | |
| 149 | 6.64 | >0.5 | | | | | |
| 150 | 8 | 0.03 | | | | | |
| 151 | 7.74 | 0.054 | 62.5 | 22.2 | 326 | 4.3 | 1 |
| 152 | 7.63 | 0.088 | 129 | 10.7 | >347 | <4 | 1 |
| 153 | <6.3 | >0.5 | | | | | |
| 154 | <6.3 | >0.5 | | | | | |
| 155 | <6.3 | >0.5 | | | | | |
| 156 | 6.72 | >0.5 | | | | | |
| 157 | 6.9 | 0.41 | | | | | |
| 191 | <6.3 | >0.5 | | | | | |
| 158 | 7.12 | 0.29 | | | | | |
| 159 | 7.96 | 0.037 | | | | | |
| 160 | 7.45 | 0.088 | | | | | |
| 161 | 7.73 | 0.056 | | | | | |
| 162 | 7.95 | 0.044 | | | | | |
| 163 | 7.18 | 0.17 | | | | | |
| 192 | 5.81 | >5 | | | | | |
| 164 | 7.77 | 0.058 | | | | | |
| 165 | 7.79 | 0.055 | | | | | |
| 166 | 6.84 | >0.5 | | | | | |
| 193 | <6.3 | >0.5 | | | | | |
| 142 | 7.64 | 0.097 | | | | | |
| 178 | 7.66 | 0.11 | <7.7 | >180 | | | 1 |
| 177 | 7.85 | 0.056 | 15.8 | 87.8 | | | 1 |
| 181 | 7.24 | 0.22 | | | | | |
| 182 | 7.28 | 0.2 | | | | | |
| 131 | <6.3 | >0.5 | | | | | |
| 183 | 6.19 | 0.48 | 10.1 | 137 | | | 1 |
| 184 | 6.78 | 0.43 | <7.7 | >180 | | | 1 |
| 185 | 8.01 | 0.034 | | | | | |
| 132 | <6.57 | >0.45 | | | | | |
| 179 | 8.48 | 0.0097 | 53.5 | 25.9 | | | 1 |
| 186 | 7.39 | 0.19 | <7.7 | >180 | | | |
| 187 | 6.82 | >0.5 | | | | | |
| 180 | 7.7 | 0.12 | 9.9 | 141 | | | |
| 188 | 7.64 | 0.23 | | | | | |
| 133 | 7.93 | 0.051 | | | | | |
| 134 | 7.59 | 0.12 | | | | | |
| 135 | <6.65 | >0.44 | | | | | |
| 189 | 7.06 | 0.33 | | | | | |
| 136 | <6.3 | >0.5 | | | | | |
| 137 | <6.3 | >0.5 | | | | | |
| 139 | <6.74 | >0.45 | | | | | |
| 140 | 6.94 | 0.33 | | | | | |
| 141 | 6.78 | >0.5 | | | | | |
| 167 | 6.88 | 0.45 | | | | | |
| 168 | 6.76 | 0.5 | | | | | |
| 126 | 7.62 | 0.12 | 47.6 | 29.1 | | | 1 |
| 127 | 7.29 | 0.28 | 39.7 | 34.9 | | | 1 |

### Compounds Disclosed Herein:

Are active using the test methods described above, and may have other potency advantages depending on the setting
May have the advantage that they are chemically more stable that other compounds (for instance in the tests described herein); this may have the advantage that they do not form degradants (e.g. that are undesired or may elicit unwanted side-effects)
May display better stability, for instance compared to compounds known in the prior art, e.g. in the stability tests described herein (including the metabolic stability in liver microsomes test described herein)
May have advantages in relation to toxicity, for instance as displayed/measured using the genotoxicity assay/mutagenicity test described, or another test described herein, for instance displaying the CVS results or due to the Glu/Gal assay results)

1. A compound of formula (IA): 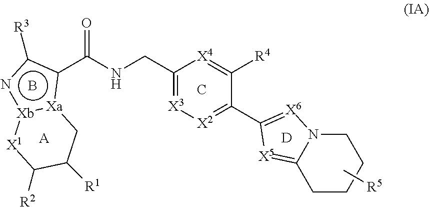
wherein:
A is a 6-membered ring, which is aromatic or non-aromatic;
B is a 5-membered aromatic ring;
one of Xₐ and X_{b} is N and the other is C;
X¹ is ═N—, —CH₂— or ═C(R^{10a})—;
X², X³, X⁴, X⁵ and X⁶ are each, independently, ═N— or ═C(R^{10b})—;
R¹ and R² are each, independently, hydrogen, halo, —R^{6c}, —O—R^{6d}, —C(═O)—R^{6e}, —C(═O)—N(R⁶)(R⁷), —CN or —N(R^{6a})R^{6b};
R³ is H, halo, or —C₁₋₃ alkyl (linear, branched or cyclic) optionally substituted by one or more halo or —O—C₁₋₃ alkyl;
R⁴ is H, F, —C₁₋₃ alkyl or —O—C₁₋₃ alkyl;
R⁵is one or more H, —OH, —R^{8a}, —C(═O)—R^{8b}, —SO₂—R⁹, or —N(R^{11a})R^{11b};
R⁶ and R⁷ are, independently, or —C₁₋₃ alkyl;
R^{6a} and R^{6b} are, independently, H or C₁₋₆ alkyl or R^{6a} and R^{6b} are linked together to form a 3- to 6-membered ring;
R^{6c} and R^{6d} are, independently, hydrogen or —C₁₋₄ alkyl optionally substituted by one or more halo, —O—CH₃, phenyl, or —N(R^{6a})R^{6b};
R^{6e} is —C₁₋₃ alkyl;
R^{8a} is —CN, —C₁₋₄ alkyl (linear, branched or cyclic; which alkyl group is optionally substituted by one or more halo, or —OC₁₋₃ alkyl (optionally substituted by one or more halo or —O—CH₃);
R^{8b} is hydrogen or —C₁₋₃ alkyl (optionally substituted by one or more fluoro atoms);
R⁹ is —C₁₋₄ alkyl optionally substituted by one or more halo or —O—CH₃;
R^{10a} and R^{10b} are, independently, H, halo, C₁₋₄ alkyl (optionally substituted by one or more fluoro, —CN, —R^{12a}, —OR^{12b}, —N(R^{12c})R^{12d} or —C(O)N(R^{12e})R^{12f}) or —O—C₁₋₄ alkyl (optionally substituted by one or more fluoro, —R^{12g}, —OR^{12h} or —N(R¹²ⁱ)R^{12j});
R^{11a} and R^{11b} are, independently, hydrogen, C₁₋₃ alkyl (optionally substituted by one or more fluoro atoms) or —S(O)₂R^{6e};
R^{12a}, R^{12b}, R^{12c}, R^{12d}, R^{12e}, R^{12f}, R^{12g}, R^{12h}, R¹²ⁱ and R^{12j} are, independently, hydrogen or C₁₋₃ alkyl (optionally substituted by one or more fluoro atoms);
or a pharmaceutically-acceptable salt thereof.

2. A compound of formula (I): 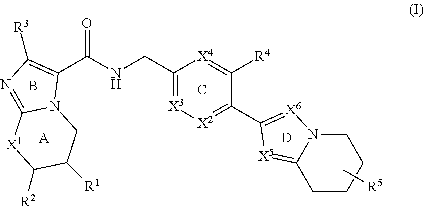
Wherein:
A is a 6-membered ring, which is aromatic or non-aromatic,
X¹ is ═N—, —CH₂— or ═C(R^{10a})—;
X², X³, X⁴, X⁵ and X⁶ are each, independently, ═N— or ═C(R^{10b})—;
R¹ and R² are each, independently, hydrogen, halo, —R^{6e}, —O—R^{6d}, —C(═O)—R^{6e}, —C(═O)—N(R⁶)(R⁷), —CN or —N(R^{6a})R^{6b};
R³ is H, halo or —C₁₋₃ alkyl (linear, branched or cyclic) optionally substituted by one or more halo or —O—C₁₋₃ alkyl;
R⁴ is H, F, —C₁₋₃ alkyl or —O—C₁₋₃ alkyl;
R⁵ is one or more H, —OH, —R^{8a}, —C(═O)—R^{8b}, —SO₂—R⁹, or —N(R^{11a})R^{11b};
R⁶ and R⁷ are, independently, H or —C₁₋₃ alkyl;
R^{6a} and R^{6b} are, independently, H or C₁₋₆ alkyl or R^{6a} and R^{6b} are linked together to form a 3- to 6-membered ring;
R^{6c} and R^{6d} are, independently, hydrogen or —C₁₋₄ alkyl optionally substituted by one or more halo, —O—CH₃, phenyl, or —N(R^{6a})R^{6b};
R^{6e} is —C₁₋₃ alkyl;
R^{8a} is —CN, —C₁₋₄ alkyl (linear, branched or cyclic; where the alkyl is optionally substituted by one or more halo or —OC₁₋₃ alkyl (optionally substituted by one or more halo or —O—CH₃);
R^{8b} is hydrogen or —C₁₋₃ alkyl (optionally substituted by one or more fluoro atoms);
R⁹ is —C₁₋₄ alkyl optionally substituted by one or more halo or —O—CH₃;
R^{10a} and R^{10b} are, independently, H, halo, C₁₋₄ alkyl (optionally substituted by one or more fluoro, —CN, —R^{12a}, —OR^{12b}, —N(R^{12c})R^{12d} or —C(O)N(R^{12e})R^{12f}) or —O—C₁₋₄ alkyl (optionally substituted by one or more —R^{12g}, —OR^{12h} or —N(R¹²ⁱ)R^{12j});
R^{11a} and R^{11b} are, independently, represent hydrogen, C₁₋₃ alkyl (optionally substituted by one or more fluoro atoms) or —S(O)₂R^{6c};
R^{12a}, R^{12b}, R^{12c}, R^{12d}, R^{12e}, R^{12f}, R^{12g}, R^{12h}, R¹²ⁱ and R^{12j} are, independently, represent hydrogen or C₁₋₃ alkyl (optionally substituted by one or more fluoro atoms);
or a pharmaceutically-acceptable salt thereof.

3. The compound according to claim 1, wherein:
A is aromatic;
X¹ is ═N—, or ═CH—; and
R¹ and R² are each, independently, hydrogen, —CH₃, —F, —Cl, —OCH₃, —NH₂, or —CH₂NH₂.

4. The compound according to claim 1, wherein:
R³ is H, —CF₃, —CHF₂, —CH₃, —CH₂CH₃, or cyclopropyl.

5. The compound according to claim 1, wherein C is a compound of formula (II), (III), (IV), or (V): 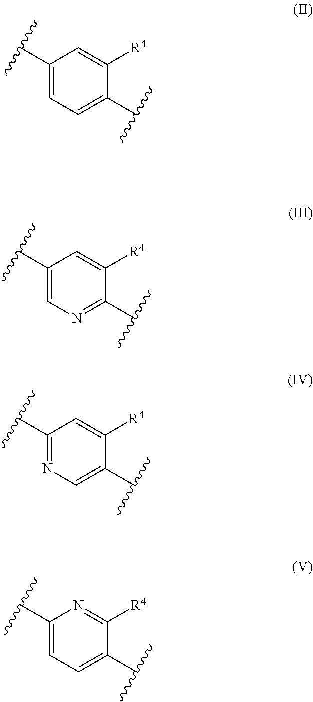
and wherein R⁴ is H, F, —C₁₋₃ alkyl or —O—C₁₋₃ alkyl.

6. The compound according to claim 1, wherein D is a compound of formula (VI), (VII), or (VIII): 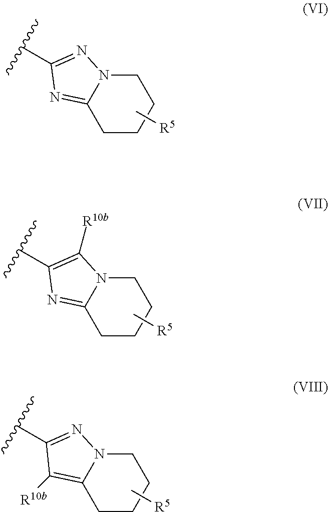
wherein:
R⁵ is one or more H, —OH, —R^{8a}, —C(═O)—R^{8b}, —SO₂—R⁹, or —N(R^{11a})R^{11b};
R^{10b} is H, halo, C₁₋₄ alkyl (optionally substituted by one or more fluoro, —CN, —R^{12a}, —OR^{12b}, —N(R^{12c})R^{12d} or —C(O)N(R^{12e})R^{12f}) or —O—C₁₋₄ alkyl (optionally substituted by one or more fluoro, —R^{12g}, —OR^{12h} or —N(R¹²ⁱ)R^{12j});

7. The compound according to claim 1, wherein R⁵ is H, —CH₃, —CH₂CH₃, —CH₂CH₂CH₃, cyclopropyl, —OH, —OCH₃, —OCF₃, —OCH₂CH₂OCH₃, —CF₃, —CHF₂, —CF₂CH₃, —NH₂, —NH(SO₂)CF₃, —N(CH₃)(SO₂)CF₃, or —SO₂CF₃.

8. The compound according to claim 1, wherein D is a compound of formula (VI′), (VIIa′), (VIIb′), (VIIIa′), or (VIIIb′): 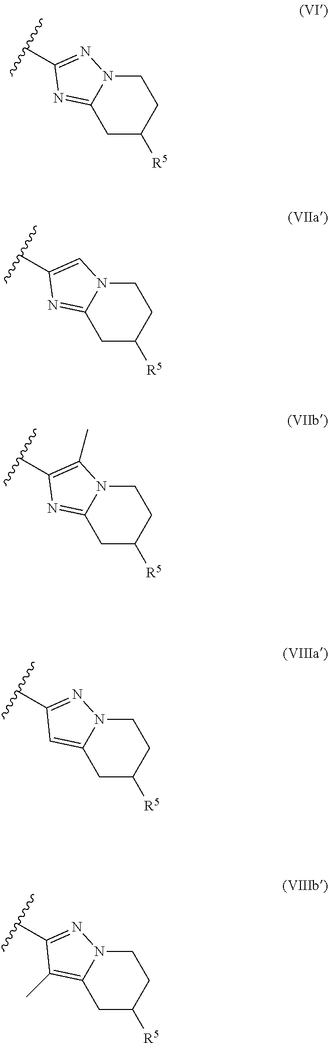
wherein R⁵is H, —CH₃, —CH₂CH₃, —CH₂CH₂CH₃, cyclopropyl, —OH, —OCH₃, —OCF₃, —OCH₂CH₂OCH₃, —CF₃, —CHF₂, —CF₂CH₃, —NH₂, —NH(SO₂)CF₃, —N(CH₃)(SO₂)CF₃, or —SO₂CF₃.

9. The compound according to claim 1, that is of formula (IX): 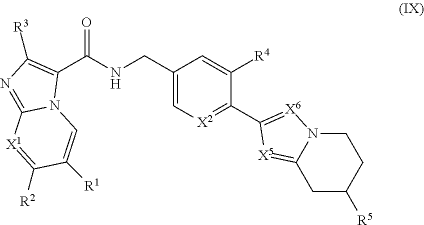
wherein:
X¹ is ═N— or ═CH—;
X², X⁵ and X⁶ are each, independently, ═N—, ═CH— or ═C(CH₃)—;
R¹ and R² are each, independently, hydrogen, —CH₃, —F, —Cl, —OCH₃, —NH₂, or —CH₂NH₂;
R³ is H, —CF₃, —CHF₂, —CH₃, —CH₂CH₃, or cyclopropyl;
R⁴ is H, F or —CH₃;
R⁵ is H, —CH₃, —CH₂CH₃, —CH₂CH₂CH₃, cyclopropyl, —OH, —OCH₃, —OCF₃, —OCH₂CH₂OCH₃, —CF₃, —CHF₂, —CF₂CH₃, —NH₂, —NH(SO₂)CF₃, —N(CH₃)(SO₂)CF₃, or —SO₂CF₃,
or a pharmaceutically-acceptable salt thereof.

10. A compound of formula (IB): 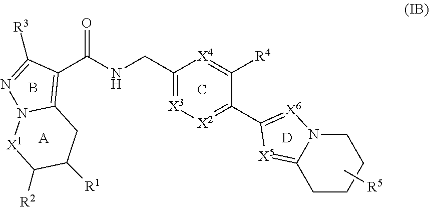
wherein:
A is a 6-membered ring, which is aromatic or non-aromatic;
B is a 5-membered aromatic ring;
X¹ is ═N—, —CH₂— or ═C(R^{10a})—;
X², X³, X⁴, X⁵ and X⁶ are each, independently, ═N— or ═C(R^{10b})—;
R¹ and R² are each, independently, hydrogen, halo, —R^{6c}, —O—R^{6d}, —C(═O)—R^{6e}, —C(═O)—N(R⁶)(R⁷), —CN or —N(R^{6a})R^{6b};
R³ is H, halo, or —C₁₋₃ alkyl (linear, branched or cyclic) optionally substituted by one or more halo or —O—C₁₋₃ alkyl;
R⁴ is H, F, —C₁₋₃ alkyl or —O—C₁₋₃ alkyl;
R⁵ is one or more H, —OH, —R^{8a}, —C(═O)—R^{8b}, —SO₂—R⁹, or —N(R^{11a})R^{11b};
R⁶ and R⁷ are, independently, H or —C₁₋₃ alkyl;
R^{6a} and R^{6b} are, independently, H or C₁₋₆ alkyl or R^{6a} and R^{6b} are linked together to form a 3- to 6-membered ring;
R^{6c} and R^{6d} are, independently, hydrogen or —C₁₋₄ alkyl optionally substituted by one or more halo, —O—CH₃, phenyl, or —N(R^{6a})R^{6b};
R^{6e} is —C₁₋₃ alkyl;
R^{8a} is —CN, —C₁₋₄ alkyl (linear, branched or cyclic; which alkyl group is optionally substituted by one or more halo), or —OC₁₋₃ alkyl (optionally substituted by one or more halo or —O—CH₃);
R^{8b} is hydrogen or —C₁₋₃ alkyl (optionally substituted by one or more fluoro atoms);
R⁹ is —C₁₋₄ alkyl optionally substituted by one or more halo or —O—CH₃;
R^{10a} and R^{10b} are, independently, H, halo, C₁₋₄ alkyl (optionally substituted by one or more fluoro, —CN, —R^{12a}, —OR^{12b}, —N(R^{12c})R^{12d} or —C(O)N(R^{12e})R^{12f}) or —O—C₁₋₄ alkyl (optionally substituted by one or more fluoro, —R^{12g}, —OR^{12h} or —N(R¹²ⁱ)R^{12j});
R^{11a} and R^{11b} are, independently, hydrogen, C₁₋₃ alkyl (optionally substituted by one or more fluoro atoms) or —S(O)₂R^{6e};
R^{12a}, R^{12b}, R^{12e}, R^{12d}, R^{12e}, R^{12f}, R^{12g}, R^{12h}, R¹²ⁱ and R^{12j} are, independently, hydrogen or C₁₋₃ alkyl (optionally substituted by one or more fluoro atoms).

11. The compound according to claim 10, wherein the bicycle containing rings A and B is a compound of formula (XX), (XXII), (XXIV), (XXI), or (XXIII):

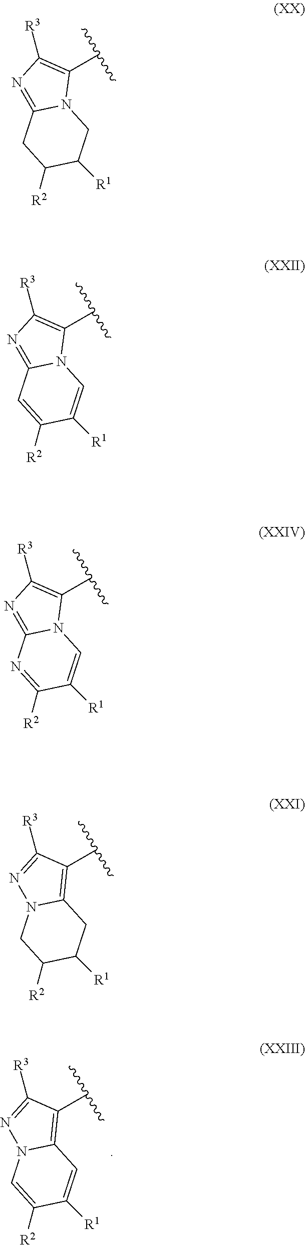

12. The compound according to claim 10, wherein R¹ and R² both are hydrogen.

13. The compound according to claim 10, wherein R³ is C₁₋₃ alkyl.

14. The compound according to claim 10, wherein the C is unsubstituted phenyl, i.e. of the formula (XXX):

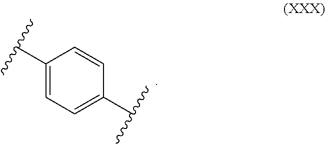 [SMILES: *c1ccc(*)cc1]

15. The compound according to claim 10, wherein the D is of the formula (XXXI) or the formula (XXXII):

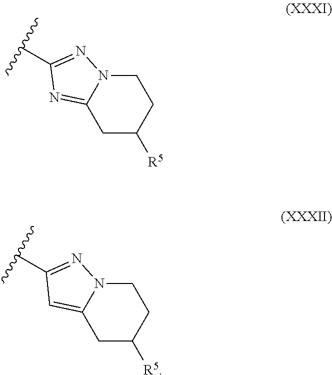

16. The compound according to claim 10, wherein R⁵ is C₁₋₃ alkyl optionally substituted by one or more fluoro atoms.

17. (canceled)

18. A pharmaceutical composition comprising a pharmaceutically acceptable carrier and, as active ingredient, a therapeutically effective amount of a compound of claim 1.

19-20. (canceled)

21. A method of treatment of a mycobacterial infection, comprising administration of a therapeutically effective amount of a compound of claim 1.

22. A combination of (a) a compound of claim 1, and (b) one or more anti-mycobacterial agent.

23. A product containing (a) the compound of claim 1, and (b) one or more anti-mycobacterial agent, as a combined preparation for simultaneous, separate or sequential use in the treatment of a bacterial infection.

24. A process for the preparation of the compound of formula (IA) of claim 1, comprising:
(i) reaction of a compound of formula (XL) or (X): 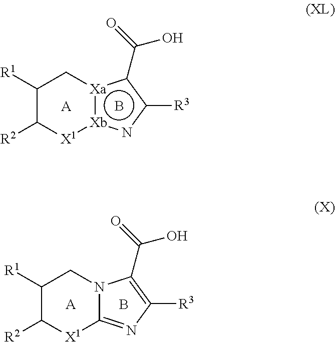
with a compound of formula (XI): 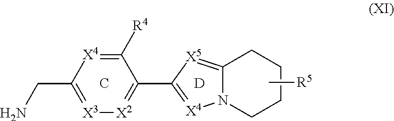
or
(ii) coupling of a compound of formula (XLI) or (XII), 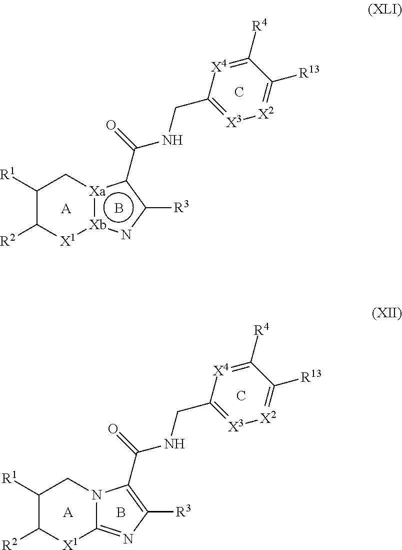
wherein R¹³ is suitable leaving group,
with a compound of formula (XIII): 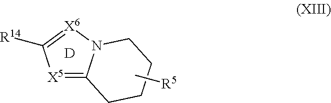
wherein R¹⁴ is a suitable leaving group.

25. The compound according to claim 1, wherein R³ is Cl or F.

26. The compound according to claim 1, wherein R³ is —C₁₋₃ alkyl substituted by F.

27. The compound according to claim 1, wherein R^{6c} and R^{6d} are, independently —C₁₋₄ alkyl substituted by one or more F.

28. The compound according to claim 1, wherein R^{8a} is —CF₃, —CHF₂, or —CF₂CH₃.

29. The compound according to claim 1, wherein R⁹ is —C₁₋₄ alkyl substituted by one or more F.

30. The compound according to claim 2, wherein R¹ and R² are, independently, Cl or F.

31. The compound according to claim 2, wherein R³ is Cl or F.

32. The compound according to claim 2, wherein R³ is —C₁₋₃ alkyl substituted by F.

33. The compound according to claim 2, wherein R^{6c} and R^{6d} are, independently —C₁₋₄ alkyl substituted by one or more F.

34. The compound according to claim 2, wherein R^{8a} is —CF₃, —CHF₂, or —CF₂CH₃.

35. The compound according to claim 2, wherein R⁹ is —C₁₋₄ alkyl substituted by one or more F.

36. The compound according to claim 16, wherein R⁵ is CF₃.

37. The method according to claim 21, wherein the mycobacterial infection is tuberculosis.

38. The combination according to claim 22, wherein the anti-mycobacterial agent is an anti-tuberculosis agent.

39. The product according to claim 23, wherein the anti-mycobacterial agent is an anti-tuberculosis agent.

40. The compound according to claim 13, wherein R³ is ethyl.
